(19) United States
(12) Patent Application Publication
(10) Pub. No.: US 2020/0298194 A1
(43) Pub. Date: Sep. 24, 2020

(54) ARAMID AMPHIPHILE SELF-ASSEMBLED NANOSTRUCTURES

(71) Applicant: MASSACHUSETTS INSTITUTE OF TECHNOLOGY, Cambridge, MA (US)

(72) Inventors: Julia Ortony, Cambridge, MA (US); Jia Tian, Cambridge, MA (US); Dae-Yoon Kim, Cambridge, MA (US); William Lindemann, Cambridge, MA (US); Ty Christoff-Tempesta, Cambridge, MA (US); Andrew Lew, Cambridge, MA (US); Yukio Cho, Cambridge, MA (US)

(73) Assignee: MASSACHUSETTS INSTITUTE OF TECHNOLOGY, Cambridge, MA (US)

(21) Appl. No.: 16/825,724

(22) Filed: Mar. 20, 2020

Related U.S. Application Data
(60) Provisional application No. 62/821,844, filed on Mar. 21, 2019.

(51) Int. Cl. B01F 17/00; B01J 13/08; C07C 271/28; C07C 309/61; C07C 233/81; D01F 8/12; D01F 1/10

(52) U.S. Cl. CPC B01F 17/0057; B01F 17/0042; B01J 13/08; C07C 271/28; D10B 2401/021; C07C 233/81; D01F 8/12; D01F 1/10; C07C 309/61

(57) ABSTRACT

Aramids and nanostructures formed from the aramids are described.

### CLAIM OF PRIORITY

This application claims priority to U.S. Provisional Patent Application No. 62/821,844, filed Mar. 21, 2019, which is incorporated by reference in its entirety.

### TECHNICAL FIELD

This invention relates to aramid compounds and nanostructures thereof.

### BACKGROUND

Nanostructures can be formed through self-assembly of molecules.

### SUMMARY

Aramids and nanostructures formed from the aramids are described. The aramid can be engineered to create specific nanostructures, for example, nanosheets, nanospheres, nanoribbons or nanofibers. The nanostructures can be hollow. The aramid can have amphiphilic properties. The aramid can have bolaamphiphilic properties. For example, the aramid can have a hydrophilic head group, a rigid core, and a hydrophobic tail group.

In one aspect, a compound has a formula I:

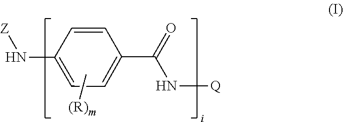

wherein
Z can be substituted arylacyl including a first substituent;
Q can be substituted aryl including a second substituent;
each R can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio;
m can be 0, 1, 2, 3 or 4;
i can be 1, 2, 3, 4, 5, 6, 7, 8, 9 or 10, wherein at least one of the first substituent and the second substituent can be a hydrophobic group.

In another aspect, an assembly can include a plurality of a compound described herein. In certain circumstances, the assembly can be a vesicle, a ribbon, a nanofiber or a micelle. In certain circumstances, the assembly can be a fiber. The fiber can have a tensile strength of at least 1 GPa.

In another aspect, a metal complex can include a metal ion and a compound described herein.

In another aspect, a method of forming an assembly can include dispersing a plurality of a compound described herein; and isolating an assembly of the plurality of the compound. In certain circumstances, the method can include encapsulating a payload in the assembly. In certain circumstances, the method can include shear aligning the assembly to form a fiber.

In certain circumstances, i can be 1, 2 or 3.

In certain circumstances, one of the first substituent and the second substituent can be a hydrophobic group and the other of the first substituent and the second substituent can be a hydrophilic group.

In certain circumstances, the substituted arylacyl can be a substituted phenyl acyl.

In certain circumstances, the substituted aryl can be a substituted phenyl.

In certain circumstances, the compound can be anion, cationic or zwitterionic.

In certain circumstances, the compound can include a metal binding moiety.

In certain circumstances, the compound can have the formula II:

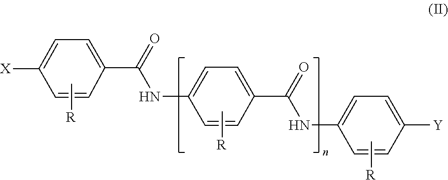

wherein

n can be 0, 1, 2, 3, 4, 5, 6, 7 or 8;

X can be a tail group; and

Y can be a head group.

In certain circumstances, X can be a substituted or unsubstituted, branched or linear alkyl group, alkenyl group, alkynyl group, fluorinated group, siloxane group, or aromatic groups.

In certain circumstances, n can be 1, 2 or 3.

In certain circumstances, X can be selected from the group consisting of:

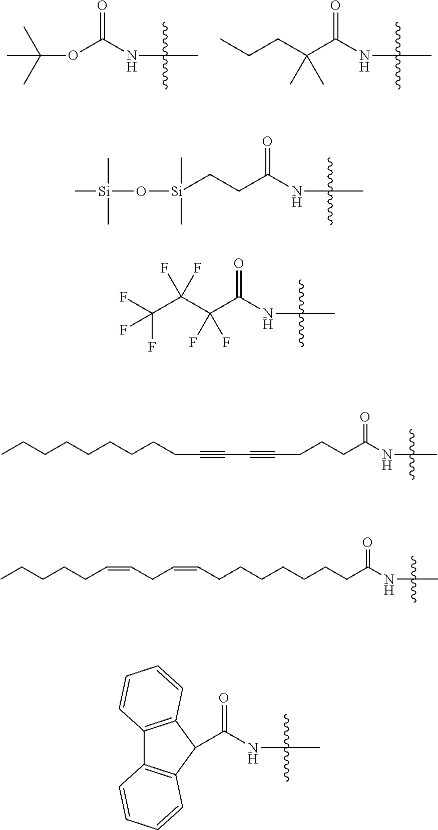

In certain circumstances, Y can be an anionic group, a cationic group, a zwitterionic group, or an uncharged hydrophilic group. Y can be a heavy metal chelator, an amino acid or a peptide.

In certain circumstances, Y can be selected from the group consisting of oligo-ethylene glycol,

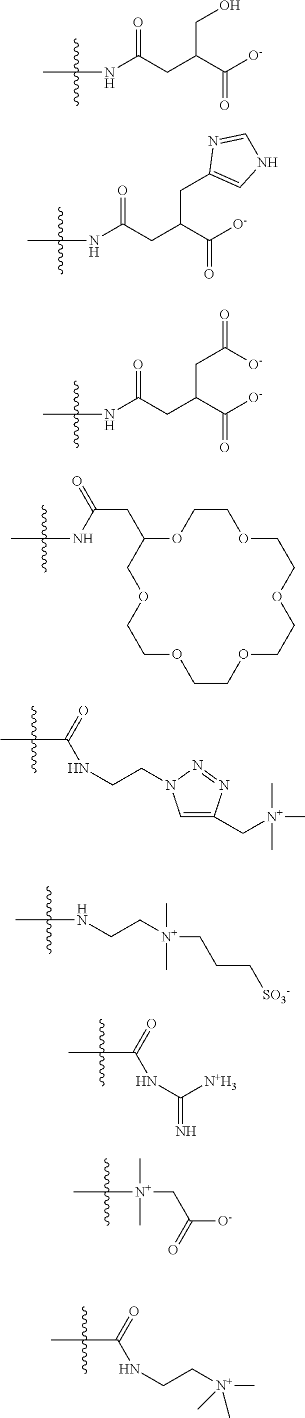

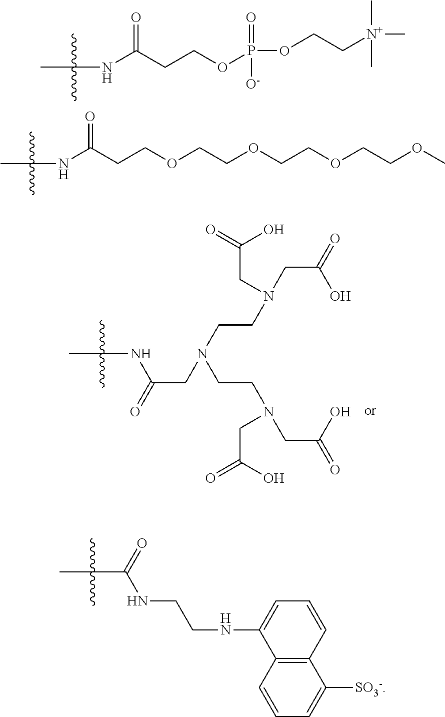

In certain circumstances, the compound can be

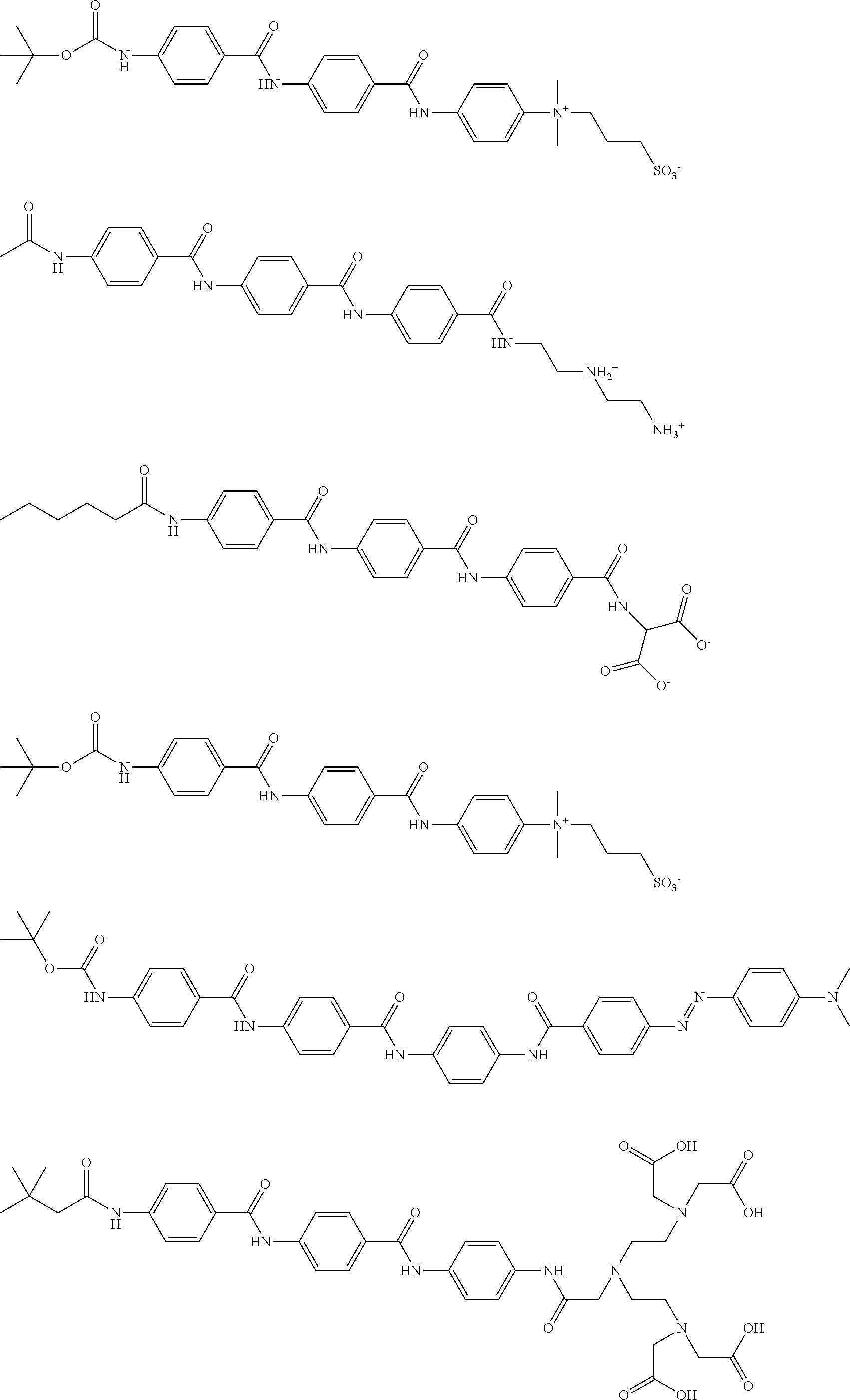

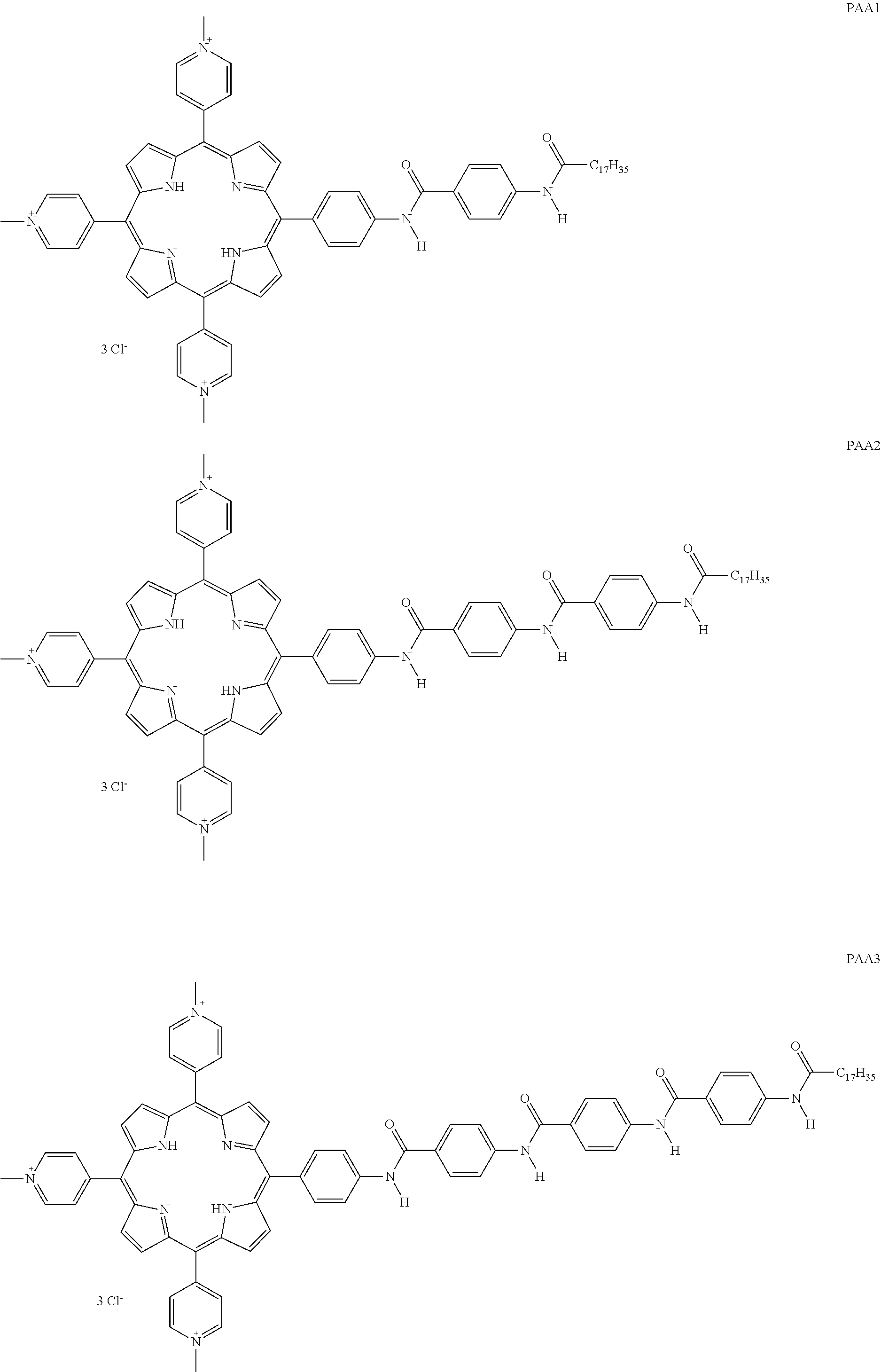

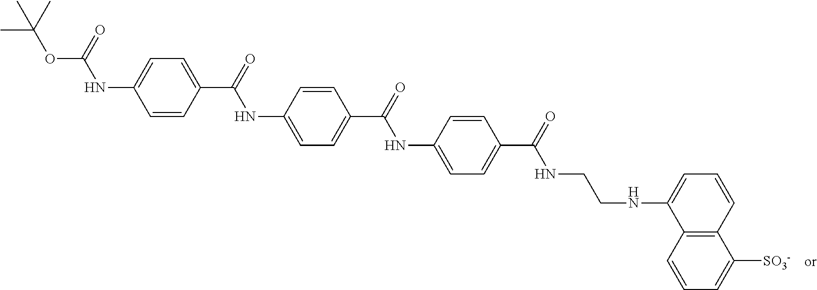

Other aspects, embodiments, and features will be apparent from the following description, the drawings, and the claims.

### BRIEF DESCRIPTION OF THE DRAWINGS

FIG. 1 shows aramid amphiphiles self-assemble into nanospheres, ribbon-like nanofibers, or 30-nm hollow spheres.

FIG. 2 shows chemical motif of aramid amphiphiles (a triaramid amphiphile is shown here) gives rise to nanostructures upon self-assembly. The choice of R group influences nanostructure geometry, and the R′ group is chosen to tune surface chemistry and charge.

FIG. 3 shows a comparison of structural stability of a reported phospholipid vesicle and high-stability oligoaramid vesicles by atomic force microscopy in air. FIG. 3 depicts a highly stable oligoaramid vesicles maintain their height after drying and imaging on mica by AFM.

FIG. 4 shows electron paramagnetic resonance spectroscopy used to characterize internal dynamics of a spherical aramid nanostructure. We found that the aramid domain exhibit very slow, 107 Hz rotational diffusion rates, consistent with solid-like motion.

FIG. 5 shows aramid amphiphile nanoparticles with 0, 1, 2, and 3 aramid groups incorporated into their structure, (a)-(d), respectively, are subject to UV light via a solar simulator for 0 h-120 h. The nanostructures with more aramid groups per molecule show enhanced stability by X-ray scattering, where the initial and final scattering profile are the same.

FIG. 6 shows ribbon-like nanofibers can be formed by co-assembly with a functionalized analogue of the aramid amphiphile. Here we have incorporated arsenic chelating groups (yellow) and/or lead chelating groups (green), for the purpose of purification of drinking water.

FIG. 7 shows ¹H NMR spectra of compound 1.

FIG. 8 shows ¹H NMR spectra of compound 2.

FIG. 9 shows ¹H NMR spectra of compound 3.

FIG. 10 shows ¹³C NMR spectra of compound 3.

FIG. 11 shows ¹H NMR spectra of compound 4.

FIG. 12 shows ¹H NMR spectra of compound 5.

FIG. 13 shows ¹H NMR spectra of compound 6.

FIG. 14 shows ¹³C NMR spectra of compound 6.

FIG. 15 shows ¹H NMR spectra of compound 7.

FIG. 16 shows ¹H NMR spectra of compound 8.

FIG. 17 shows ¹H NMR spectra of compound 9.

FIG. 18 shows ¹H NMR spectra of compound 10.

FIG. 19 shows ¹³C NMR spectra of compound 10.

FIG. 20 shows a TEM micrograph of the assembly of compound 3.

FIG. 21 shows aramid amphiphiles self-assemble into nanospheres, ribbon-like nanofibers, or hollow spheres.

FIG. 22 shows ¹H NMR spectra of compound 13.

FIG. 23 shows ¹H NMR spectra of compound 14.

FIG. 24 shows an SEM micrograph of compound 3 after assembly in water followed by the evaporation of water.

FIG. 25 shows ¹H NMR spectra of compound 15.

FIG. 26 shows ¹H NMR spectra of compound 16.

FIG. 27 shows ¹H NMR spectra of compound 17.

FIG. 28 shows ¹H NMR spectra of compound 18.

FIG. 29 shows ¹H NMR spectra of compound 19.

FIG. 30 shows ¹³C NMR spectra of compound 19.

FIG. 31 shows a TEM micrograph of the assembly of compound 19.

FIG. 32 shows the elution profile (left) and mass spectrogram (center/right) of the compound 21.

FIG. 33 shows negative staining TEM images of compound (a) PAA0 and (b) ZnPAA0 assemblies in water.

FIG. 34 shows negative staining TEM images of compound (a) PAA1 and (b) ZnPAA1 assemblies in water.

FIG. 35 shows negative staining TEM images of compound (a) PAA2 and (b) ZnPAA2 assemblies in water.

FIG. 36 shows negative staining TEM images of compound (a) PAA4 and (b) ZnPAA4 assemblies in water.

FIG. 37 shows Synchrotron SAXS profiles of (a) ZnPAA0, (b) ZnPAA1, (c) ZnPAA2, and (d) ZnPAA3 in water with [Zn]=5 mM at room temperature. (Black dots: experimental SAXS data; red line: corresponding fit by core-shell model by Irena 2.63 (https://usaxs.xray.aps.anl.gov/software/irena)).

FIG. 38 shows synchrotron SAXS profiles of (a) ZnPAA0, (b) ZnPAA1, (c) ZnPAA2, and (d) ZnPAA3 in water with [Zn]=5 mM, with different irradiation time.

FIG. 39 shows synchrotron SAXS profiles of (a) ZnPAA0, (b) ZnPAA1, (c) ZnPAA2, and (d) ZnPAA3 in water with [Zn]=5 mM, at variable temperatures from 303 to 313K.

FIG. 40 shows UV-vis spectra of 0.2 mM (a) PC and ZnPC, (b) PAA0 and ZnPAA0, (c) PAA1 and ZnPAA1, (d) PAA2 and ZnPAA2, (e) PAA3 and ZnPAA3, and (f) merged images of all zinc complexes in water, at room temperature.

FIG. 41 shows fluorescence spectra of 0.02 mM (a) PC and ZnPC, (b) PAA0 and ZnPAA0, (c) PAA1 and ZnPAA1, (d) PAA2 and ZnPAA2, (e) PAA3 and ZnPAA3, and (f) merged images of all zinc complexes in water, at room temperature.

FIG. 42 shows fluorescence spectra of (a) dilution of ZnPAA3 solution, (b) titration of C2 to a 0.02 mM ZnPAA3 solution, and (c) Stern-Volmer analysis of ZnPAA3 with different concentration of C2.

FIG. 43 shows UV-vis spectra of 0.02 mM (a) C2, (b) ZnPC, (c) ZnPAA0, (d) ZnPAA1, (e) ZnPAA2, and (f) ZnPAA3 in water, at room temperature.

FIG. 44 shows cyclic voltammetry of 0.2 mM (a) C2, (b) ZnPC, (c) ZnPAA0, (d) ZnPAA1, (e) ZnPAA2, and (f) ZnPAA3 with a scan rate of 100 mV/s. Working, reference, and counter electrodes are gold, Ag/AgCl, and Pt, respectively.

FIG. 45 shows UV-vis spectra of 0.02 mM (a) ZnPC, (b) ZnPAA0, (c) ZnPAA1, (d) ZnPAA2, (d) ZnPAA3, at different irradiation time, and (f) plots of A/A₀ versus irradiation time for all zinc complexes in water, where A₀ is the initiate absorbance at 425 nm.

FIG. 46 shows GC-MS experiments during long-term irradiation of a solution containing 2 μM of catalyst C2, 20 mM of TEA and 0.2 mM of sensitizer ZnPAA3, under ¹³CO₂ or ¹²CO₂ atmosphere (a) typical gas chromatogram observed and the mass spectra of methane generated under a (b)¹³CO₂ or (c)¹²CO₂ atmosphere.

FIG. 47 shows gas chromatogram profiles of 0.2 mM (a) ZnPAA0, (b) ZnPAA1, (c) ZnPAA2, and (d) ZnPAA3 in water with 2 μM of catalyst C2, 20 mM of TEA, under saturated CO₂ atmosphere in water after 24 h irradiation.

FIG. 48 shows the pictures of centrifuge tubes, which contained SPAs solution of ZnPAA3, [Zn]=0.5 mM, before and after treatment of high speed centrifugation and sonication.

FIG. 49 shows the cryo-TEM of 0.2 mM ZnPAA3 assemblies after 12^{th} catalytic cycles, comparison of (b) synchrotron SAXS profiles and (c) DLS results of 0.2 mM ZnPAA3 in water, with 2 μM of catalyst C2, 20 mM of TEA.HCl, under saturated CO₂ atmosphere in water before and after 24 h irradiation.

FIG. 50 shows an ¹H NMR spectrum (400 MHz) of compound 3 in DMSO-d₆ (2 mM).

FIG. 51 shows a ¹³C NMR spectrum (100 MHz) of compound 3 in DMSO-d₆ (6 mM).

FIG. 52 shows an ¹H NMR spectrum (400 MHz) of compound 4 in DMSO-d₆ (1 mM).

FIG. 53 shows a ¹³C NMR spectrum (100 MHz) of compound 4 in DMSO-d₆ (8 mM).

FIG. 54 shows an ¹H NMR spectrum (400 MHz) of compound 5 in DMSO-d₆ (5 mM).

FIG. 55 shows a ¹³C NMR spectrum (100 MHz) of compound 5 in DMSO-d₆ (10 mM).

FIG. 56 shows an ¹H NMR spectrum (400 MHz) of compound ZnPC in DMSO-d₆ (5 mM).

FIG. 57 shows a ¹³C NMR spectrum (100 MHz) of compound ZnPC in DMSO-d₆ (10 mM).

FIG. 58 shows an ¹H NMR spectrum (400 MHz) of compound PAA0 in DMSO-d₆ (2 mM).

FIG. 59 shows a ¹³C NMR spectrum (100 MHz) of compound PAA0 in DMSO-d₆ (10 mM).

FIG. 60 shows an ¹H NMR spectrum (400 MHz) of compound PAA1 in DMSO-d₆ (5 mM).

FIG. 61 shows a ¹³C NMR spectrum (100 MHz) of compound PAA1 in DMSO-d₆ (10 mM).

FIG. 62 shows an ¹H NMR spectrum (400 MHz) of compound PAA2 in DMSO-d₆ (5 mM).

FIG. 63 shows a ¹³C NMR spectrum (100 MHz) of compound PAA2 in DMSO-d₆ (10 mM).

FIG. 64 shows an ¹H NMR spectrum (400 MHz) of compound PAA3 in DMSO-d₆ (5 mM).

FIG. 65 shows a ¹³C NMR spectrum (100 MHz) of compound PAA3 in DMSO-d₆ (12 mM).

FIG. 66 shows an ESI-mass spectrometry of compound 3.

FIG. 67 shows an ESI-mass spectrometry of compound 4.

FIG. 68 shows an ESI-mass spectrometry of compound 5.

FIG. 69 shows an ESI-mass spectrometry of compound ZnPC.

FIG. 70 shows an ESI-mass spectrometry of compound PAA0.

FIG. 71 shows an ESI-mass spectrometry of compound ZnPAA0.

FIG. 72 shows an ESI-mass spectrometry of compound PAA1.

FIG. 73 shows an ESI-mass spectrometry of compound ZnPAA1.

FIG. 74 shows an ESI-mass spectrometry of compound PAA2.

FIG. 75 shows an ESI-mass spectrometry of compound ZnPAA2.

FIG. 76 shows an ESI-mass spectrometry of compound PAA3.

FIG. 77 shows an ESI-mass spectrometry of compound ZnPAA3.

FIG. 78 shows a schematic illustration of supramolecular photocatalytic assemblies. (a) The formation of hydrogen-bond enhanced micelles by self-assembling zinc porphyrin amphiphiles in water. (b) The photocatalytic hydrogen production and carbon dioxide reduction process over cobalt catalyst.

FIG. 79 shows structural information of compounds. The structures of amphiphile compounds ZnPAA0-3, control molecule ZnPC and catalysts C1 and C2.

FIG. 80 shows Morphology study of SPAs. Cryo-TEM image of (a) ZnPAA0, (b) ZnPAA0, (c) ZnPAA1, and (d) ZnPAA3 assembly in water with [Zn]=0.2 mM. Scale bar, 200 nm.

FIG. 81 shows size and surface charge characterization by SAX and DLS. (a) Synchrotron SAXS profiles of ZnPAA0-3 in water with [Zn]=5 mM at room temperature. (b) Hydrodynamic diameters of ZnPAA0-3 micelles ([Zn]=0.2 mM) acquired by DLS in water. (c) Concentration-dependent DLS experiments of ZnPAA0-3 in water. (d) Zeta-potential of ZnPAA0-3 ([Zn]=0.2 mM) in water.

FIG. 82 shows photocatalytic H₂ production and CO₂ reduction under visible light irradiation. Shown is the formation of gaseous products (in terms of turnover numbers) as a function of irradiation time, using an aqueous solution with 1 atm Air (a) and with saturated 1 atm CO₂ (b, c) or 1 atm CO (d) and containing 2 μM of catalyst C2 and 0.2 mM ZnPAA3 with sacrificial reagent (a) ascorbic acid (AA), (b) sodium ascorbate (SA), (c) triethylamine (TEA), and (d) triethylamine hydrochloride (TEA.HCl). All the data points contain at least three individual experiments and typical uncertainty on turnover numbers were shown as error bar.

FIG. 83 shows sketch of the proposed mechanism for CO₂ reduction to CH₄ by anionic catalyst C2. Initially, the starting [Co^{III}(dmgH)₂(py)Cl] is reduced with two electrons to the catalytically active Co^{I} species. The Co^{I} species reduces CO₂, with the resultant Co^{II} regenerated through electron transfer from the excited photosensitizer. The CO produced binds to Co^{II} and is further reduced with a total of six electrons and six protons to generate methane, via a postulated Co^{I}-formaldehyde intermediate. hv represents light irradiation.

FIG. 84 shows a schematic of compositions described herein.

FIG. 85A depicts Kevlar-inspired aramid amphiphiles self-assemble into ultra-stable planar nanofibers reinforced by collective hydrogen bonding. Aramid amphiphiles, composed of a charged head group, an aramid structural domain, and an aliphatic tail, are designed to spontaneously self-assemble in water with suppressed exchange dynamics.

FIG. 85B depicts aramid amphiphiles 1, 2, and 3 have anionic, zwitterionic, and cationic head groups, respectively.

FIG. 85C depicts small angle X-ray scattering of 1, 2, and 3 nanofibers in water shows slope of −2 in the Guinier regime, indicating high-aspect-ratio structures, and is best fit to a lamellar model (black line) giving a 3.9 nm nanofiber thickness.

FIG. 85D depicts representative transmission electron micrograph (TEM) of dried nanofibers of 2.

FIG. 85E depicts representative cryogenic TEM of nanofibers of 2 in water indicates planar nanofiber widths of approx. 5 nm.

FIG. 86A depicts aramid amphiphile nanofibers exhibit minimal molecular exchange, a Young's modulus of E=1.7 GPa, and a tensile strength of σ*=1.9 GPa. Molecular exchange between nanofibers is measured by FRET dark quenching upon mixing with donor-labeled and quencher-labeled nanofiber suspensions.

FIG. 86B depicts fluorescence of the donor and quencher nanofiber mixture remains nearly constant over 55 days, indicating minimal molecular exchange. As a control, complete co-assembly of donor and quencher amphiphiles reaches a 76% decrease in fluorescence intensity.

FIG. 86C depicts FRET is not observed when donor and quencher nanofibers are mixed at 80° C.

FIG. 86D depicts a representative subset of AFM images of AA nanofibers in water is traced to produce contours for statistical topographical analysis.

FIG. 86E depicts nanofiber secant lengths from contour traces (inset) are used to calculate persistence length, P=3.9±0.7 μm and Young's modulus, E=1.7±0.7 GPa.

FIG. 86F depicts sonication-induced scission of nanofibers is illustrated by TEM.

FIG. 86G depicts the threshold length below which a fibril will not break under sonication is determined from plotting sonicated fragment lengths against cross-sectional size, C. AA nanofiber tensile strength is calculated to be σ*=1.87±1.00 GPa.

FIG. 86H depicts AA nanofiber mechanical properties plotted on an Ashby chart place it among the strongest and stiffest biological materials.

FIG. 87A depicts that aramid amphiphile nanofibers are aligned by shear forces and dried to form flexible threads. A nanofiber suspension is pulled out of a pipet tip by tweezers into sodium sulfate solution to form a 1-dimensional gel. The gel is removed from water and dried to form a thread composed of aligned nanofibers.

FIG. 87B depicts SEM of a 20 μm-diameter nanofiber thread shows long-range alignment of nanofiber bundles.

FIG. 87C depicts nanofiber threads can be bent and handled easily.

FIG. 87D depicts a 5 cm nanofiber thread whose mass totals 0.1 mg is suspended over a trough and supports a 20 mg weight.

FIG. 88A depicts that X-ray scattering of solid-state nanofiber threads demonstrates organized molecular packing with extended hydrogen bonding networks and long-range hierarchical order within nanofiber threads. Meridional and equatorial scattering directions are depicted in X-ray scattering measurements of solid, aligned AA nanofiber threads.

FIG. 88B depicts a WAXS pattern of AA nanofiber thread shows that precise molecular organization is maintained in the solid state, with significant anisotropy indicating nanofiber alignment.

FIG. 88C depicts al-D scattering profile obtained by integrating meridional and equatorial axes of (B). Black dotted lines are simulated peak positions of a unit cell with a=7.22 Å, b=5.05 Å, and c=11.10 Å, and space group 26:Pmc2₁ based on poly(p-benzamide).

FIG. 88D depicts molecular packing in AA nanofibers is illustrated as informed by the simulated unit cell in FIG. 88C. 2.08 Å H-bonds form a hydrogen bonding network down the long-axis of the nanofiber with 3.61 Å π-π stacking orthogonal to the hydrogen bonding plane. Based on these distances, the surface area of AA nanofibers within the thread is 200 m²/g.

FIG. 88E depicts SAXS showing lamellar peaks corresponding to 4.8 nm interfiber spacing.

FIG. 88F depicts individual AA nanofibers align to form semi-crystalline domains with 4.8 nm lamellar spacings informed by FIG. 88E.

FIG. 89A depicts sonicated and annealed solution of 3 is extruded into a sodium sulfate bath to produce a macroscopic thread, which is then (FIG. 89B) removed from solution and dried.

FIG. 90 depicts 1D-WAXS scattering profile of meridional axis with tilting on the X-ray beam direction.

FIG. 91 depicts ¹H NMR spectra of compound 1 in DMSO-d with increasing D₂O content.

FIGS. 92A-92C depict TEM images of assemblies of compounds (FIG. 92A) 1, (FIG. 92A) 2, and (FIG. 92A) 3.

FIGS. 93A-93C depict cryo-TEM images of assemblies of compounds (FIG. 93A) 1, (FIG. 93B) 2, and (FIG. 93C) 3.

FIG. 94 depicts a representative AFM profile of compound 3 nanofibers deposited on mica illustrates nanofiber lengths up to 20 μm as determined by ImageJ analysis.

FIGS. 95A-95C depict SEM images of compounds (FIG. 95A) 1, (FIG. 95B) 2, and (FIG. 95C) 3 at 5000× and (FIG. 95D) 3 at 25000× magnifications.

FIG. 96 depicts the fluorescence intensity of nanofibers labeled with the FRET donor EDANS is quenched by 76% when co-assembled with FRET quencher DABCYL.

FIGS. 97A-97B depict the distribution of fragment lengths after sonication-induced scission of nanofibers of 3 as measured by (FIG. 97A) TEM and (FIG. 97B) AFM.

FIGS. 98A-98B depict yield strength analysis of nanofibers of 3 from sonication-induced scission identifies strengths of (FIG. 98A) 1.87±1.00 GPa based on TEM and (FIG. 98B) 1.96±0.99 GPa based on AFM analysis of the nanofiber fragments, showing a close convergence between the two techniques.

### DETAILED DESCRIPTION

In general, the compound can be an aramid. A plurality of the aramid can form an assembly.

The moieties described below can be substituted or unsubstituted. “Substituted” refers to replacement of a hydrogen atom of a molecule or an R-group with one or more additional R-groups such as halogen, alkyl, haloalkyl, alkenyl, alkoxy, alkoxyalkyl, alkylthio, trifluoromethyl, acyloxy, hydroxy, hydroxyalkyl, mercapto, carboxy, cyano, acyl, aryloxy, aryl, arylalkyl, heteroaryl, amino, aminoalkyl, alkylamino, dialkylamino, morpholino, piperidino, pyrrolidin-1-yl, piperazin-1-yl, nitro, phosphine, phosphinate, phosphonate, sulfato, ═O, ═S, or other R-groups. Unless otherwise indicated, an optionally substituted group may have a substituent at each substitutable position of a group. Combinations of substituents contemplated herein are preferably those that result in the formation of stable (e.g., not substantially altered for a week or longer when kept at a temperature of 40° C. or lower in the absence of moisture or other chemically reactive conditions), or chemically feasible, compounds.

“Hydroxy”, “thiol”, “cyano”, “nitro”, and “formyl” refer, respectively, to —OH, —SH, —CN, —NO₂, and —CHO.

“Acyl” refers to a RC(═O)— radical, wherein R is alkyl, cycloalkyl, aryl, heteroalkyl, heteroaryl, or heterocycloalkyl, which are as described herein. In some embodiments, it is a C₁-C₁₂ acyl radical, which refers to the total number of chain or ring atoms of the alkyl, cycloalkyl, aryl, heteroalkyl, heteroaryl, or heterocycloalkyl portion of the acyloxy group plus the carbonyl carbon of acyl, i.e., the other ring or chain atoms plus carbonyl. If the R radical is heteroaryl or heterocycloalkyl, the hetero ring or chain atoms contribute to the total number of chain or ring atoms. An “arylacyl” group is an aryl substituted acyl group.

“Acyloxy” refers to a RC(═O)O— radical, wherein R is alkyl, cycloalkyl, aryl, heteroalkyl, heteroaryl, or heterocycloalkyl, which are as described herein. In some embodiments, it is a C₁-C₄ acyloxy radical, which refers to the total number of chain or ring atoms of the alkyl, cycloalkyl, aryl, heteroalkyl, heteroaryl, or heterocycloalkyl portion of the acyloxy group plus the carbonyl carbon of acyl, i.e., the other ring or chain atoms plus carbonyl. If the R radical is heteroaryl or heterocycloalkyl, the hetero ring or chain atoms contribute to the total number of chain or ring atoms.

“Alkyl” refers to a group of 1-18, 1-16, 1-12, 1-10, preferably 1-8, more preferably 1-6 unsubstituted or substituted hydrogen-saturated carbons connected in linear, branched, or cyclic fashion, including the combination in linear, branched, and cyclic connectivity. Non-limiting examples include methyl, ethyl, propyl, isopropyl, butyl, and pentyl.

“Cycloalkyl” refers to a monocyclic or polycyclic non-aromatic radical that contains carbon and hydrogen, and may be saturated, or partially unsaturated. Cycloalkyl groups include groups having from 3 to 10 ring atoms (e.g., C₃-C₁₀ cycloalkyl). Whenever it appears herein, a numerical range such as “3 to 10” refers to each integer in the given range; e.g., “3 to 10 carbon atoms” means that the cycloalkyl group may consist of 3 carbon ring atoms, 4 carbon ring atoms, 5 carbon ring atoms, etc., up to and including 10 carbon ring atoms. In some embodiments, it is a C₃-C₈ cycloalkyl radical. In some embodiments, it is a C₃-C₅ cycloalkyl radical. Examples of cycloalkyl group include, but are not limited to, cyclopropyl, cyclobutyl, cyclopentyl, cyclopentenyl, cyclohexyl, cyclohexenyl, cycloseptyl, cyclooctyl, cyclononyl, cyclodecyl, and norbornyl. The term “cycloalkyl” also refers to spiral ring system, in which the cycloalkyl rings share one carbon atom.

“Heterocycloalkyl” refers to a 3- to 18-membered nonaromatic ring (e.g., C₃-C₁₈ heterocycloalkyl) radical that comprises two to twelve ring carbon atoms and from one to six heteroatoms selected from nitrogen, oxygen and sulfur. Whenever it appears herein, a numerical range such as “3 to 18” refers to each integer in the given range; e.g., “3 to 18 ring atoms” means that the heterocycloalkyl group may consist of 3 ring atoms, 4 ring atoms, etc., up to and including 18 ring atoms. In some embodiments, it is a C₅-C₁₀ heterocycloalkyl. In some embodiments, it is a C₄-C₁₀ heterocycloalkyl. In some embodiments, it is a C₃-C₁₀ heterocycloalkyl. The heterocycloalkyl radical may be a monocyclic, bicyclic, tricyclic or tetracyclic ring system, which may include fused or bridged ring systems. The heteroatoms in the heterocycloalkyl radical may be optionally oxidized. One or more nitrogen atoms, if present, may optionally be quaternized. The heterocycloalkyl radical may be partially or fully saturated. The heterocycloalkyl may be attached to the rest of the molecule through any atom of the ring(s). Examples of such heterocycloalkyl radicals include, but are not limited to, 6,7-dihydro-5H-cyclopenta[b]pyridine, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. In some embodiments, the heterocycloalkyl group is aziridinyl, azetidinyl, pyrrolidinyl, piperidinyl, homopiperidinyl, morpholinyl, thiomorpholinyl, piperazinyl, tetrahydrofuranyl, tetrahydrothienyl, dihydrooxazolyl, tetrahydropyranyl, tetrahydrothiopyranyl, indolinyl, tetrahydroquinolyl, tetrahydroisoquinolinyl and benzoxazinyl, preferably dihydrooxazolyl and tetrahydrofuranyl.

“Halo” refers to any of halogen atoms fluorine (F), chlorine (Cl), bromine (Br), or iodine (I). Examples of such halo groups can be fluorine.

“Haloalkyl” refers to an alkyl substituted by one or more halo(s).

“Alkenyl” refers to a group of unsubstituted or substituted hydrocarbons containing 2-18, 2-16, 2-12, 2-10, preferably 2-8, more preferably 2-6 carbons, which are linear, branched, cyclic, or in combination thereof, with at least one carbon-to-carbon double bond.

“Haloalkenyl” refers to an alkenyl substituted by one or more halo(s).

“Alkynyl” refers to a group of unsubstituted or substituted hydrocarbons containing 2-18, 2-16, 2-12, 2-10, preferably 2-8, more preferably 2-6 carbons, which are linear, branched, cyclic, or in combination thereof, with at least one carbon-to-carbon triple bond.

“Haloalkynyl” refers to an alkynyl substituted by one or more halo(s).

“Amino” refers to amino and substituted amino groups, for example, primary amines, secondary amines, tertiary amines and quaternary amines. Specifically, “amino” refers to —NRₐR_{b}, wherein Rₐ and R_{b}, both directly connected to the N, can be independently selected from hydrogen, deuterium, halo, hydroxy, cyano, formyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, a nitrogen protective group, —(CO)-alkyl, —(CO)—O-alkyl, or —S(O)ₙR_{c} (n=0 to 2, R_{c} is directly connected to S), wherein R_{c} is independently selected from hydrogen, deuterium, halo, amino, hydroxy, thiol, cyano, formyl, alkyl, haloalkyl, alkenyl, haloalkenyl, alkynyl, haloalkynyl, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

An “ammonium” can be a quaternary amine, for example, a cation of primary amine, secondary amine, tertiary amines or quaternary amine. For example, an ammonium can be a cation of an alkyl amine, such as an alkoxyalkyl amine, e.g., tris(hydroxymethyl)aminomethane or meglumine (methylglucamine).

“Aryl” refers to a C₆-C₁₄ aromatic hydrocarbon. For example, aryl can be phenyl, napthyl, or fluorenyl.

“Heteroaryl” refers to a C₆-C₁₄ aromatic hydrocarbon having one or more heteroatoms, such as N, O or S. The heteroaryl can be substituted or unsubstituted. Examples of a heteroaryl include, but are not limited to, azaindole, azepinyl, acridinyl, benzimidazolyl, benzindolyl, 1,3-benzodioxolyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzofurazanyl, benzothiazolyl, benzothienyl, benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). In some embodiments, the heteroaryl can be dithiazinyl, furyl, imidazolyl, azaindolyl, indolyl, isoquinolinyl, isoxazolyl, oxadiazolyl (e.g., (1,3,4)-oxadiazolyl, (1,2,3)-oxadiazolyl, or (1,2,4)-oxadiazolyl), oxazolyl, pyrazinyl, pyrazolyl, pyrazyl, pyridazinyl, pyridinyl, pyrimidinyl, pyrimidyl, pyrrolyl, quinolinyl, tetrazolyl, thiazolyl, thienyl, triazinyl, (1,2,3)-triazolyl, or (1,2,4)-triazolyl. The substituent on the heteroaryl group can be amino, alkylamino, or methyleneamino.

“Carbocycle” refers to a C₆-C₁₄ cyclic hydrocarbon. For example, aryl can be phenyl, napthyl, or fluorenyl.

“Heterocycle” refers to a C₆-C₁₄ cyclic hydrocarbon having one or more heteroatoms, such as N, O or S.

“Alkoxy” refers to an alkyl connected to an oxygen atom (—O— alkyl).

“Haloalkoxy” refers to a haloalkyl connected to an oxygen atom (—O— haloalkyl).

“Thioalkoxy” refers to an alkyl connected to a sulfur atom (—S— alkyl).

“Halothioalkoxy” refers to a haloalkyl connected to a sulfur atom (—S— haloalkyl).

“Carbonyl” refers to —(CO)—, wherein (CO) indicates that the oxygen is connected to the carbon with a double bond.

“Alkanoyl (or acyl)” refers to an alkyl connected to a carbonyl group [—(CO)— alkyl].

“Haloalkanoyl” or “haloacyl” refers to a haloalkyl connected to a carbonyl group [—(CO)— haloalkyl].

“Thiocarbonyl” refers to —(CS)—, wherein (CS) indicates that the sulfur is connected to the carbon with a double bond.

“Thioalkanoyl (or thioacyl)” refers to an alkyl connected to a thiocarbonyl group [—(CS)— alkyl].

“Halothioalkanoyl” or “halothioacyl” refers to a haloalkyl connected to a thiocarbonyl group [—(CS)— haloalkyl].

“Carbonyloxy” refers to an alkanoyl (or acyl) connected to an oxygen atom [—O—(CO)— alkyl].

“Halocarbonyloxy” refers to a haloalkanoyl (or haloacyl) connected to an oxygen atom [—O—(CO)— haloalkyl].

“Carbonylthio” refers to an alkanoyl (or acyl) connected to a sulfur atom [—S—(CO)— alkyl].

“Halocarbonylthio” refers to a haloalkanoyl (or haloacyl) connected to a sulfur atom [—S—(CO)— haloalkyl].

“Thiocarbonyloxy” refers to a thioalkanoyl (or thioacyl) connected to an oxygen atom [—O—(CS)— alkyl].

“Halothiocarbonyloxy” refers to a halothioalkanoyl (or halothioacyl) connected to an oxygen atom [—O—(CS)— haloalkyl].

“Thiocarbonylthio” refers to a thioalkanoyl (or thioacyl) connected to a sulfur atom [—S—(CS)— alkyl].

“Halothiocarbonylthio” refers to a halothioalkanoyl (or halothioacyl) connected to a sulfur atom [—S—(CS)— haloalkyl].

In one aspect, a compound has a formula I:

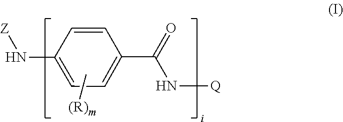

wherein
Z can be substituted arylacyl including a first substituent;
Q can be substituted aryl including a second substituent;
each R can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio;
m can be 0, 1, 2, 3 or 4;
i can be 1, 2, 3, 4, 5, 6, 7, 8, 9 or 10, wherein at least one of the first substituent and the second substituent can be a hydrophobic group.

In another aspect, an assembly can include a plurality of a compound described herein. In certain circumstances, the assembly can be a vesicle, a ribbon, or a micelle.

In another aspect, a metal complex can include a metal ion and a compound described herein.

In another aspect, a method of forming an assembly can include dispersing a plurality of a compound described herein; and isolating an assembly of the plurality of the compound. In certain circumstances, the method can include encapsulating a payload in the assembly. The assembly can be of amphiphilic compounds.

In certain circumstances, one of the first substituent and the second substituent can be a hydrophobic group and the other of the first substituent and the second substituent can be a hydrophilic group.

In certain circumstances, the substituted arylacyl can be a substituted phenyl acyl.

In certain circumstances, the substituted aryl can be a substituted phenyl.

In certain circumstances, the compound can be anion, cationic or zwitterionic.

In certain circumstances, the compound can include a metal binding moiety.

In certain circumstances, the compound can have the formula II:

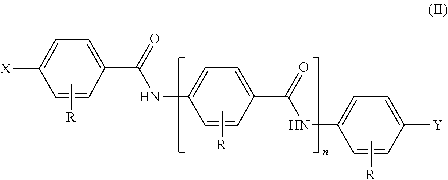

wherein

n can be 0, 1, 2, 3, 4, 5, 6, 7 or 8;

X can be a tail group; and

Y can be a head group.

In certain circumstances, X can be a substituted or unsubstituted, branched or linear alkyl group, alkenyl group, alkynyl group, fluorinated group, siloxane group, or aromatic groups.

In certain circumstances, X can be selected from the group consisting of:

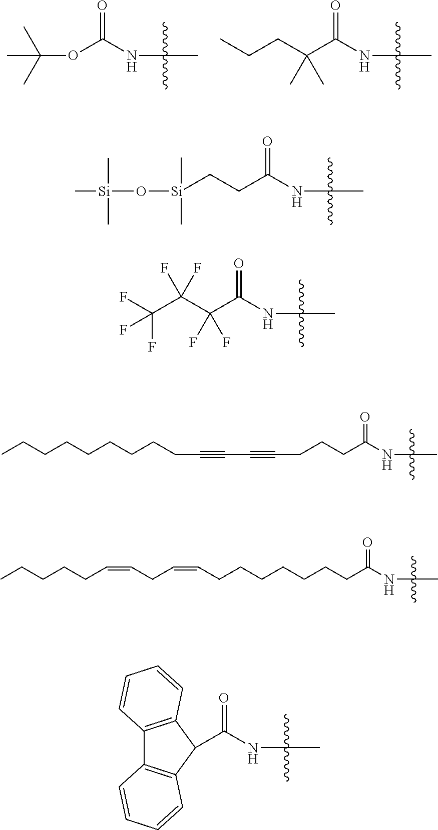

In certain circumstances, Y can be an anionic group, a cationic group, a zwitterionic group, or an uncharged hydrophilic group.

In certain circumstances, Y can be selected from the group consisting of oligo-ethylene glycol,

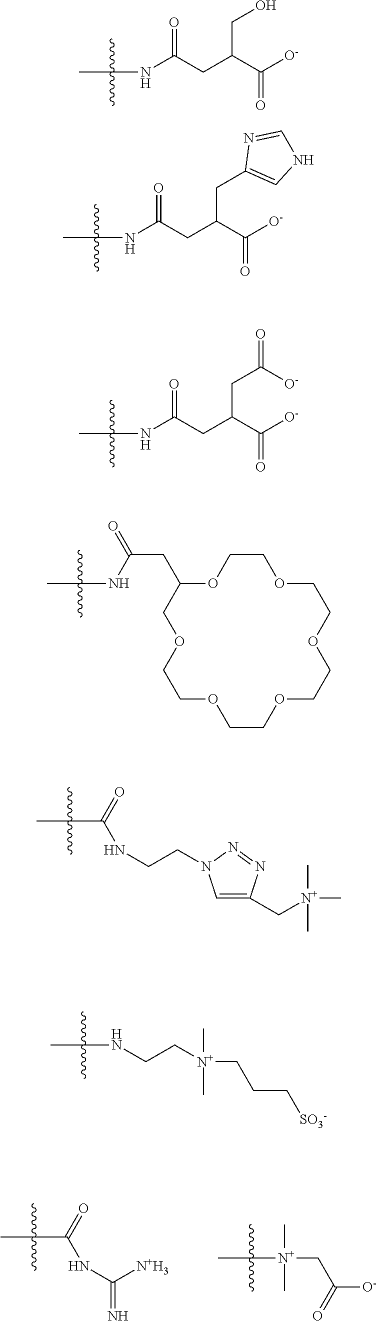

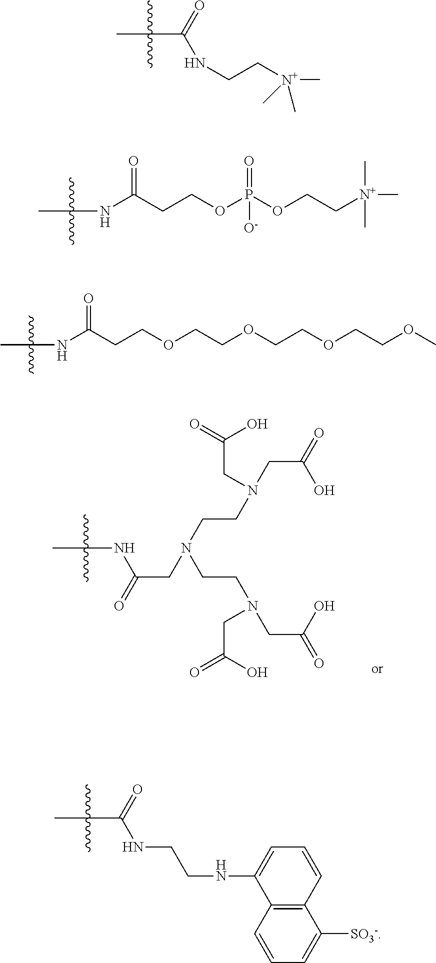

In certain circumstances, the compound can be

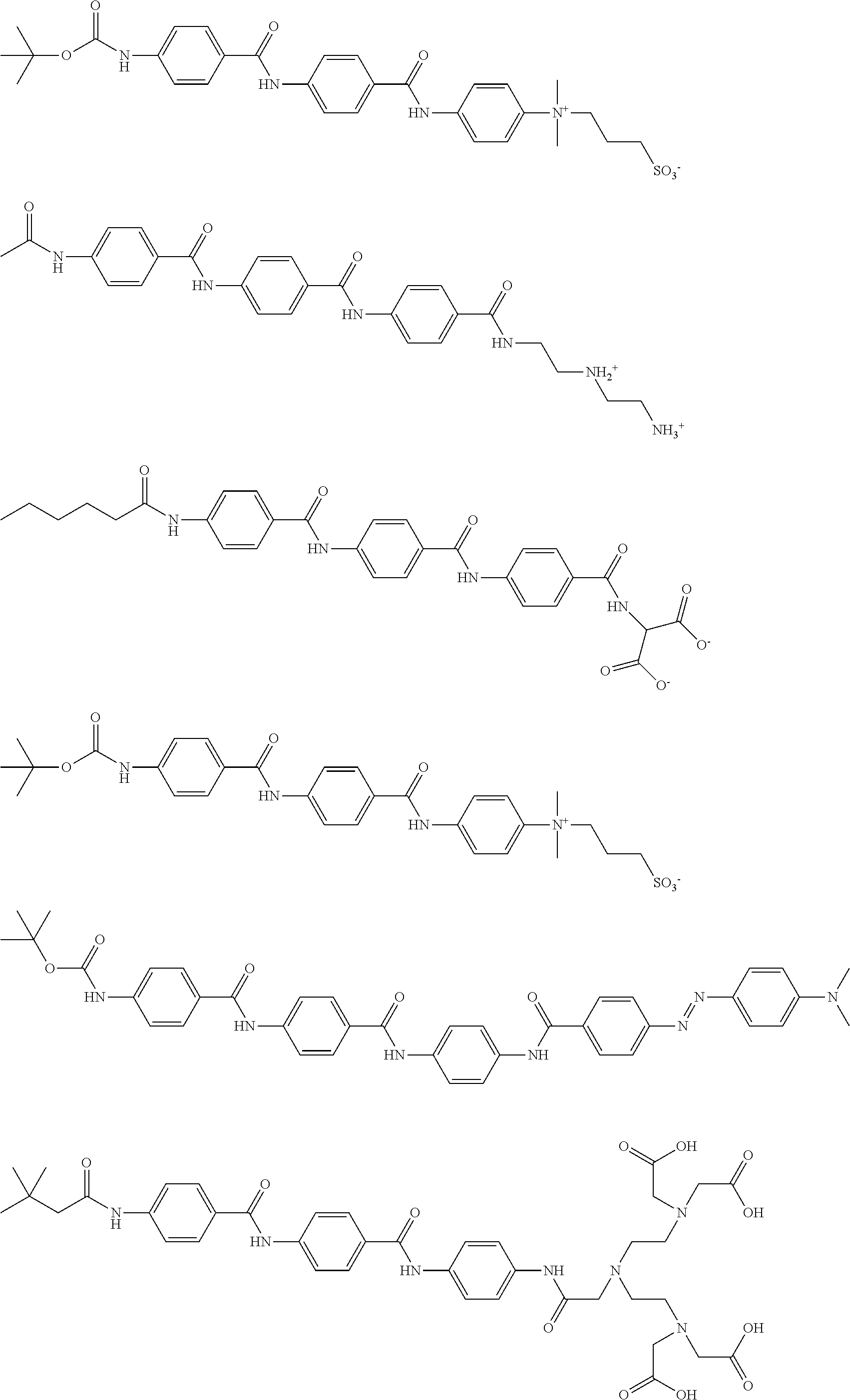

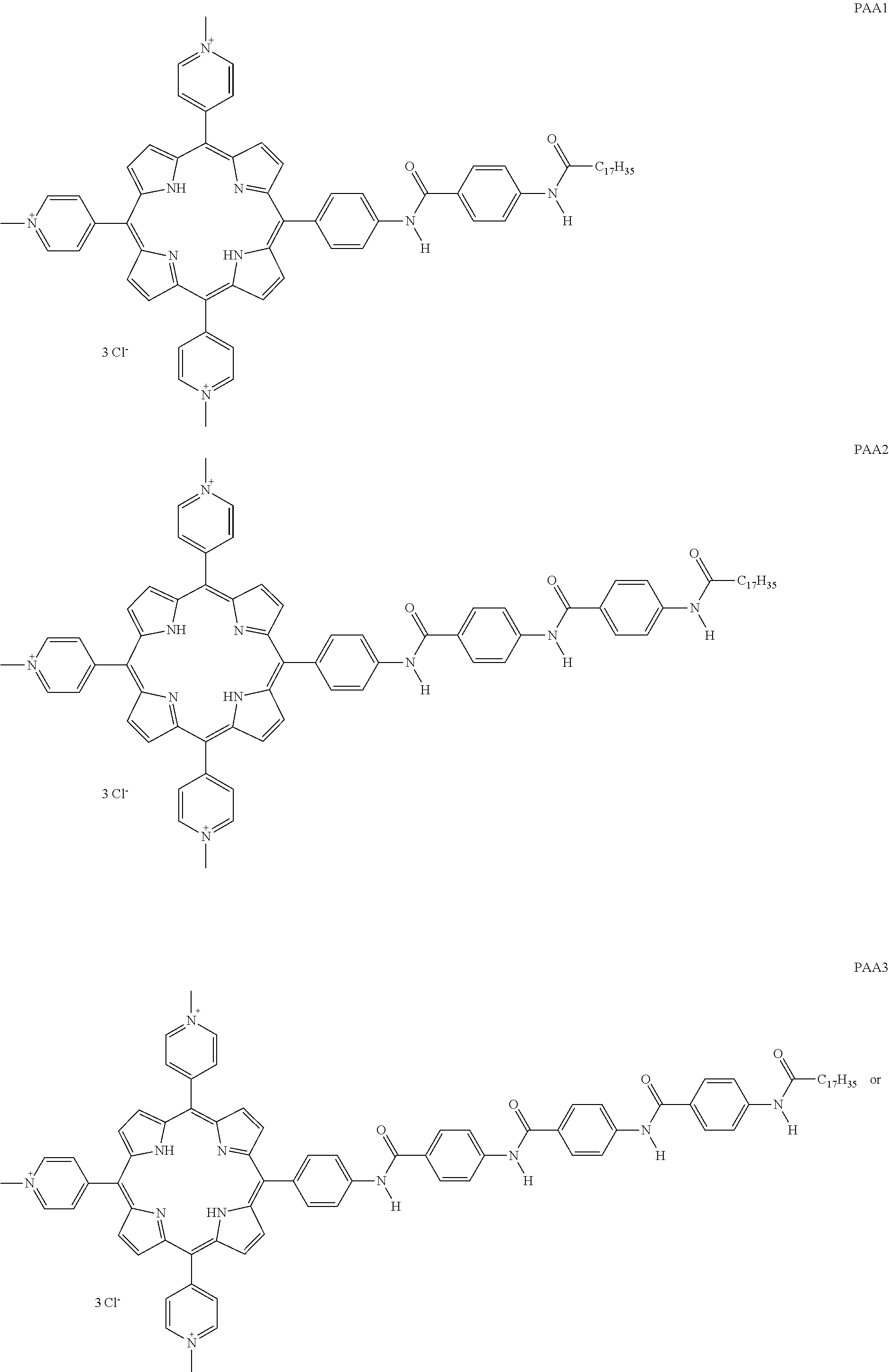

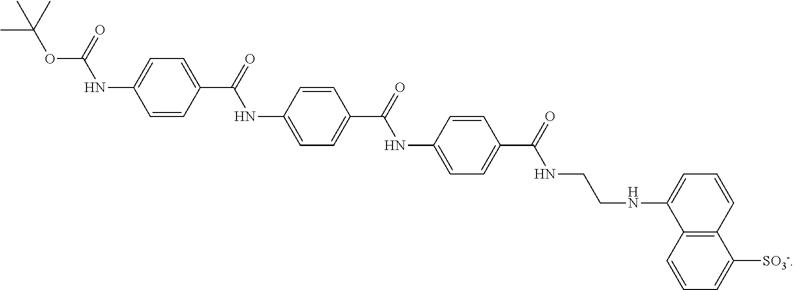

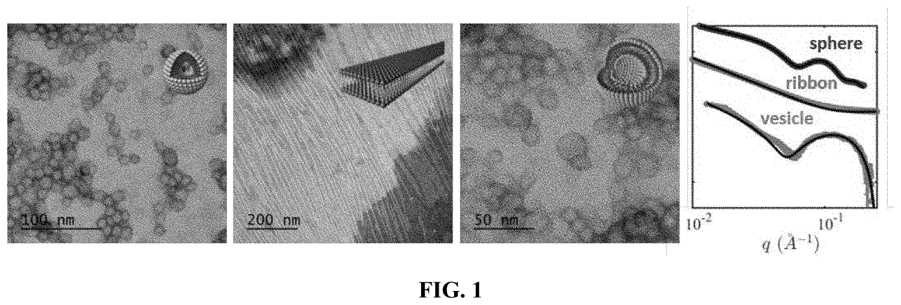

A materials platform based on molecular self-assembly that generates nanostructured materials with extraordinary mechanical stability has been developed. By adjusting the molecular design, tuned nanostructure geometries are demonstrated, resulting in spherical molecular nanoparticles, ribbon-like nanofibers, and vesicle-like hollow spheres (FIG. 1). This platform is unique because the nanostructures do not undergo decomposition or dissociation on surfaces or under harsh conditions.

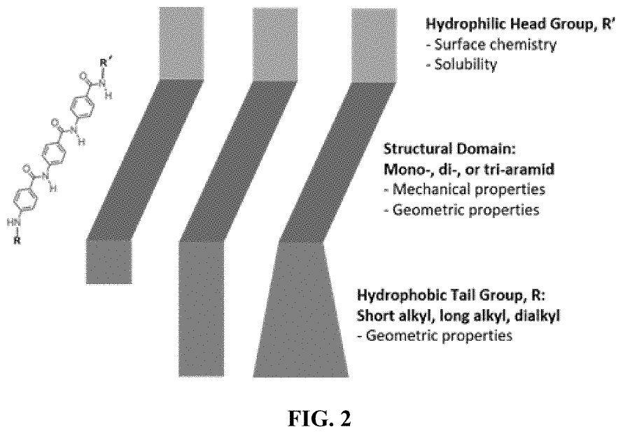

The chemical motif that gives rise to these nanostructures include monoaramid, diaramid, triaramid, etc. structural domains, as shown in FIG. 2. The hydrophilic head group, R′, can be modified to include cationic, anionic, zwitterionic, and uncharged moieties to create charged nanostructures. The head group can be a heavy metal chelator. The hydrophilic head group can be an amino acid or a peptide, for example, a peptide of two, three, four, five, six, seven, eight, nine or ten amino acids. The amino acids can be the same or different. When the hydrophilic head group is a peptide, the peptide can have an affinity for a surface of a cell or a protein. The hydrophobic alkyl tail groups can be an arbitrary length or include multiple alkyl groups.

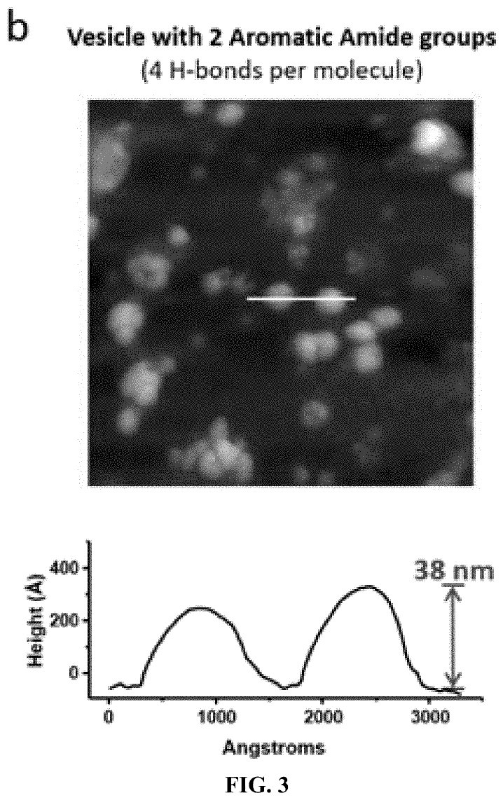

These nanostructures are unique because they have strong mechanical properties and a low degree of molecular exchange. Compared to standard phospholipid vesicles, aramid vesicles have robust membranes that can support their weight in air. FIG. 3 shows (a) the height images of a standard phospholipid vesicle after collapsing on a substrate into bilayers, and (b) air-stable aramid nanostructures in the form of hollow spheres that retain their height even after drying on a substrate. These air-stable structures also retain water or solutions of drugs or other solutes upon drying. Molecular species can be encapsulated such as drugs or water in these structures and observe that the solution remains inside until the nano-vesicle is burst via a trigger, like an incident electron beam.

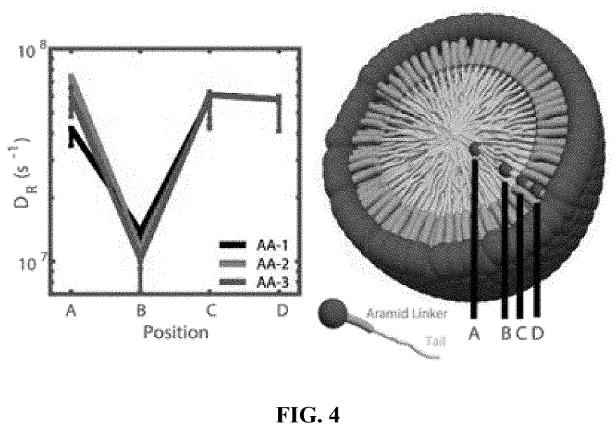

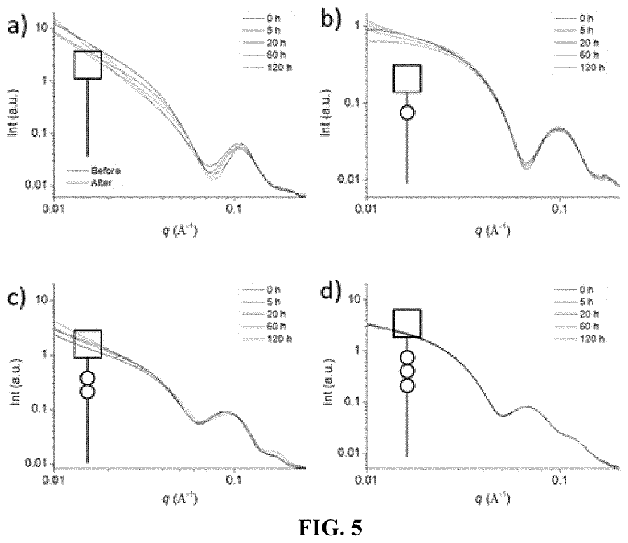

EPR spectroscopy was used to characterize the conformational dynamics within 8 nm nanospheres, shown in FIG. 4. The aramid domain of the spherical nanostructures exhibits very slow conformational dynamics, indicating solid-like behavior. This result suggests that rather than behaving as micelles, these structures are solid-like, similar to molecular nanoparticles.

Stability measurements were carried out on aramid amphiphile molecular nanoparticles in water by subjecting the samples to UV light for varying amounts of time and then measuring the X-ray scattering profiles. The results show that by adding aramid domains, the nanoparticles retain their geometric structure upon irradiation and are therefore resistant to degradation.

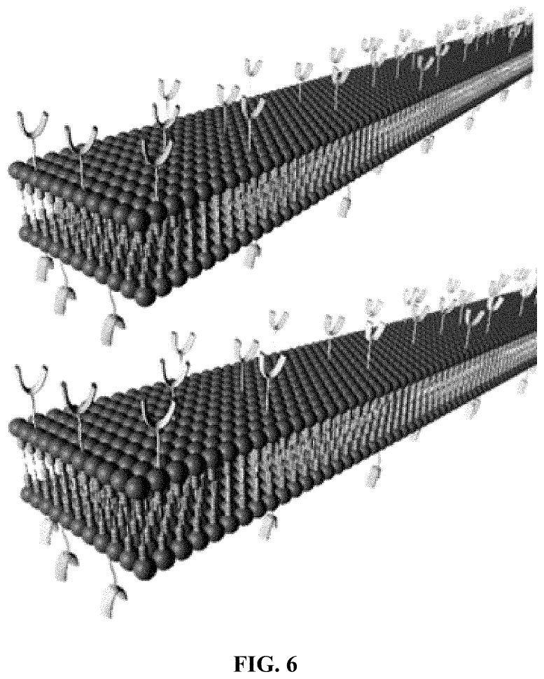

The nanostructure surfaces can be functionalized with arbitrary surface groups by co-assembly. As a result, robust, mechanically stable, nanostructures can be formed with one or more functionality presented at the surface with chosen ratios, concentrations, and chemistries. Ribbon-like nanofibers have been synthesized with chelating groups at the surface for removing heavy metals (arsenic or lead) from drinking water as shown in FIG. 6.

The compounds described herein feature robust amphiphiles with end-substituted hydrophobic and hydrophilic moieties.

Such amphiphiles can construct highly ordered nanostructures that include micelles, vesicles, and ribbons, nanofibers, etc. The nanofiber can have a diameter of 5-6 nm and a length of up to 20 microns. The nanofiber can have an aspect ratio of 5,000:1

In certain embodiments, the ordered nanostructures can form a fiber. The fiber can be drawn from a solution, extruded, or shear aligned. The fiber can have a length of 1 to 100 cm, or longer. The fiber can be formed of a plurality of amphiphiles, for example, a donor amphiphile and an acceptor amphiphile.

The aramid-based structural motifs can have the following general structure.

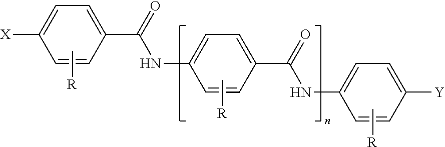

n is the number of rigid aramid repeating units. For example, n can be 0, 1, 2, 3, 4, 5, 6, 7 or 8. The aramid can be a monoaramid, diaramid, triaramid, or quadraramid or higher aramid.

The solubility of the amphiphiles can be tuned by controlling the length of hydrophobic and hydrophilic building blocks.

X can be a tail group. The tail group can be hydrophobic. X can be substituted or unsubstituted, branched or linear form of alkyl, alkenyl, alkynyl, fluorinated, siloxane, and aromatic groups.

Example tail groups are represented here:

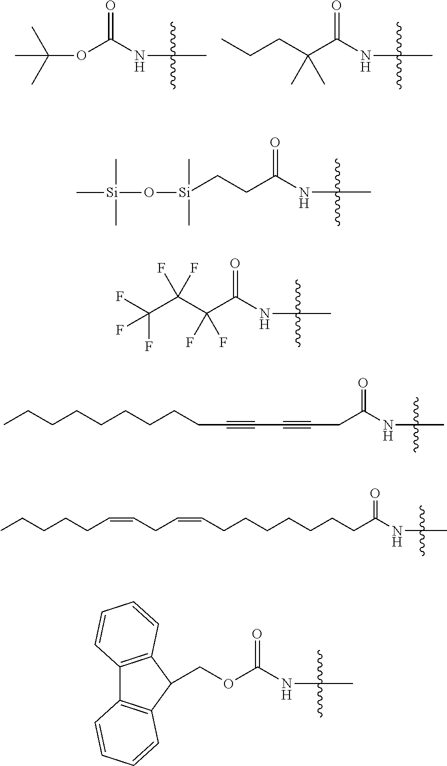

Y can be a head group. The head group can be hydrophilic. Y can be selected from the group consisting of charged functionalities which may have anionic, cationic, and zwitterionic character. Y may also be an uncharged, hydrophilic group like oligo-ethylene glycol. With sufficiently large hydrophilic head groups, aramid amphiphiles become water soluble, a significant advantage for large scale processing of materials by ecofriendly methods.

Example head groups are represented here:

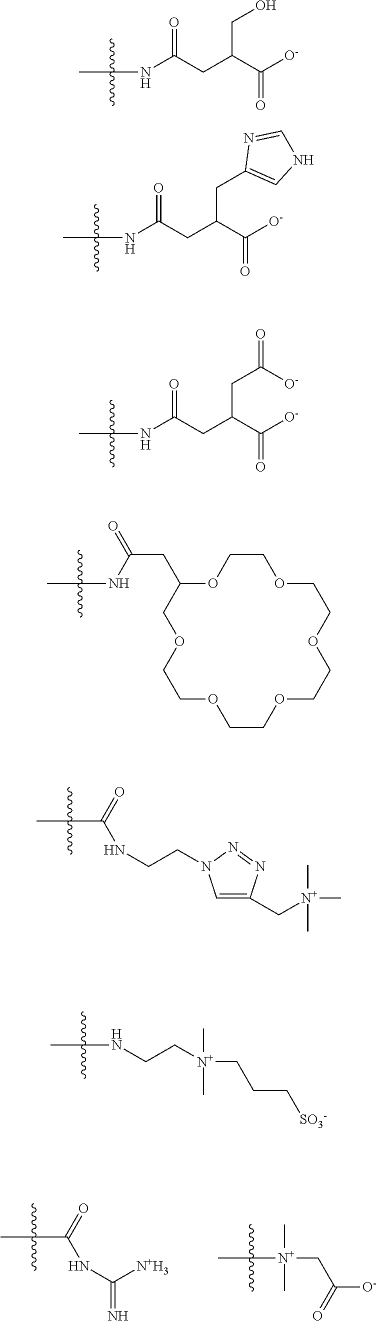

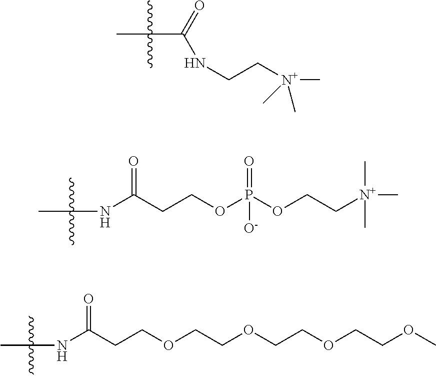

The aramid compounds can be cationic, anionic, or zwitterionic. The aramid compounds can have the following exemplary structures. An example of a zwitterionic amphiphile is:

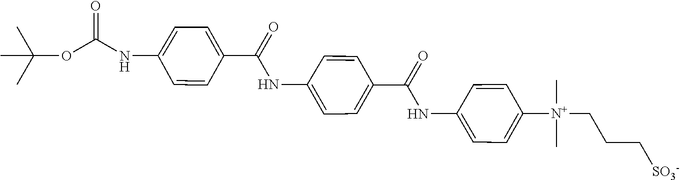

An example of a cationic amphiphile is:

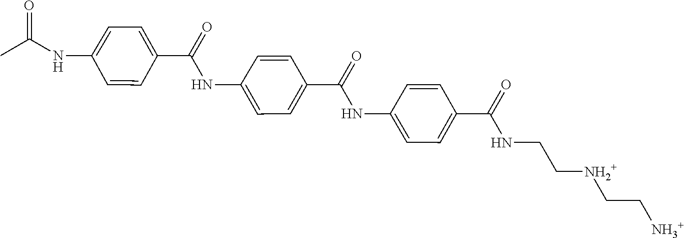 [SMILES: CC(=O)Nc1ccc(C(=O)Nc2ccc(C(=O)Nc3ccc(C(=O)NCC[NH2+]CC[NH3+])cc3)cc2)cc1]

An example of an anionic is:

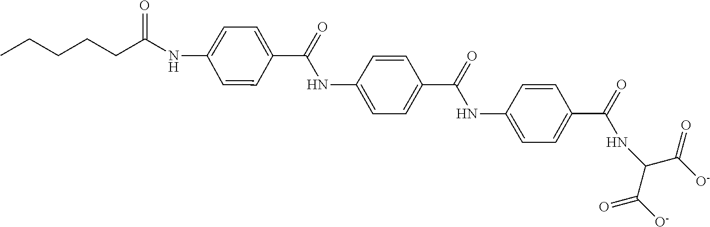 [SMILES: CCCCCC(=O)Nc1ccc(C(=O)Nc2ccc(C(=O)Nc3ccc(C(=O)NC(C(=O)[O-])C(=O)[O-])cc3)cc2)cc1]

Syntheses of several aramid amphiphiles are described more fully below, in the following examples.

In a representative synthesis, the aramid building block was formed using an amidation reaction between carboxylic acid and amine moieties to obtain the amide bond.

### EXPERIMENTAL SECTION

Tuning the molecular structure of the aramid amphiphiles allows desired nanostructure assemblies to be accessed.

Materials:

Methyl 4-aminobenzoate, 4-acetamidobenzoic acid, hexanoic acid, 1,3-propanesultone, dimethyl-para-phenylenediamine, Boc-4-aminobenzoic acid, 1,4-bis-Boc-1,4,7-triazaheptane, dimethyl 2-aminomalonate, triethylamine, 1-ethyl-3-(3-dimethylaminopropyl)carbodiimide hydrochloride, 4-dimethylaminopyridine, 1-hydroxybenzotriazole hydrate, lithium hydroxide, sodium hydroxide, trifluoroacetic acid, hydrochloric acid, methylene chloride, ethyl acetate, dimethylformamide, tetrahydrofuran, ethanol, and methanol.

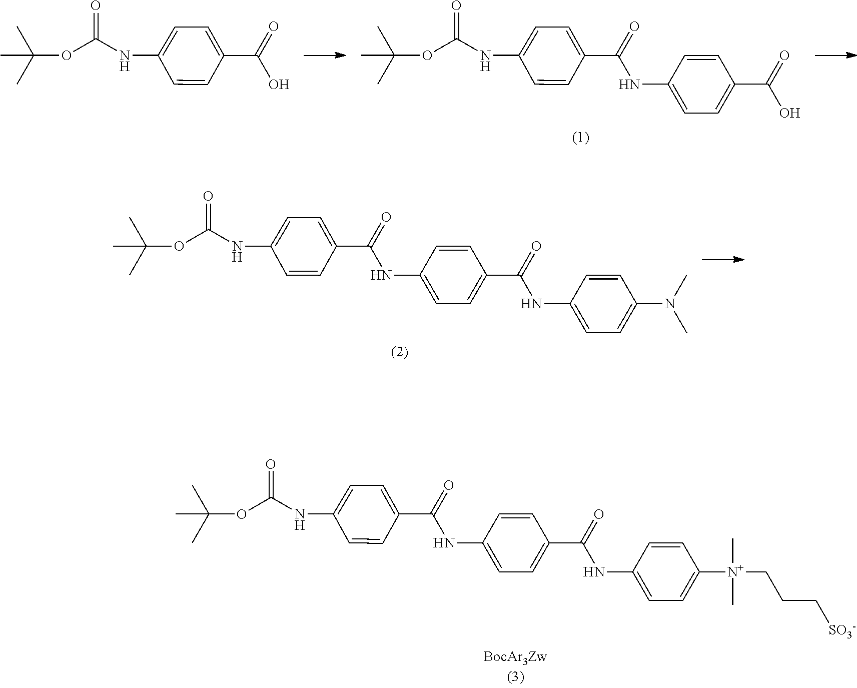

Synthesis:

Synthesis of compound 1: The solution of Boc-4-aminobenzoic acid (1.0 g, 4.21 mmol), methyl 4-aminobenzoate (1.27 g, 8.42 mmol), 1-ethyl-3-(3-dimethylaminopropyl)carbodiimide hydrochloride (1.61 g, 8.42 mmol), and 4-dimethylaminopyridine (1.0 g, 8.42 mmol) in 50 mL chloroform was stirred at room temperature for 24 h. After the reaction, the solvent was removed in vacuum and the remaining residue was precipitated with water. The mixture was filtered and the precipitate was washed with chloroform several times. The solid material was dissolved in 40 mL tetrahydrofuran and 20 mL ethanol. To this was added lithium hydroxide (0.5 g, 21.1 mmol) in 10 mL water and refluxed at 70° C. for 6 h. The reaction mixture was shifted to room temperature and acidified to pH 2 with the addition of 5M hydrochloric acid solution. The precipitate was filtered thorough Buchner funnel, washed with water several times, and dried in vacuum to obtain the pure product as a white solid. Yield: 87%. ¹H NMR (400 MHz, DMSO-d): δ=7.93 (m, 6H), 7.66 (d, 2H), 1.49 (s, 9H) ppm.

Synthesis of compound 2: The solution of compound 1 (0.3 g, 0.84 mmol), dimethyl-para-phenylenediamine (0.34 g, 2.54 mmol), 1-ethyl-3-(3-dimethylaminopropyl)carbodiimide hydrochloride (0.49 g, 2.53 mmol), and 1-hydroxybenzotriazole hydrate (0.34 g, 2.53 mmol) in 20 mL dimethylformamide was stirred at 60° C. for 24 h. After the reaction, the solvent was removed in vacuum and the remaining residue was precipitated with water. The crude mixture was collected with filter flask. The filtered solid was washed with copious amount of methanol, and dried in vacuum to obtain the pure product as a pale purple solid. Yield: 78%. ¹H NMR (400 MHz, DMSO-d): δ=7.91 (m, 4H), 7.58 (m, 4H), 7.02 (m, 2H), 6.75 (d, 2H), 2.79 (s, 6H), 1.49 (s, 9H) ppm.

Synthesis of compound BocAr₃Zw (3): The compound 2 (0.15 g, 0.86 mmol) was dissolved in 15 mL dimethylformamide. At 70° C., the 1,3-propanesultone (1.05 g, 8.61 mmol) was slowly injected via a syringe and stirred for 48 h in a pressure vessel. The volatile was removed under reduced pressure and 30 mL tetrahydrofuran was added. The resulting precipitate was filtered and dried in vacuum to obtain the pure product as a pale grey solid. Yield: 82%. ¹H NMR (400 MHz, DMSO-d): δ=7.95 (m, 10H), 7.60 (d, 2H), 3.99 (t, 2H), 3.58 (s, 6H), 2.41 (t, 2H), 1.67 (t, 2H), 1.49 (s, 9H) ppm. ¹³C NMR (400 MHz, DMSO-d): δ=165.8, 153.1, 143.4, 140.9, 139.5, 129.3, 128.1, 122.2, 121.1, 119.8, 117.6, 80.1, 68.1, 54.4, 47.9, 28.5, 20.3 ppm.

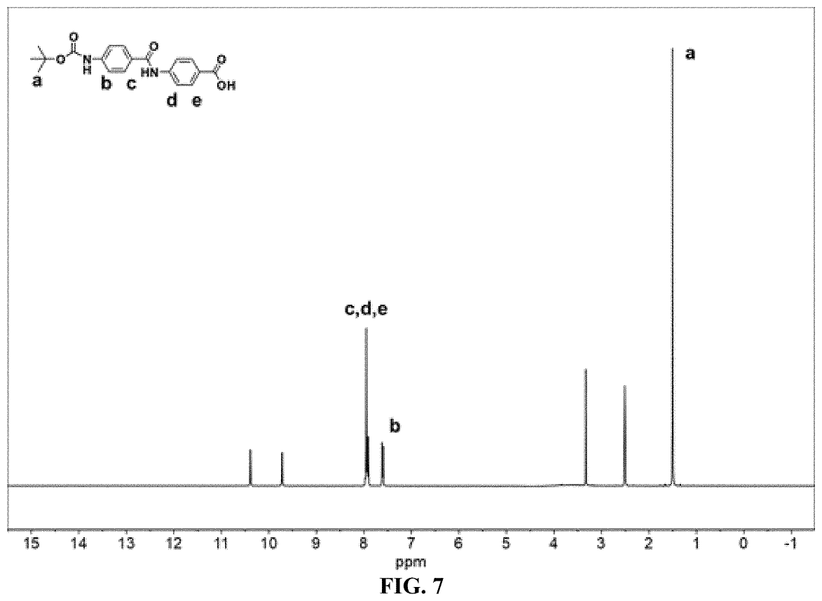

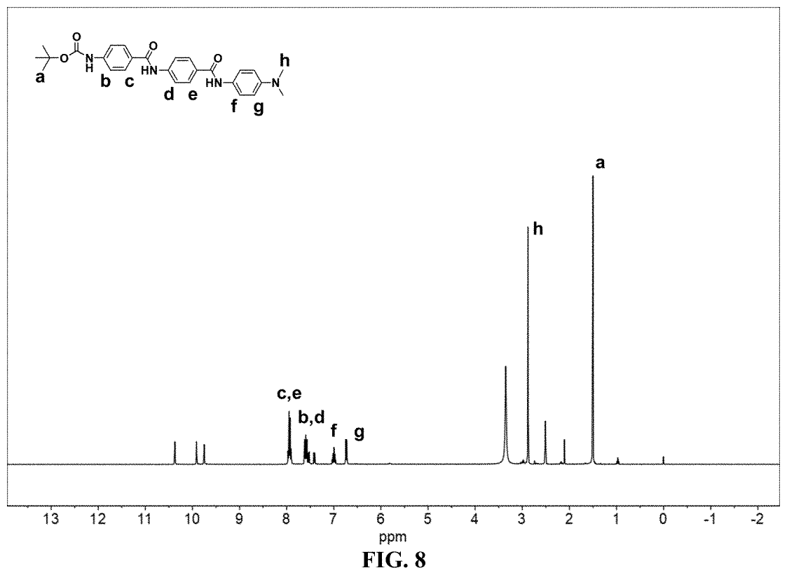

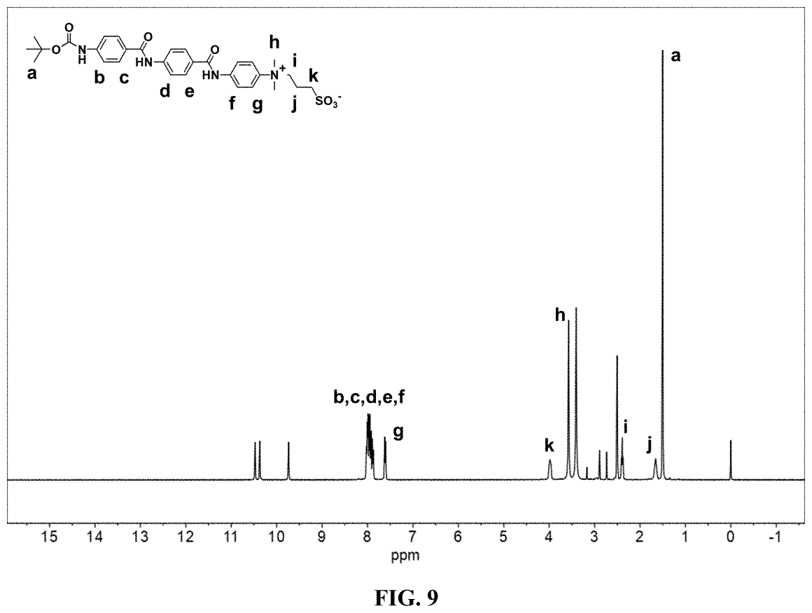

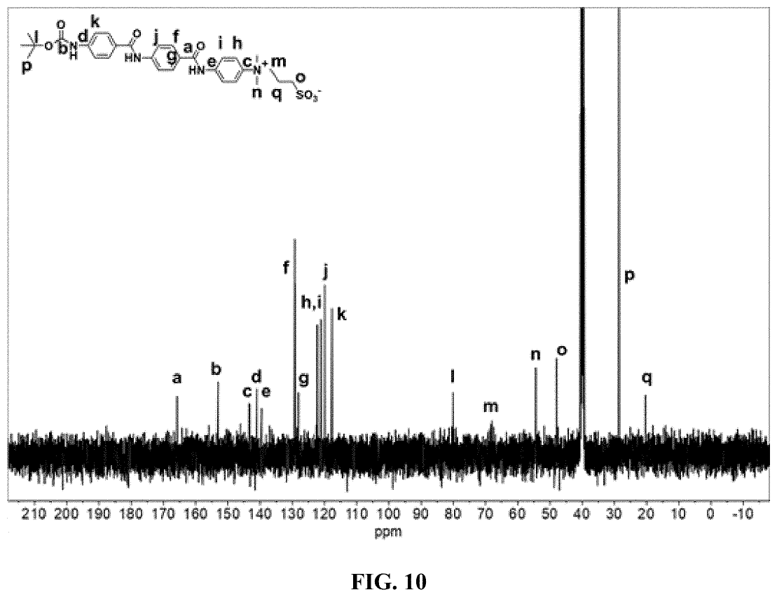

FIGS. 7-10 show ¹H NMR spectra of compound 1, ¹H NMR spectra of compound 2, ¹H NMR spectra of compound 3, and ¹³C NMR spectra of compound 3.

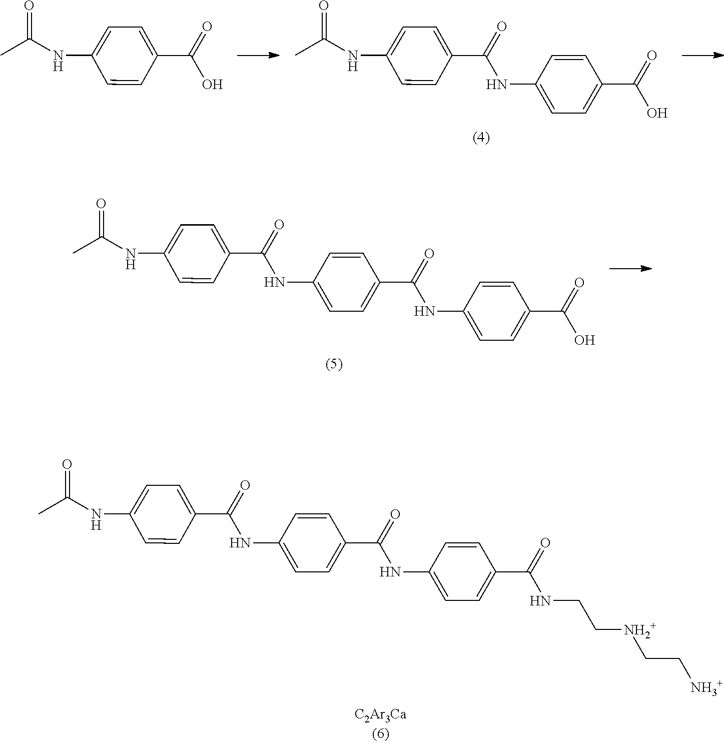 [SMILES: CC(=O)Nc1ccc(C(=O)O)cc1 ; CC(=O)Nc1ccc(C(=O)Nc2ccc(C(=O)O)cc2)cc1 ; CC(=O)Nc1ccc(C(=O)Nc2ccc(C(=O)Nc3ccc(C(=O)O)cc3)cc2)cc1 ; CC(=O)Nc1ccc(C(=O)Nc2ccc(C(=O)Nc3ccc(C(=O)NCC[NH2+]CC[NH3+])cc3)cc2)cc1]

Synthesis of compound 4: The solution of compound 4-acetamidobenzoic acid (1.5 g, 8.37 mmol), methyl 4-aminobenzoate (2.53 g, 16.74 mmol), 1-ethyl-3-(3-dimethylaminopropyl)carbodiimide hydrochloride (4.81 g, 25.11 mmol), and 4-dimethylaminopyridine (3.39 g, 25.11 mmol) in 50 mL dimethylformamide was stirred at 60° C. for 24 h. After the reaction, the solvent was removed in vacuum and the remaining residue was precipitated with water. The mixture was filtered off and washed with methanol several times. The solid material was dissolved in 40 mL tetrahydrofuran and 20 mL ethanol. To this was added lithium hydroxide (1.0 g, 41.8 mmol) in 10 mL water and refluxed at 70° C. for 12 h. The reaction mixture was shifted to room temperature and acidified to pH 2 with the addition of 5M hydrochloric acid solution. The precipitate was filtered thorough Buchner funnel, washed with water several times, and dried in vacuum to obtain the pure product as a white solid. Yield: 91%. ¹H NMR (400 MHz, DMSO-d): δ=7.92 (m, 6H), 7.74 (d, 2H), 2.09 (s, 3H) ppm.

Synthesis of compound 5: The solution of compound 4 (0.2 g, 0.67 mmol), methyl 4-aminobenzoate (0.3 g, 2.01 mmol), 1-ethyl-3-(3-dimethylaminopropyl)carbodiimide hydrochloride (0.39 g, 2.01 mmol), and 4-dimethylaminopyridine (0.25 g, 2.01 mmol) in 20 mL dimethylformamide was stirred at 60° C. for 24 h. After the reaction, the solvent was removed in vacuum and the remaining residue was precipitated with water. The mixture was filtered off and washed with chloroform several times. The solid material was dissolved in 40 mL tetrahydrofuran and 20 mL ethanol. To this was added lithium hydroxide (0.08 g, 3.35 mmol) in 10 mL water and refluxed at 70° C. for 12 h. The reaction mixture was shifted to room temperature and acidified to pH 2 with the addition of 5M hydrochloric acid solution. The precipitate was filtered thorough Buchner funnel, washed with water several times, and dried in vacuum to obtain the pure product as a white solid. Yield: 84%. ¹H NMR (400 MHz, DMSO-d): δ=7.99 (m, 10H), 7.75 (d, 2H), 2.10 (s, 3H) ppm.

Synthesis of compound C₂Ar₃Ca (6): The solution of compound 5 (0.15 g, 0.36 mmol), 1,4-bis-Boc-1,4,7-triazaheptane (0.3 g, 0.72 mmol), 1-ethyl-3-(3-dimethylaminopropyl)carbodiimide hydrochloride (0.21 g, 1.08 mmol), and 4-dimethylaminopyridine (0.14 g, 1.08 mmol) in 10 mL dimethylformamide was stirred at 60° C. for 48 h. After the reaction, the solvent was removed in vacuum and the remaining residue was washed with ethyl acetate several times. Continuously, the above synthesized compounds were reacted with 0.5 mL trifluoroacetic acid in 10 mL methylene chloride for 12 h and the solvents were removed under reduced pressure. The 50 mL diethyl ether was added and stirred for another 1 h, and white color solid products were collected and dried to obtain the final products. Yield: 84%. ¹H NMR (400 MHz, DMSO-d): δ=7.96 (m, 10H), 7.75 (d, 2H), 3.56 (t, 2H), 3.18 (m, 6H), 2.10 (s, 3H) ppm. ¹³C NMR (400 MHz, DMSO-d): δ=165.7, 143.1, 129.3, 128.5, 119.8, 118.6, 47.5, 44.7, 35.9, 24.6, 10.52 ppm.

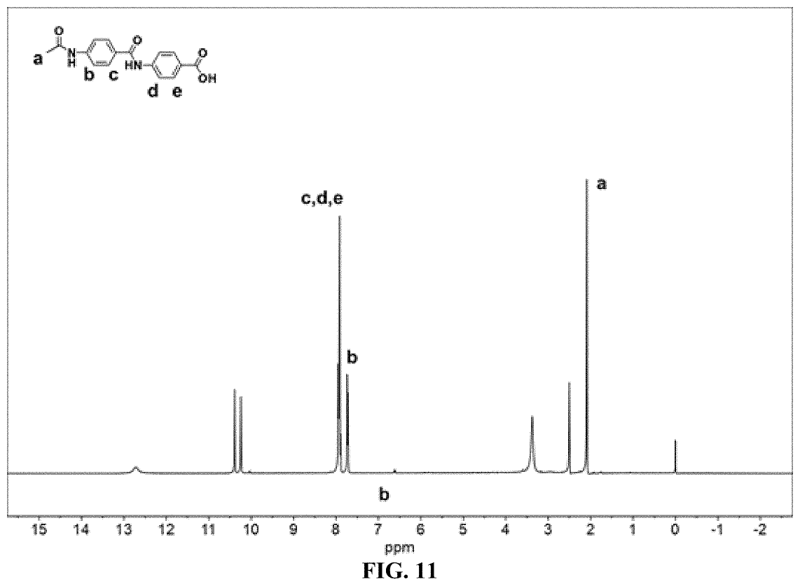

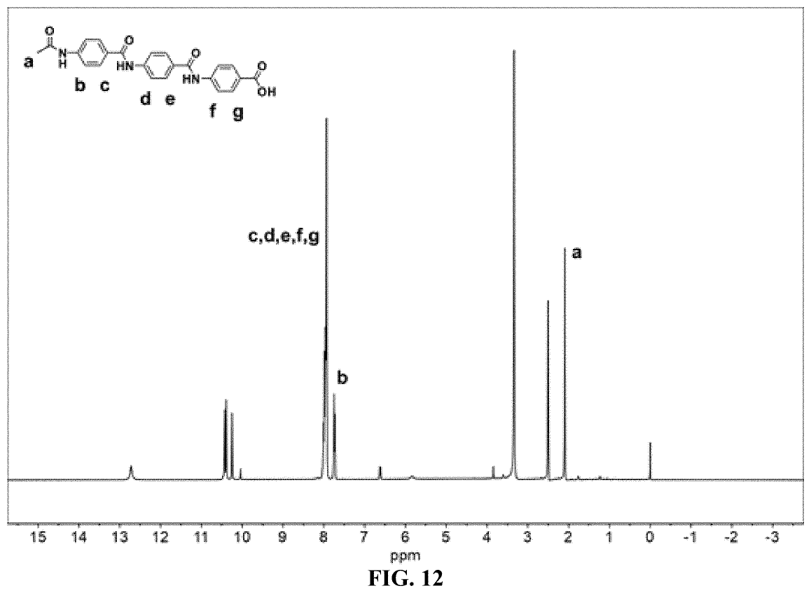

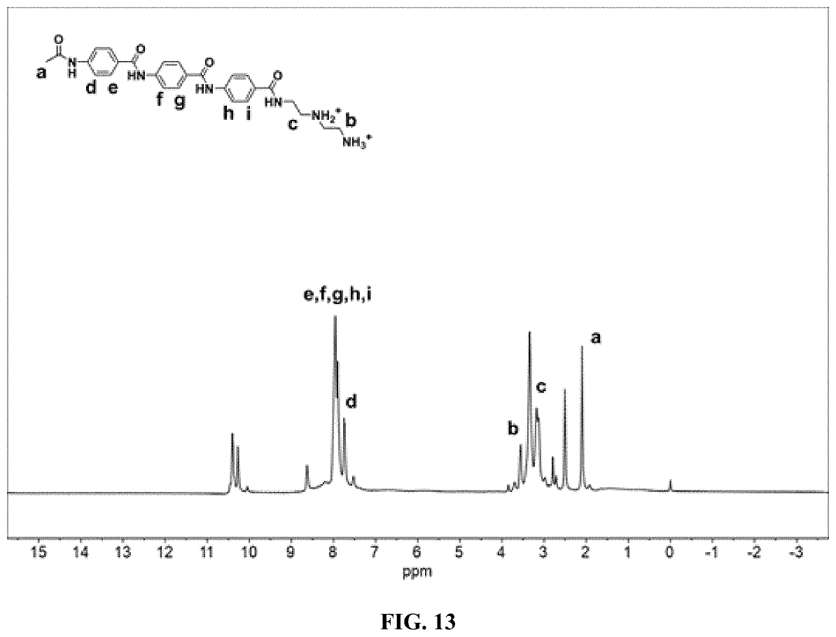

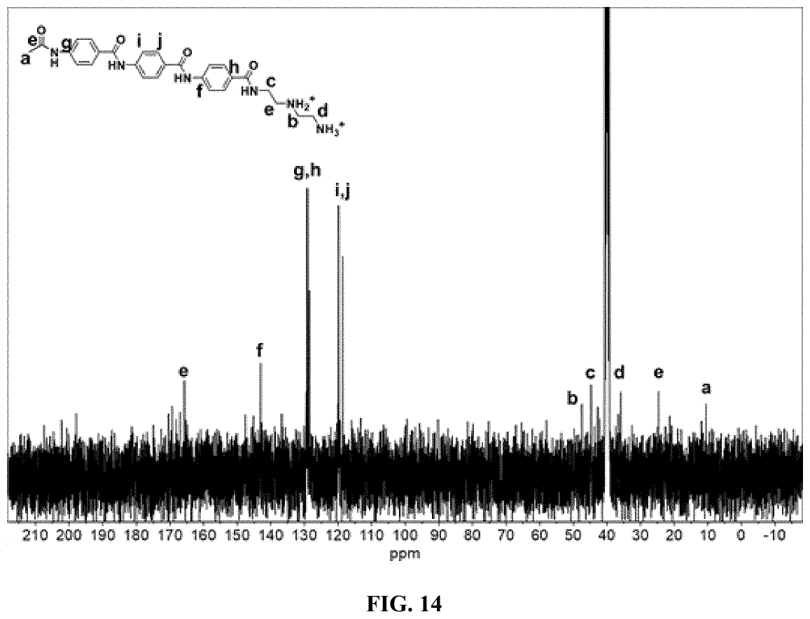

FIGS. 11-14 show ¹H NMR spectra of compound 4, ¹H NMR spectra of compound 5, ¹H NMR spectra of compound 6, and ¹³C NMR spectra of compound 6.

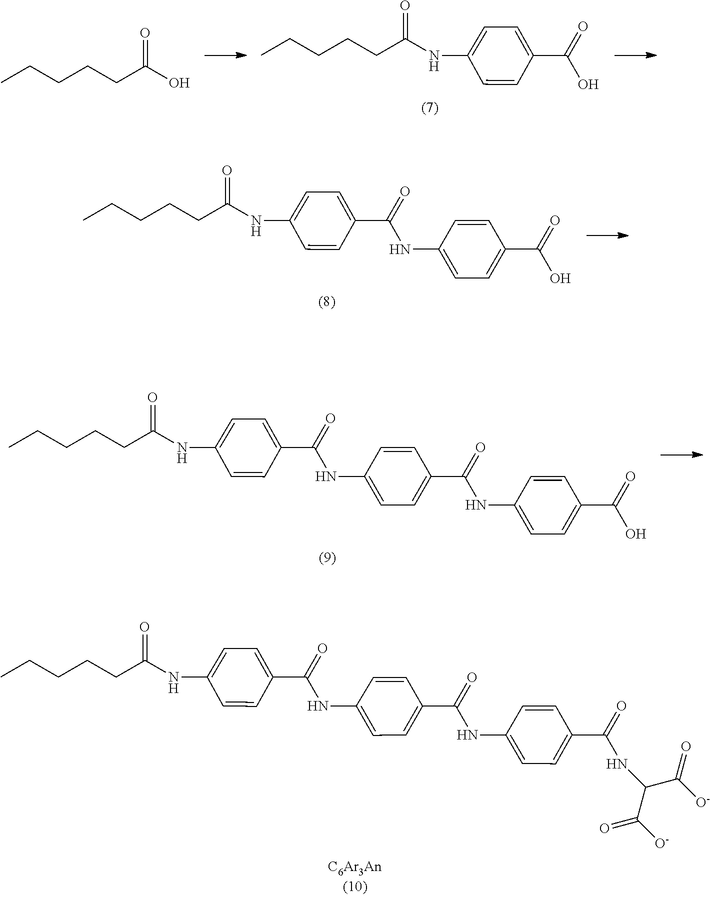

Synthesis of compound 7: The solution of hexanoic acid (1.5 g, 11.01 mmol), methyl 4-aminobenzoate (4.69 g, 16.5 mmol), 1-ethyl-3-(3-dimethylaminopropyl)carbodiimide hydrochloride (6.33 g, 33.04 mmol), and 4-dimethylaminopyridine (4.46 g, 33.04 mmol) in 80 mL tetrahydrofuran was stirred at room temperature for 24 h. After the reaction, the solvent was removed in vacuum. The crude mixture was washed with water and extracted with chloroform. The organic layer was separated and distilled off. The solid material was dissolved in 40 mL tetrahydrofuran and 20 mL ethanol. To this was added lithium hydroxide (1.32 g, 55.05 mmol) in 15 mL water and refluxed at 65° C. for 3 h. The reaction mixture was shifted to room temperature and acidified to pH 2 with the addition of 5M hydrochloric acid solution. The precipitate was filtered thorough Buchner funnel, washed with water several times, and dried in vacuum to obtain the pure product as a white solid. Yield: 91%. ¹H NMR (400 MHz, DMSO-d): δ=7.86 (d, 2H), 7.71 (d, 2H), 2.33 (t, 2H), 1.61 (m, 2H), 1.28 (m, 4H), 0.85 (t, 3H) ppm.

Synthesis of compound 8: The solution of compound 7 (0.3 g, 1.27 mmol), methyl 4-aminobenzoate (0.59 g, 3.83 mmol), 1-ethyl-3-(3-dimethylaminopropyl)carbodiimide hydrochloride (0.74 g, 3.83 mmol), and 4-dimethylaminopyridine (0.47 g, 3.83 mmol) in 15 mL dimethylformamide was stirred at 60° C. for 24 h. After the reaction, the solvent was removed in vacuum and the remaining residue was precipitated with water. The mixture was filtered off and washed with methanol several times. The solid material was dissolved in 20 mL tetrahydrofuran and 10 mL ethanol. To this was added lithium hydroxide (0.15 g, 6.35 mmol) in 10 mL water and refluxed at 70° C. for 12 h. The reaction mixture was shifted to room temperature and acidified to pH 2 with the addition of 5M hydrochloric acid solution. The precipitate was filtered thorough Buchner funnel, washed with water several times, and dried in vacuum to obtain the pure product as a white solid. Yield: 89%. ¹H NMR (400 MHz, DMSO-d): δ=7.92 (m, 6H), 7.76 (d, 2H), 2.35 (t, 2H), 1.62 (m, 2H), 1.31 (m, 4H), 0.89 (t, 3H) ppm.

Synthesis of compound 9: The solution of compound 8 (0.4 g, 0.84 mmol), methyl 4-aminobenzoate (0.38 g, 2.52 mmol), 1-ethyl-3-(3-dimethylaminopropyl)carbodiimide hydrochloride (0.48 g, 2.52 mmol), and 4-dimethylaminopyridine (0.31 g, 2.52 mmol) in 20 mL dimethylformamide was stirred at 60° C. for 24 h. After the reaction, the solvent was removed in vacuum and the remaining residue was precipitated with water. The mixture was filtered off and washed with methanol several times. The solid material was dissolved in 20 mL tetrahydrofuran and 10 mL ethanol. To this was added lithium hydroxide (0.1 g, 4.2 mmol) in 10 mL water and refluxed at 70° C. for 12 h. The reaction mixture was shifted to room temperature and acidified to pH 2 with the addition of 5M hydrochloric acid solution. The precipitate was filtered thorough Buchner funnel, washed with water several times, and dried in vacuum to obtain the pure product as a white solid. Yield: 84%. ¹H NMR (400 MHz, DMSO-d): δ=7.94 (m, 10H), 7.77 (d, 2H), 2.36 (t, 2H), 1.62 (m, 2H), 1.31 (m, 4H), 0.89 (t, 3H) ppm.

Synthesis of compound C₆Ar₃An (10): Into 20 mL dimethylformamide, compound 9 (0.15 g, 0.32 mmol), dimethyl aminomalonate (0.14 g, 0.95 mmol), 1-ethyl-3-(3-dimethylaminopropyl)carbodiimide hydrochloride (0.18 g, 0.95 mmol), and 4-dimethylaminopyridine (0.12 g, 0.95 mmol) were added. The well dissolved solution was stirred at room temperature for 48 h. After solvent evaporation, the crude mixture was washed with water and methanol. The resulting residue was then basified by addition of sodium hydroxide (0.01 g, 0.35 mmol) in 15 ml tetrahydrofuran and 2 ml water at 70° C. for 12 h, and then precipitated in diethylether three times to give the corresponding desired white solid compound. Yield: 78%. ¹H NMR (400 MHz, DMSO-d): δ=7.97 (m, 10H), 7.71 (d, 2H), 5.45 (s, 6H), 2.36 (t, 2H), 1.62 (m, 2H), 1.31 (m, 4H), 0.89 (t, 3H) ppm. ¹³C NMR (400 MHz, DMSO-d): 6=172.2, 167.6, 166.3, 165.6, 143.1, 129.2, 119.8, 118.6, 56.7, 36.9, 31.3, 25.1, 22.4, 14.4 ppm.

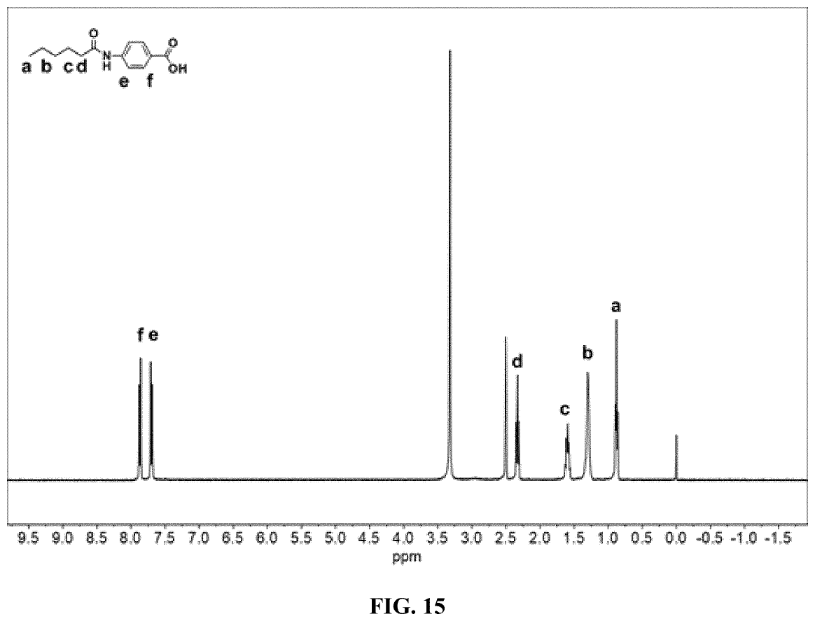

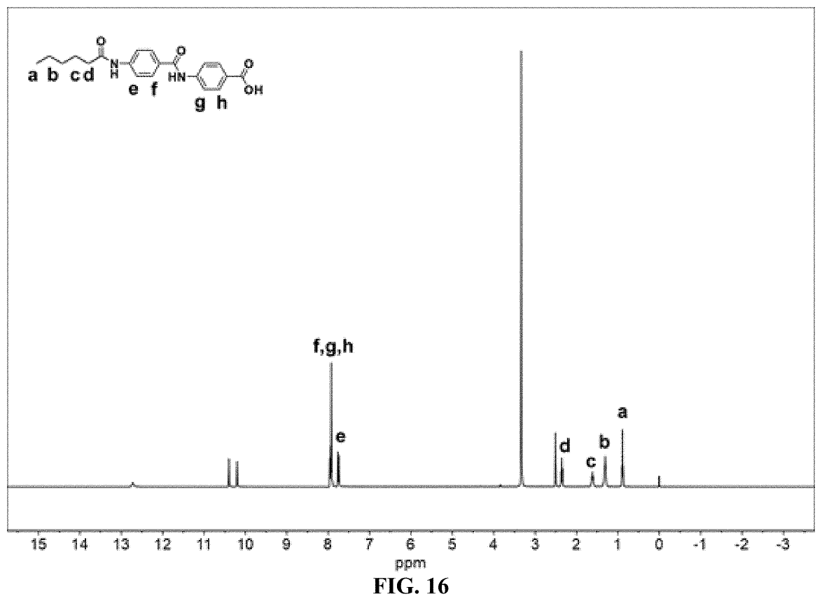

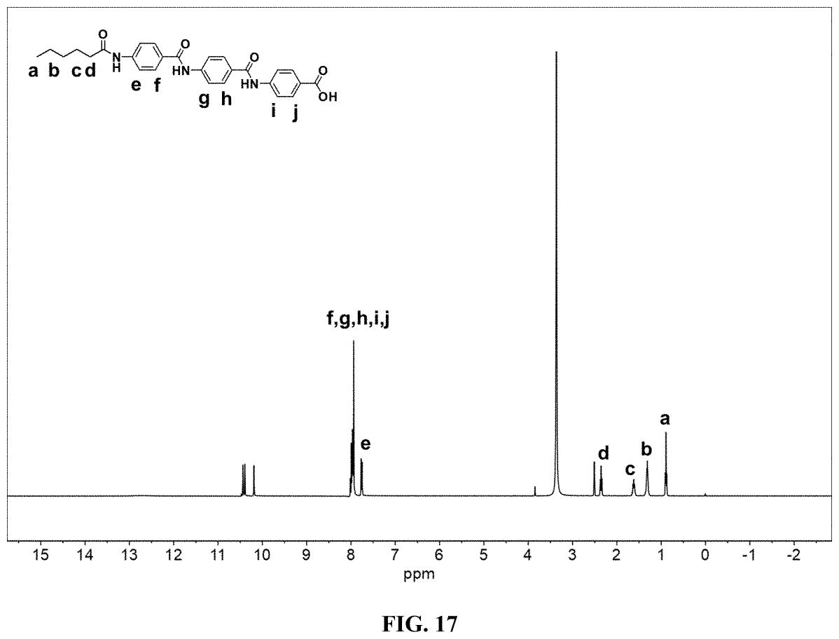

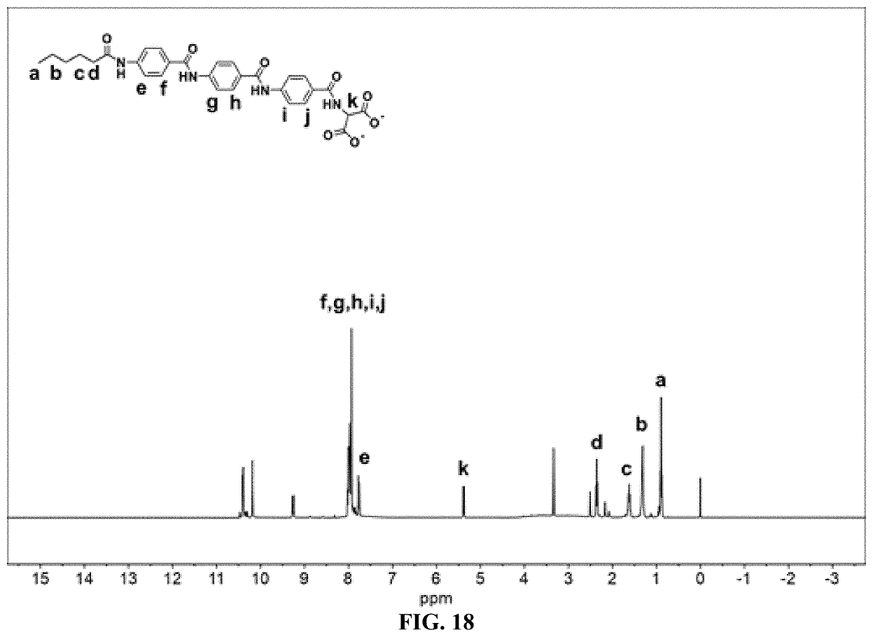

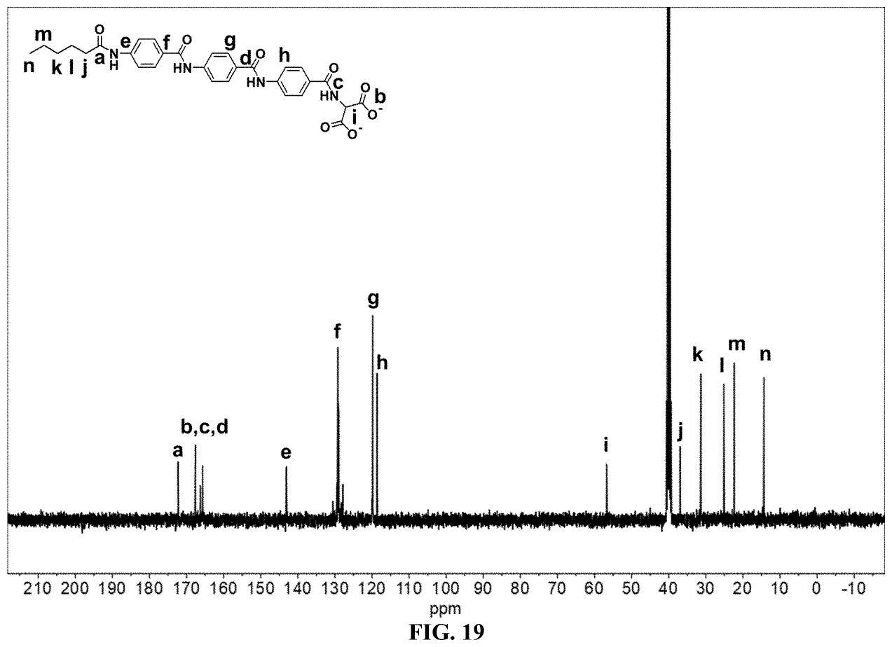

FIGS. 15-19 show ¹H NMR spectra of compound 7, ¹H NMR spectra of compound 8, ¹H NMR spectra of compound 9, ¹H NMR spectra of compound 10, and ¹³C NMR spectra of compound 10.

Self-Assembly Behaviors:

The amphiphiles were studied under transmission electron microscopy (TEM) to determine their assembled structure. By varying the number of aramids in the structural domain and the relative hydrophilic and hydrophobic strengths of the corresponding groups on the amphiphile, spherical molecular nanoparticles, ribbon-like nanofibers, and vesicle-like hollow spheres were all observed.

Consistent with broader aspects of description of the compounds and structures described herein, it was found that representative aramid amphiphiles for BocAr₃Zw, when dissolved at neutral pH and dried onto surfaces, self-assembled into nanoribbons.

### Example

Compound 3, a Boc-terminated, three aramid-containing amphiphile with a zwitterionic head group was added to deionized water (3 mg amphiphile/mL water), and the pH was adjusted to 7 using 10 mM sodium hydroxide and 10 mM hydrochloric acid solutions. The sample was prepared for TEM by pipetting 15 μL of the solution onto a carbon coated TEM grid, removing the droplet by blotting the grid edge after 10 seconds of contact with the grid, pipetting 15 μL of a 0.1% (by volume) phosphotungstic acid negative stain onto the grid, and removing the droplet by blotting the grid edge after 10 seconds of contact with the grid. This compound was observed to from nanoribbons with uniform dimensions and morphologies. Amphiphile solutions analyzed without the negative stain also exhibited assembly.

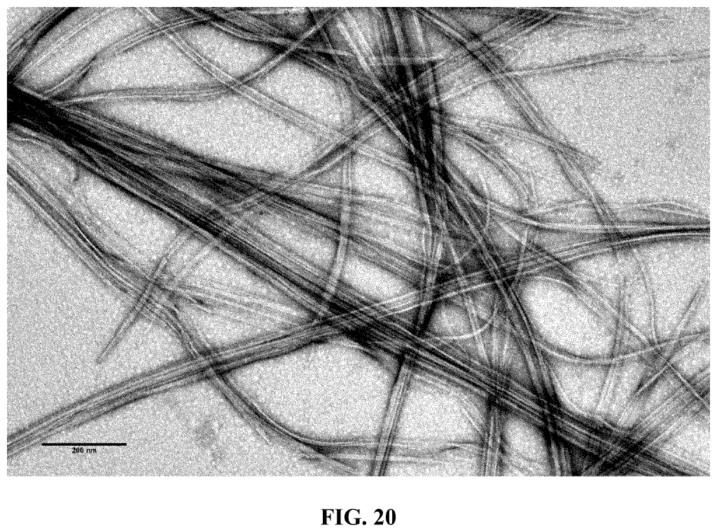

FIG. 20 shows a TEM micrograph of the assembly of compound 3.

### Example

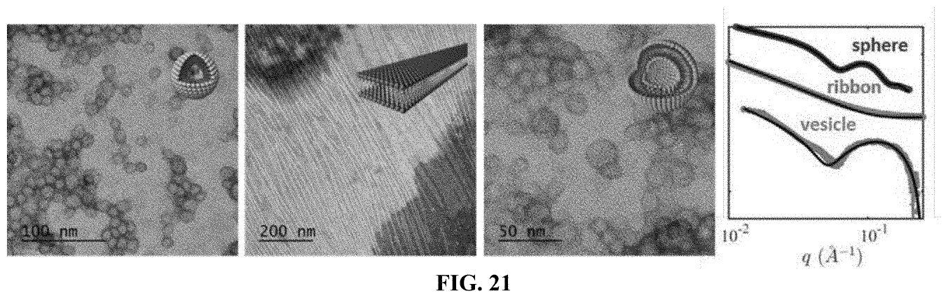

FIG. 21 shows aramid amphiphiles self-assemble into nanospheres, ribbon-like nanofibers, or hollow spheres.

Amphiphiles with aramid-containing structural domains exhibit enhanced mechanical stability and post-assembly robustness in air compared to traditional amphiphiles.

Materials:

Methyl 4-aminobenzoate, 4-acetamidobenzoic acid, hexanoic acid, 1,3-propanesultone, dimethyl-para-phenylenediamine, Boc-4-aminobenzoic acid, 1,4-bis-Boc-1,4,7-triazaheptane, dimethyl 2-aminomalonate, triethylamine, 1-ethyl-3-(3-dimethylaminopropyl)carbodiimide hydrochloride, 4-dimethylaminopyridine, 1-hydroxybenzotriazole hydrate, lithium hydroxide, sodium hydroxide, trifluoroacetic acid, hydrochloric acid, methylene chloride, ethyl acetate, dimethylformamide, tetrahydrofuran, ethanol, and methanol.

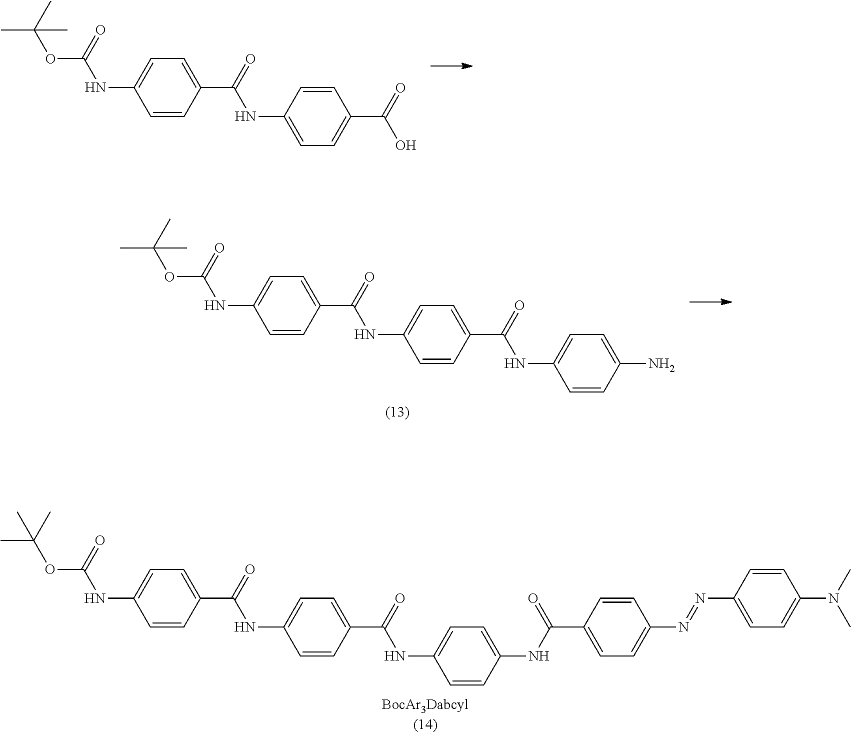 [SMILES: CC(C)(C)OC(=O)Nc1ccc(C(=O)Nc2ccc(C(=O)O)cc2)cc1 ; CC(C)(C)OC(=O)Nc1ccc(C(=O)Nc2ccc(C(=O)Nc3ccc(N)cc3)cc2)cc1 ; CN(C)c1ccc(/N=N/c2ccc(C(=O)Nc3ccc(NC(=O)c4ccc(NC(=O)c5ccc(NC(=O)OC(C)(C)C)cc5)cc4)cc3)cc2)cc1]

Synthesis:

Synthesis of compound 13: The solution of compound 1 (0.50 g, 1.40 mmol), p-phenylenediamine (3.03 g, 2.81 mmol), 1-ethyl-3-(3-dimethylaminopropyl)carbodiimide hydrochloride (0.32 g, 1.68 mmol), and 4-dimethylaminopyridine (0.21 g, 1.68 mmol) in 20 mL dimethylformamide was stirred at 70° C. for 24 h. After the reaction, the solvent was removed in vacuum and the remaining residue precipitated with water. The crude mixture was collected with filter flask. The filtride was washed with copious amount of chloroform, and dried in vacuum to obtain the pure product as a pale purple solid. Yield: 31%. ¹H NMR (400 MHz, DMSO-d): δ=10.29 (s, 1H), 9.77 (s, 1H), 9.72 (s, 1H), 7.92 (m, 6H), 7.60 (d, 2H), 7.36 (d, 2H), 6.54 (d, 2H), 4.91 (s, 2H), 1.51 (s, 9H) ppm.

Synthesis of compound 14: The solution of compound 13 (0.30 g, 0.67 mmol), [4-((4-(dimethylamino)phenyl)azo)benzoic acid] (0.22 g, 0.81 mmol), 1-ethyl-3-(3-dimethylaminopropyl) carbodiimide (0.15 g, 0.81 mmol), and 4-dimethylaminopyridine (0.10 g, 0.81 mmol) in 20 mL dimethylformamide was stirred at 70° C. for 24 h. After the reaction, the solvent was removed in vacuum and the remaining residue precipitated with chloroform. The crude mixture was collected with filter flask. The filtride was washed with copious amount of chloroform, and dried in vacuum to obtain the pure product as a brown solid. Yield: 67.8%. ¹H NMR (400 MHz, DMSO-d): δ=10.34 (s, 1H), 10.30 (s, 1H), 9.84 (s, 1H), 9.72 (s, 1H), 7.93 (m, 10H), 7.60 (d, 4H), 7.45 (d, 2H), 6.89 (d, 2H), 6.64 (d, 2H), 3.10 (s, 6H), 1.51 (s, 9H) ppm.

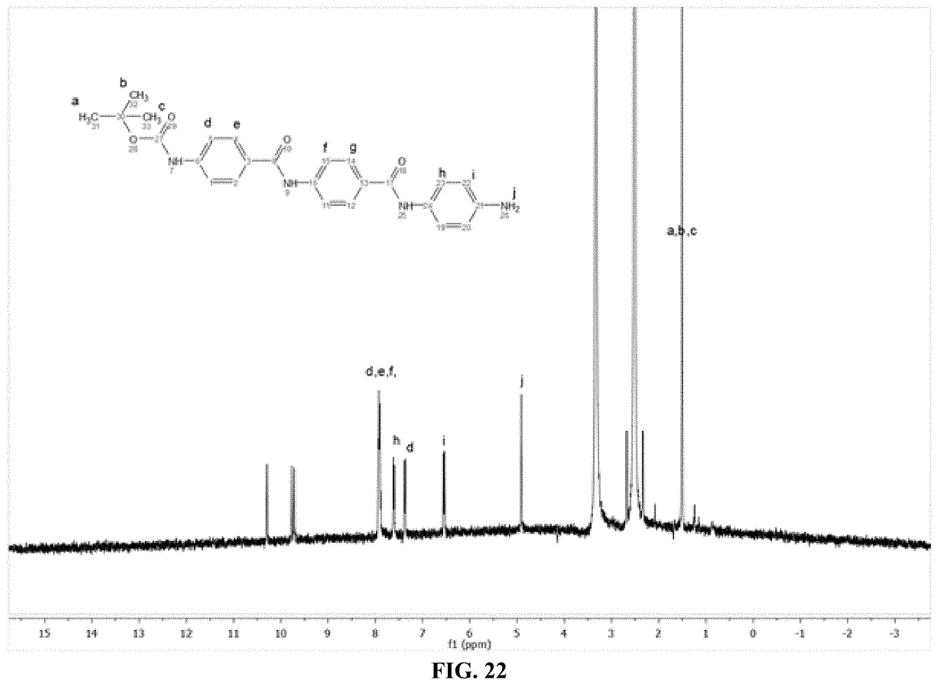

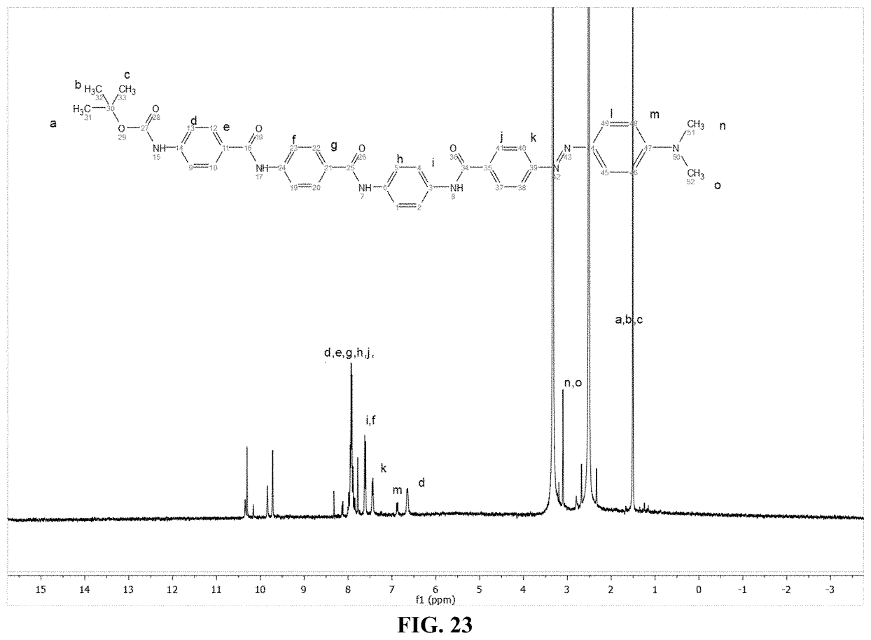

FIGS. 22-23 show ¹H NMR spectra of compound 13 and ¹H NMR spectra of compound 14.

Stability in Air:

The stability of the nanostructures in air was further verified through scanning electron microscopy (SEM). The visualization of topographical features by this method demonstrated the maintenance of the three-dimensional structure of the assembled amphiphiles after water was removed.

### Example

Compound 3, a Boc-terminated, three aramid-containing amphiphile with a zwitterionic head group was added to deionized water (3 mg amphiphile/mL water), and the pH was adjusted to 7 using 10 mM sodium hydroxide and 10 mM hydrochloric acid solutions. A 15 μL droplet of this solution was pipetted onto an SEM stub, and the water was evaporated at room temperature for 48 hours. The stub was then directly analyzed by SEM.

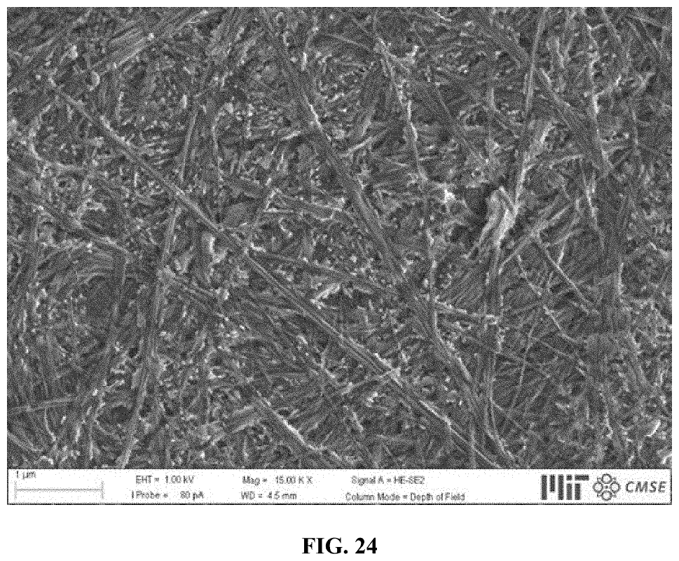

FIG. 24 shows SEM micrograph of compound 3 after assembly in water followed by the evaporation of water.

The aramid amphiphile assemblies can be modified to include arbitrary surface functionalization for engineering applications.

As a proof of principle, we demonstrated the ability of our aramid amphiphile nanostructures to be arbitrarily functionalized by incorporating chelators at the surface for removal of heavy metals from drinking water, and also by incorporation of peptides for biological applications.

Protocol for synthesis of aramid amphiphiles with metal-coordinating chelating groups

Chelating agents: The structural feature of the compounds is that they can contain ethylenedinitrilo-tetraacetic acid, lipoic acid, 3-hydroxy-N-methyl-2-pyridinone, and catechol acetonide moiety in their molecules.

In the process of preparation, our aramid amphiphiles are reacted with amidation coupling agents, and the obtained products are acidified to give pure product, which can be used in water purification for accelerating the coordination of heavy metals including Pb, Cd, Hg, Al, Sb, As, or other heavy metals. The heavy metal can be a heavy metal ion.

The heavy metal chelating agents are expressed in diethylenetriamine-tetra-tert-butyl acetate acetic acid.

Materials:

Methyl 4-aminobenzoate, 3,3-dimethylbutyric acid, 1,4-phenylenediamine, diethylenetriamine-tetra-tert-butyl acetate acetic acid, dicyclohexylcarbodiimide, 1-ethyl-3-(3-dimethylaminopropyl)carbodiimide hydrochloride, 4-dimethylaminopyridine, 1-[bis(dimethylamino)methylene]-1H-1,2,3-triazolo[4,5-b]pyridinium 3-oxide hexafluorophosphate, N-[(dimethylamino)-1H-1,2,3-triazolo-[4,5-b]pyridin-1-ylmethylene]-N-methylmethanaminium hexafluorophosphate N-oxide, N,N-diisopropylethylamine, lithium hydroxide, trifluoroacetic acid, 1,2-ethanedithiol, triisopropylsilane, hydrochloric acid, methylene chloride, dimethylformamide, tetrahydrofuran, ethanol, diethyl ether, acetonitrile, and methanol.

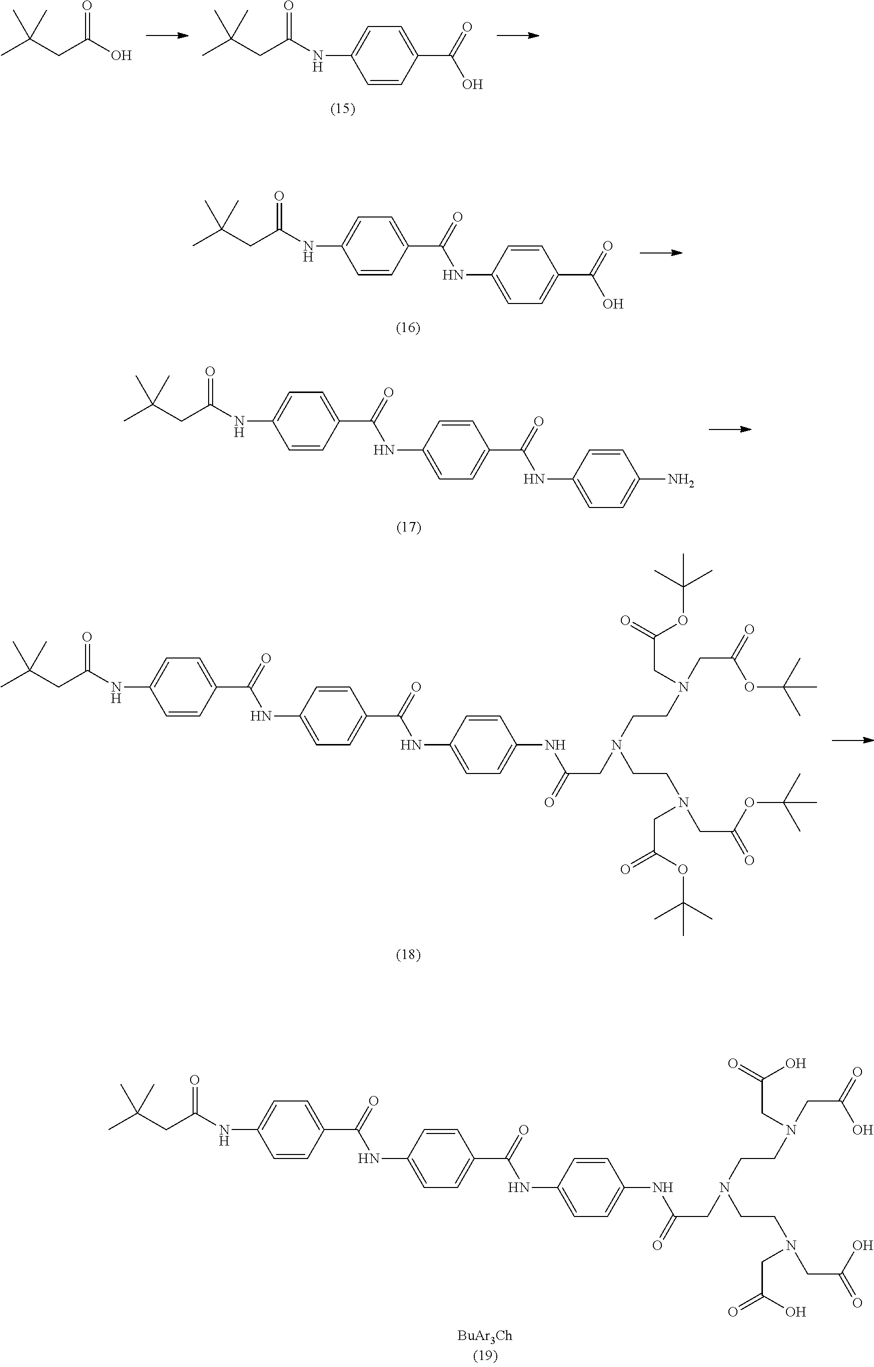

Synthesis:

Synthesis of compound 15: The solution of 3,3-dimethylbutyric acid (1.92 g, 16.5 mmol), methyl 4-aminobenzoate (1.5 g, 11.01 mmol), 1-ethyl-3-(3-dimethylaminopropyl)carbodiimide hydrochloride (6.32 g, 33.0 mmol), and 4-dimethylaminopyridine (4.46 g, 33.0 mmol) in 80 mL tetrahydrofuran was stirred at room temperature for 24 h. After the reaction, the solvent was removed in vacuum. The crude mixture was washed with water and extracted with chloroform. The organic layer was separated and distilled off. The solid material was dissolved in 40 mL tetrahydrofuran and 20 mL ethanol. To this was added lithium hydroxide (1.3 g, 54.28 mmol) in 15 mL water and refluxed at 65° C. for 3 h. The reaction mixture was shifted to room temperature and acidified to pH 2 with the addition of 5M hydrochloric acid solution. The precipitate was filtered thorough Buchner funnel, washed with water several times, and dried in vacuum to obtain the pure product as a white solid. Yield: 94%. ¹H NMR (400 MHz, DMSO-d): δ=7.87 (d, 2H), 7.72 (d, 2H), 2.23 (s, 2H), 1.03 (s, 9H) ppm.

Synthesis of compound 16: The solution of compound 15 (0.5 g, 2.13 mmol), methyl 4-aminobenzoate (0.96 g, 6.37 mmol), 1-ethyl-3-(3-dimethylaminopropyl)carbodiimide hydrochloride (1.22 g, 6.37 mmol), and 4-dimethylaminopyridine (0.77 g, 6.37 mmol) in 25 mL dimethylformamide was stirred at 50° C. for 24 h. After the reaction, the solvent was removed in vacuum and the remaining residue was precipitated with water. The mixture was filtered off and washed with methanol several times. The solid material was dissolved in 20 mL tetrahydrofuran and 10 mL ethanol. To this was added lithium hydroxide (1.15 g, 48.01 mmol) in 10 mL water and refluxed at 70° C. for 12 h. The reaction mixture was shifted to room temperature and acidified to pH 2 with the addition of 5M hydrochloric acid solution. The precipitate was filtered thorough Buchner funnel, washed with water several times, and dried in vacuum to obtain the pure product as a white solid. Yield: 88%. ¹H NMR (400 MHz, DMSO-d): δ=7.93 (m, 4H), 7.76 (d, 2H), 2.24 (s, 2H), 1.04 (s, 9H) ppm.

Synthesis of compound 17: The solution of compound 16 (0.3 g, 0.84 mmol), 1,4-phenylenediamine (1.36 g, 12.6 mmol), 1-ethyl-3-(3-dimethylaminopropyl)carbodiimide hydrochloride (0.49 g, 2.55 mmol), and 4-dimethylaminopyridine (0.34 g, 2.55 mmol) in 50 mL dimethylformamide was stirred at 25° C. for 12 h. After the reaction, the solvent was removed in vacuum and the remaining residue was precipitated with water. The crude mixture was collected with filter flask. The filtered solid was washed with copious amount of methanol, and dried in vacuum. Yield: 64%. ¹H NMR (400 MHz, DMSO-d): δ=7.96 (m, 6H), 7.77 (d, 2H), 7.64 (d, 2H), 7.41 (d, 2H), 2.25 (s, 2H), 1.05 (s, 9H) ppm.

Synthesis of compound 18: Into 10 mL dimethylformamide, compound 17 (0.13 g, 0.29 mmol), diethylenetriamine-tetra-tert-butyl acetate acetic acid (0.34 g, 0.58 mmol), dicyclohexylcarbodiimide (0.12 g, 0.58 mmol), and 4-dimethylaminopyridine (0.14 g, 1.17 mmol) were added. The well dissolved solution was stirred at room temperature for 48 h. After solvent evaporation, the crude mixture was washed with water. It was purified by column chromatography with silica gel by using tetrahydrofuran/chloroform=7:1 to give the corresponding desired white solid compound. Yield: 71%. ¹H NMR (400 MHz, DMSO-d): δ=7.95 (m, 6H), 7.77 (d, 2H), 7.70 (d, 2H), 7.64 (d, 2H), 3.40 (s, 8H), 3.24 (s, 2H), 2.76 (s, 4H), 2.65 (s, 4H), 2.25 (s, 2H), 1.40 (s, 36H), 1.05 (s, 9H) ppm.

Synthesis of compound BuAr₃Ch (19): The compound 18 (0.09 g, 0.08 mmol) was dissolved in 5 mL methylene chloride. At 25° C., the 5 mL trifluoroacetic acid was slowly injected via a syringe and stirred for 48 h. The volatile was removed under reduced pressure and 50 mL diethyl ether was added. The resulting precipitate was filtered and washed with water to obtain the pure product as a brown solid. Yield: 95%. ¹H NMR (400 MHz, DMSO-d): δ=7.97 (m, 6H), 7.75 (m, 4H), 7.60 (d, 2H), 3.46 (m, 12H), 2.91 (m, 6H), 2.25 (s, 2H), 1.05 (s, 9H) ppm. ¹³C NMR (400 MHz, DMSO-d): δ=170.9, 165.6, 162.3, 158.4, 142.9, 129.2, 128.8, 121.2, 119.8, 118.7, 65.3, 56.4, 55.3, 50.1, 31.7, 30.1 ppm.

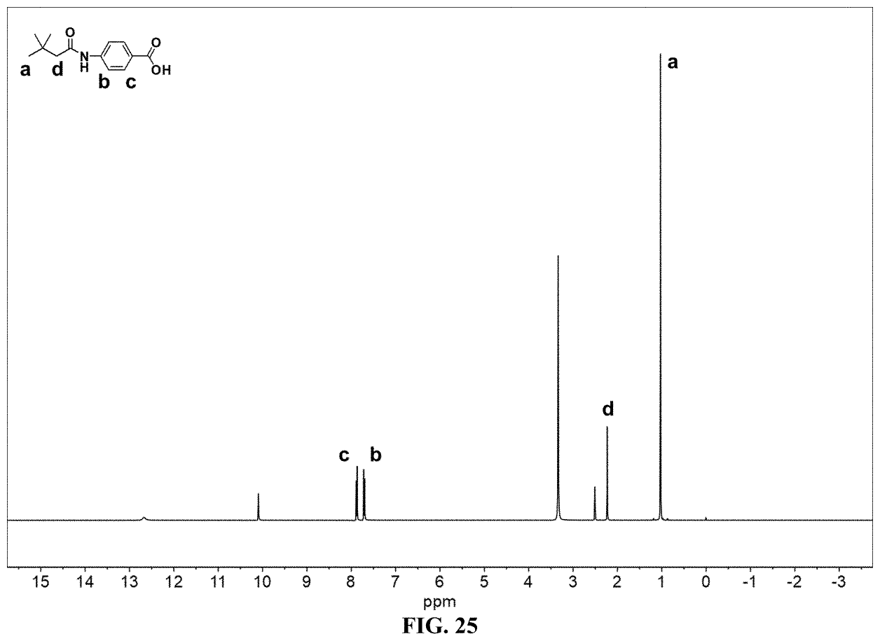

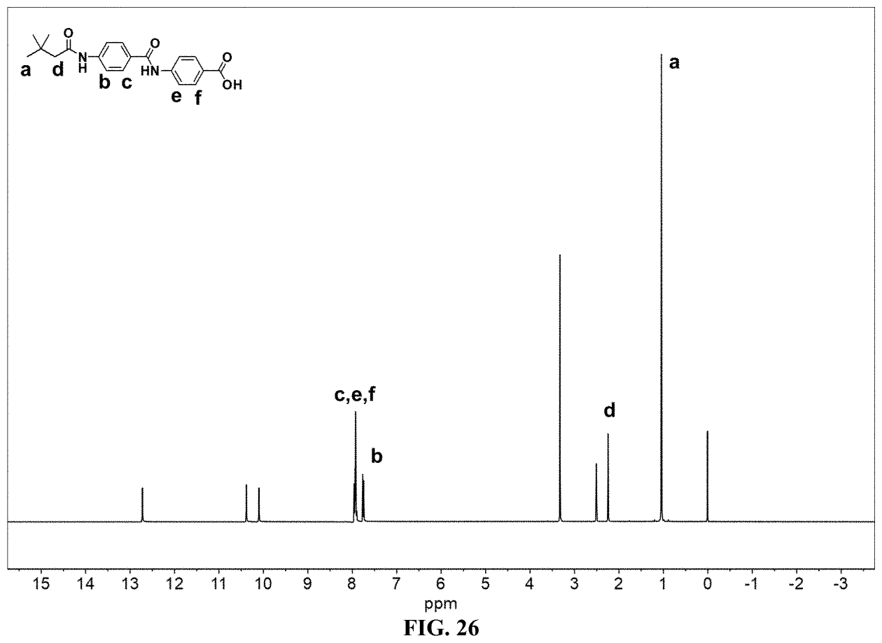

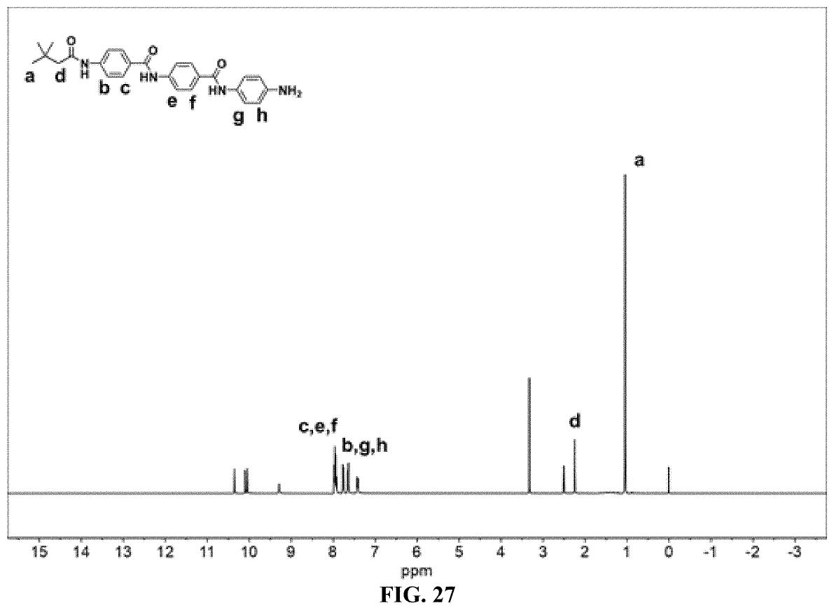

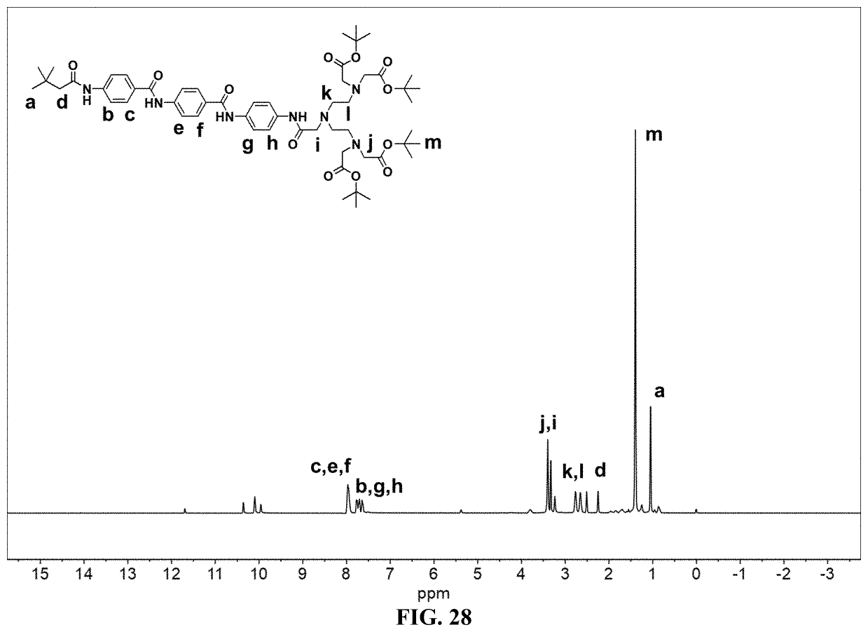

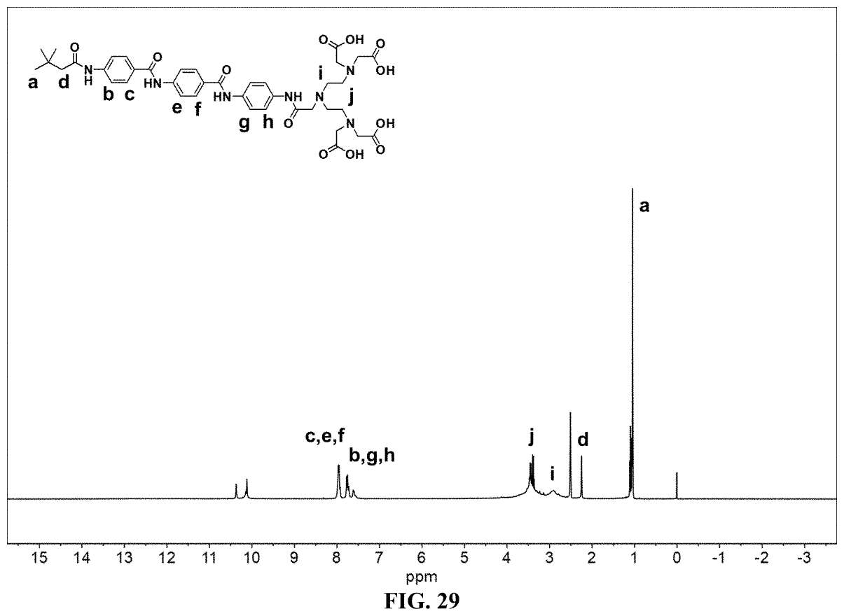

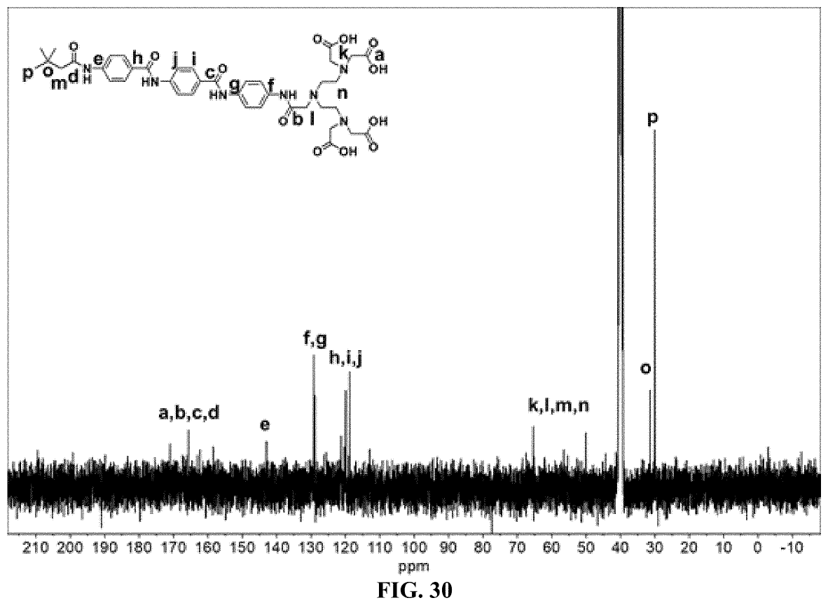

FIGS. 25-31 show ¹H NMR spectra of compound 15, ¹H NMR spectra of compound 16, ¹H NMR spectra of compound 17, ¹H NMR spectra of compound 18, ¹H NMR spectra of compound 19, ¹³C NMR spectra of compound 19, and a TEM micrograph of the assembly of compound 19.

Protocol for Synthesis of Aramid Amphiphiles with Peptide Head-Groups

To synthesize aramid amphiphiles with peptide head-groups, the peptide is first synthesized on an H-Rink Amide Resin (0.5 meq/g) using solid state peptide synthesis protocols which are standard in the literature. Rather than cleaving the peptide from the resin, the resin is swelled in DMF. A solution of 150 mM HATU (1-[Bis(dimethylamino)methylene]-1H-1,2,3-triazolo[4,5-b]pyridinium 3-oxide hexafluorophosphate, N-[(Dimethylamino)-1H-1,2,3-triazolo-[4,5-b]pyridin-1-ylmethylene]-N-methylmethanaminium hexafluorophosphate N-oxide) is prepared in DMF, and a volume containing 5 meq is used to dissolve 5 meq of the amphiphile, which is added to the swelled resin. Finally, the solution is spiked with 15 meq of DIPEA (N,N-Diisopropylethylamine) and the mixture is stored overnight at 70° C. Then, the resin is washed 3× with DMF and 3× with DCM to remove all old reagents. The reaction is then repeated by the addition of a freshly prepared, identical reaction cocktail to the re-swelled resin, and is allowed to proceed for 4 hours at 70° C. before washing with DMF and DCM.

The washed resin is dried under vacuum for 2 hours, before the resin is cleaved using a cocktail comprised of 94% TFA, 2.5% water, 2.5% 1,2-Ethanedithiol (EDT), and 1% triisopropylsilane (TIPS) at room temperature for 2 hours. The resin is then removed by filtration, and the product is collected by TFA evaporation and precipitation in diethyl ether (−80° C.). After centrifugation (4 min, 4,000 RPM), the precipitate is washed 3× with diethyl ether and subsequently dried under vacuum. The product may then be separated from any residual impurities using reverse-phase high performance liquid chromatography (HPLC) using a water/acetonitrile solvent system.

This synthesis was performed by coupling a two-ring linker to a sequence of 11 proline residues at the 0.1 mmol scale (200 mg resin). The reaction scheme is presented below, and liquid-chromatography electrospray ionization mass spectrometry (LC-ESI-MS) data collected using a C3 column are shown below, demonstrating the synthesis of the desired molecule. Expected [MH⁺] peaks: 1323.6651, 1324.6728, 1325.6807, 1326.6885, and 1327.6963; observed [MH⁺] peaks: 1323.68, 1324.68, 1325.67, 1326.68, and 1327.68. The [MH²⁺] peaks also show good agreement with theory.

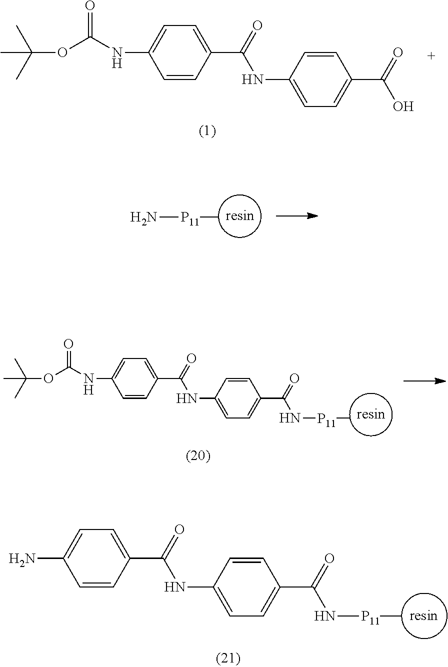

Synthesis of compound 20: The peptide is first synthesized on an H-Rink Amide Resin (0.5 meq/g) using solid state peptide synthesis protocols. Rather than cleaving the peptide from the resin, the resin is swelled in dimethylformamide. A solution of 150 mM 1-[Bis(dimethylamino)methylene]-1H-1,2,3-triazolo[4,5-b]pyridinium 3-oxide hexafluorophosphate, N-[(dimethylamino)-1H-1,2,3-triazolo-[4,5-b]pyridin-1-ylmethylene]-N-methylmethanaminium hexafluorophosphate N-oxide is prepared in dimethylformamide, and a volume containing 5 meq is used to dissolve 5 meq of the compound 1, which is added to the swelled resin. Finally, the solution is spiked with 15 meq of N,N-diisopropylethylamine and the mixture is stored overnight at 70° C. Then, the resin is washed with methylene chloride to remove all old reagents. The reaction is then repeated by the addition of a freshly prepared, identical reaction cocktail to the re-swelled resin, and is allowed to proceed for 4 hours at 70° C. before washing.

Synthesis of compound 21: The washed resin is dried under vacuum for 2 h, before the resin is cleaved using a cocktail comprised of 94% trifluoroacetic acid, 2.5% water, 2.5% 1,2-ethanedithiol, and 1% triisopropylsilane at room temperature for 2 h. The resin is then removed by filtration, and the product is collected by evaporation of volatile, and precipitation in diethyl ether. After centrifugation (4 min, 4,000 RPM), the precipitate is washed with diethyl ether and subsequently dried under vacuum. The product may then be separated from any residual impurities using reverse-phase high performance liquid chromatography using a water/acetonitrile solvent system. The reaction is performed by coupling to a sequence of 11 proline residues at the 0.1 mmol scale (200 mg resin). Expected [MH⁺] peaks: 1323.6651, 1324.67, 1325.68, 1326.68, and 1327.69; observed [MH⁺] peaks: 1323.68, 1324.68, 1325.67, 1326.68, and 1327.68.

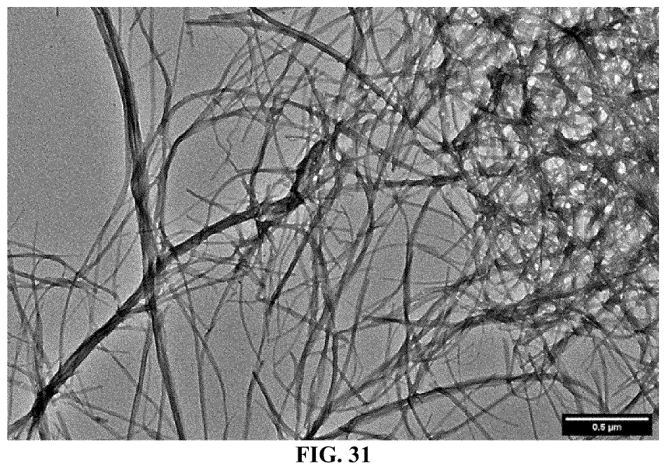

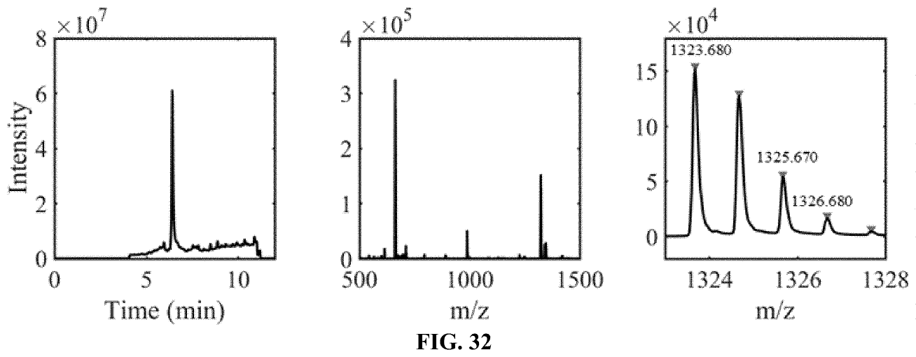

FIG. 32 shows the elution profile (left) and mass spectrogram (center/right) of the compound 21.

Supplementary Methods

Compounds 1 and 2 were purchased from Sigma-Aldrich Chemical Co. Compounds 6 and 7, and catalysts C1 and C2 were synthesized according to literature. See, for example, Bryden, F.; Boyle, R. W., A Mild, Facile. Synlett. 2013, 24, 1978 and Panagiotopoulos, A.; Ladomenou, K.; Sun, D.; Artero, V.; Coutsolelos, A. G. Dalton Trans. 2016, 45, 6732, each of which is incorporated by reference in its entirety.

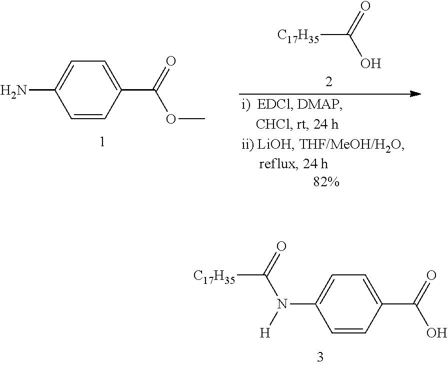

Compound 3. To CHCl₃ (150 mL), a mixture of compounds 1 (0.50 g, 3.3 mmol), 2 (0.94 g, 3.3 mmol), 1-Ethyl-3-(3-dimethylaminopropyl)carbodiimide hydrochloride (EDCI, 0.78 g, 4.0 mmol) and 4-(Dimethylamino)pyridine (DMAP, 0.49 g, 4.0 mmol) was added and stirred at room temperature for 24 hours. The formed precipitate was filtrated, washed by CHCl₃ (20 mL), and dried under vacuum to give a white solid. Then the obtained solid was suspended into mixture of THF/MeOH/H₂O (4:2:1) solution, the LiOH.H₂O (0.72 g, 17.0 mmol) was added and reaction system was stirred at reflux for 24 hours. After the solvent was removed by vacuum, the obtained solid was washed by water (20 mL), then by 1 M HCl aqueous solution (20 mL) and water (20 mL). The product was dried under vacuum for 24 hours to afford compound 3 as a white solid (1.09 g, 82%). M.p.>250° C. (decomp). ¹H NMR (400 MHz, DMSO-d₆): δ 10.09 (s, 1H), 7.84 (d, J=8.4 Hz, 2H), 7.65 (d, J=8.0 Hz, 2H), 2.32 (t, J=7.3 Hz, 2H), 1.58 (quint, J=6.7 Hz, 2H), 1.31-1.25 (m, 28H), 0.85 (t, J=6.5 Hz, 3H). ¹³C NMR (100 MHz, DMSO-d₆): δ 171.92, 167.00, 143.39, 130.39, 124.90, 118.27, 36.52, 31.33, 29.05, 28.91, 28.77, 28.73, 28.63, 25.00, 22.13, 13.99. MS (ESI): m/z 404.3 [M+H]⁺. HRMS (ESI): Calcd for C₂₅H₄₂NO₃ [M+H]⁺: 404.3165. Found: 404.3156.

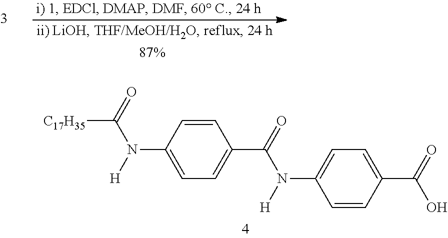

Compound 4 was prepared in 87% yield as a white solid from the reaction of compounds 3 and 1 according to a procedure as described for compound 3. M.p.>250° C. (decomp). ¹H NMR (400 MHz, DMSO-d₆): δ 10.11 (s, 1H), 9.96 (s, 1H), 7.95-7.71 (m, 8H), 2.34 (t, J=7.7 Hz, 2H), 1.63 (quint, J=6.7 Hz, 2H), 1.31-1.25 (m, 28H), 0.86 (t, J=6.3 Hz, 3H). ¹³C NMR (100 MHz, DMSO-d₆): δ 171.22, 166.34, 164.77, 142.89, 142.14, 129.46, 128.35, 128.03, 125.39, 119.15, 118.05, 36.09, 30.67, 28.39, 28.29, 28.16, 28.12, 28.07, 24.44, 21.38, 13.12. MS (ESI): m/z 523.4 [M+H]t HRMS (ESI): Calcd for C₃₂H₄₆N₂O₄ [M+H]⁺: 523.3536. Found: 523.3548.

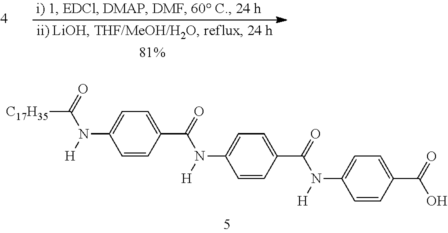

Compound 5 was prepared in 81% yield as a white solid from the reaction of compounds 4 and 1 according to a procedure as described for compound 3. M.p.>250° C. (decomp). ¹H NMR (400 MHz, DMSO-d₆): δ 10.15 (s, 1H), 10.12 (s, 1H), 9.88 (s, 1H), 8.05-7.85 (m, 10H), 7.72 (d, J=8.8 Hz, 2H), 2.35 (t, J=7.4 Hz, 2H), 1.63 (quint, J=6.6 Hz, 2H), 1.36-1.26 (m, 28H), 0.86 (t, J=6.9 Hz, 3H). ¹³C NMR (100 MHz, DMSO-d₆): δ 171.44, 166.48, 164.98, 164.91, 143.00, 142.23, 142.19, 129.63, 128.95, 128.48, 128.14, 128.05, 125.40, 119.33, 119.32, 118.23, 36.20, 30.77, 28.48, 28.38, 28.25, 28.20, 28.12, 24.55, 21.49, 13.24. MS (ESI): m/z 642.4 [M+H]t HRMS (ESI): Calcd for C₃₉H₅₂N₃O₅ [M+H]⁺: 642.3907. Found: 642.3889.

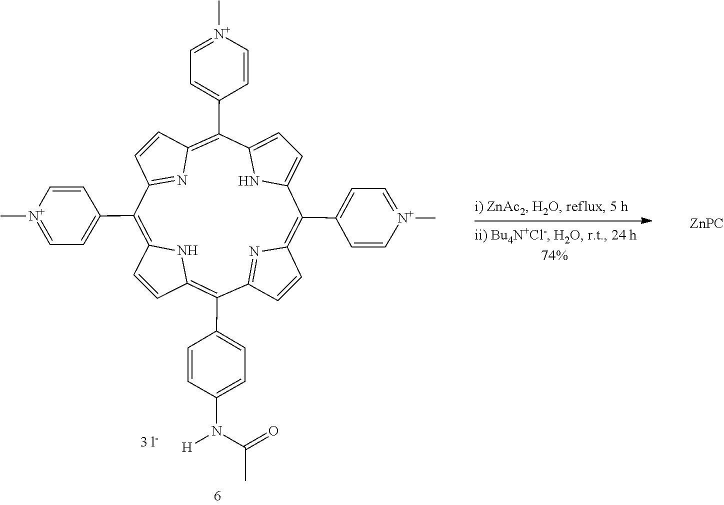

Compound ZnPC. To a 20 mL pressure vessel, the compound 6 (100 mg, 0.091 mmol) was dissolved into water (5 mL) and zinc acetate (69 mg, 0.45 mmol) was added, the system was stirred at reflux for 5 hours and then added tetrabutylammonium chloride (252 mg, 0.91 mmol) to exchange the anions. The mixture was stirred at room temperature for 24 hours, and then solvent was evaporated under reduced pressure until the red solid precipitate was formed and the resulting green solid was further recrystallized from water to give compound ZnPC as a dark green solid (60 mg, 74%). M.p.>300° C. (decomp). ¹H NMR (400 MHz, DMSO-d₆): δ 10.40 (s, 1H), 9.40 (d, J=6.6 Hz, 6H), 9.12-8.87 (m, 14H), 8.11 (d, J=8.5 Hz, 2H), 8.05 (d, J=8.5 Hz, 2H), 4.71 (s, 9H), 2.23 (s, 3H). ¹³C NMR (100 MHz, DMSO-d₆): δ 169.32, 158.64, 150.94, 148.80, 148.58, 148.28, 144.14, 139.73, 136.67, 135.06, 134.03, 133.00, 132.57, 132.14, 123.92, 117.68, 116.05, 115.17, 48.29, 24.72. MS (MALDI): m/z 781.2 [M]⁺. HRMS (ESI): Calcd for C₆₂H₇₁N₈O [M]³⁺: 260.4122. Found: 260.4130.

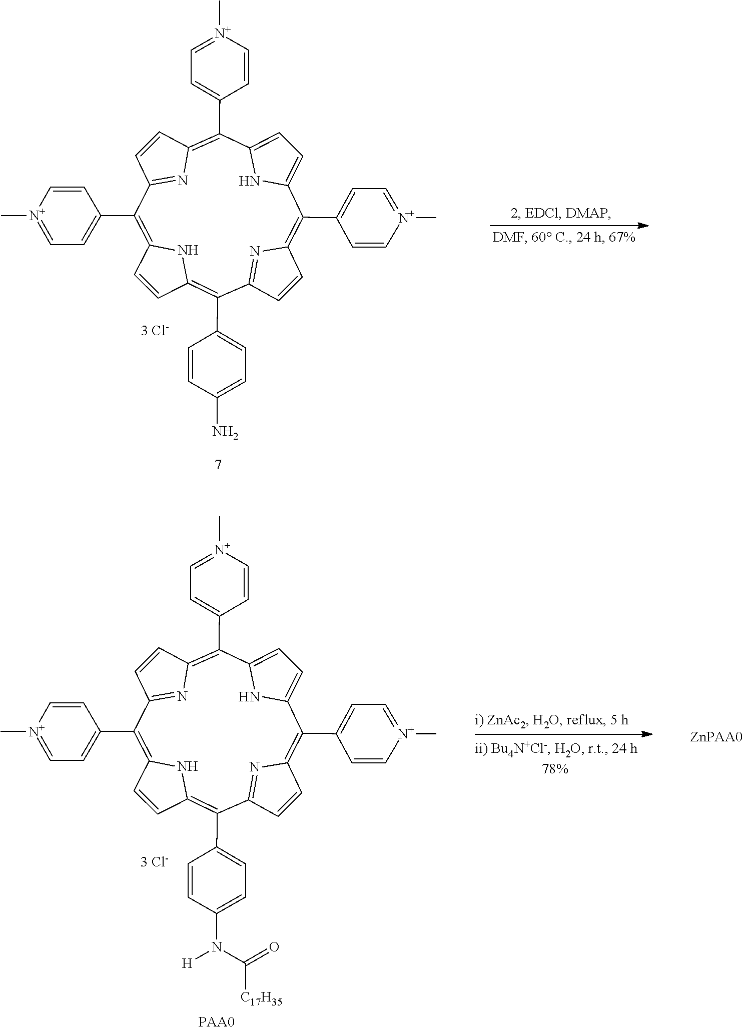

Compound PAA0. A mixture of compound 2 (27.2 mg, 0.096 mmol), 7 (50.0 mg, 0.064 mmol), EDCI (18.3 mg, 0.096 mmol), and DMAP (13.0 mg, 0.096 mmol) was stirred in DMF (5 mL) at 60° C. for 24 hours, after cooling to room temperature the solvent was removed under reduced pressure. The result red solid was washed by CHCl₃ (1 mL) three times to remove the excess EDCI and DMAP. The precipitate was isolated and the crude product was purified using flash chromatography (MeOH:MeCN:H₂O 8:1:1). The obtained fractions were evaporated to dryness to give product PAA0 as a red solid (44 mg, 67%). M.p.>300° C. (decomp). ¹H NMR (400 MHz, DMSO-d₆): δ 10.38 (s, 1H), 9.47 (d, J=6.4 Hz, 6H), 9.18-8.92 (m, 14H), 8.15 (d, J=8.7 Hz, 2H), 8.11 (d, J=8.9 Hz, 2H), 4.71 (s, 9H), 1.73 (quint, J=7.1 Hz, 2H), 1.30 (m, 30H), 0.80 (t, J=6.9 Hz, 3H), −3.00 (s, 2H). ¹³C NMR (100 MHz, DMSO-d₆): δ 171.88, 156.57, 144.15, 139.74, 134.81, 132.07, 122.90, 117.51, 115.24, 114.39, 47.94, 36.65, 31.22, 29.02, 28.95, 28.84, 28.73, 28.63, 25.24, 22.02, 13.90. MS (ESI): m/z 943.6 [M]⁺. HRMS (ESI): Calcd for C₆₂H₇₁N₈O [M]³⁺: 314.5245. Found: 314.5255. Compound ZnPAA0 was prepared in 78% yield as a dark green solid from the reaction of PAA0 with ZnAc₂ according to a procedure as described for compound 7. M.p.>300° C. (decomp). MS (MALDI): m/z 1005.5 [M]⁺. HRMS (ESI): Calcd for C₆₂H₆₉N₈OZn [M]³⁺: 335.1623. Found: 335.1630. Elemental analysis calcd for C₆₂H₆₉N₈OZn (%): C, 75.41; H, 5.01; N, 10.99. Found: C, 74.87; H, 5.07; N, 10.53.

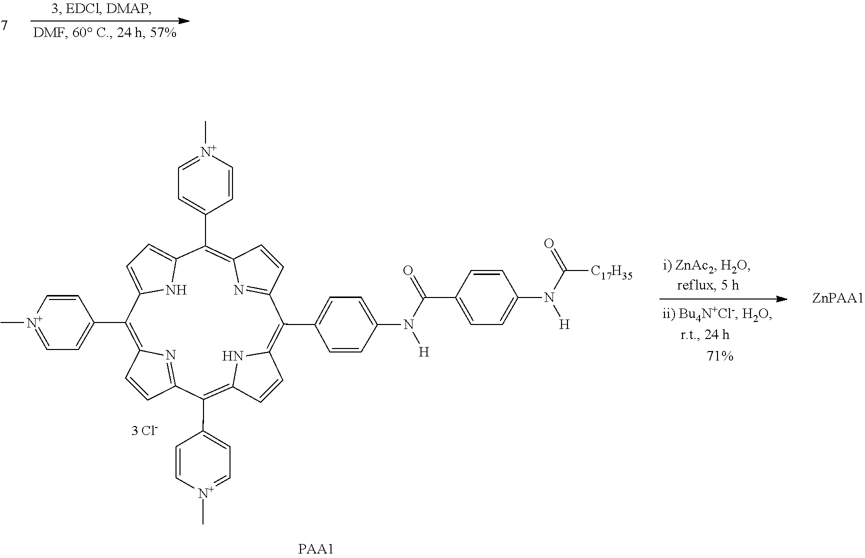

Compound PAA1 was prepared in 57% yield as a red solid from the reaction of compound 7 and 3 according to a procedure as described for compound PAA0. M.p.>300° C. (decomp). ¹H NMR (400 MHz, DMSO-d₆): δ 10.62 (s, 1H), 10.23 (s, 1H), 9.47 (d, J=6.5 Hz, 6H), 9.20-8.96 (m, 14H), 8.32 (d, J=8.4 Hz, 2H), 8.21 (d, J=8.5 Hz, 2H), 8.09 (d, J=8.6 Hz, 2H), 7.84 (d, J=8.8 Hz, 2H), 4.72 (s, 9H), 2.39 (t, J=7.4 Hz, 2H), 1.63 (quint, J=6.7 Hz, 2H), 1.32-1.24 (m, 28H), 0.84 (t, J=7.1 Hz, 3H), −2.98 (s, 2H). ¹³C NMR (100 MHz, DMSO-d₆): δ 172.32, 165.96, 157.10, 156.99, 144.67, 143.05, 140.34, 135.68, 135.24, 132.60, 129.67, 129.43, 129.27, 123.42, 119.15, 118.79, 115.77, 114.91, 48.41, 36.99, 31.77, 29.52, 29.40, 29.27, 29.18, 29.14, 25.48, 22.57, 14.43. Compound ZnPAA1 was prepared in 71% yield as a dark green solid from the reaction of PAA1 with ZnAc₂ according to a procedure as described for compound 7. M.p.>300° C. (decomp). MS (MALDI): m/z 1062.6 [M]⁺. HRMS (ESI): Calcd for C₆₉H₇₆N₉O₂ [M]³⁺: 354.2035. Found: 354.2034. MS (MALDI): m/z 1124.5 [M]⁺. HRMS (ESI): Calcd for C₆₉H₇₄N₉O₂Zn [M]³⁺: 374.8413. Found: 374.8425. Elemental analysis calcd for C₆₉H₇₄N₉O₂Zn (%): C, 73.55; H, 6.62; N, 11.19. Found: C, 73.21; H, 6.79; N, 11.25.

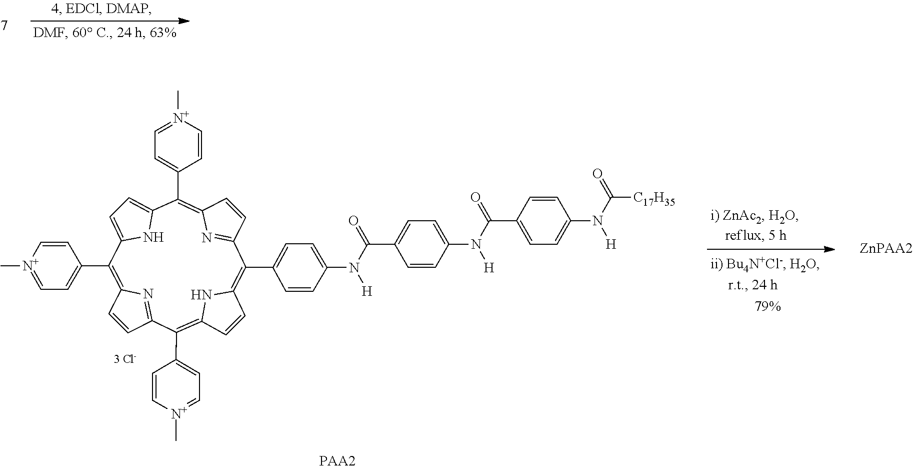

Compound PAA2 was prepared in 63% yield as a red solid from the reaction of compound 7 and 4 according to a procedure as described for compound PAA0. M.p.>300° C. (decomp). ¹H NMR (400 MHz, DMSO-d₆): δ 10.68 (s, 1H), 10.46 (s, 1H), 10.21 (s, 1H), 9.49 (d, J=6.5 Hz, 6H), 9.22-8.97 (m, 14H), 8.35 (d, J=8.4 Hz, 2H), 8.23 (d, J=8.4 Hz, 2H), 8.15 (d, J=8.7 Hz, 2H), 8.06 (d, J=8.7 Hz, 2H), 8.01 (d, J=8.7 Hz, 2H), 7.79 (d, J=8.5 Hz, 2H), 4.73 (s, 9H), 2.37 (t, J=7.3 Hz, 2H), 1.62 (quint, J=6.7 Hz, 2H), 1.25-1.22 (m, 27H), 0.87 (t, J=7.1 Hz, 3H), −2.97 (s, 2H). ¹³C NMR (100 MHz, DMSO-d₆): δ 171.86, 165.57, 165.26, 156.63, 156.54, 144.19, 142.65, 139.88, 135.23, 134.79, 132.14, 129.46, 128.80, 128.66, 128.53, 122.97, 119.56, 118.69, 118.23, 115.30, 114.43, 47.92, 36.51, 31.30, 29.04, 28.92, 28.78, 28.71, 28.65, 28.52, 24.98, 22.10, 13.96. Compound ZnPAA2 was prepared in 79% yield as a dark green solid from the reaction of PAA2 with ZnAc₂ according to a procedure as described for compound 7. M.p.>300° C. (decomp). MS (MALDI): m/z 1181.6 [M]⁺. HRMS (ESI): Calcd for C₇₆H₈₁N₁₀O₃ [M]³⁺: 393.8826. Found: 393.8840. MS (MALDI): m/z 1243.6 [M]⁺. HRMS (ESI): Calcd for C₇₆H₇₉N₁₀O₃Zn [M]³⁺: 414.5204. Found: 414.5216. Elemental analysis calcd for C₆₉H₇₄N₉O₂Zn (%): C, 73.27; H, 6.39; N, 11.24. Found: C, 73.45; H, 6.48; N, 11.28.

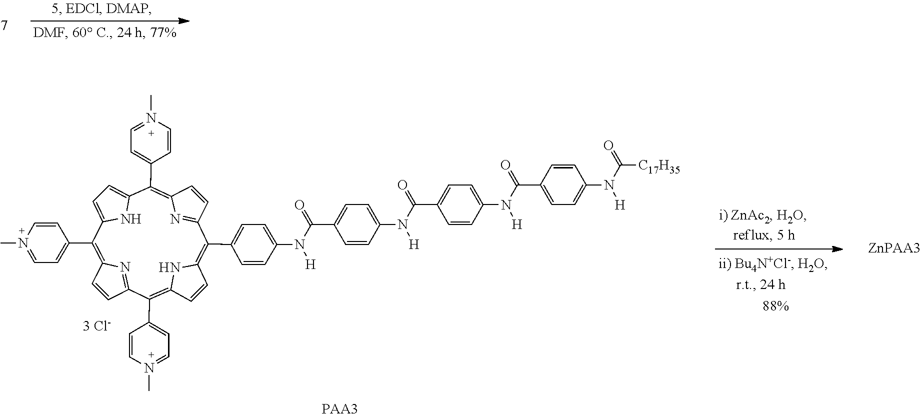

Compound PAA3 was prepared in 77% yield as a red solid from the reaction of compound 7 and 5 according to a procedure as described for compound PAA0. M.p.>300° C. (decomp). ¹H NMR (400 MHz, DMSO-d₆): δ 10.67 (s, 1H), 10.49 (s, 1H), 10.41 (s, 1H), 10.18 (s, 1H), 9.48 (d, J=6.5 Hz, 6H), 9.22-8.94 (m, 14H), 8.35 (d, J=8.6 Hz, 2H), 8.23 (d, J=8.5 Hz, 2H), 8.15 (d, J=8.7 Hz, 2H), 8.08-8.05 (m, 4H), 8.02-7.96 (m, 4H), 7.77 (d, J=8.6 Hz, 2H), 4.72 (s, 9H), 2.36 (t, J=7.4 Hz, 2H), 1.61 (quint, J=6.7 Hz, 2H), 1.27 (m, 28H), 0.85 (t, J=6.7 Hz, 3H), −2.98 (s, 2H). ¹³C NMR (100 MHz, DMSO-d₆): δ 171.81, 165.55, 165.28, 165.20, 156.62, 156.53, 144.18, 142.60, 139.87, 135.20, 134.78, 132.12, 129.48, 128.99, 128.76, 128.66, 128.64, 128.49, 122.96, 119.54, 119.40, 118.66, 118.18, 115.28, 114.41, 47.90, 36.48, 31.28, 29.02, 28.99, 28.89, 28.76, 28.69, 28.63, 24.96, 22.08, 13.95. Compound ZnPAA3 was prepared in 88% yield as a dark green solid from the reaction of PAA3 with ZnAc₂ according to a procedure as described for compound 7. M.p.>300° C. (decomp). MS (MALDI): m/z 1300.7 [M]⁺. FIRMS (ESI): Calcd for C₈₃H₈₆N₁₁O₄ [M]³⁺: 433.5616. Found: 433.5628. MS (MALDI): m/z 1362.6 [M]⁺. HRMS (ESI): Calcd for C₈₃H₈₄N₁₁O₄Zn [M]³⁺: 454.1994. Found: 454.1994. Elemental analysis calcd for C₆₉H₇₄N₉O₂Zn (%): C, 73.03; H, 6.20; N, 11.29. Found: C, 73.05; H, 6.28; N, 11.31.

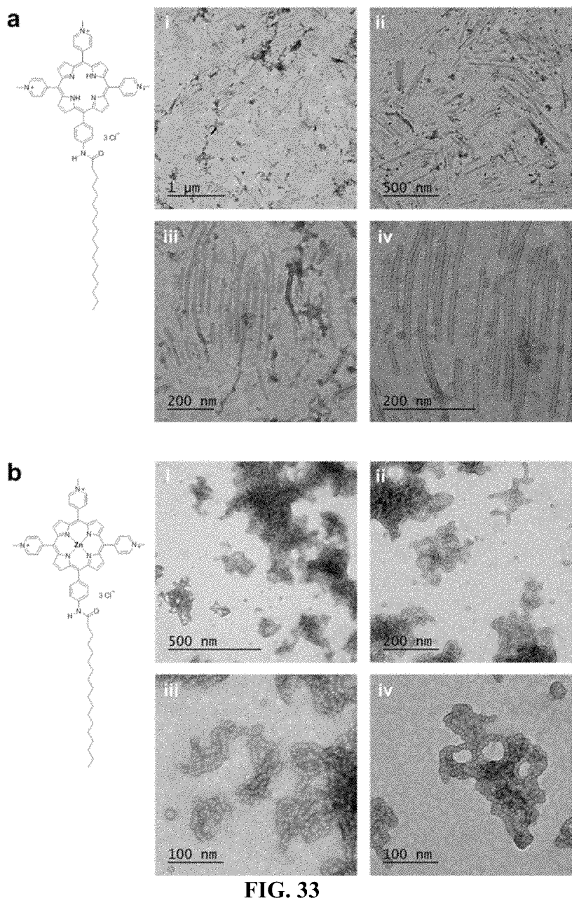

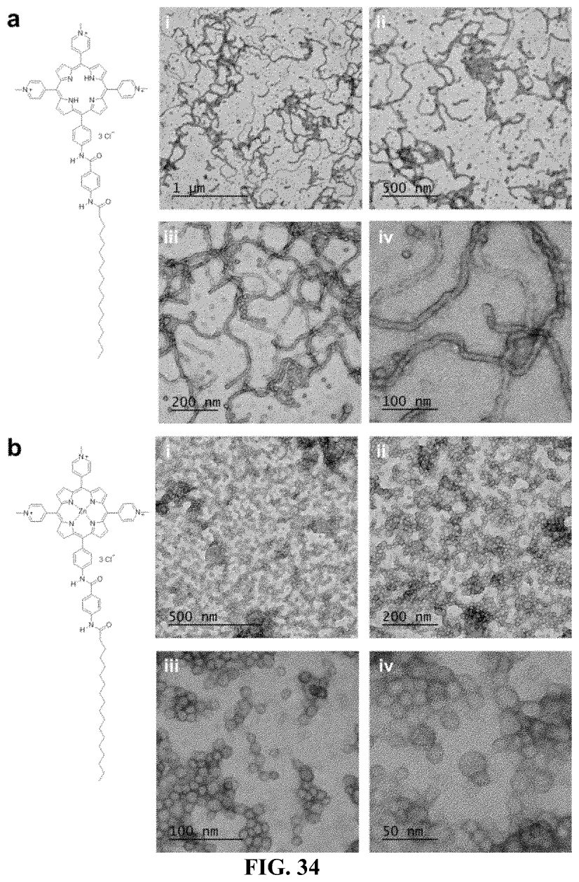

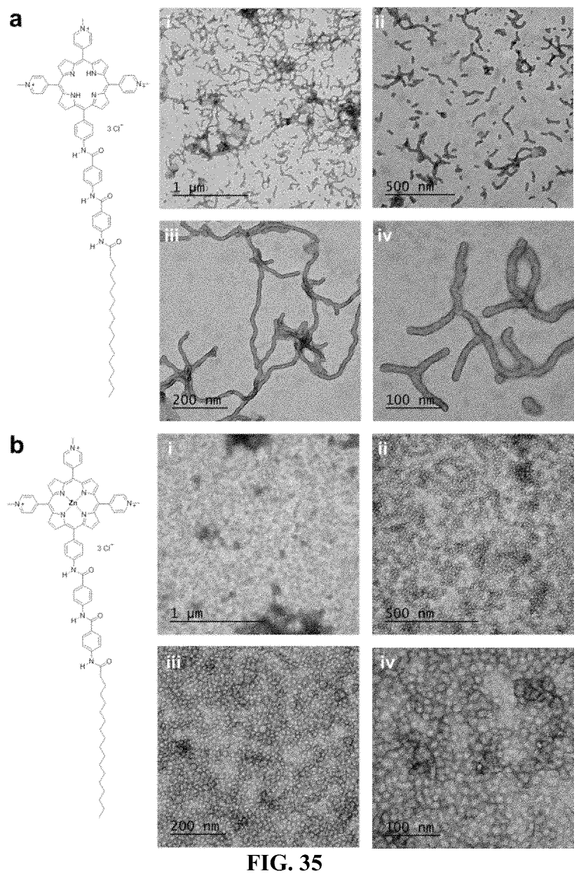

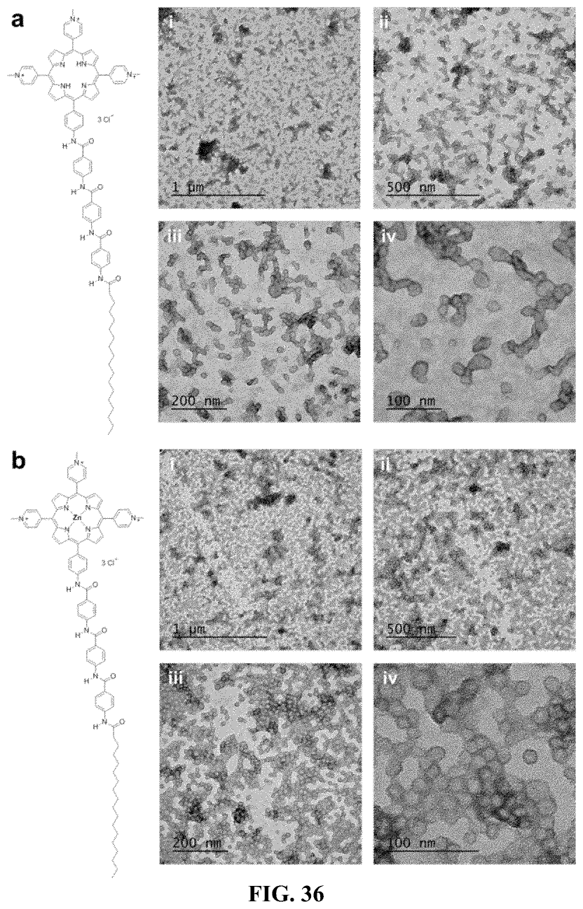

FIGS. 33A-49 show negative staining TEM images of compound (a) PAA0 and (b) ZnPAA0 assemblies in water, negative staining TEM images of compound (a) PAA1 and (b) ZnPAA1 assemblies in water, negative staining TEM images of compound (a) PAA2 and (b) ZnPAA2 assemblies in water, negative staining TEM images of compound (a) PAA4 and (b) ZnPAA4 assemblies in water, synchrotron SAXS profiles of (a) ZnPAA0, (b) ZnPAA1, (c) ZnPAA2, and (d) ZnPAA3 in water with [Zn]=5 mM at room temperature. (Black dots: experimental SAXS data; red line: corresponding fit by core-shell model by Irena 2.63, synchrotron SAXS profiles of (a) ZnPAA0, (b) ZnPAA1, (c) ZnPAA2, and (d) ZnPAA3 in water with [Zn]=5 mM, with different irradiation time, synchrotron SAXS profiles of (a) ZnPAA0, (b) ZnPAA1, (c) ZnPAA2, and (d) ZnPAA3 in water with [Zn]=5 mM, at variable temperatures from 303 to 313K, UV-vis spectra of 0.2 mM (a) PC and ZnPC, (b) PAA0 and ZnPAA0, (c) PAA1 and ZnPAA1, (d) PAA2 and ZnPAA2, (e) PAA3 and ZnPAA3, and (f) merged images of all zinc complexes in water, at room temperature, fluorescence spectra of 0.02 mM (a) PC and ZnPC, (b) PAA0 and ZnPAA0, (c) PAA1 and ZnPAA1, (d) PAA2 and ZnPAA2, (e) PAA3 and ZnPAA3, and (f) merged images of all zinc complexes in water, at room temperature, fluorescence spectra of (a) dilution of ZnPAA3 solution, (b) titration of C2 to a 0.02 mM ZnPAA3 solution, and (c) Stern-Volmer analysis of ZnPAA3 with different concentration of C2, UV-vis spectra of 0.02 mM (a) C2, (b) ZnPC, (c) ZnPAA0, (d) ZnPAA1, (e) ZnPAA2, and (f) ZnPAA3 in water, at room temperature, cyclic voltammetry of 0.2 mM (a) C2, (b) ZnPC, (c) ZnPAA0, (d) ZnPAA1, (e) ZnPAA2, and (f) ZnPAA3 with a scan rate of 100 mV/s. Working, reference, and counter electrodes are gold, Ag/AgCl, and Pt, respectively, UV-vis spectra of 0.02 mM (a) ZnPC, (b) ZnPAA0, (c) ZnPAA1, (d) ZnPAA2, (d) ZnPAA3, at different irradiation time, and (f) plots of A/A₀ versus irradiation time for all zinc complexes in water, where A₀ is the initiate absorbance at 425 nm, GC-MS experiments during long-term irradiation of a solution containing 2 μM of catalyst C2, 20 mM of TEA and 0.2 mM of sensitizer ZnPAA3, under ¹³CO₂ or ¹²CO₂ atmosphere (a) typical gas chromatogram observed and the mass spectra of methane generated under a (b)¹³CO₂ or (c)¹²CO₂ atmosphere, gas chromatogram profiles of 0.2 mM (a) ZnPAA0, (b) ZnPAA1, (c) ZnPAA2, and (d) ZnPAA3 in water with 2 μM of catalyst C2, 20 mM of TEA, under saturated CO₂ atmosphere in water after 24 h irradiation, pictures of centrifuge tubes, which contained SPAs solution of ZnPAA3, [Zn]=0.5 mM, before and after treatment of high speed centrifugation and sonication, and cryo-TEM images of 0.2 mM ZnPAA3 assemblies after 12^{th} catalytic cycles, comparison of (b) synchrotron SAXS profiles and (c) DLS results of 0.2 mM ZnPAA3 in water, with 2 μM of catalyst C2, 20 mM of TEA.HCl, under saturated CO₂ atmosphere in water before and after 24 h irradiation.

TABLE 1
Estimated molecular orbital energy of C2, ZnPC and ZnPAAs

| | E_{g}^{o} | E_{1/2}^{re} | E_{1/2}^{ox} | HOMO | LUMO |
|---|---|---|---|---|---|
| Compound | (eV)^{a} | (V)^{b} | (V)^{b} | (eV)^{c} | (eV)^{c} |
| C2 (Co^{III/II}) | 2.90^{d} | 0.04^{e} | — | −7.44 | −4.54 |
| C2 (Co^{II/I}) | — | −0.62^{e} | — | — | −3.88 |
| ZnPC | 1.93^{d} | — | 0.66^{e} | −5.16 | −3.23 |
| ZnPAA0 | 1.91^{d} | — | 0.66^{e} | −5.16 | −3.25 |
| ZnPAA1 | 1.88^{d} | — | 0.67^{e} | −5.17 | −3.29 |
| ZnPAA2 | 1.87^{d} | — | 0.67^{e} | −5.17 | −3.30 |
| ZnPAA3 | 1.84^{d} | — | 0.68^{e} | −5.18 | −3.34 |

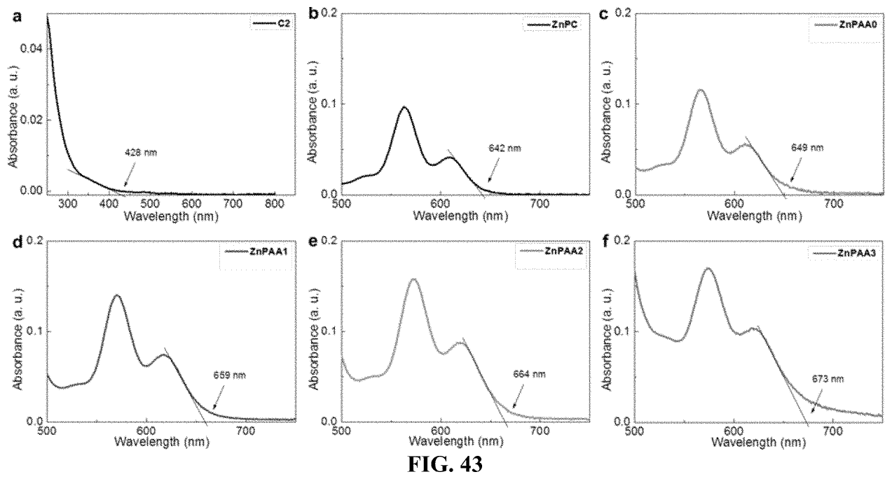

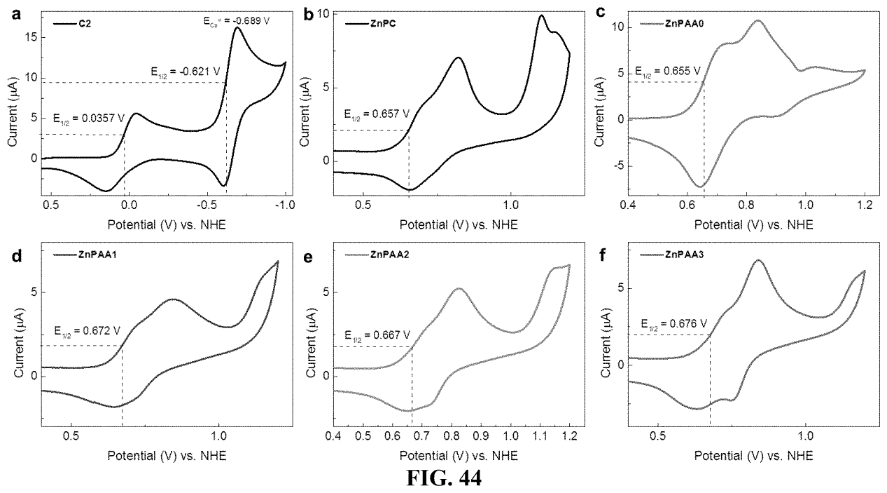

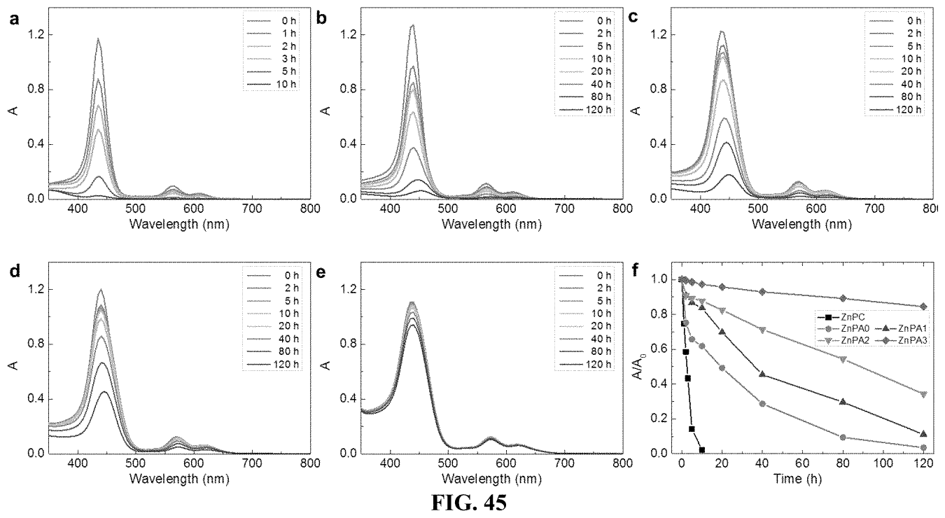

^{a}Optical band gap E_{g}^{o} = 1240/ λₒₙₛₑₜ^{abs}
^{b}Potentials versus normal hydrogen electrode (NHE).
^{c}HOMO and LUMO energies were calculated with reference to NHE (4.50 eV) LUMO = −(4.50 + E_{1/2}^{re}); HOMO = −(4.50 + E_{1/2}^{ox} ); HOMO = LUMO − E_{g}^{o}
^{d}The optical band gap estimated from the tangents of the absorption edges of their UV/Vis spectra (FIG. 45).
^{e}The reduction and oxidation potentials were obtained from the cyclic voltammetry (FIG. 44).

TABLE 2
Catalytic performance and structures of catalyst C2 and sensitizer (AA: ascorbic acid).

| | | | [Zn] | [Co] | AA | λ | Time | H2 |
|---|---|---|---|---|---|---|---|---|
| Entry | Compound | Gas | (mM) | (μM) | (mM) | (nm) | (h) | (TON) |
| 1 | ZnPC | Air | 0.2 | 2 | 20 | >400 | 20 | 34 ± 5 |
| 2 | ZnPC | Air | 0.2 | 2 (C1) | 20 | >400 | 20 | 36 ± 3 |
| 3 | ZnPAA0 | Air | 0.2 | 2 | 20 | >400 | 20 | 164 ± 19 |
| 4 | ZnPAA1 | Air | 0.2 | 2 | 20 | >400 | 20 | 188 ± 25 |
| 5 | ZnPAA2 | Air | 0.2 | 2 | 20 | >400 | 20 | 201 ± 14 |
| 6 | ZnPAA3 | Air | 0.2 | 2 | 20 | >400 | 20 | 217 ± 31 |
| 7 | ZnPAA3 | Air | 0.2 | 2 (C1) | 20 | >400 | 20 | 41 ± 4 |
| 8 | ZnPAA3 | Air | — | 2 | 20 | >400 | 20 | 3 ± 1 |
| 9 | ZnPAA3 | Air | 0.2 | — | 20 | >400 | 20 | 1 |
| 10 | ZnPAA3 | Air | 0.2 | 2 | — | >400 | 20 | — |
| 11 | ZnPAA3 | Air | 0.2 | 2 | 20 | dark | 20 | — |

TABLE 3
Catalytic performance and structures of catalyst C2 and sensitizer (SA: sodium ascorbate).

| | | | [Zn] | [Co] | SA | λ | Time | H₂ | CO |
|---|---|---|---|---|---|---|---|---|---|
| Entry | Compound | Gas | (mM) | (μM) | (mM) | (nm) | (h) | (TON) | (TON) |
| 1 | ZnPC | CO₂ | 0.2 | 2 | 20 | >400 | 96 | 29 ± 6 | 12 ± 2 |
| 2 | ZnPAA0 | CO₂ | 0.2 | 2 | 20 | >400 | 96 | — | 387 ± 25 |
| 3 | ZnPAA1 | CO₂ | 0.2 | 2 | 20 | >400 | 96 | — | 549 ± 42 |
| 4 | ZnPAA2 | CO₂ | 0.2 | 2 | 20 | >400 | 96 | — | 672 ± 28 |
| 5 | ZnPAA3 | CO₂ | 0.2 | 2 | 20 | >400 | 96 | — | 712 ± 22 |
| 6 | ZnPAA3 | CO₂ | — | 2 | 20 | >400 | 24 | — | 5 ± 2 |
| 7 | ZnPAA3 | CO₂ | 0.2 | — | 20 | >400 | 24 | — | 2 ± 2 |
| 8 | ZnPAA3 | CO₂ | 0.2 | 2 | — | >400 | 24 | — | 1 ± 1 |
| 9 | ZnPAA3 | CO₂ | 0.2 | 2 | 20 | dark | 24 | — | — |
| 10 | ZnPAA3 | Air | 0.2 | 2 | 20 | >400 | 24 | 34 ± 4 | — |
| 11 | ZnPAA3(Na₂CO₃)^{a} | Air | 0.2 | 2 | 20 | >400 | 24 | 31 ± 7 | — |
| 12 | ZnPAA3(NaHCO3)^{b} | Air | 0.2 | 2 | 20 | >400 | 24 | 37 ± 3 | — |

^{a}100 mM Na₂CO₃, ^{b}100 MM NaHCO₃.

TABLE 4
Catalytic performance and structures of catalyst C2 and sensitizer (TEA: triethylamine).

| | | | [Zn] | [Co] | TEA | λ | Time | H₂ | CO | CH4 |
|---|---|---|---|---|---|---|---|---|---|---|
| Entry | Compound | Gas | (mM) | (μM) | (mM) | (nm) | (h) | (TON) | (TON) | (TON) |
| 1 | ZnPC | CO₂ | 0.2 | 2 | 20 | >400 | 32 | 30 ± 3 | 35 ± 6 | — |
| 2 | ZnPC | CO₂ | 0.2 | 2(C1) | 20 | >400 | 32 | 17 ± 5 | 21 ± 3 | — |
| 3 | ZnPAA0 | CO₂ | 0.2 | 2 | 20 | >400 | 32 | 22 ± 4 | 44 ± 5 | 16 ± 2 |
| 4 | ZnPAA1 | CO₂ | 0.2 | 2 | 20 | >400 | 32 | 33 ± 3 | 89 ± 17 | 33 ± 5 |
| 5 | ZnPAA2 | CO₂ | 0.2 | 2 | 20 | >400 | 32 | 49 ± 5 | 83 ± 19 | 79 ± 11 |
| 6 | ZnPAA3 | CO₂ | 0.2 | 2 | 20 | >400 | 32 | 54 ± 9 | 77 ± 14 | 106 ± 17 |
| 7 | ZnPAA3 | CO₂ | 0.2 | 2 (C1) | 20 | >400 | 32 | 13 ± 3 | 33 ± 7 | 14 ± 3 |
| 8 | ZnPAA3 | CO₂ | — | 2 | 20 | >400 | 32 | — | 4 ± 1 | 1 |
| 9 | ZnPAA3 | CO₂ | 0.2 | — | 20 | >400 | 32 | — | 7 ± 3 | — |
| 10 | ZnPAA3 | CO₂ | 0.2 | 2 | — | >400 | 32 | — | 1 | — |
| 11 | ZnPAA3 | CO₂ | 0.2 | 2 | 20 | dark | 32 | — | — | — |

TABLE 5
Catalytic performance and structures of catalyst C2 and sensitizer (TEA-HCl: triethylamine hydrochloride).

| | | | [Zn] | [Co] | TEA-HCl | λ | Time | H₂ | CO | CH₄ |
|---|---|---|---|---|---|---|---|---|---|---|
| Entry | Compound | Gas | (mM) | (μM) | (mM) | (nm) | (h) | (TON) | (TON) | (TON) |
| 1 | ZnPC | CO₂ | 0.2 | 2 | 20 | >400 | 24 | 75 ± 21 | 13 ± 3 | — |
| 2 | ZnPAA0 | CO₂ | 0.2 | 2 | 20 | >400 | 24 | 19 ± 5 | 106 ± 29 | 83 ± 16 |
| 3 | ZnPAA1 | CO₂ | 0.2 | 2 | 20 | >400 | 24 | 24 ± 7 | 95 ± 21 | 96 ± 19 |
| 4 | ZnPAA2 | CO₂ | 0.2 | 2 | 20 | >400 | 24 | 21 ± 4 | 72 ± 19 | 119 ± 22 |
| 5 | ZnPAA3 | CO₂ | 0.2 | 2 | 20 | >400 | 24 | 22 ± 6 | 42 ± 15 | 129 ± 23 |
| 6 (1st run) | ZnPAA3 | CO₂ | 0.2 | 2 | 20 | >400 | 10 | 13 ± 3 | 35 ± 9 | 55 ± 11 |
| 7 (2nd run) | ZnPAA3 | CO₂ | 0.2 | 2 | 20 | >400 | 10 | 12 ± 2 | 36 ± 7 | 57 ± 12 |
| 8 (3rd run) | ZnPAA3 | CO₂ | 0.2 | 2 | 20 | >400 | 10 | 13 ± 2 | 35 ± 6 | 54 ± 10 |
| 9 (4th run) | ZnPAA3 | CO₂ | 0.2 | 2 | 20 | >400 | 10 | 13 ± 4 | 34 ± 9 | 55 ± 13 |
| 10 (5th run) | ZnPAA3 | CO₂ | 0.2 | 2 | 20 | >400 | 10 | 11 ± 2 | 35 ± 6 | 56 ± 11 |
| 11 (6th run) | ZnPAA3 | CO₂ | 0.2 | 2 | 20 | >400 | 10 | 11 ± 2 | 37 ± 7 | 53 ± 11 |
| 12 (7th run) | ZnPAA3 | CO₂ | 0.2 | 2 | 20 | >400 | 10 | 12 ± 3 | 34 ± 6 | 55 ± 12 |
| 13 (8th run) | ZnPAA3 | CO₂ | 0.2 | 2 | 20 | >400 | 10 | 10 ± 1 | 35 ± 5 | 52 ± 10 |
| 14 (9th run) | ZnPAA3 | CO₂ | 0.2 | 2 | 20 | >400 | 10 | 11 ± 3 | 36 ± 4 | 50 ± 13 |
| 15 (10th run) | ZnPAA3 | CO₂ | 0.2 | 2 | 20 | >400 | 10 | 11 ± 3 | 34 ± 4 | 50 ± 9 |
| 16 (11th run) | ZnPAA3 | CO₂ | 0.2 | 2 | 20 | >400 | 10 | 10 ± 2 | 32 ± 5 | 48 ± 5 |
| 17 (12th run) | ZnPAA3 | CO₂ | 0.2 | 2 | 20 | >400 | 10 | 10 ± 3 | 31 ± 2 | 48 ± 4 |

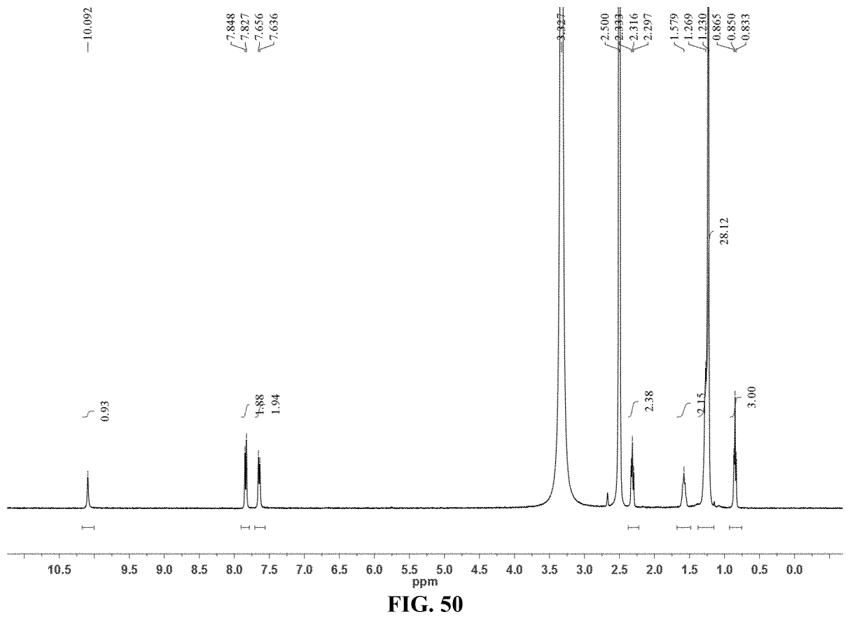

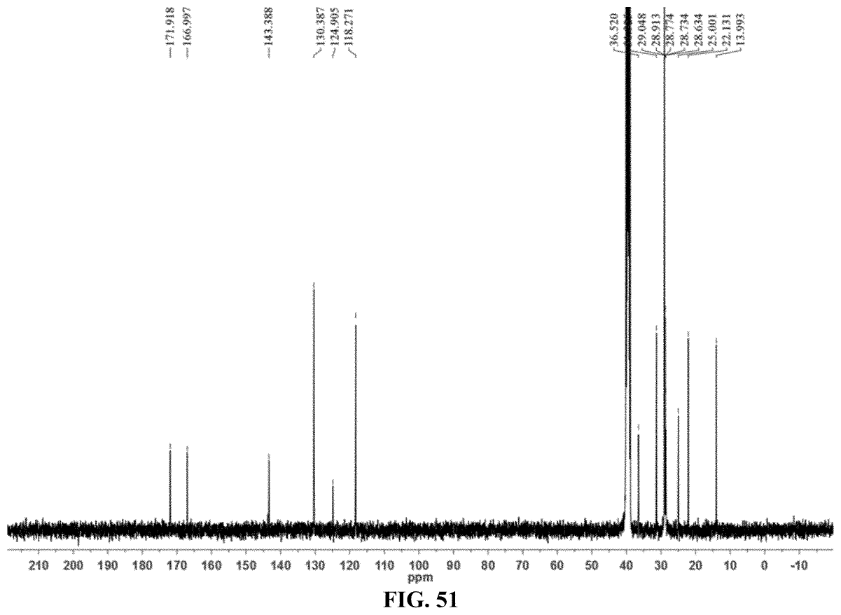

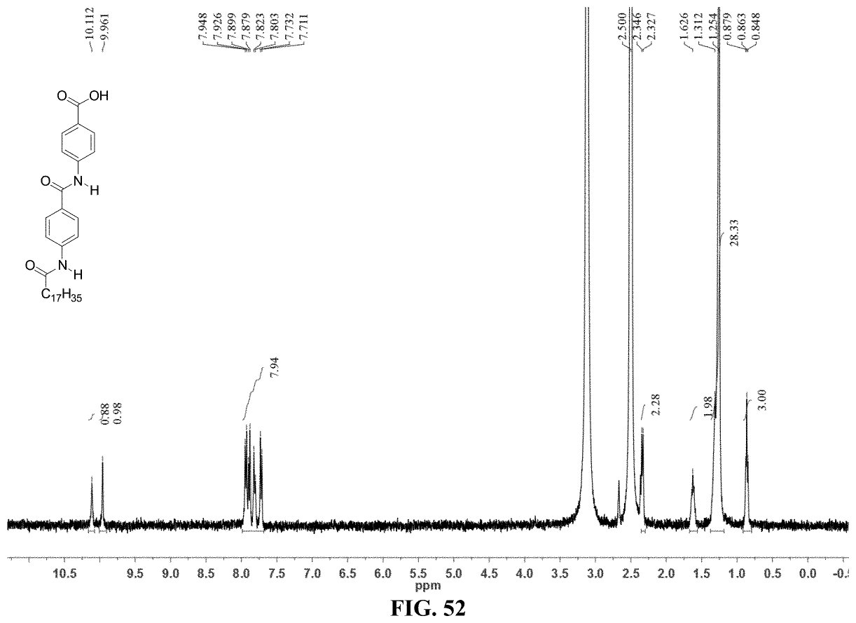

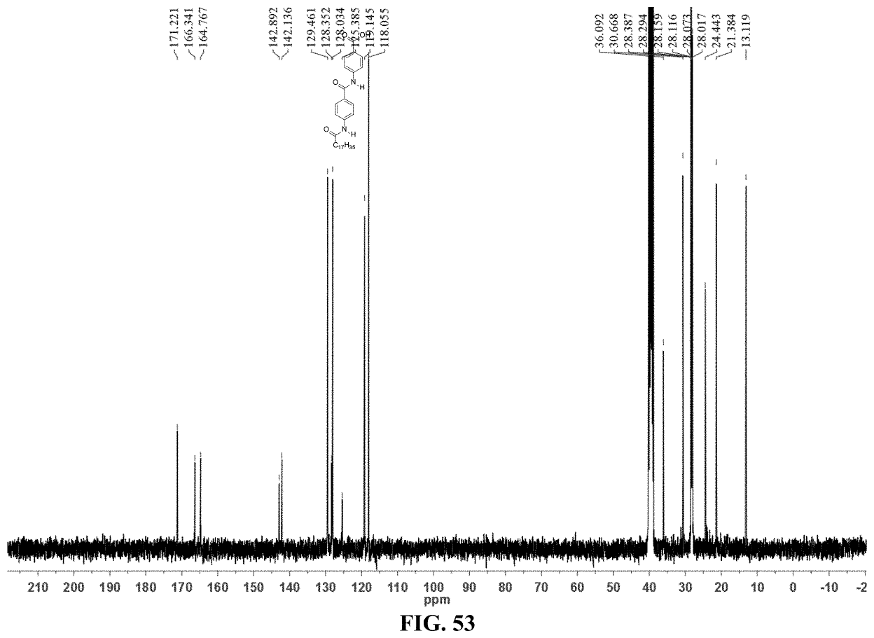

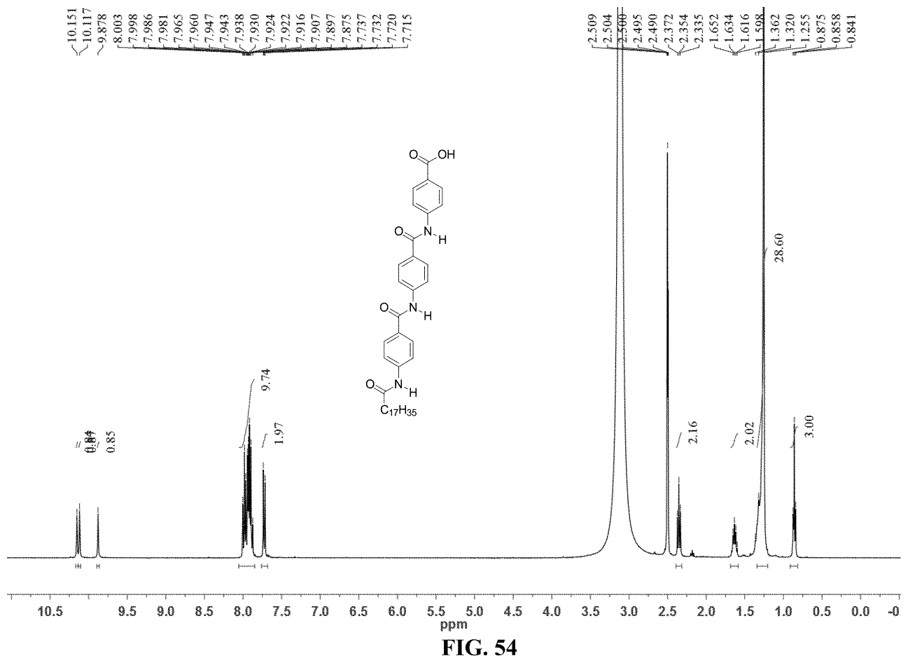

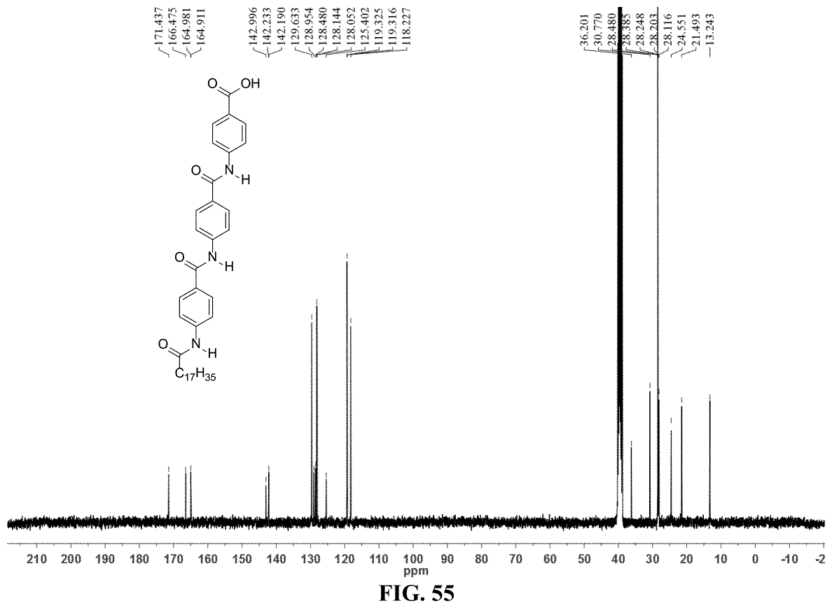

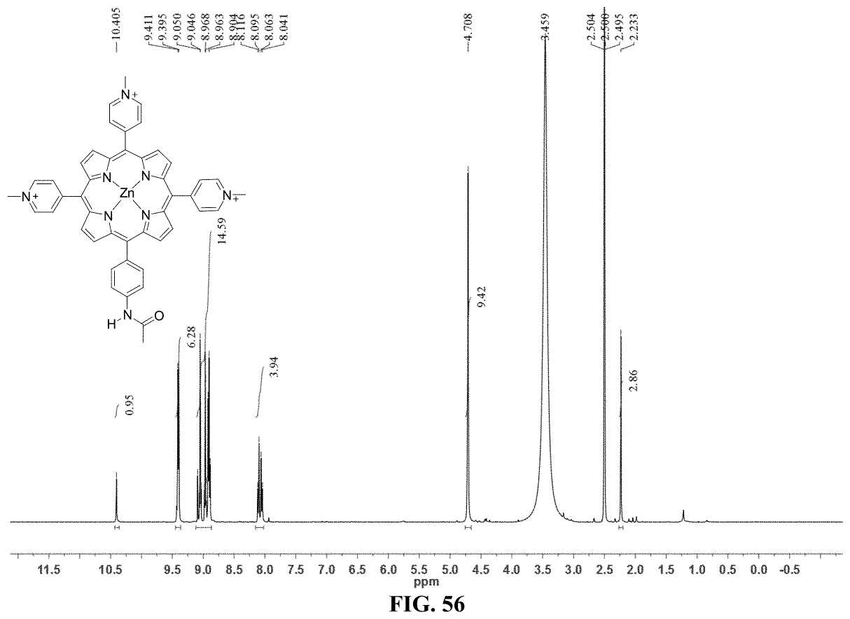

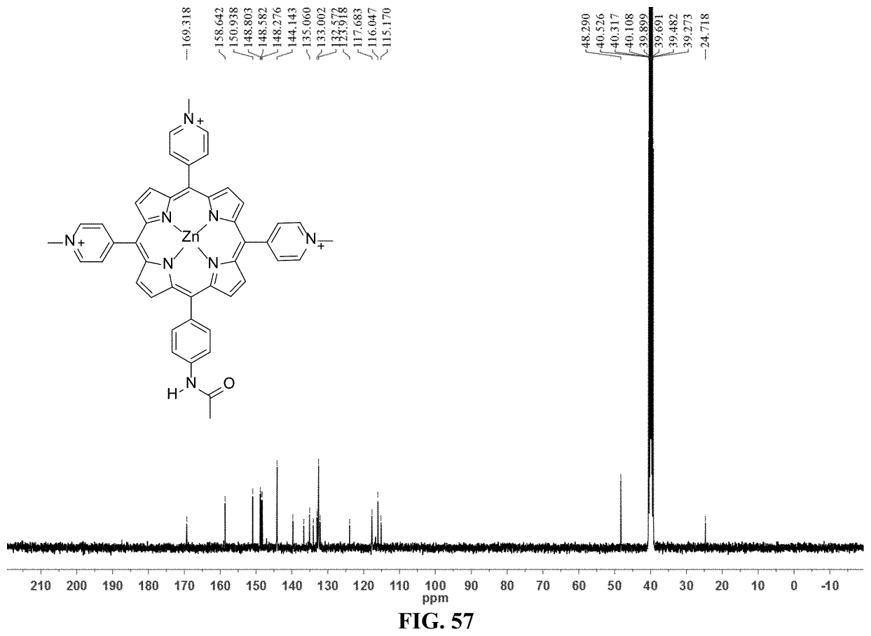

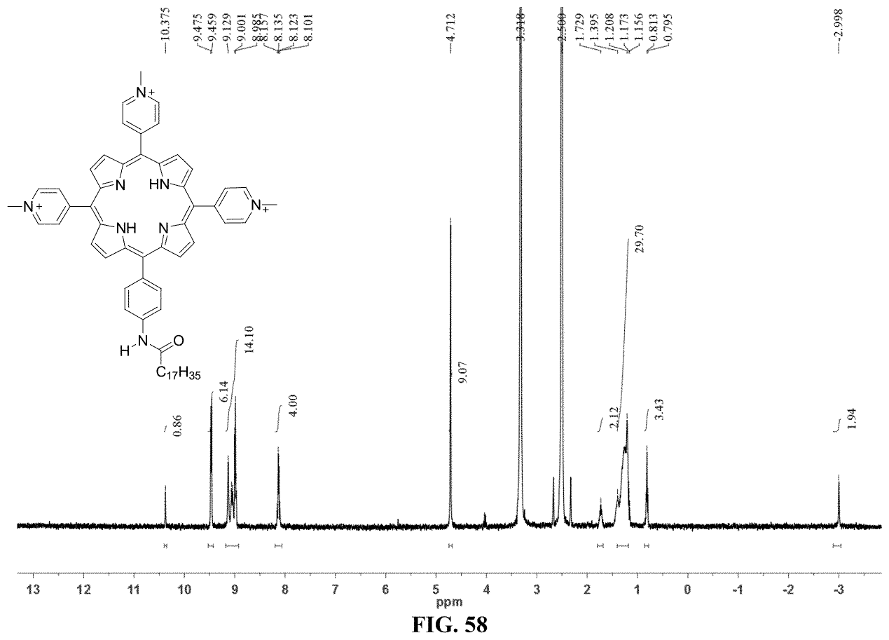

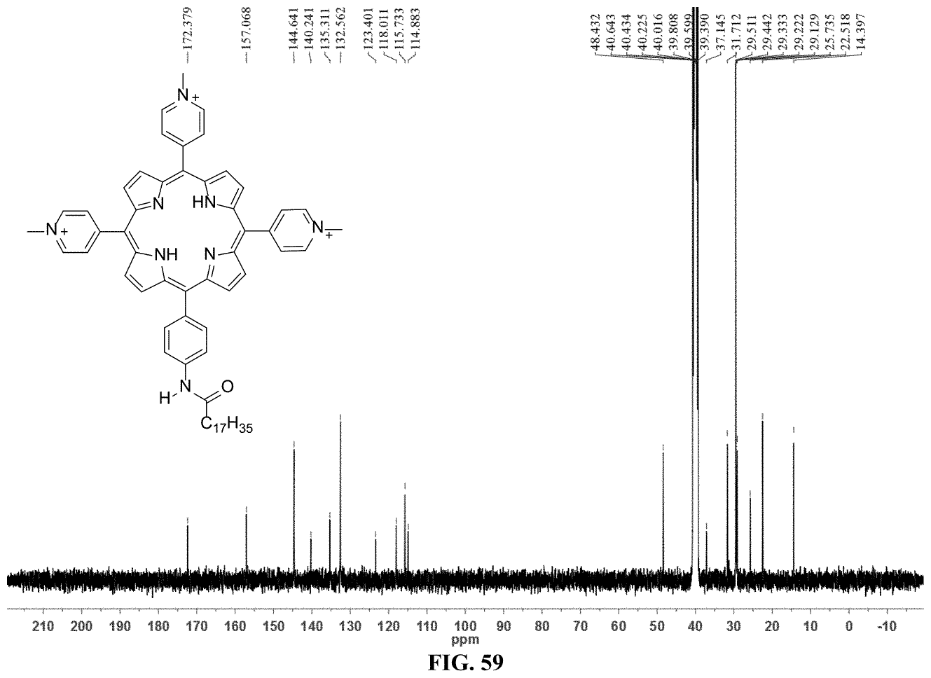

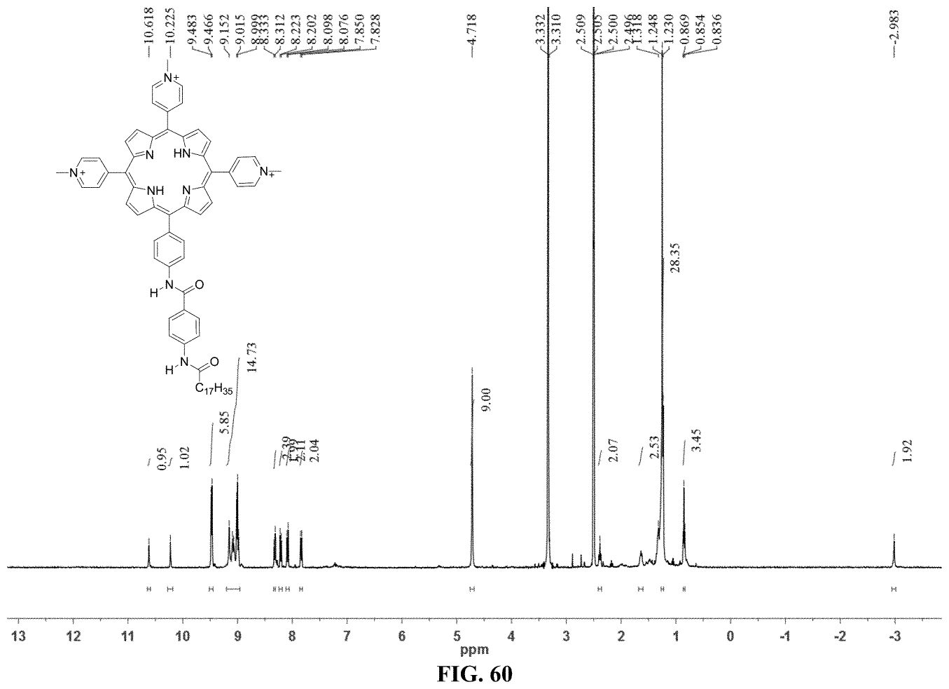

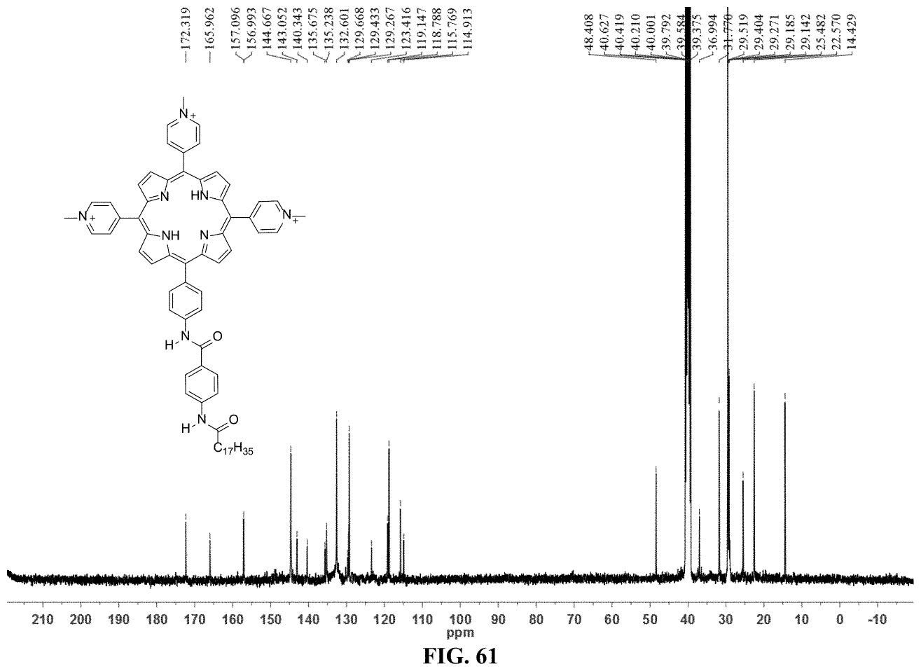

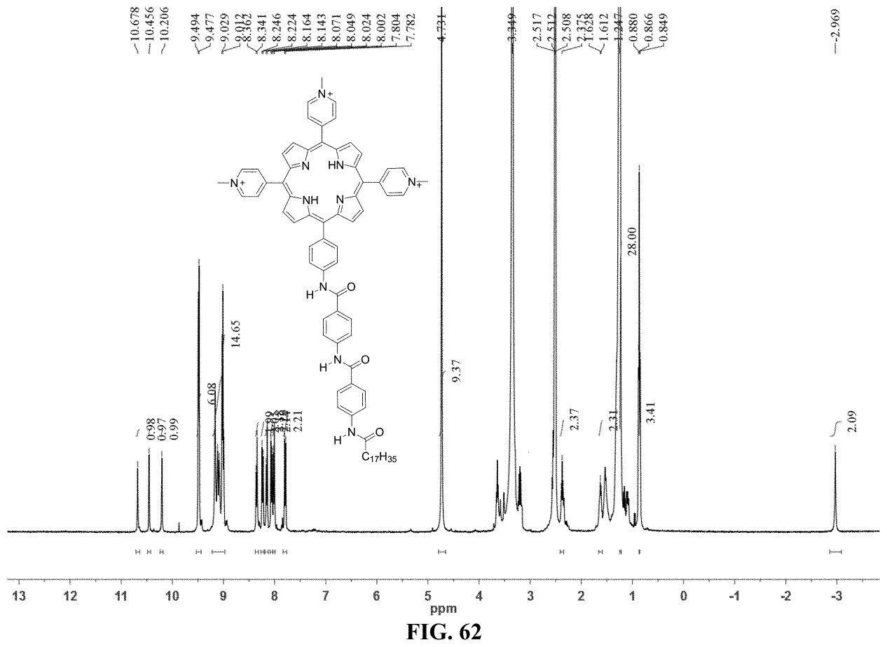

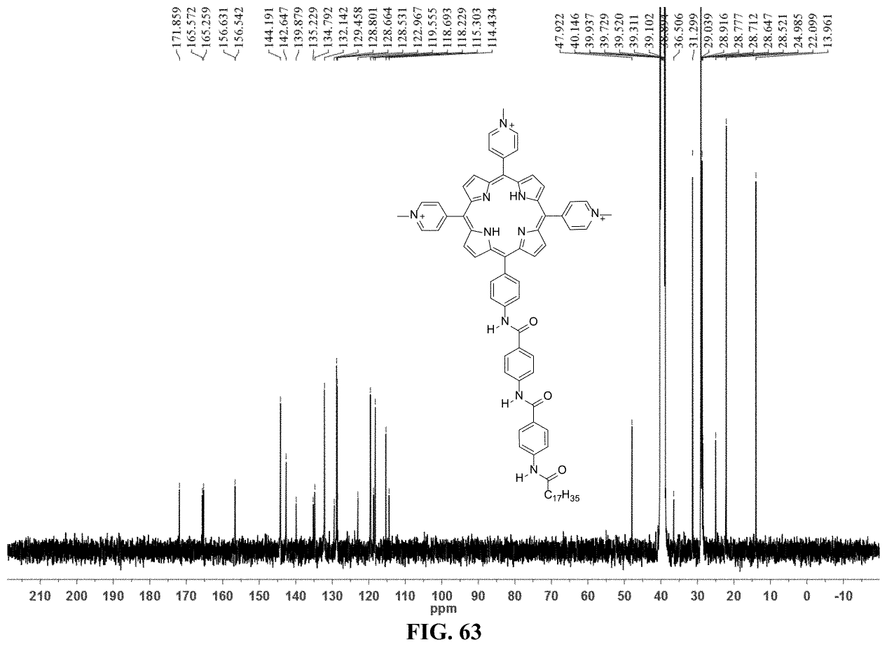

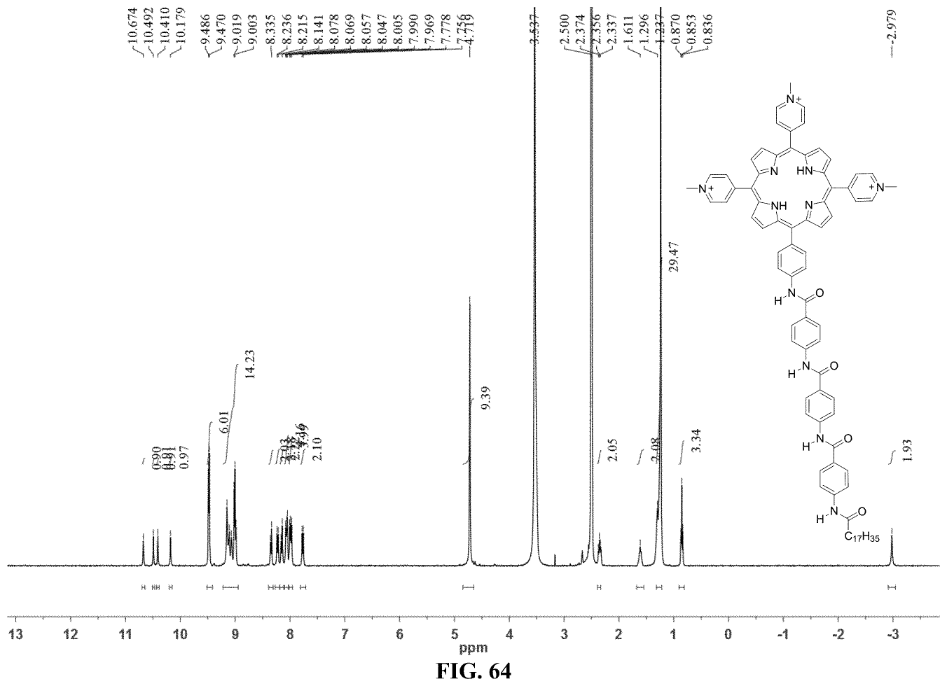

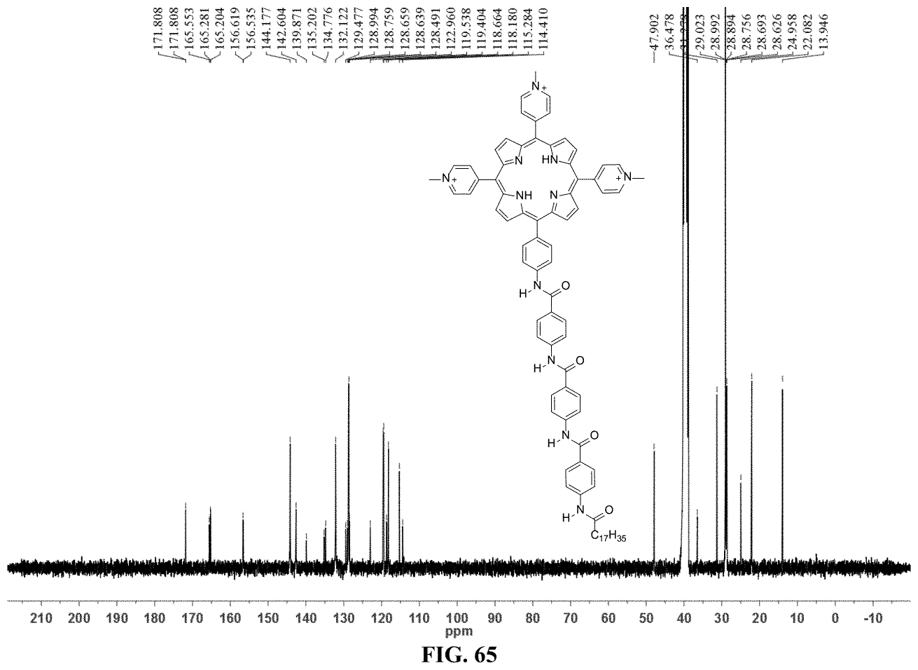

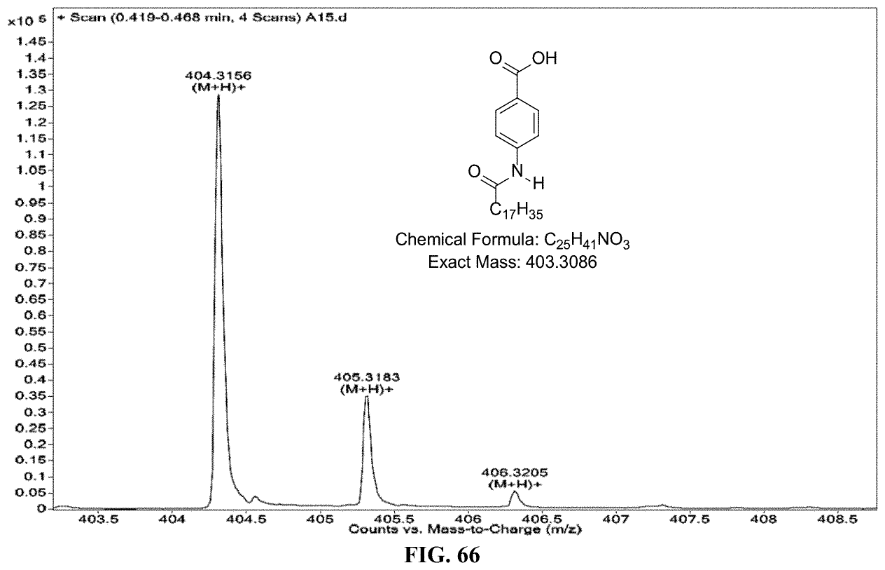

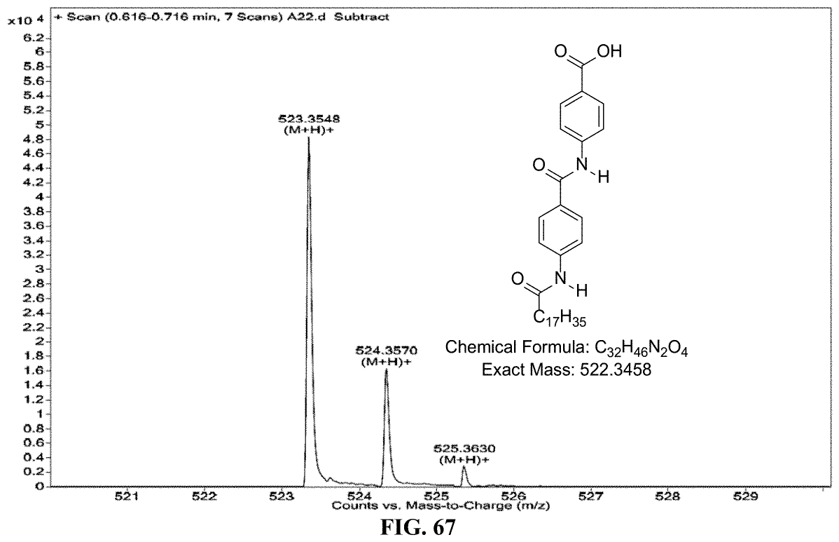

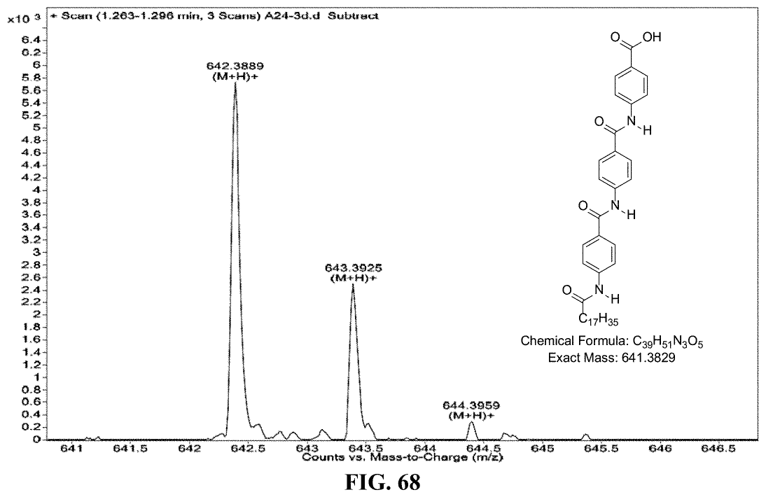

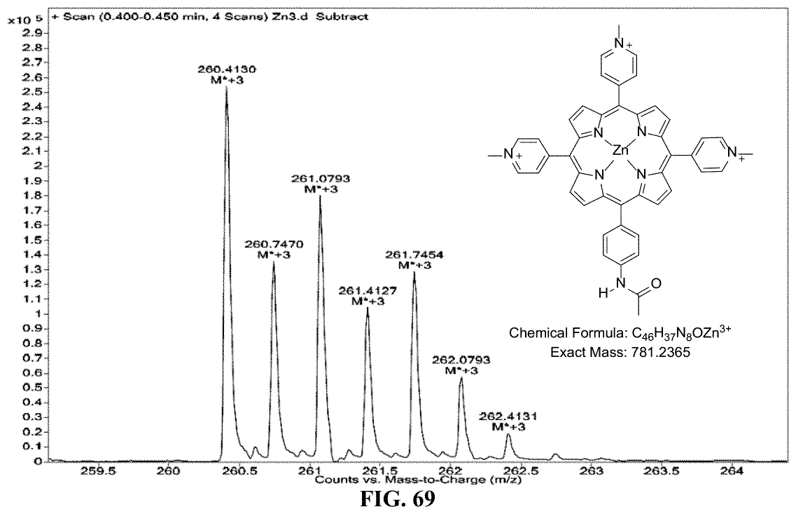

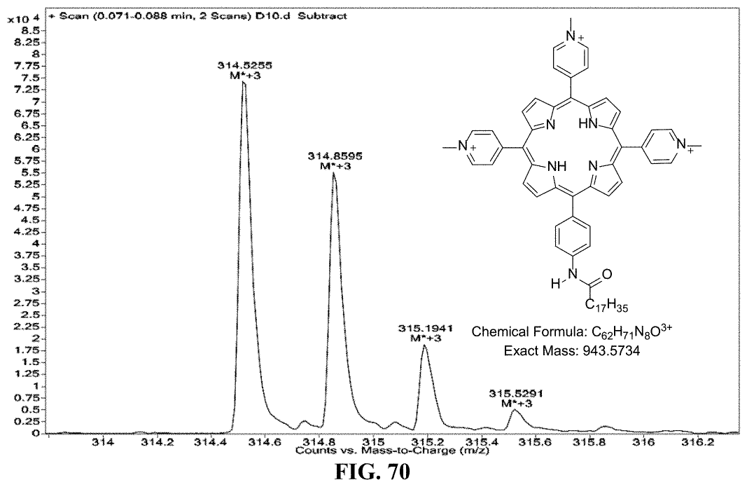

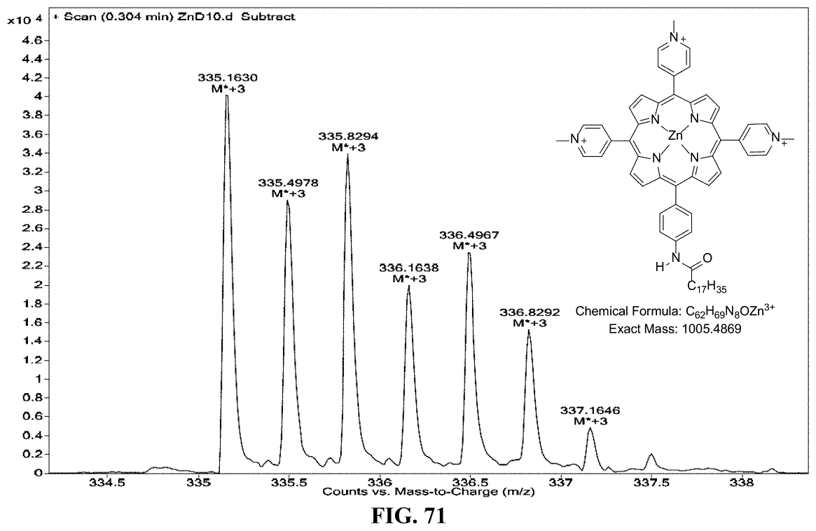

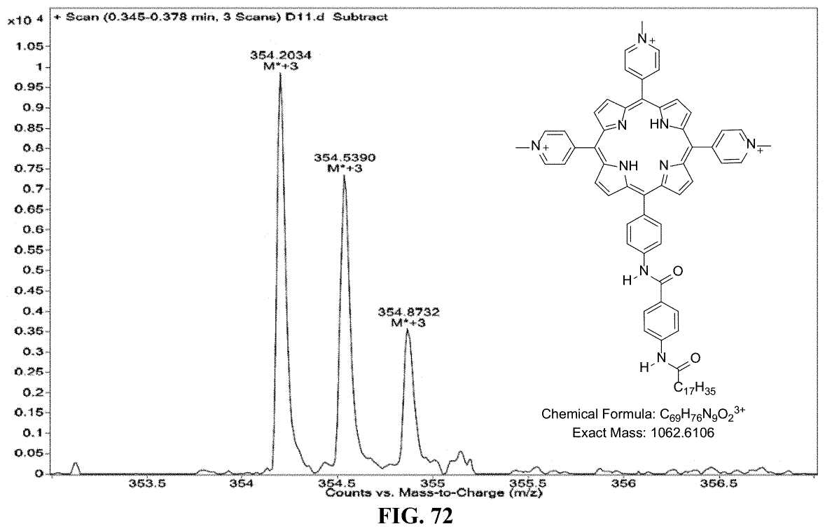

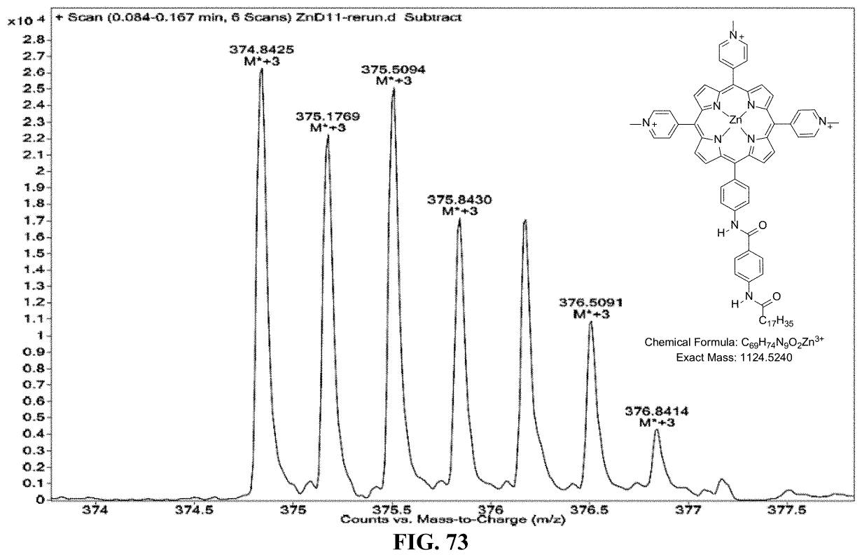

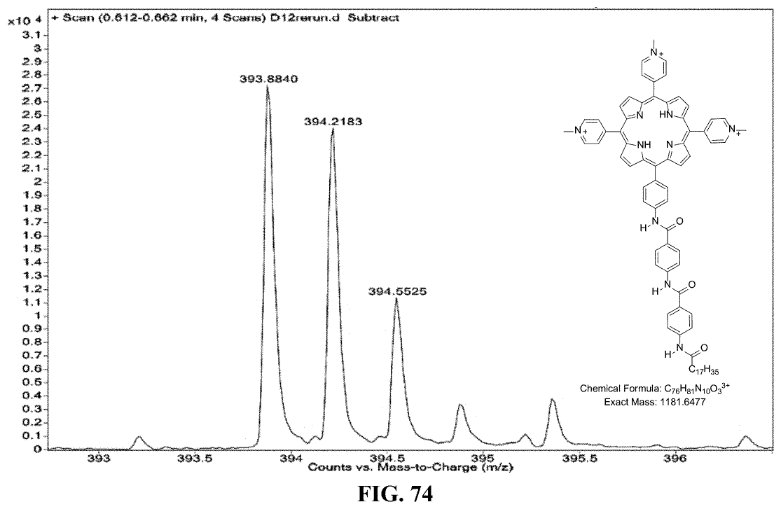

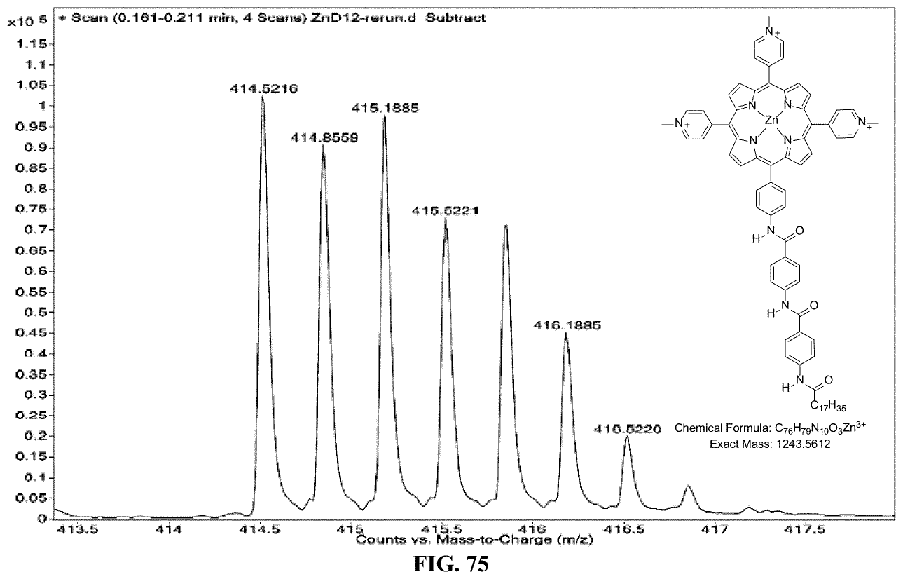

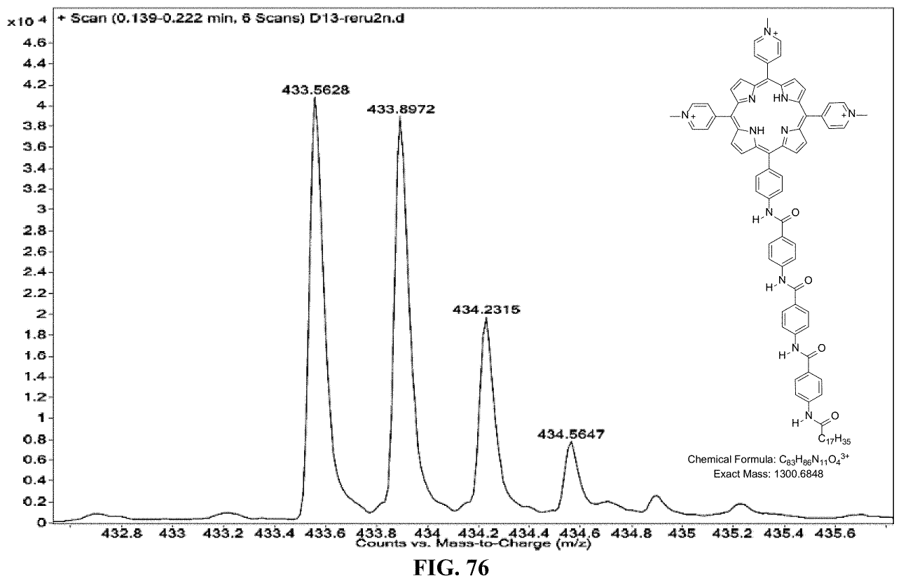

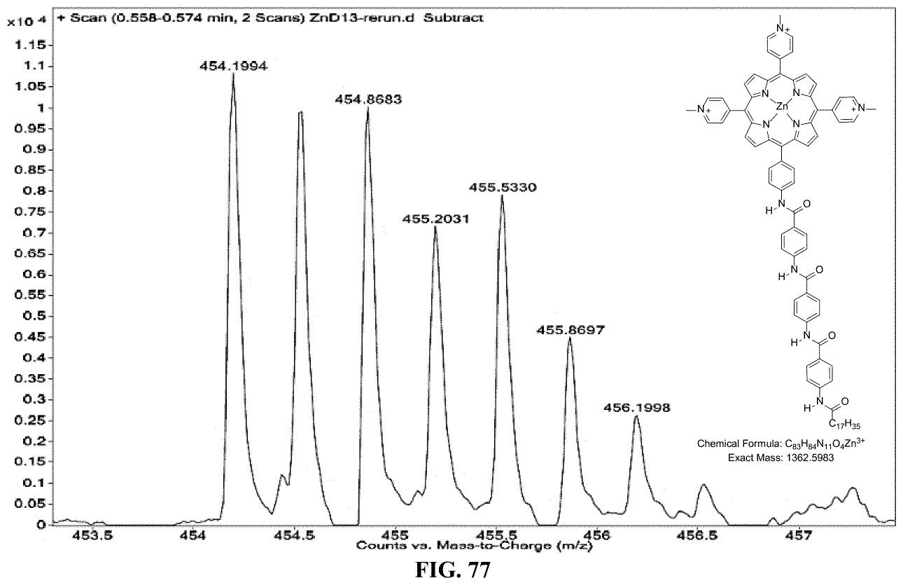

FIGS. 50-77 depict the following: ¹H NMR spectrum (400 MHz) of compound 3 in DMSO-d₆ (2 mM); ¹³C NMR spectrum (100 MHz) of compound 3 in DMSO-d₆ (6 mM); ¹H NMR spectrum (400 MHz) of compound 4 in DMSO-d₆ (1 mM); ¹³C NMR spectrum (100 MHz) of compound 4 in DMSO-d₆ (8 mM); ¹H NMR spectrum (400 MHz) of compound 5 in DMSO-d₆ (5 mM); ¹³C NMR spectrum (100 MHz) of compound 5 in DMSO-d₆ (10 mM); ¹H NMR spectrum (400 MHz) of compound ZnPC in DMSO-d₆ (5 mM); ¹³C NMR spectrum (100 MHz) of compound ZnPC in DMSO-d₆ (10 mM); ¹H NMR spectrum (400 MHz) of compound PAA0 in DMSO-d₆ (2 mM); ¹³C NMR spectrum (100 MHz) of compound PAA0 in DMSO-d₆ (10 mM); ¹H NMR spectrum (400 MHz) of compound PAA1 in DMSO-d₆ (5 mM); ¹³C NMR spectrum (100 MHz) of compound PAA1 in DMSO-d₆ (10 mM); ¹H NMR spectrum (400 MHz) of compound PAA2 in DMSO-d₆ (5 mM); ¹³C NMR spectrum (100 MHz) of compound PAA2 in DMSO-d₆ (10 mM); ¹H NMR spectrum (400 MHz) of compound PAA3 in DMSO-d₆ (5 mM); ¹³C NMR spectrum (100 MHz) of compound PAA3 in DMSO-d₆ (12 mM); ESI-mass spectrometry of compound 3; ESI-mass spectrometry of compound 4, ESI-mass spectrometry of compound 5, ESI-mass spectrometry of compound ZnPC, ESI-mass spectrometry of compound PAA0, ESI-mass spectrometry of compound ZnPAA0, ESI-mass spectrometry of compound PAA1, ESI-mass spectrometry of compound ZnPAA1, ESI-mass spectrometry of compound PAA2, ESI-mass spectrometry of compound ZnPAA2, and ESI-mass spectrometry of compound PAA3, ESI-mass spectrometry of compound ZnPAA3.

The compositions and assemblies described herein can be used in a variety of different ways that are unique compared to phospholipid vesicles.

Producing carbon-neutral solar fuels presents a promising approach toward confronting the global warming and the fossil fuels crises from the source. Supramolecular assemblies play critical roles in natural photosynthesis that supplied most energy of the Earth by far. Mimicking the natural behavior of light harvesting complexes that precisely manipulate the photocatalytic process is a great challenge. Herein, by using the power of self-assembly, a recyclable noble-metal free supramolecular photocatalytic assemblies system has an effectiveness that was significantly enhanced by the incorporation of aramid-linkers into their structure. These artificial assemblies powered by visible-light are highly stabile, highly efficient, and can easily switch between H₂ (TON, 652; selectivity, 100%), CO (TON, 712; selectivity, 100%) and CH₄ (TON, 396; selectivity, 88%) production at ambient temperature and pressure. The formation of methane from CO₂ occurs via a two-step procedure, first by reduction of CO₂ to CO and then reduction of CO to CH₄ with a 1.2% quantum yield. The water-soluble catalytic system operates stably over 12 recycles throughout several days. This strategy provides unique insight for the design of artificial photocatalytic materials.

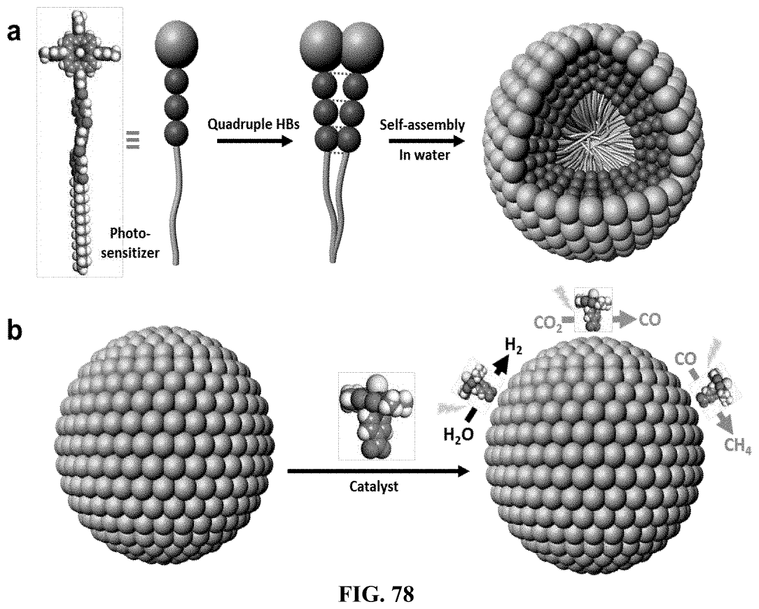

The Sun constantly provides Earth with 120,000 terawatts of power, which is roughly 4000 times higher than primary power needs for human civilization by 2050. Storing solar energy into chemical fuels through carbon-neutral strategies, for instance, splitting water into H₂ and O₂ or reducing CO₂ to valuable organic compounds, provides a potential solution to the fossil fuels crisis with net-zero greenhouse gas emissions. Artificial photocatalysis for achieving this purpose typically follows two major pathways: one is the heterogeneous catalysis (HTC), which is typically represented by photoelectrochemical (PEC) cells; and the other is homogeneous catalysis (HMC), where the photosensitizer and catalyst function in molecular forms in solution. Differs from the above two strategies, nature employs supramolecular assemblies to realize photosynthesis by converting photons to carbohydrates. Organisms promote light-harvesting efficiency via highly ordered assemblies of photofunctional components within proteins that provide tailored catalytic environments for reactions. In the chloroplast of plants, the cyclic multi-porphyrin arrays in light-harvesting complexes display an “antenna effect” to enable precise excitation energy transfer (EET) during the photocatalysis process. The high stability, selectivity, and efficiency of natural photocatalysis rely on the accurate control of orientation, distance, and delocalization of chromophore molecules and metalloporphyrin catalytic centers. See, FIG. 78.

Mimicking the natural behavior of plant chloroplasts that precisely control the orientation and distance between chromophores, electron relay complexes and enzymes is always challenging. The past few decades saw great development of supramolecular self-assembly at multiple scales and wide spread application fields. Self-assembling chromophore molecules and fine-tuning their catalytic properties by non-covalent interactions in water become a promising strategy for mimicking natural photocatalytic systems.

However, even charge separation and transport properties of self-assembly structures has been studied for decades, only few of attempts have developed to realize integrated artificial systems, in particular self-assembling hydrogel scaffolds, supramolecular metal-organic frameworks (SMOFs), and co-assembling photosensitizers and catalysts in natural lipid systems. The studies of artificial photosynthesis based on supramolecular assemblies remain rare, and none of them could achieve CO₂ reduction. Furthermore, the development of real industrialized applications for supramolecular photocatalytic materials is restricted by the low catalytic efficiency, the high cost of noble-metal catalytic components, the photobleaching of photosensitizers, and low photocatalytic stabilities.

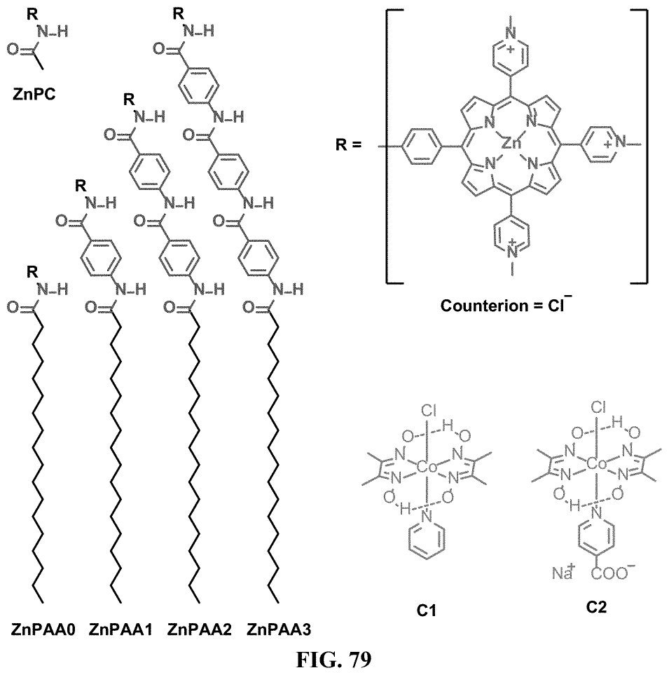

Herein, a series of hydrogen bonds (HBs) enhanced novel amphiphiles that self-assemble into ultra-uniform micelles with extraordinarily high chemical and structural stability in water have been synthesized. Independent of homogeneous and heterogeneous pathways, the concept of supramolecular photocatalytic assemblies (SPAs) is introduced, in which the self-assembly was employed as a powerful tool to control the distance, orientation, size and shape. As illustrated in FIG. 78, the SPA system displays highly selective and switchable water and CO₂ reduction. Much like natural photosynthetic antennae, the surface of micelles can absorb light, split excitons and transport the energy to catalytic reaction centers. The noble-metal free photocatalytic system was constructed by using cationic porphyrin hydrophilic head groups as photosensitizers and anionic cobalt complexes as catalysts (FIG. 79).

Results

To synthesize the target molecules (FIG. 79), a series of aramid-based tail groups were synthesized and attached to porphyrin photosensitizer via condensation reactions to obtain the porphyrin aramid amphiphile (PAA) with different numbers of aromatic amide units PAA0-PAA3 (see supporting information for the details of synthesis and characterization data for new compounds, FIGS. 50-77). The interactions for driving monomer assembly were engineered by increasing the number of aromatic amide units that strongly interact through HB formation and aromatic stacking, and which may lead to the formation of hydrogen-bonding networks (HBN). See, FIG. 79.

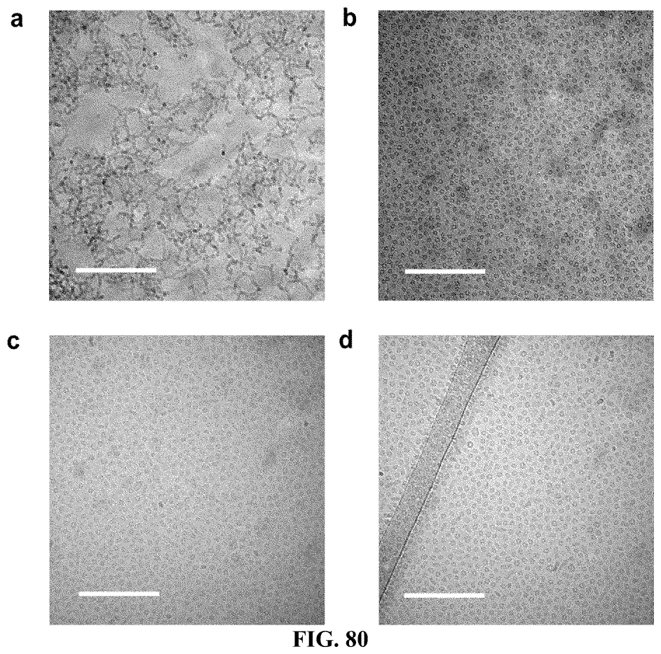

The PAA assemblies were initially characterized by negative staining-transmission electron microscopy (NG-TEM) experiments (FIGS. 33-36). From PAA0 to PAA3, the shape of PAs changed from near-straight nano-tubes (PAA0) to curved nano-wires (PAA1, PAA2), and then to peanut-like particles (PAA3). These phenomena indicate that the interactions of PAs were increased by adding aromatic amide units, resulting in increased hydrogen-bonding and aromatic stacking. Then the zinc porphyrin aramid amphiphiles (ZnPAAs) were obtained by chelation of zinc (FIG. 78). Interestingly, NG-TEM revealed that all the ZnPAAs formed extremely uniform micelles with sub-20 nm diameters (FIGS. 33-36). Cryogenic transmission electron microscopy (cryo-TEM) also revealed the uniform micelle assembly structures of ZnPAAs with sub-20 nm diameter features in water (FIG. 79). See, FIGS. 79 and 80.

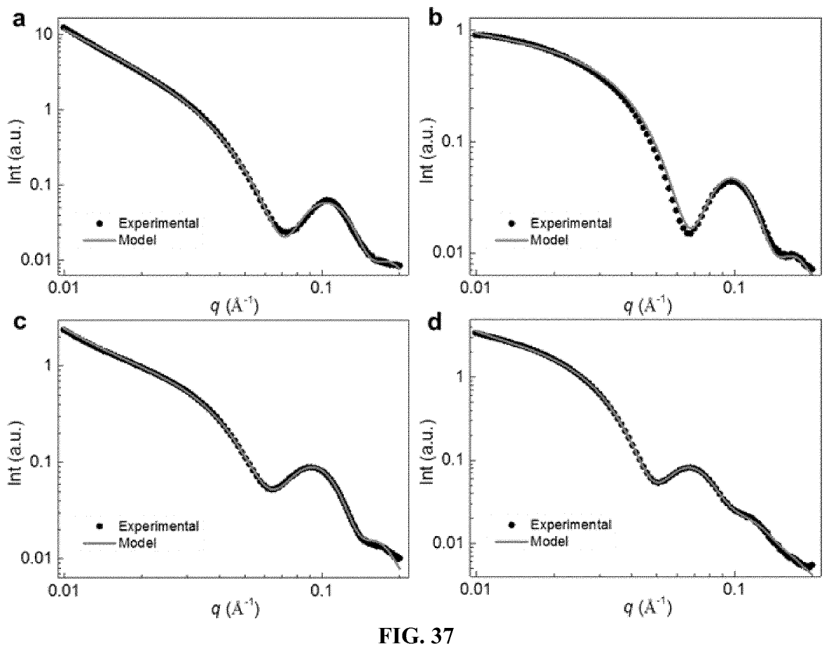

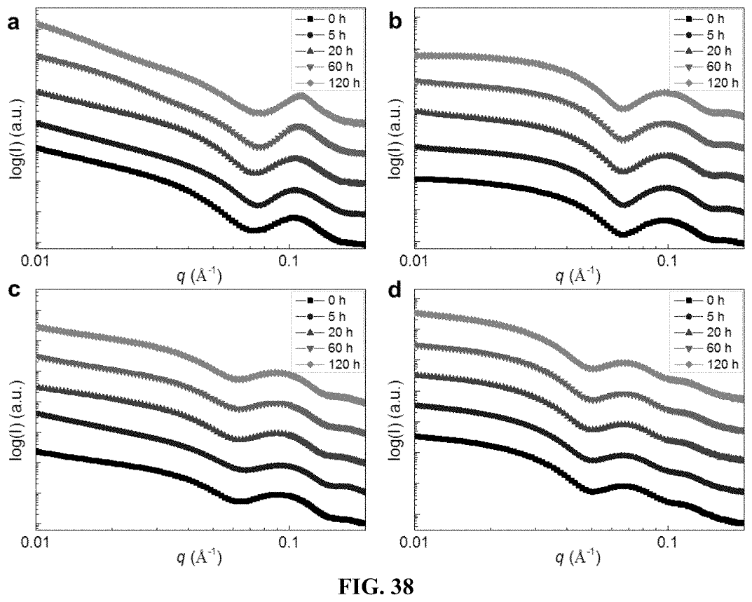

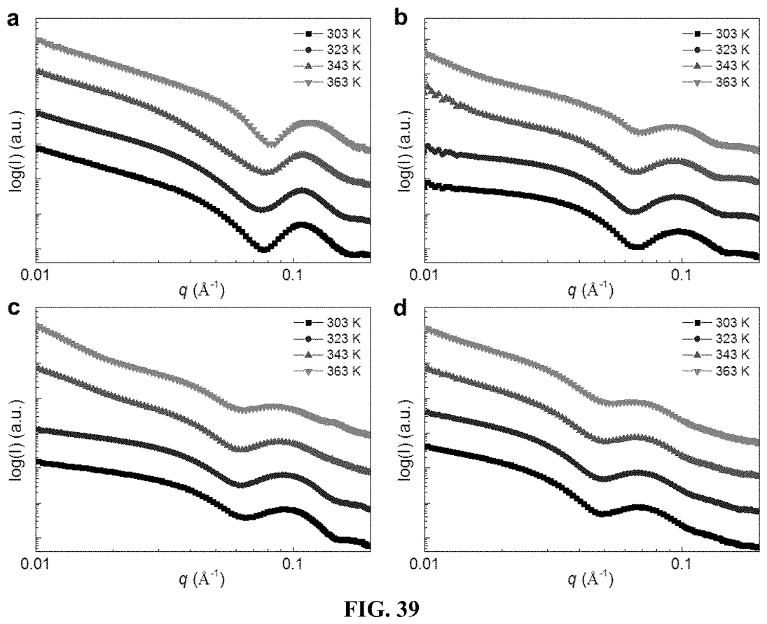

Synchrotron small-angle X-ray scattering (SAXS) (FIG. 80) was conducted and scattering curves were fit for various ZnPAA nanostructures (FIG. 37). After fitting by core-shell models, the mean diameters of micelles of ZnPAA0 to ZnPAA3 were determined to be 9.7±0.5, 10.3±0.5, 11.5±0.5 and 14.2±1.0 nm, respectively. Furthermore, the stability of SPAs was monitored by SAXS at different visible-light irradiation time of up to 120 h (FIG. 38) and variable temperature from 303 to 363K (FIG. 39). As increase of the aramid units from ZnPAA0 to ZnPAA3 the less morphology changes were observed, which indicate the stability increase from ZnPAA0 to ZnPAA3.

Dynamic light scattering (DLS) experiments were also performed to determine the hydrodynamic diameter (D_{H}) of micelles. The four ZnPAAs gave rise to a DH value of 7.8, 10.0, 12.6 and 16.0 nm at [Zn]=0.2 mM (where [Zn] represents the concentration of zinc amphiphile), respectively (FIG. 4). This result suggested an increasing trend of micelle sizes and very narrow size distributions for all the ZnPAAs assemblies. DH values of micelles at different concentrations from [Zn]=0.05 to 5 mM were almost identical (FIG. 80), which indicates that the particle shape of SPAs remains very stable against concentration changes.

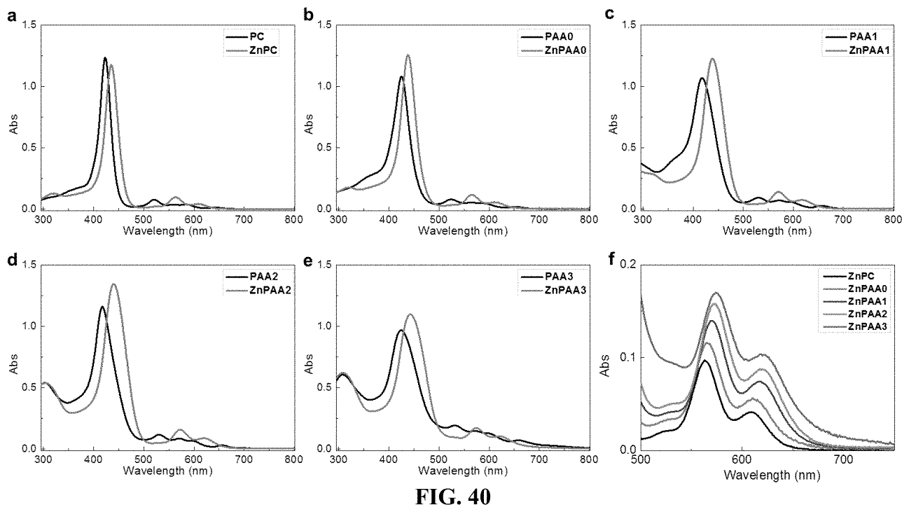

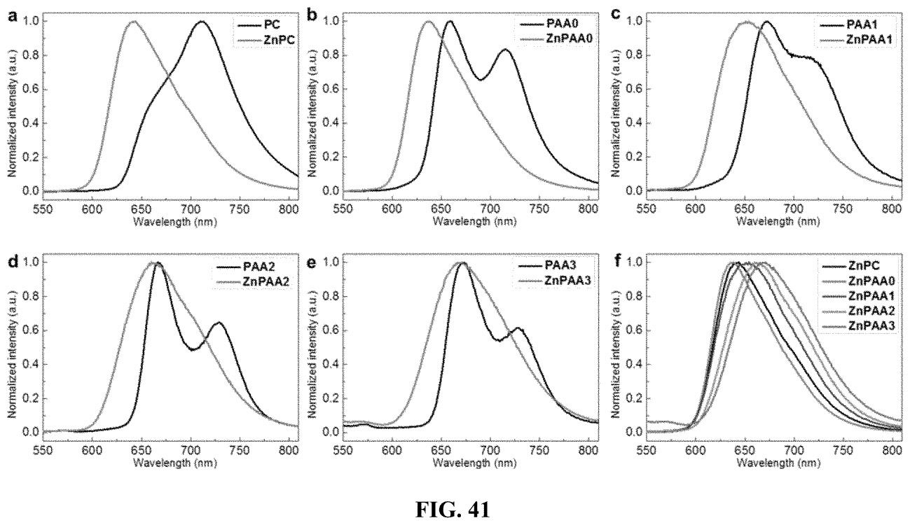

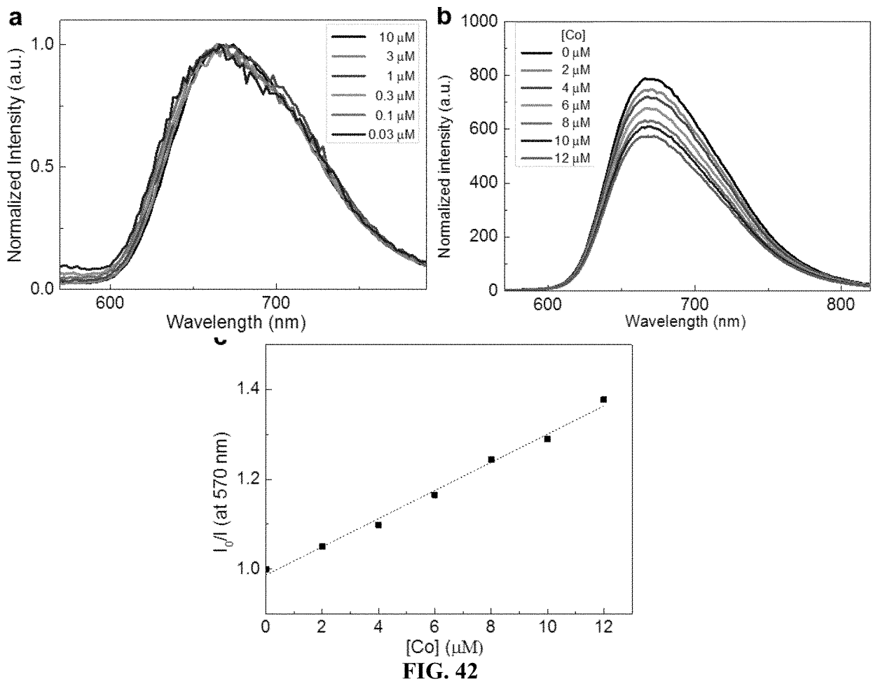

Along with absorptivity increases, absorption red-shifts (FIG. 40) and emission red-shifts (FIG. 41) of porphyrins were observed from ZnPAA0 to ZnPAA3 (0.02 mM, at room temperature), which indicate the interaction improvement between chromophores. During the dilution from 10 μM to 30 nM of ZnPAA3, no obvious changes were observed in emission spectra (FIG. 42) indicating an extremely low critical micelle concentration (CMC) of SPAs.

The zeta potential of ZnPAA0-3 was measured to be 45.6±2.8, 56.3±3.4, 62.3±5.0 and 88.4±7.3 mV, respectively. This phenomenon not only reveals that all the micelles formed by ZnPAAs have positive surface charges (FIG. 80) that can be used as an electrostatic driving force to attract anionic catalyst C2, but also indicates the high dispersibility of these SPA micelles. Remarkably, the ZnPAA3 has outstanding anticoagulant properties as its zeta potential is larger than 60.0 mV. Photobleaching of ZnPAAs was also monitored by measuring A/A₀ of zinc porphyrin chromophores, where A₀ is the initial absorbance at 425 nm (FIG. 45). After 120 h irradiation, the A/A₀ of ZnPAA0-3 was determined to be 3.5%, 11.1%, 34.3%, and 84%, respectively, while the A/A₀ of control molecular ZnPC quickly decreased to 2.2% in 10 hours. This confirmed that SPAs formed by ZnPAA3, which has quadruple hydrogen bonds on two sides, are highly stable under long-term irradiation.

For designing a noble metal-free supramolecular photocatalytic system, a cobalt bisdimethylglyoximate complex [Co^{III}(dmgH)₂(py)Cl] (C1), which is commonly used to catalyze proton reduction. In order to promote the interaction of chromophore and catalyst was chosen, negatively charged complex C2 was synthesized (FIG. 78). The anionic nature of C2 endows it with better water solubility and attraction to the cationic surface of the ZnPAA micelles. As was observed in other cases, the anionic form of the catalyst localizes on the chromophore-dense surface of micelles via electrostatic attraction, as predicted by the hard-soft-acid-base (HSAB) theory. Fluorescence quenching experiments demonstrated electron transfer from the photo-excited state of the zinc porphyrin units to cobalt complexes. The C2 quenched fluorescence of ZnPAA3 efficiently with a Stern-Volmer constant (K_{SV}=3.1×10⁴ M⁻¹, FIG. 42), providing evidence for a strong Coulombic coupling with the light-harvesting assemblies. The observation of this relationship between concentration of the quencher and emission intensity constitutes evidence that the molecule engages in single-electron transfer with the photocatalyst. Based on above observations, the electrostatic binding sites for catalysts will present on the surface of micelle assemblies with methylpyridinium terminal groups. The electrostatic coupling may induce the delocalization of zinc porphyrin photosensitizer and catalysts, thereby facilitating the electron-transfer reactions for fuel production.

The potentials of the reduction reactions from proton to Hz, CO₂ to CO, and CH₄ were reported to be E₀ (H⁺/H₂)=−0.41 V, E₀ (CO₂/CO)=−0.53 V, and E₀ (CO₂/CH₄)=−0.24 V versus NHE at pH=7 in aqueous solution, 25° C., under 1 atm CO₂. The potentials for the Co^{II} to Co^{I} was determined to be E₀ (Co^{II}/Co^{I})=−0.69 V by cyclic voltammetry (FIG. 44), which is more negative than all reduction potentials (H⁺/H₂, CO₂/CO, and CO₂/CH₄). The highest occupied molecular orbital (HOMO) energy and the lowest unoccupied molecular orbital (LUMO) energy of C2, ZnPC, and ZnPAAs were also determined by their absorption spectra (FIG. 43) and cyclic voltammetry (Table 1 and FIG. 44). The LUMO energy level of catalyst C2 (CoII/I) was determined to be −3.88 eV. And the LUMO energy level of ZnPC and ZnPAA0-ZnPAA3 was determined to be −3.23 eV, −3.25 eV, −3.29 eV, −3.30 eV, and −3.34 eV, respectively. This also support the possibility of using SPAs for catalyzing visible light-driven proton and CO₂ reduction. Remarkably, the SPA of ZnPAA3 has the lowest LUMO energy compare to ZnPAA0-2 and control ZnPC, which is closer to the LUMO energy level of C2.

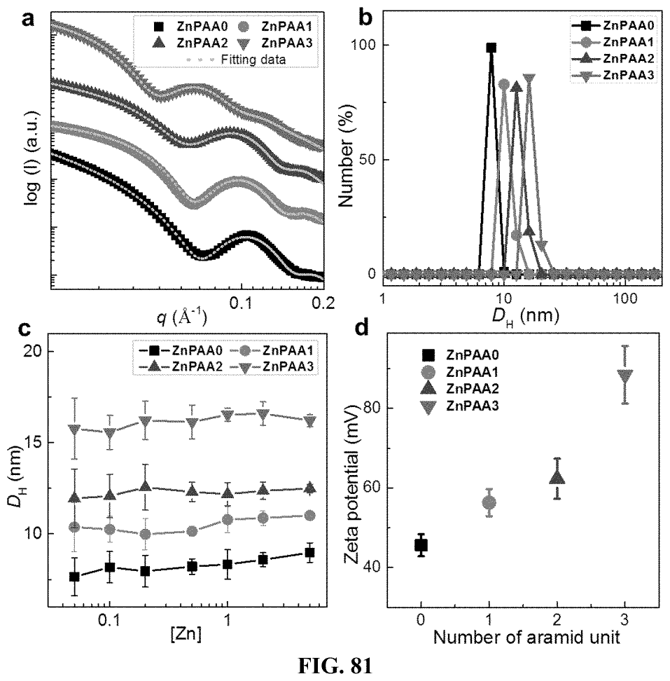

The hydrogen evolution reaction (HER) was first investigated for ZnPAAs and the control ([Zn]=0.2 mM) with catalyst C2 (2 μM) (Table 2) under visible light irradiation (wavelength λ>400 nm) with ascorbic acid (20 mM) as a sacrificial electron donor. After 20 hours illumination with air as atmosphere at room temperature, the headspace of ZnPC and ZnPAA0-3 were analyzed by gas chromatography (GC) showed turnover number (TON) 34±5, 164±19, 188±25, 201±14, and 217±31 respectively. However, similar HER efficiencies were observed for neutral catalyst C1 with control ZnPC (36±3) and ZnPAA3 assembly (41±4), which is much lower than combinations of C2 with micelle SPAs. This may attribute to electrostatic attraction induced local concentration increases of catalyst and distance reduce between catalysts and sensitizers for electron conveys. Interestingly, using C2 as catalyst all the micelle assemblies exhibited remarkably high HER efficiencies, which were 4 to 6 time higher than the efficiency of the control molecule ZnPC. In order from most to least efficient, the amphiphile HER efficiencies were determined to be ZnPAA3>ZnPAA2>ZnPAA1>ZnPAA0. This trend may arise from several factors. First, the assemblies with higher intermolecular forces may have more dense chromophores on surface and hence possess relatively higher apparent chromophore concentration, which would improve the efficiency of electron transfer from photosensitizer to catalyst. Second, the surface cationic charge density increased as the number of intermolecular hydrogen bonds increased, as was shown by zeta-potential experiments (FIG. 80), thereby attracted more catalyst C2 molecules. Third, the stability of assemblies and chromophores increased as the interaction between amphiphile molecules increased from ZnPAA0 to ZnPAA3, which was confirmed by the SAXS experiments with different irradiation times (FIG. 38) and photo bleaching experiments (FIG. 45). Further, as the most stable micelle system observed, the time-dependent hydrogen production was investigated for ZnPAA3. As shown in FIG. 81, the ZnPAA3 micelles ([Zn]=0.2 mM) in aqueous solution that contained the cobalt catalyst C2 (2 μM) with ascorbic acid (20 mM) as sacrificial electron donor produced H₂ with a TON in H₂ relative to catalyst concentration of 652±27 after 96 hours irradiation (entry 1 in Table 6, and FIG. 81). The linear production of H₂ versus time indicates good stability of this photocatalytic system. In control experiments (entries 8-11 in Table 2), no obvious hydrogen production was observed in absence of photo-sensitizer (ZnPAA3), catalyst (C2), ascorbic acid or light source, which indicates that all the above components are necessary to give rise to the HER of the SPA system.

Under a 1 atm saturated CO₂ atmosphere, selective CO₂ to CO reduction was observed by irradiating the ZnPAA3 solution ([Zn]=0.2 mM) for 108 hours in water with C2 (2 μM). This process exhibited a TON of 712±22 when sodium ascorbate (20 mM) was employed as a sacrificial electron donor (entry 2 in Table 6, FIG. 81). The selective CO production was also observed in other three SPA systems, but with slightly lower TONs of 387±25, 549±42 and 672±28 for ZnPAA0, ZnPAA1, and ZnPAA2 respectively (Table 3). However, the selectivity for CO production was not observed for the ZnPC control as molecular catalyst, where the TONs for the HER and CO production reactions were determined to be 29±6 and 12±2, respectively (entry 1 in Table 3). The difference between the SPAs and molecular photosensitizer/catalyst system may indicate that the surface micro-environments created by assemblies could enhance the photocatalytic process and lead to different production rates. The control experiments also showed that the presence of ZnPAA3, catalyst, sodium ascorbate, and light are necessary for generating CO from CO₂ reduction (Table 3). In control experiments, only H₂ was produced when CO₂ was not fed (entry 10 in Table 3), even in cases that Na₂CO₃ or NaHCO₃ were added to simulate components of CO₂ in aqueous solution (entry 11 and 12 in Table 3). This excluded the possibility the carbon source was from other components during reaction. Even the electrochemical behavior of cobalt dimethylglyoxime complex in the CO₂ reduction process had been studied. The photocatalytic CO₂ reduction property for cobaloxime derivates has never been reported.

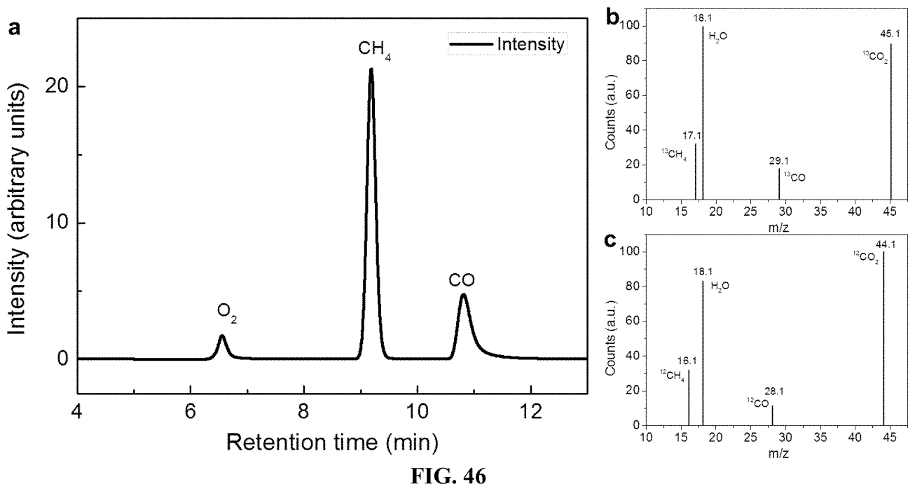

Interestingly, when replacing the sacrificial reagent by triethylamine (TEA), methane was produced selectively. For a solution of ZnPAA3 (0.2 mM), C1 (2 μM) and TEA (20 mM), the irradiation time of 32 h produced H₂, CO and CH₄ with turnover numbers (and selectivities) of 13±3 (13%), 33±7 (32%), and 14±3 (55%) (entry 7 in Table 4). When C2 was employed as catalyst, after 32 h irradiation, the catalytic efficiencies of H₂, CO and CH₄ production were significantly increase to 54±9 (10%), 77±14 (14%), and 106±17 (76%) (entry 8 in Table 6). In Table 4, the ZnPAA0-2 SPAs and control ZnPC were also tested at the same catalytic and sacrificial reagent concentrations. The ZnPAA0-2 SPAs produce H₂, CO and CH₄ in low efficiency, but the ZnPC only produced H₂ and CO, and with relatively low TONs. This is not surprising because the ZnPAA3 formed SPA is more stable and with larger effective cationic concentration on surface. An isotope labeling experiment was further performed under ¹³CO₂ atmosphere, gas chromatography-mass spectrometry (GC-MS) analysis (FIG. 46) identified the production of ¹³CH₄ (m/z=17) and ¹³C0 (m/z=29), confirming that these compounds originate from CO₂ reduction. Only ¹²CH₄ (m/z=16) and ¹²CO (m/z=28) were produced when the reaction was performed under ¹²CO₂ atmosphere (FIG. 46). See, FIG. 81.

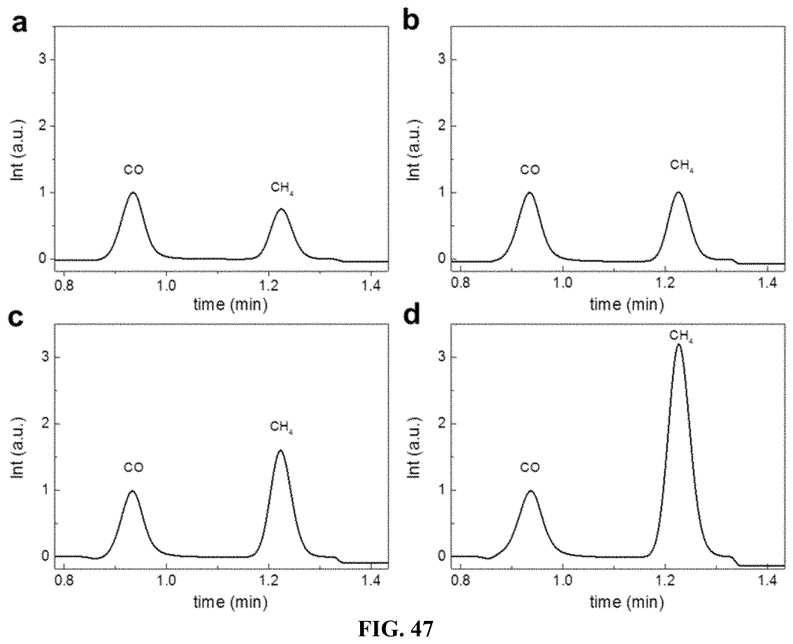

When 20 mM triethylamine hydrochloride (TEA.HCl) was used instead of TEA for ZnPAA3 assembly system, after 32 h irradiation, the turnover number of methane formation was considerably increased from 106±17 (76%) to 156±11 (86%) (entry 9 in Table 6). This increase probably results from the participation of sacrificial reagent TEAH⁺, which could also be a proton donor and facilitating the reaction, as a similar phenomenon also has been reported³². Blank experiments in the absence of photosensitizer, catalyst, sacrificial reagent or light fail to show notable reduction productions (entries 10-13 in Table 6). Interestingly, the GC results showed that ZnPAA3 produced more CH₄ (TON=129±23, 89%), which exceeds the TON of methane production of other SPAs with turnover numbers (and selectivities) 83±16 (73%) for ZnPAA0, 96±19, (76%) for ZnPAA1, and 119±22 (84%) for ZnPAA2 after 24 h irradiation (Table 5, FIG. 47). This result indicates that the ratio of products is tunable by engineering the monomer interactions, which results in photocatalytic micro-environment changes on nanostructure surface. No other gaseous product was formed, and analysis of the liquid phase failed to detect methanol or formaldehyde by ¹H NMR or formate (HCOO⁻) by ion chromatography.

A long-term irradiation experiment up to 106 hours was performed for ZnPAA3 SPA system, for a solution of ZnPAA3 (0.2 mM), C2 (2 μM), TEA (20 mM) and TEA.HCl (20 mM), the longest irradiation time of 106 h produced H₂, CO and CH₄ with turnover numbers (and selectivities) of 76±17 (4%), 141±19 (8%) and 396±26 (88%, calculated by electron selectivity), respectively (Table 6, entry 3 and FIG. 81). These values correspond to a methane production rate of 7,954 μmol per hour per gram of catalyst (μmol h⁻¹ g⁻¹), which exceeds the rate of many other catalysts that generate methane from CO₂. The linear evolution of H₂, CO and CH₄ over more than 106 h and the stable absorption spectrum of the system under irradiation (FIG. 81), with no evidence for degradation of the sensitizer ZnPAA3 or catalyst C2, illustrate the stability of the catalytic system.

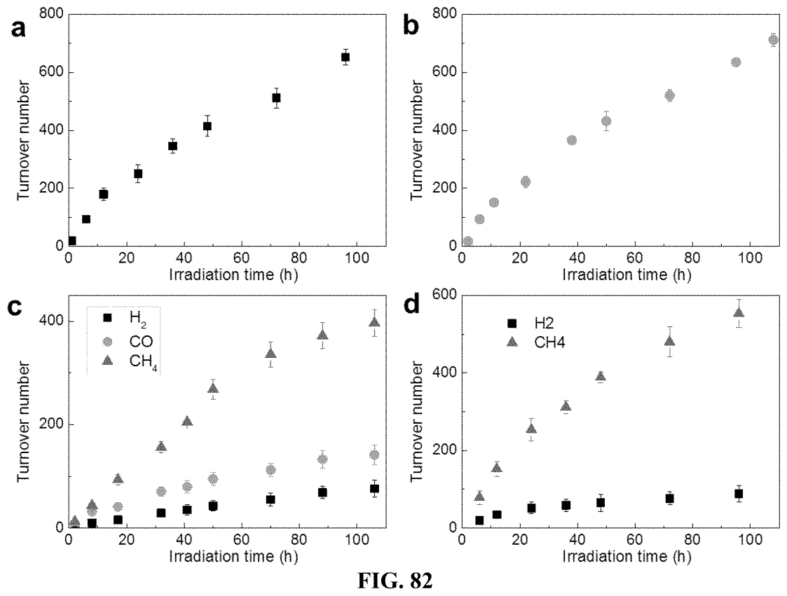

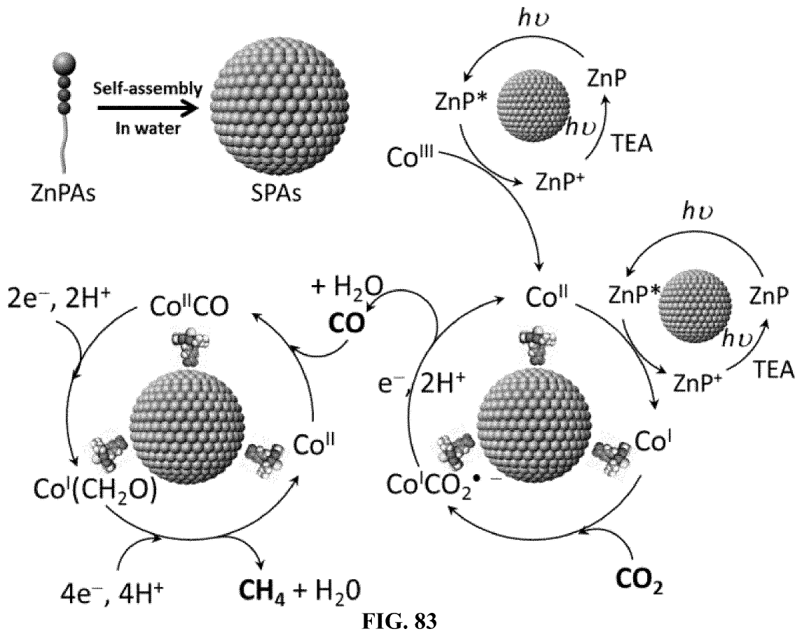

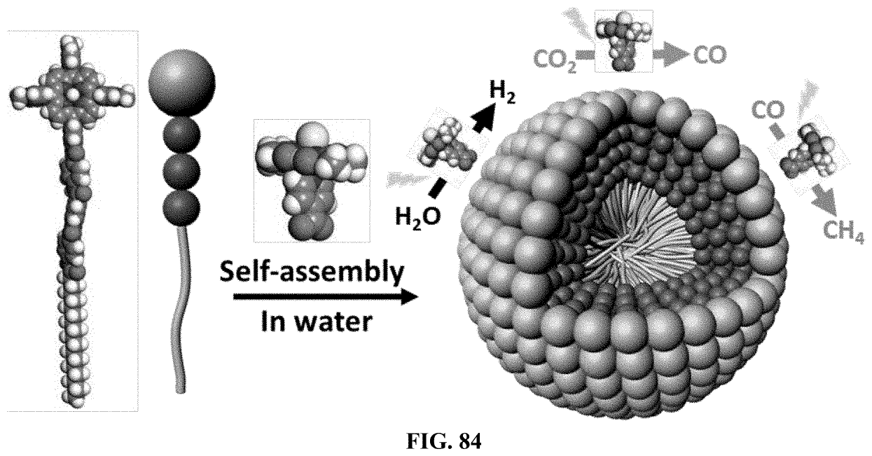

The influence of CO was studied by conducing the reaction under 1 atm CO atmosphere with visible light irradiation (λ>400 nm), with ZnPAA3 assemblies as photosensitizer, C2 as catalyst and TEA.HCl as sacrificial electron donor (FIG. 81). After 96 h irradiation, H₂ and CH₄ were produced with TONs (and selectivities) of 88±21 (14%) and 554±37 (86%), respectively. The total CH₄ formation is roughly 1.4 times that of the experiment using CO₂-saturated solution (entries 15 in Table 6), with a methane production rate of 12,286 μmol h⁻¹ g⁻¹, while the level of H₂ production was almost identical. See, FIG. 82.

Based on above experimental data, a plausible mechanism was sketched in FIG. 82. The zinc porphyrin on the surface of SPAs was excited by visible-light and injected electrons to Co^{III} of catalyst C2 to form the Co^{I}, which is considered as the active center for the CO₂ reduction catalytic cycle. In the first step, one electron is transferred from Co^{I} to a CO₂ molecule and forms a Co^{II}CO₂.⁻ intermediate species that can be stabilized by cationic surface of micelles. Then, the CO₂ anion takes two protons and one electron, and forms a CO and a H₂O molecule. Previous studies suggest that the first step proceeds at a much more negative potential than the following steps and is therefore rate determining for the whole process. The subsequent steps of the catalytic cycle involves a postulated formaldehyde intermediate Co^{I}(CH₂O) that may be stabilized by through-space interactions between the positive charges on surface of SPAs and the partial negative charge on the HCHO species bound to the metal. With complete reduction of the Co^{II}CO adduct necessitating six electrons, the quantum yield for CH₄ formation is Φ=1.2% (see Methods), which exceeded the natural photo-synthesis efficiency of crop plants.

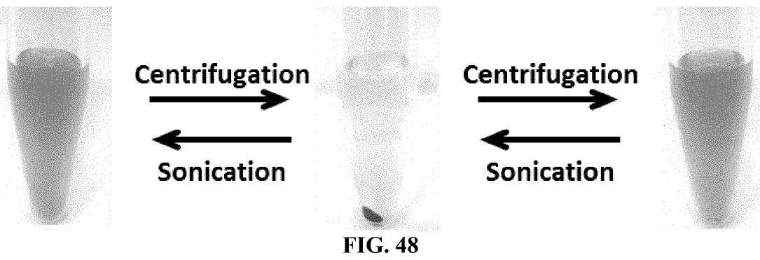

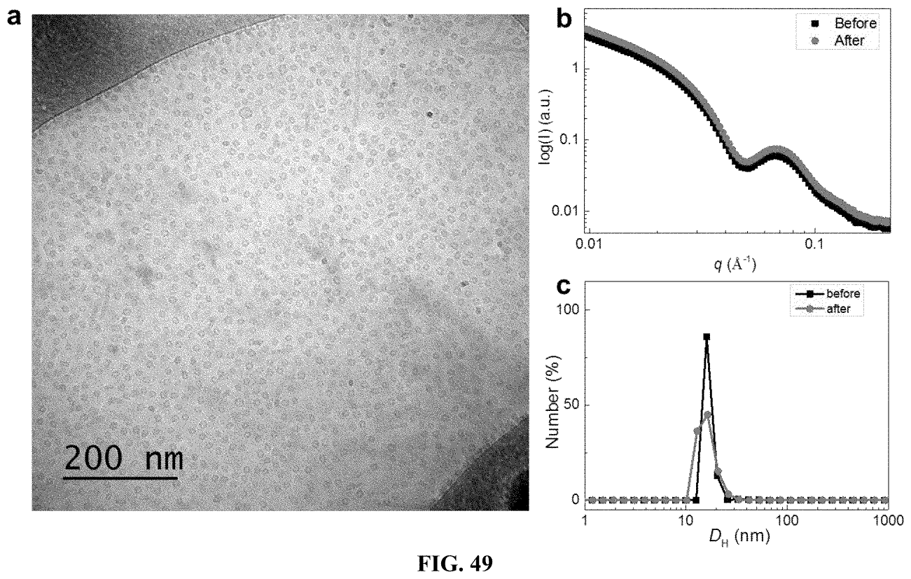

The SPAs were recoverable by high speed centrifugation. After 1 minute centrifugation at 15000 rpm, the ZnPAA3 settled down to the bottom of the centrifuge tube (FIG. 48). After re-dissolving into the supernatant solution, the photosensitizer could be reused for catalysis. After 12 repetitions of this process, the TONs of CH₄ production was reduced from 55±11 (Entry 6 in Table 5) to 48±4 (Entry 17 in Table 5), which indicates that the SPA photocatalytic system could be recycled without significant loss of catalytic ability. Cryo-TEM (FIG. 49) and SAXS (FIG. 49) experiments further revealed that ZnPAA3 remains its morphology after 12 repetitions of the centrifugation process, and DLS experiments showed that ZnPAA3 maintained its DH and size distribution (FIG. 49). This property enables the easy recovery of the SPA photocatalytic system in water. And the SPA scaffold and embedded components are thus easily dried and stored in powder form, then recovered by re-dissolving assemblies in aqueous solution.

Discussion

In conclusion, highly stable and uniform SPA micelles were constructed by self-assembling ZnPAAs in water with Earth-abundant Co-based catalysts. Powering by visible-light, SPAs display remarkable photocatalytic properties toward product-switchable proton and CO₂ reduction with high efficiency and selectivity. Independent of the heterogeneous and homogeneous catalytic systems, the SPA offer a unique solution to artificial photosynthesis with value added products. The water-solubility, high stability, recyclability, and lightweight, device-free, easy storage property of SPAs make them promising for real industrial use. SPAs can be open many possibilities for the design and synthesis of future photocatalytic materials.

Methods

Materials and Measurements.

All reagents were obtained from commercial suppliers and used without further purification unless otherwise noted. ¹H and ¹³C NMR spectra were recorded on a Bruker 400 MHz spectrometer in DMSO-d₆. Chemical shifts were referenced to the residual solvent peaks. Melting points (m.p.) were obtained on a Mel-Temp capillary melting point apparatus. Elemental analyses were performed for carbon, hydrogen, and nitrogen by Atlantic Microlabs Inc., Norcross, Ga. TEM Images were recorded on a Gatan UltraScan CCD camera by a JEOL 2100 FEG microscope operated at 200 kV, negative staining samples were prepared using 1% phosphotungstic acid (PTA) aqueous solution. High resolution mass spectra were measured on an Agilent 6210 Time of Flight (TOF) LC-MS, using an ESI source, coupled with Agilent 1100 HPLC stack, using direct infusion (0.6 mL/min). Luminescence measurements were performed on a VARIAN CARY Eclipse Fluorescence Spectrophotometer. Ultraviolet-visible spectra were performed on a Perkin-Elmer 650 instrument. Dynamic light scattering (DLS) experiments were performed using a DynaPro instrument (20 mW He—Ne laser, λ₀=780 nm, scattering angle θ=90°), Wyatt. Technology. Zeta potential data were obtained on a Möbiuζ Mobility Instrument, Wyatt. Technology.

Cryo-Transmission Electron Microscopy.

Cryo-TEM images were acquired on a JEM-2100-FEG transmission electron microscope (JEOL, Japan) operating at 200 KeV utilizing a Gatan 626 cryo-transfer holder (Gatan, USA). A small volume (3 μL) of ZnPAAs suspension at 2 mM in water was deposited on a copper TEM grid with holey carbon support film (Electron Microscopy Sciences) and held in place with tweezers mounted to the Vitrobot. The specimen was blotted in an environment with 90-100% humidity and plunged into a liquid ethane reservoir that was cooled by liquid nitrogen. The vitrified samples were transferred in a nitrogen environment into liquid nitrogen, and then transferred to a Gatan 626 cryo-holder using a cryo-transfer stage. Micrographs were recorded at nominal magnifications on a 4,096×4,096 pixel Tietz CCD camera.

Small Angle X-Ray Scattering.

Measurements in transmission mode were performed using beam line 12ID-B beamline (photon energy E=14.0 keV and wavelength λ=0.8856 Å) at the Advanced Photon Source (APS), Argonne National Laboratory. SAXS data are collected by a 2D Pilatus2m detector and the scattering vector magnitude Q, (Q=4π sin(θ)/λ, 2θ being the scattering angle), is calibrated with a silver behenate standard. The sample-to-detector distance was set such that the detecting range of momentum transfer Q was 0.004-0.93 Å⁻¹. The ZnPAAs samples are carefully loaded into 1.5 mm quartz capillary tubes, and multiple spots on precipitates are chosen and examined. All data were corrected for background scattering. Temperature control of samples was achieved using a custom-built Peltier device. In order to obtain good signal-to-noise ratios, twenty images were taken for each sample and buffer. The 2-D scattering images were converted to 1-D SAXS curves through azimuthally averaging after solid angle correction and then normalizing with the intensity of the transmitted X-ray beam, using software developed at beamline 12ID-B of APS for further data analysis.

Electrochemical Measurements.

All electrochemical measurements were run at 25° C. in a 20 mL customized glass vial with 0.2 M Na₂SO₄ aqueous solution. A BioLogic VMP3 work station was employed to record the electrochemical response. In a typical three-electrode test system, 2 mm diameter gold, a platinum foil (Beantown Chemical, 99.99%) and an Ag/AgCl/KCl (sat. in water), were used as the working, counter and reference electrode, respectively. The working electrode was cleaned by polishing with 0.05 μm polishing alumina followed by sonication. The scanning rate was 100 mV/s. All potentials measured against Ag/AgCl electrode were converted to the normal hydrogen electrode (NHE) scale in this work using E (versus NHE)=E (versus Ag/AgCl)+0.197 V.

Visible Light Irradiation Experiments.

The photocatalytic hydrogen production and carbon dioxide reduction experiments were carried out in an external illumination type reaction vessel with a magnetic stirrer. Samples for photocatalytic hydrogen production were prepared in 5 mL septum-sealed glass vials. Each sample was made up to a volume of 1.0 mL aqueous solution. Samples typically contained 0.2 mM of ZnPAAs and 0.002 mM of cobalt catalysts. The solution was irradiated by a 500 W solid state light source with a λ>400 nm filter. After the reaction, the gas in the headspace of the vial was analyzed by GC to determine the amount of gases generated.

Gaseous Product Analysis.

Electrochemical experimental yields were analyzed by GC in SRI 8610C GC system equipped with 72×⅛-inch S.S. molecular sieve-packed column and a thermal conductivity detector. Production of H₂, CO, and CH₄ was examined separately. A thermal conductivity detector (TCD) was mainly used to quantify H₂ concentration, and a flame ionization detector (FID) with a methanizer was used for a quantitative analysis of CO and other alkane contents. Ultrahigh-purity CO₂ (purchased from AirGas) was used as a carrier gas for CO and CH₄ detection, whereas ultrahigh-purity nitrogen (AirGas) was utilized for H₂ detection. Initially, GC system was calibrated for H₂, CO, and CH₄. To confirm that the CO and CH₄ products were derived from CO₂, an isotope ¹³CO₂ (Sigma Aldrich) was used as atmosphere gas for visible light irradiation experiments and GC-Mass spectroscopy was used for gas detection. The ^{D}C-labeled samples were analyzed on an Agilent 7890A gas chromatographer (GC) coupled with an Agilent 5975C mass spectrometer (MS). DB-5MS column (60 m×0.25 mm×2.5 μm) was used for the analysis. The inlet and the GC oven were set at 100° C. The transfer line, source, and MS were set at 270° C., 230° C., and 150° C., respectively. The MS was in full scan mode with m/z scan range of 14-50 amu. Samples were injected manually using a gas tight syringe. Air was injected as the instrument background.

Quantum Yield Calculation.

The number of incident photons was measured using the classical iron ferrioxalate (K₃Fe(C₂O₄)₃) chemical actinometer, following the procedure reported previously and using known parameters for calculations⁴. Using three independent measurements, it was determined that the number of incident photons to the sample was (9.76±0.47)×10¹⁸ photons per hour. The CO-to-CH₄ reduction being a six-electron process, the overall quantum yield 1 of the process was determined using the following equation:

$${φ}_{C {H}_{4}} ( \% ) = \frac{Number of Methane formed \times 6}{Number of incident photons} \times 100$$

Taking 554 as the highest turnover number for CH₄ (Table 6 entry 15), we obtain a quantum yield Φ of CH₄ about 1.2% after 96 h of irradiation.

TABLE 6
Summary of catalytic performance of catalysts and photosensitizers with reaction conditions.

| | Photo- | | Sacrificial | [Zn] | [Co] | λ | Time | Turnover numbers | | |
|---|---|---|---|---|---|---|---|---|---|---|
| Entry | sensitizer | Gas | reagent* | (mM) | (μM) | (nm) | (h) | H₂ | CO | CH₄ |
| 1 | ZnPAA3 | Air | AA | 0.2 | 2 | >400 | 96 | 652 ± 27 | — | — |
| 2 | ZnPAA3 | CO₂ | SA | 0.2 | 2 | >400 | 108 | — | 712 ± 22 | — |
| 3 | ZnPAA3 | CO₂ | TEA-HCl | 0.2 | 2 | >400 | 106 | 76 ± 17 | 141 ± 19 | 396 ± 26 |
| 4 | ZnPC | CO₂ | TEA-HCl | 0.2 | 2 | >400 | 106 | 178 ± 14 | 37 ± 10 | — |
| 5 | ZnPAA0 | CO₂ | TEA-HCl | 0.2 | 2 | >400 | 106 | 57 ± 11 | 193 ± 27 | 165 ± 25 |
| 6 | ZnPAA1 | CO₂ | TEA-HCl | 0.2 | 2 | >400 | 106 | 69 ± 15 | 185 ± 32 | 177 ± 28 |
| 7 | ZnPAA2 | CO₂ | TEA-HCl | 0.2 | 2 | >400 | 106 | 65 ± 18 | 161 ± 21 | 240 ± 31 |
| 8 | ZnPAA3 | CO₂ | TEA | 0.2 | 2 | >400 | 32 | 54 ± 9 | 77 ± 14 | 106 ± 17 |
| 9 | ZnPAA3 | CO₂ | TEA-HCl | 0.2 | 2 | >400 | 32 | 29 ± 7 | 70 ± 9 | 156 ± 11 |
| 10 | ZnPAA3 | CO₂ | TEA-HCl | — | 2 | >400 | 32 | — | 5 ± 1 | 2 ± 1 |
| 11 | ZnPAA3 | CO₂ | TEA-HCl | 0.2 | — | >400 | 32 | — | 2 ± 1 | — |
| 12 | ZnPAA3 | CO₂ | — | 0.2 | 2 | >400 | 32 | — | 3 ± 1 | — |
| 13 | ZnPAA3 | CO₂ | TEA-HCl | 0.2 | 2 | dark | 32 | — | — | — |
| 14 | ZnPAA3 | CO₂ | TEA-HCl | 0.2 | 2 (C1) | >400 | 32 | 15 ± 3 | 33 ± 5 | 27 ± 3 |
| 15 | ZnPAA3 | CO | TEA-HCl | 0.2 | 2 | >400 | 96 | 88 ± 21 | — | 554 ± 37 |

*AA represents ascorbic acid; SA represents sodium ascorbate; TEA represents triethylamine; TEA-HCl represents triethylamine hydrochloride.

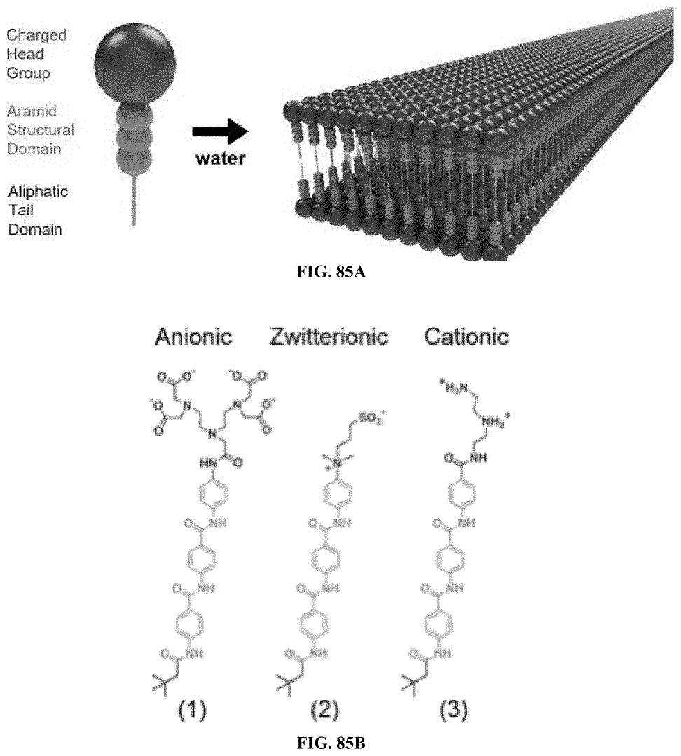

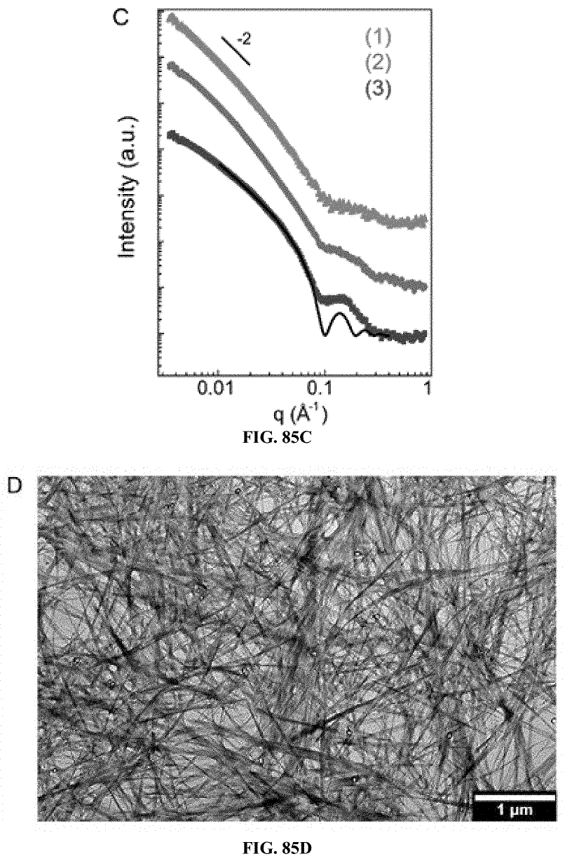

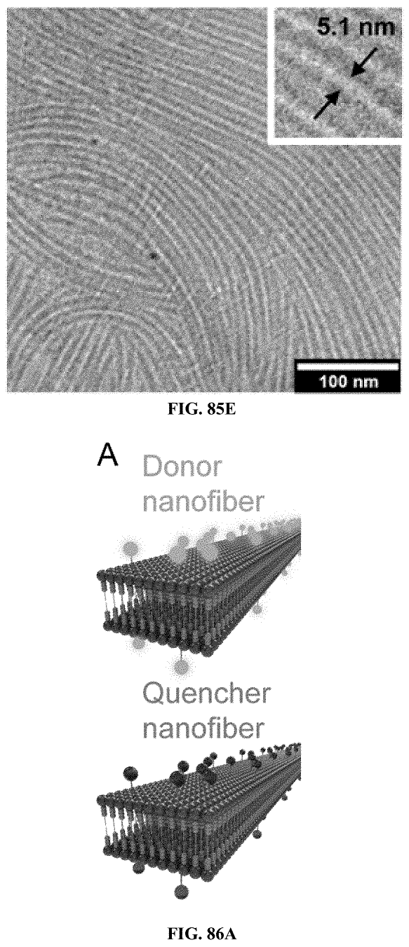

The compositions and methods described herein can give rise to novel materials with novel properties. Reference to compound numbers in this section and FIGS. 85A-98B are for this section only and do not cross-reference compound numbers in earlier sections of this description. For example, self-assembly of small molecules in water yields high surface area nanofibers with precise molecular organization. However, these structures are fragile, exhibiting molecular exchange, migration, and rearrangement, among other dynamic instabilities, and disassociation upon drying. Here a small molecule platform, the aramid amphiphile (AA), is introduced that overcomes these dynamic instabilities by incorporating a Kevlar-inspired domain into the molecular structure. This platform self-assembles in water to form planar nanofibers with a Young's modulus of 1.7 GPa and tensile strength of 1.9 GPa, surpassing the stability required for translation to the solid-state. Aqueous shear alignment to form flexible threads that support 200 times their weight when dried is demonstrated. This platform represents the first example of small molecule self-assembly to form aligned nanofiber materials for solid-state applications.

Spontaneous self-assembly of small amphiphilic molecules in water provides a powerful route to nanoscale fibers with molecular-scale dimensions and pristine internal organization. See, for example, J.-M. Lehn, Supramolecular chemistry: receptors, catalysts, and carriers. Science 227, 849-856 (1985), G. M. Whitesides, J. P. Mathias, C. T. Seto, Molecular self-assembly and nanochemistry: a chemical strategy for the synthesis of nanostructures. Science 254, 1312-1319 (1991), S. Zhang, Fabrication of novel biomaterials through molecular self-assembly. Nature biotechnology 21, 1171 (2003), and T. Aida, E. Meijer, S. I. Stupp, Functional supramolecular polymers. Science 335, 813-817 (2012), each of which is incorporated by reference in its entirety. The high-aspect-ratios afforded by molecular self-assembly allow nanofibers to be entangled or aligned, while maintaining high surface areas and tunable surface chemistries. See, for example, S. Zhang et al., A self-assembly pathway to aligned monodomain gels. Nature materials 9, 594-601 (2010), and S. Koutsopoulos, L. D. Unsworth, Y. Nagai, S. Zhang, Controlled release of functional proteins through designer self-assembling peptide nanofiber hydrogel scaffold. Proceedings of the National Academy of Sciences 106, 4623-4628 (2009), each of which is incorporated by reference in its entirety. However, supramolecular nanofibers are generally fragile due to their weak intermolecular interactions and pervasive dynamic instabilities—i.e. molecular exchange, migration, insertion, rearrangement, and transpositions. See, for example, F. Tantakitti et al., Energy landscapes and functions of supramolecular systems. Nature materials 15, 469 (2016), J. H. Ortony et al., Internal dynamics of a supramolecular nanofibre. Nature materials 13, 812 (2014), C. J. Newcomb et al., Cell death versus cell survival instructed by supramolecular cohesion of nanostructures. Nature communications 5, 3321 (2014), W. Schief, L. Touryan, S. Hall, V. Vogel, Nanoscale topographic instabilities of a phospholipid monolayer. The Journal of Physical Chemistry B 104, 7388-7393 (2000), R. M. Da Silva et al., Super-resolution microscopy reveals structural diversity in molecular exchange among peptide amphiphile nanofibres. Nature communications 7, 11561 (2016), and W. C. Wimley, T. E. Thompson, Transbilayer and interbilayer phospholipid exchange in dimyristoylphosphatidylcholine/dimyristoylphosphatidylethanolamine large unilamellar vesicles. Biochemistry 30, 1702-1709 (1991), each of which is incorporated by reference in its entirety. Further, internal transient water contributes to the biodegradability of amphiphilic nanofibers through enzymatic or hydrolytic degradation. See, for example, J. H. Ortony et al., Water Dynamics from the Surface to the Interior of a Supramolecular Nanostructure. Journal of the American Chemical Society 139, 8915-8921 (2017), and D. Yuan, J. Shi, X. Du, N. Zhou, B. Xu, Supramolecular glycosylation accelerates proteolytic degradation of peptide nanofibrils. Journal of the American Chemical Society 137, 10092-10095 (2015), each of which is incorporated by reference in its entirety. Because of these limitations, supramolecular nanofibers are generally developed for biomaterials applications where fast dynamics and biodegradability are harnessed as key design features. See, for example, S. Toledano, R. J. Williams, V. Jayawarna, R. V. Ulijn, Enzyme-triggered self-assembly of peptide hydrogels via reversed hydrolysis. Journal of the American Chemical Society 128, 1070-1071 (2006), R. Freeman et al., Reversible self-assembly of superstructured networks. Science 362, 808-813 (2018), and R. J. Williams et al., Enzyme-assisted self-assembly under thermodynamic control. Nature nanotechnology 4, 19 (2009), each of which is incorporated by reference in its entirety. These properties preclude their use in air, where they lack the structural stability imposed via the hydrophobic effect that is required to hold them together. Therefore, a new amphiphile self-assembly platform that dramatically minimizes dynamics is a critical target and could provide a groundbreaking approach to solid-state applications for which precise molecular organization, nanoscale structure, tunable surface chemistries, and water-processability are desirable.

A reliable strategy for enhancing mechanical properties of molecular materials is to incorporate hydrogen bonding domains into the molecular design. See, for example, D. C. Sherrington, K. A. Taskinen, Self-assembly in synthetic macromolecular systems via multiple hydrogen bonding interactions. Chemical Society Reviews 30, 83-93 (2001), and C. M. Paleos, D. Tsiourvas, Thermotropic liquid crystals formed by intermolecular hydrogen bonding interactions. Angewandte Chemie International Edition in English 34, 1696-1711 (1995), each of which is incorporated by reference in its entirety. For example, the collective hydrogen bonding between aromatic amides (aramids) in Kevlar (poly(p-phenylene terephthalamide), PPTA) lead to its renowned strength and impact resistance. See, for example, M. Dobb, D. Johnson, B. Saville, Supramolecular structure of a high-modulus polyaromatic fiber (Kevlar 49). Journal of Polymer Science: Polymer Physics Edition 15, 2201-2211 (1977), which is incorporated by reference in its entirety. Similar aramid chemical motifs have been incorporated into the design of biomimetic peptide-based amphiphiles (see, for example, H. Seyler, C. Storz, R. Abbel, A. F. Kilbinger, A facile synthesis of aramide-peptide amphiphiles. Soft Matter 5, 2543-2545 (2009), S. Sur, F. Tantakitti, J. B. Matson, S. I. Stupp, Epitope topography controls bioactivity in supramolecular nanofibers. Biomaterials science 3, 520-532 (2015), and R. C. Claussen, B. M. Rabatic, S. I. Stupp, Aqueous self-assembly of unsymmetric peptide bolaamphiphiles into nanofibers with hydrophilic cores and surfaces. Journal of the American Chemical Society 125, 12680-12681 (2003), each which is incorporated by reference in its entirety); however, the molecular packing was not adjusted in these cases to promote assembly into nanofibers or to optimize mechanical behavior. In contrast to small amphiphilic molecules, polymeric aramid nanofibers composed of PPTA have shown strong mechanical behavior, even upon drying, but neither control over nanofiber surface chemistry nor the internal organization is achievable. See, for example, M. Yang et al., Dispersions of aramid nanofibers: a new nanoscale building block. ACS nano 5, 6945-6954 (2011), which is incorporated by reference in its entirety. Despite these efforts, structural stability of nanofibers composed of small amphiphilic molecules has never been demonstrated outside of water.

Here, a versatile small molecule platform for self-assembly in water that maintains three attributes to achieve extraordinary mechanical stability have been designed: (1) a high hydrogen bond density, with six hydrogen bonds per molecule; (2) in-register organization within each hydrogen bond network and the ability to form interplane π-π stacking (see, for example, A. Johansson, P. Kollman, S. Rothenberg, J. McKelvey, Hydrogen bonding ability of the amide group. Journal of the American Chemical Society 96, 3794-3800 (1974), which is incorporated by reference in its entirety); and (3) minimal steric packing strain and torsion to minimize H-bond distances (see, for example, D. A. Dixon, K. D. Dobbs, J. J. Valentini, Amide-water and amide-amide hydrogen bond strengths. The Journal of Physical Chemistry 98, 13435-13439 (1994), which is incorporated by reference in its entirety), achieved by incorporating unobtrusive amphiphile head and tail groups into the molecular design. These three attributes can be exploited to produce self-assembled nanofibers with dramatically suppressed exchange dynamics, giving rise to unprecedented mechanical durability. As a result, these nanofibers are candidates for alignment and removal from water while maintaining their structure to demonstrate the first macroscopic air-stable small molecule nanofiber threads.

A new molecular design motif, aramid amphiphiles (AA), and their self-assembly into ultra-stable planar nanofibers is introduced (FIG. 85A). This platform is designed to be intrinsically hydrolysis-resistant, containing amides that are buried in the hydrophobic interior of the nanostructure, away from water. See, for example, J. H. Ortony et al., Water Dynamics from the Surface to the Interior of a Supramolecular Nanostructure. Journal of the American Chemical Society 139, 8915-8921 (2017), which is incorporated by reference in its entirety. AAs with three different head group chemistries to tune the surface charge of the nanofibers were synthesized: compound 1, an anionic pentetic acid amphiphile, compound 2, a zwitterionic ammonium sulfonate amphiphile, and compound 3, a cationic triazaheptane amphiphile (FIG. 85B, see below). Small angle X-ray scattering (SAXS) of compounds 1-3 in water shows a slope of −2 in the Guinier regime (FIG. 85C), characteristic of one-dimensional fiber-like structures in solution. The best fit to the SAXS scattering profiles of 1-3 corresponds to a lamellar bilayer model, giving a nanofiber thickness of 3.9 nm, which is corroborated by atomic force microscopy (AFM) (see below). Lamellar models do not provide lateral dimensions, cryogenic transmission electron microscopy (TEM) measurements were used to designate the widths of nanofibers of 1, 2, and 3 as 5.5 nm, 5.1 nm, and 5.8 nm, respectively (see below). Interestingly, self-assembled AA nanofibers are unusually long, reaching up to 20 μm, corresponding to width-to-length aspect-ratios of 5,000:1 (FIGS. 85D-85E, and FIGS. 92A-94). See, for example, H. Yokoi, T. Kinoshita, S. Zhang, Dynamic reassembly of peptide RADA16 nanofiber scaffold. Proceedings of the National Academy of Sciences 102, 8414-8419 (2005), J. D. Hartgerink, E. Beniash, S. I. Stupp, Self-assembly and mineralization of peptide-amphiphile nanofibers. Science 294, 1684-1688 (2001), each of which is incorporated by reference in its entirety.

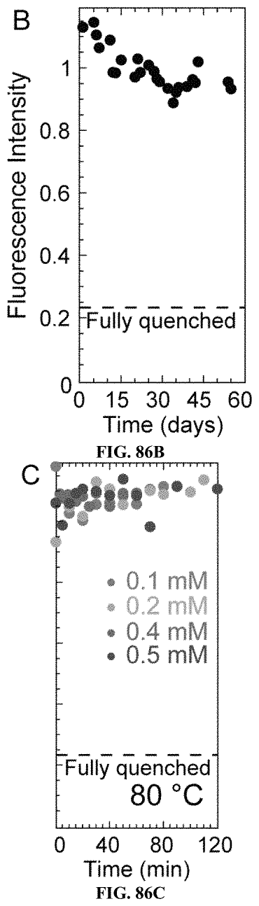

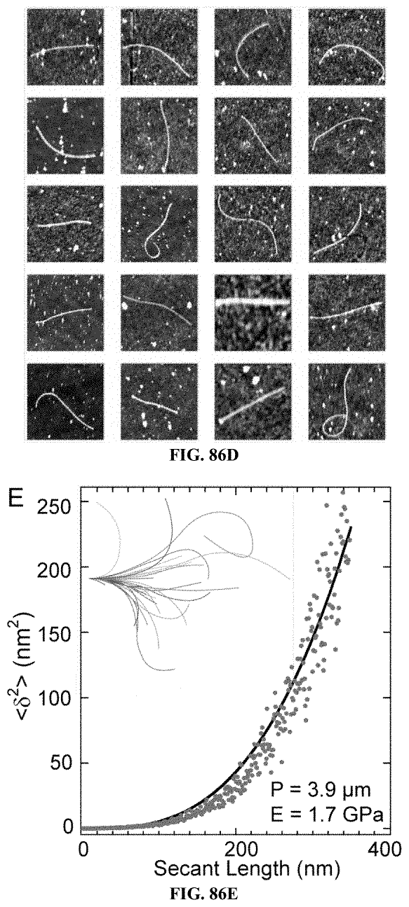

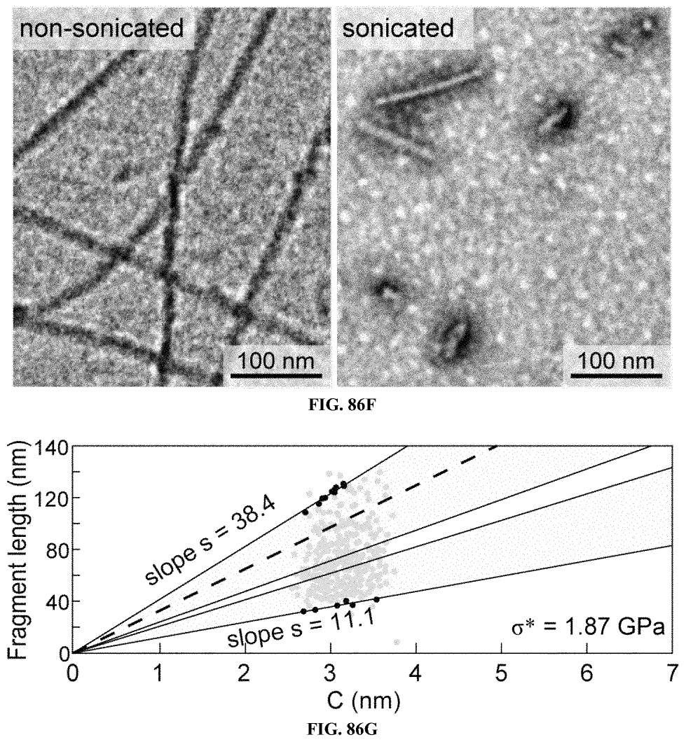

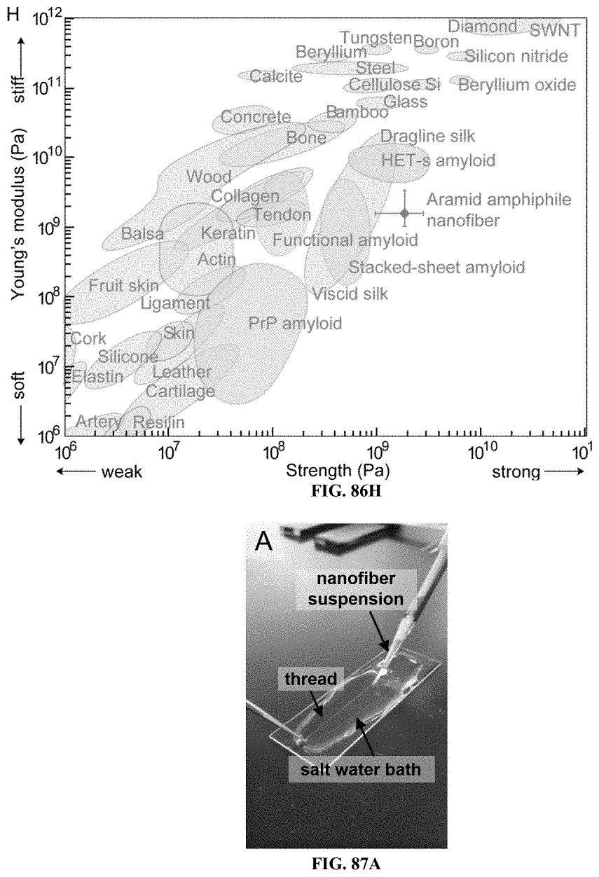

AA nanofibers are expected to exhibit strong collective hydrogen bonding that leads to internal cohesion and therefore slow molecular exchange dynamics. See, for example, J. H. Ortony et al., Internal dynamics of a supramolecular nanofibre. Nature materials 13, 812 (2014), which is incorporated by reference in its entirety. The rate at which individual aramid amphiphile molecules exchange between adjacent nanofibers was probed by Förster resonant energy transfer (FRET) dark quenching (SM Section S3f). See, for example, E. D. Matayoshi, G. T. Wang, G. A. Krafft, J. Erickson, Novel fluorogenic substrates for assaying retroviral proteases by resonance energy transfer. Science 247, 954-958 (1990), which is incorporated by reference in its entirety. Separate nanofiber suspensions containing either fluorophore- or quencher-tagged amphiphiles were mixed (FIG. 86A) and observed minimal molecular exchange between adjacent nanofibers over 55 days (FIG. 86B). Further, no changes in peak fluorescence intensity were observed when mixtures of fluorophore- and quencher-labeled nanofibers were heated to 80° C. over a range of concentrations (FIG. 86C). These results highlight the stability that aramid hydrogen bonding imparts on amphiphilic nanofiber assemblies, representing a substantial departure from the typical exchange rates of 1-2 hours reported in phospholipid membranes and supramolecular peptide assemblies. See, for example, R. M. Da Silva et al., Super-resolution microscopy reveals structural diversity in molecular exchange among peptide amphiphile nanofibres. Nature communications 7, 11561 (2016), W. C. Wimley, T. E. Thompson, Transbilayer and interbilayer phospholipid exchange in dimyristoylphosphatidylcholine/dimyristoylphosphatidylethanolamine large unilamellar vesicles. Biochemistry 30, 1702-1709 (1991), each of which is incorporated by reference in its entirety.

The slow exchange dynamics of AA nanofibers allow us to perform single-nanofiber mechanical characterization experiments, which have not previously been demonstrated on self-assembled nanofibers due to their intrinsic dynamic instabilities. Here, the Young's elastic modulus and tensile strength of compound 3 nanofibers was accessed using topographical analyses recently introduced and demonstrated on solid-state nanofilaments with diameters of around 10 nm including silver nanowires, carbon nanotubes, and amyloid fibrils. See, for example, T. P. Knowles et al., Role of intermolecular forces in defining material properties of protein nanofibrils. Science 318, 1900-1903 (2007), Y. Y. Huang, T. P. Knowles, E. M. Terentjev, Strength of nanotubes, filaments, and nanowires from sonication-induced scission. Advanced materials 21, 3945-3948 (2009), and G. Lamour et al., Mapping the broad structural and mechanical properties of amyloid fibrils. Biophysical journal 112, 584-594 (2017), each of which is incorporated by reference in its entirety.

The Young's modulus is determined by evaluating the shape fluctuations of resting nanofibers (n=29) from AFM images to extract their bending rigidity (FIG. 86D). See, for example, T. P. Knowles et al., Role of intermolecular forces in defining material properties of protein nanofibrils. Science 318, 1900-1903 (2007), and G. Lamour, J. B. Kirkegaard, H. Li, T. P. Knowles, J. Gsponer, Easyworm: an open-source software tool to determine the mechanical properties of worm-like chains. Source code for biology and medicine 9, 16 (2014), each of which is incorporated by reference in its entirety. Parametric splines to the contours of each nanofiber were traced (FIG. 86E, inset) and fit to determine a persistence length, P=3.9±0.7 μm, and therefore the Young's modulus of E=1.7±0.7 GPa (FIG. 86E, see below). The ultimate tensile strength is probed through sonicated-induced scission which produces nanostructure fragments below a threshold length, Lₗᵢₘ. See, for example, Y. Y. Huang, T. P. Knowles, E. M. Terentjev, Strength of nanotubes, filaments, and nanowires from sonication-induced scission. Advanced materials 21, 3945-3948 (2009), which is incorporated by reference in its entirety. From visualizing hundreds of post-sonication fragments by TEM (FIG. 86F), Lₗᵢₘ for the nanofibers was evaluated from their fragment length distribution as 98±26 nm, which corresponds to a tensile strength of σ*=1.87±1.00 GPa (FIG. 86G, see below). These mechanical properties rival the performance of silk and place AA nanofibers in a region of the Ashby plot viable for solid state applications (FIG. 86H). See, for example, G. Lamour et al., Mapping the broad structural and mechanical properties of amyloid fibrils. Biophysical journal 112, 584-594 (2017), which is incorporated by reference in its entirety.

Macroscopic materials consisting of small molecule amphiphile nanofibers can take advantage of high surface areas on the order of 10² m²/g dictated by molecular size, tunable surface chemistries for targeted interactions, and the capacity for co-assembly of different amphiphiles to perform multiple functions on the same surface. See, for example, X. Zhao et al., Molecular self-assembly and applications of designer peptide amphiphiles. Chemical Society Reviews 39, 3480-3498 (2010), and K. L. Niece, J. D. Hartgerink, J. J. Donners, S. I. Stupp, Self-assembly combining two bioactive peptide-amphiphile molecules into nanofibers by electrostatic attraction. Journal of the American Chemical Society 125, 7146-7147 (2003), each of which is incorporated by reference in its entirety. However, these materials have been historically limited to solvated environments. The suppressed exchange dynamics and robust mechanical properties observed for AA nanofibers could, for the first time, extend these advantages to solid-state applications.

Previously, 1-dimensional threads have been formed from small molecule peptide amphiphile systems by shear alignment for applications including cell scaffolding and protein delivery. See, for example, S. Zhang et al., A self-assembly pathway to aligned monodomain gels. Nature materials 9, 594-601 (2010), and N. L. Angeloni et al., Regeneration of the cavernous nerve by Sonic hedgehog using aligned peptide amphiphile nanofibers. Biomaterials 32, 1091-1101 (2011), each of which is incorporated by reference in its entirety. Inspired by this processing strategy, cationic (compound 3) nanofibers were annealed in water to form liquid-crystalline bundles, which were then pulled into a divalent (Na₂SO₄) salt solution to form a 1-dimensional nanofiber gel (FIG. 87A, see below). The unique ability of the nanofiber gel to withstand drying in air, forming a stable solid thread was demonstrated. Scanning electron microscopy (SEM) imaging of the AA threads revealed thread diameters near 20 μm and striations consistent with the presence of nanofiber bundles (FIG. 87B). The thread can be handled, bent without breaking, and support over 200 times its own weight (FIGS. 87C-87D).

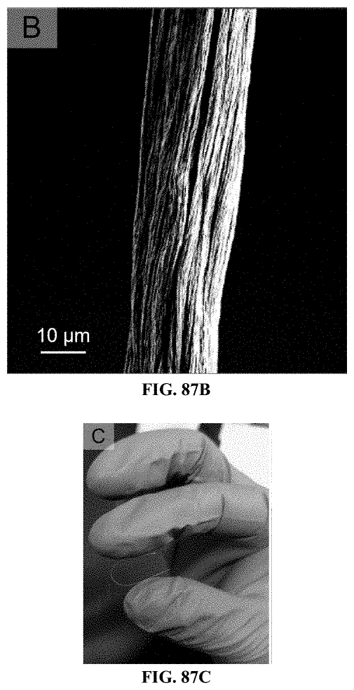

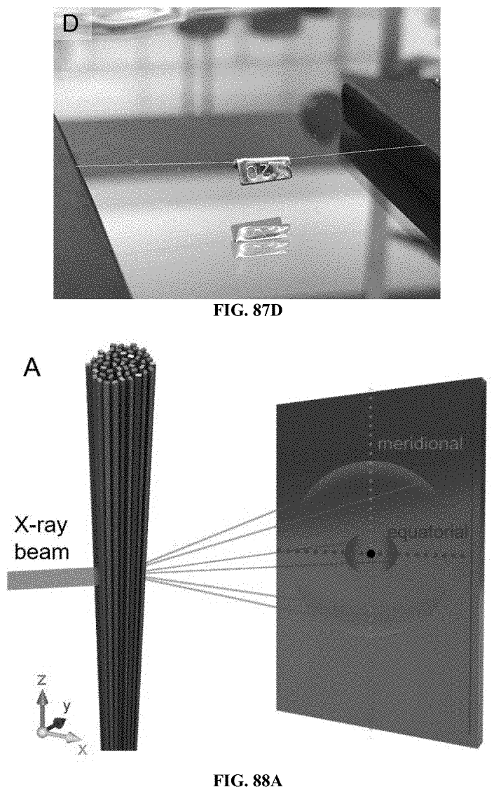

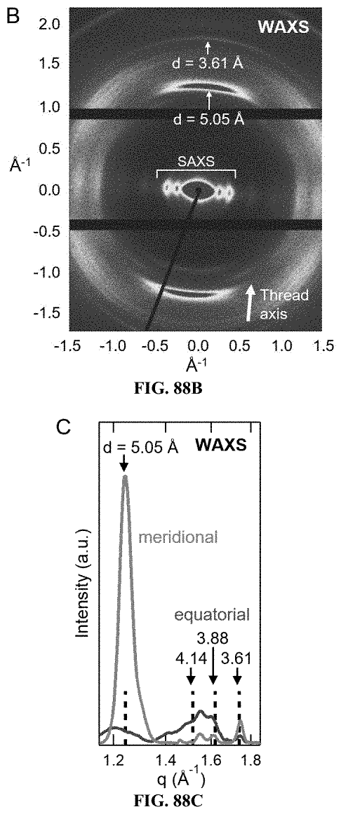

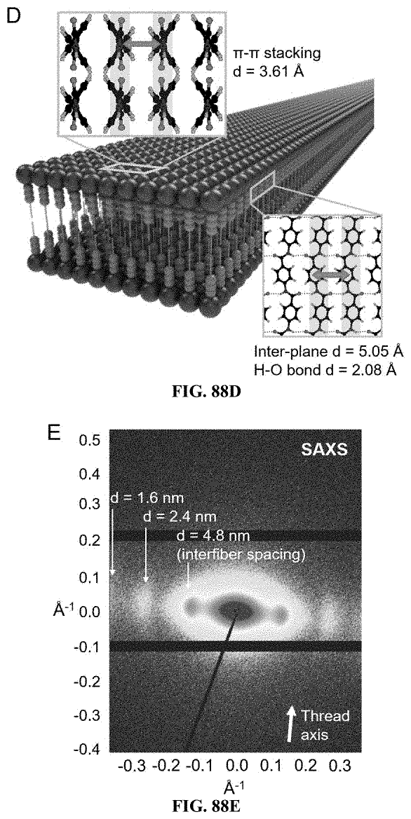

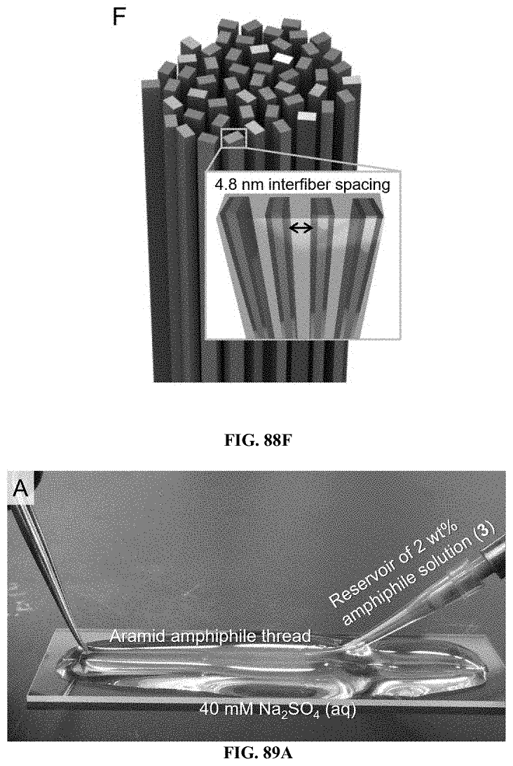

X-ray scattering was performed to determine the structure within the nanofiber thread and to confirm that the planar nanofibers persist after processing (FIG. 88A). Wide-angle X-ray scattering (WAXS) shows anisotropic peaks indicating nanofiber alignment within a vertically oriented thread, with the strongest WAXS peak occurring at a d-spacing of 5.05 Å (FIG. 88B). By integrating the WAXS pattern in the meridional and equatorial directions, peaks were isolated to simulate a unit cell that shows molecular packing resembling poly(p-benzamide) (FIG. 88C, see below). See, for example, Y. Takahashi, Y. Ozaki, M. Takase, W. Krigbaum, Crystal structure of poly (p-benzamide). Journal of Polymer Science Part B: Polymer Physics 31, 1135-1143 (1993), which is incorporated by reference in its entirety. This structure reveals that intermolecular aramid hydrogen bonding is dominant along the nanofiber long axis with H—O hydrogen bond distances of 2.08 Å. π-π stacking at an interplane distance of 3.61 Å laterally holds together hydrogen bonding sheets across the nanofiber width (FIG. 88D). Using these dimensions, a surface area of AA nanofibers within the dried thread was calculated as 200 m²/g. At longer length scales, SAXS peaks in the equatorial direction result from AA nanofibers aligned along the thread axis (FIG. 88E), with 4.8 nm lamellar spacings between nanofibers within the thread (FIG. 88F). The AA thread represents the first demonstration of small molecule amphiphilic self-assembly to form macroscopic solid-state 1-dimensional materials, with internal structure relevant to a range of previously inaccessible applications. See, for example, P. Russell, Photonic crystal fibers. Science 299, 358-362 (2003), Y. Xu et al., Nanostructured polymer films with metal-like thermal conductivity. Nature communications 10, 1-8 (2019), and H. L. Tuller, Ionic conduction in nanocrystalline materials. Solid State Ionics 131, 143-157 (2000), each of which is incorporated by reference in its entirety.

Here, a new self-assembly platform is presented, the aramid amphiphile nanofiber. Six hydrogen bonds fix each molecule within an extended network, which, when combined with lateral π-π stacking, give rise to nanofibers with exceptional mechanical properties. These nanofibers exhibit unusually slow molecular exchange dynamics and tensile strengths surpassing nature's strongest filaments. A shear alignment technique was applied to form macroscopic threads composed of aligned nanofiber bundles with uniform 4.8 nm interfiber spacings and surface areas of 200 m²/g. Further, these nanofiber threads are flexible, can be handled, and can support 200 times their weight. The aramid amphiphile platform extends supramolecular small molecule assemblies beyond fragile and biodegradable structures. This work pushes the state-of-the-art past aqueous-only applications and provides a novel route to nanostructured, robust, solid-state materials.

Synthesis and Methods

Synthesis Overview:

The syntheses used in this study involve: 1) carbodiimide-mediated coupling reactions to form amide linkages, 2) conventional deprotection reactions of tert-butyloxycarbonyl (Boc), and 3) hydrolysis of ester functionalities to produce carboxylic acid moieties. As the only exception, the zwitterionic head group of 2 is obtained by quaternization of a tertiary amine with a propanesultone. ¹H and ¹³C nuclear magnetic resonance (NMR, SM described below) and mass spectroscopy (MS, described below) were used to confirm the chemical composition of intermediates and products. Details on each of compounds 1, 2, and 3 and their intermediates are given below.

Materials

Methyl 4-aminobenzoate (Sigma Aldrich, 98%), 3,3-dimethylbutyric acid (Sigma Aldrich, 98%), N,N-dimethyl-p-phenylenediamine (DPP, Sigma Aldrich, 97%), N-Boc-p-phenylenediamine (BPP, Sigma Aldrich, 97%), 1,3-propanesultone (PPS, Sigma Aldrich, 99%), 1,4-bis-Boc-1,4,7-triazaheptane (BBT, Chem Impex, 100%), diethylenetriamine-N,N,N″,N″-tetra-tert-butyl acetate-N′-acetic acid (DPTA, Combi Blocks, 95%), 1-ethyl-3-(3-dimethylaminopropyl)carbodiimide hydrochloride (EDC, TCI Chemicals, 98%), 4-dimethylaminopyridine (DMAP, TCI Chemicals, 99%), 1-hydroxybenzotriazole hydrate (HOBt, TCI Chemicals, 97%), lithium hydroxide (LiOH, Alfa Aesar, 98%), sodium bicarbonate (NaHCO₃, Alfa Aesar, 99%), hydrochloric acid (HCl, Alfa Aesar, 36%), sodium sulfate (Na₂SO₄, Fisher Scientific, 99%), and trifluoroacetic acid (TFA, Alfa Aesar, 99%) were used as received without further purification.

### Anionic Amphiphile and its Intermediates

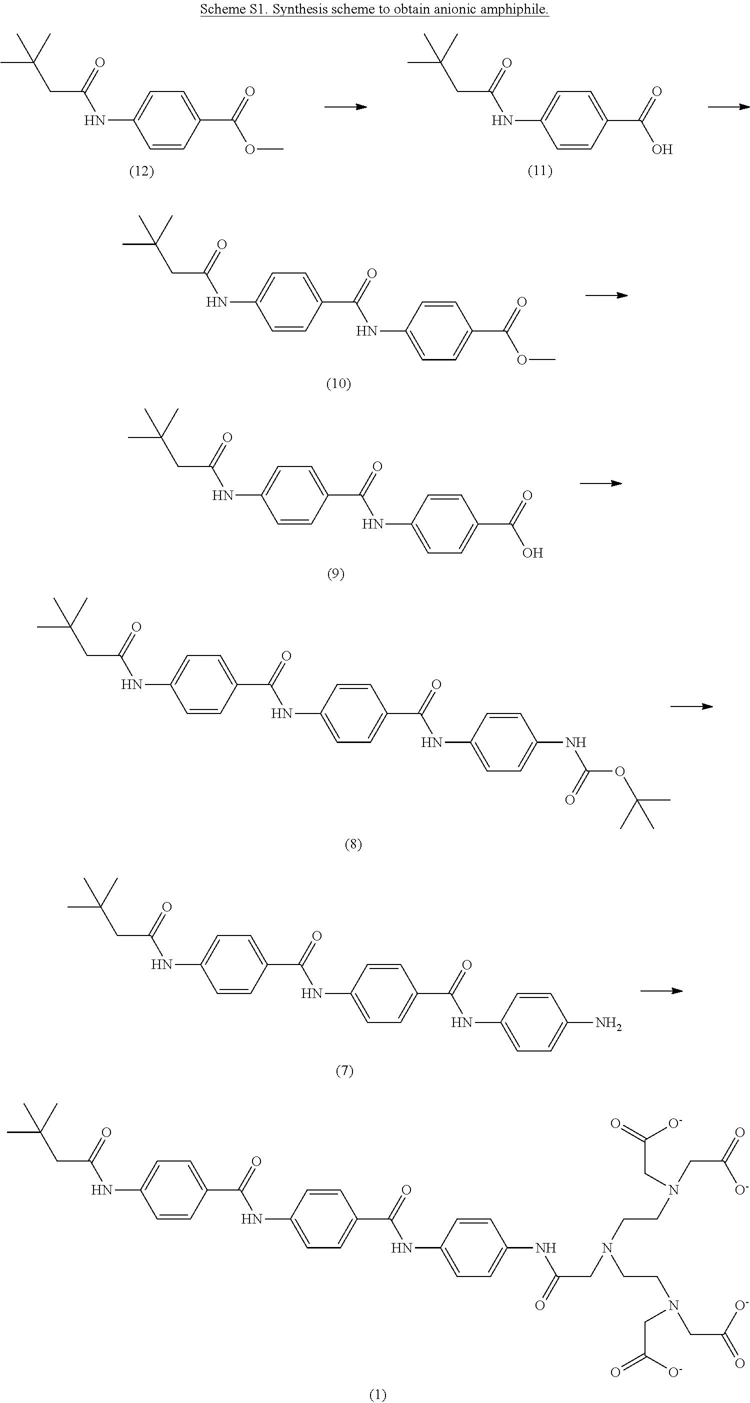

Methyl 4-(3,3-dimethylbutanamido)benzoate (12): A solution of methyl 4-aminobenzoate (11.01 mmol), 3,3-dimethylbutyric acid (16.52 mmol), EDC (33.03 mmol), and DMAP (33.03 mmol) in tetrahydrofuran (50 mL) was stirred at room temperature for 24 h. After the reaction, the solvent was removed in vacuum, and the residue was washed with distilled water and extracted in chloroform. The organic layer was purified by column chromatography with silica gel by using 1:1 ethyl acetate:hexane by volume (yield: 72%). ¹H NMR (400 MHz, DMSO-d): δ=7.89 (d, 2H), 7.75 (d, 2H), 3.82 (s, 3H), 2.23 (s, 2H), 1.03 (s, 9H) ppm.

4-(3,3-dimethylbutanamido)benzoic acid (11): 10 M LiOH (10 mL) was added to a stirred solution of compound 12 (4.25 mmol) in ethanol (40 mL). The mixture was heated to 60° C. and refluxed for 3 h, and then neutralized with an aqueous HCl solution. The precipitate was filtered off, and washed with water several times. The crude product was purified by reprecipitation from chloroform and methanol and dried under vacuum (yield: 98%). ¹H NMR (400 MHz, DMSO-d): δ=7.87 (d, 2H), 7.72 (d, 2H), 2.23 (s, 2H), 1.03 (s, 9H) ppm.

Methyl 4-(4-(3,3-dimethylbutanamido)benzamido)benzoate (10): EDC (6.37 mmol), and DMAP (6.37 mmol) were added to a solution of compound 11 (2.13 mmol), and methyl 4-aminobenzoate (6.37 mmol) in dimethylformamide (30 mL). The solution was stirred for 24 h at 50° C. After the reaction, the solvent was removed in vacuum, and the remaining residue was precipitated in water. The crude mixture was collected with filter flask. The filtered solid was washed with excess methanol and dried in vacuum (yield: 83%). ¹H NMR (400 MHz, DMSO-d): δ=7.95 (m, 6H), 7.77 (d, 2H), 3.84 (s, 3H), 2.24 (s, 2H), 1.04 (s, 9H) ppm.

4-(4-(3,3-dimethylbutanamido)benzamido)benzoic acid (9): 10M LiOH (10 mL) was added to a stirred solution of compound 10 (2.55 mmol) in tetrahydrofuran (20 mL) and ethanol (10 mL). The mixture was refluxed for 6 h and then neutralized with an aqueous HCl solution. The precipitate was filtered off, washed with water, and dried under vacuum to afford the product (yield: 98%). ¹H NMR (400 MHz, DMSO-d): δ=7.93 (m, 6H), 7.76 (d, 2H), 2.24 (s, 2H), 1.04 (s, 9H) ppm.

tert-Butyl 4-(4-(4-(3,3-dimethylbutanamido)benzamido)benzamido)phenylcarbamate (8): Into dimethylformamide (20 mL), compound 9 (0.85 mmol), BPP (2.55 mmol), EDC (2.55 mmol), and DMAP (2.55 mmol) were added. The well-dissolved solution was stirred at room temperature for 24 h. After solvent evaporation, the crude mixture was washed with water and methanol to give the desired white solid product (yield: 81%). ¹H NMR (400 MHz, DMSO-d): δ=7.96 (m, 6H), 7.77 (d, 2H), 7.64 (d, 2H), 7.41 (d, 2H), 2.25 (s, 2H), 1.46 (s, 9H), 1.05 (s, 9H) ppm.

N-(4-(amino)phenyl)-4-(4-(3,3-dimethylbutanamido)benzamido)benzamide (7): TFA (500 μL) was added dropwise into the solution of compound 8 (0.55 mmol) in methylene chloride (15 mL). After stirring the mixture for 6 h at room temperature, the volatiles were distilled off and the remaining mixture was washed with saturated NaHCO₃ solution. The solid precipitate was filtered and dried in vacuum (yield: 99%). ¹H NMR (400 MHz, DMSO-d): 6=7.95 (m, 6H), 7.75 (d, 2H), 7.48 (d, 2H), 6.72 (d, 2H), 2.25 (s, 2H), 1.05 (s, 9H) ppm.

2,2′,2″,2′″-((((2-((4-(4-(4-(3,3-dimethylbutanamido)benzamido)benzamido)phenyl)amino)-2-ox-oethyl)azanediyl)bis(ethane-2,1-diyl))bis(azanetriyl))tetraacetate (1): A solution of compound 7 (0.29 mmol), DPTA (058 mmol), EDC (1.17 mmol), and DMAP (1.17 mmol) in dimethylformamide (20 mL) was stirred at 50° C. for 72 h. After the reaction, the solvent was removed in vacuum. The remaining residue was purified by flash column chromatography with silica gel by using 7:1 tetrahydrofuran: chloroform by volume as an eluent. The isolated compound was then reacted with TFA (500 μL) in methylene chloride (15 mL) for 48 h. The volatile fraction was removed under reduced pressure. Tetrahydrofuran was added to suspend the product and the product was collected by filtration (yield: 67%). ¹H NMR (400 MHz, DMSO-d): δ=7.97 (m, 6H), 7.75 (m, 4H), 7.61 (d, 2H), 4.06 (s, 2H), 3.51 (s, 8H), 3.21 (t, 4H), 3.01 (t, 4H), 2.25 (s, 2H), 1.05 (s, 9H) ppm. ¹³C NMR (400 MHz, DMSO-d): δ=173.2, 170.9, 165.6, 165.1, 142.9, 135.7, 134.4, 128.9, 121.2, 119.8, 118.7, 55.1, 52.8, 50.1, 31.4, 30.1 ppm. MS (MALDI-ToF) m/z calculated: 819.34; [M+H]⁺. Found: 820.35.

### Zwitterionic Amphiphile and its Intermediates

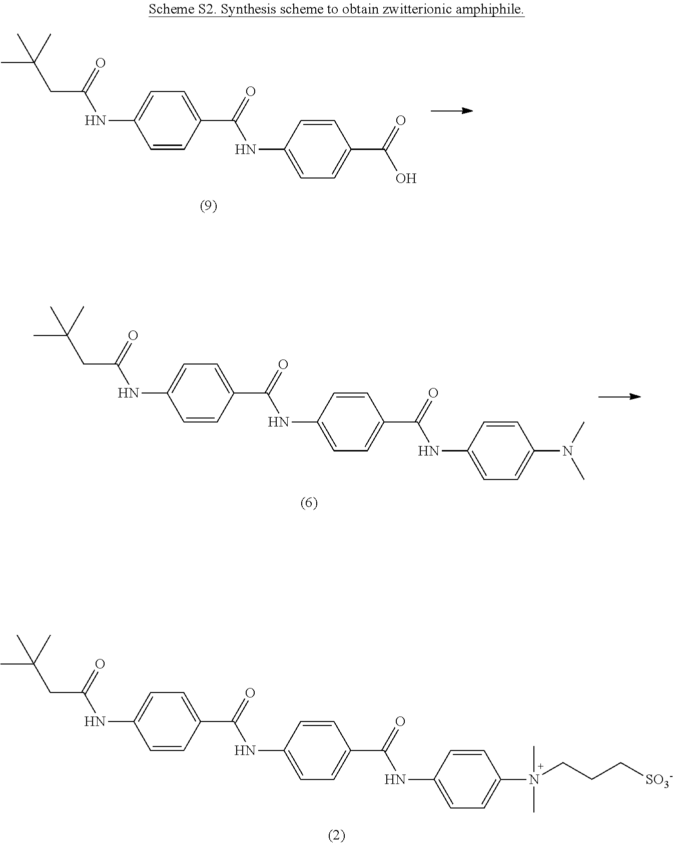

N-(4-(dimethyl amino)phenyl)-4-(4-(3,3-dimethylbutanamido)benzamido)benzamide (6): A solution of compound 9 (0.85 mmol), DPP (2.55 mmol), EDC (2.55 mmol), and HOBt (2.55 mmol) in dimethylformamide (20 mL) was stirred at 50° C. for 24 h. After the reaction, the solvent was distilled off and the remaining residue was precipitated with water. The crude mixture was collected and washed with chloroform several times (yield: 78%). ¹H NMR (400 MHz, DMSO-d): δ=7.95 (m, 6H), 7.77 (d, 2H), 7.57 (d, 2H), 6.73 (d, 2H), 2.88 (s, 6H), 2.25 (s, 2H), 1.05 (s, 9H) ppm.

3-((4-(4-(4-(3,3-dimethylbutanamido)benzamido)benzamido)phenyl)dimethylammonio)-propane-1-sulfonate (2): Compound 6 (1.85 mmol) was dissolved in dimethylformamide (15 mL) and tetrahydrofuran (15 mL). PPS (5 mL) was slowly injected using a syringe and the clear solution was stirred for 48 h in a sealed pressure tube at 70° C. The volatile fraction was removed under reduced pressure and acetonitrile (50 mL) was added. The resulting precipitate was filtered and dried in vacuum (yield: 85%). ¹H NMR (400 MHz, DMSO-d): δ=7.98 (m, 8H), 7.90 (d, 2H), 7.78 (d, 2H), 3.99 (m, 2H), 3.58 (s, 6H), 2.39 (t, 2H), 1.66 (m, 2H), 1.05 (s, 9H) ppm. ¹³C NMR (400 MHz, DMSO-d): δ=170.9, 165.8, 143.2, 140.9, 139.6, 129.1, 122.2, 121.1, 119.8, 118.7, 68.1, 54.4, 50.1, 47.9, 34.4, 30.1, 20.3 ppm. MS (MALDI-ToF) m/z calculated: 595.26; [M+H]⁺. Found: 595.41.

Cationic Amphiphile and its Intermediates

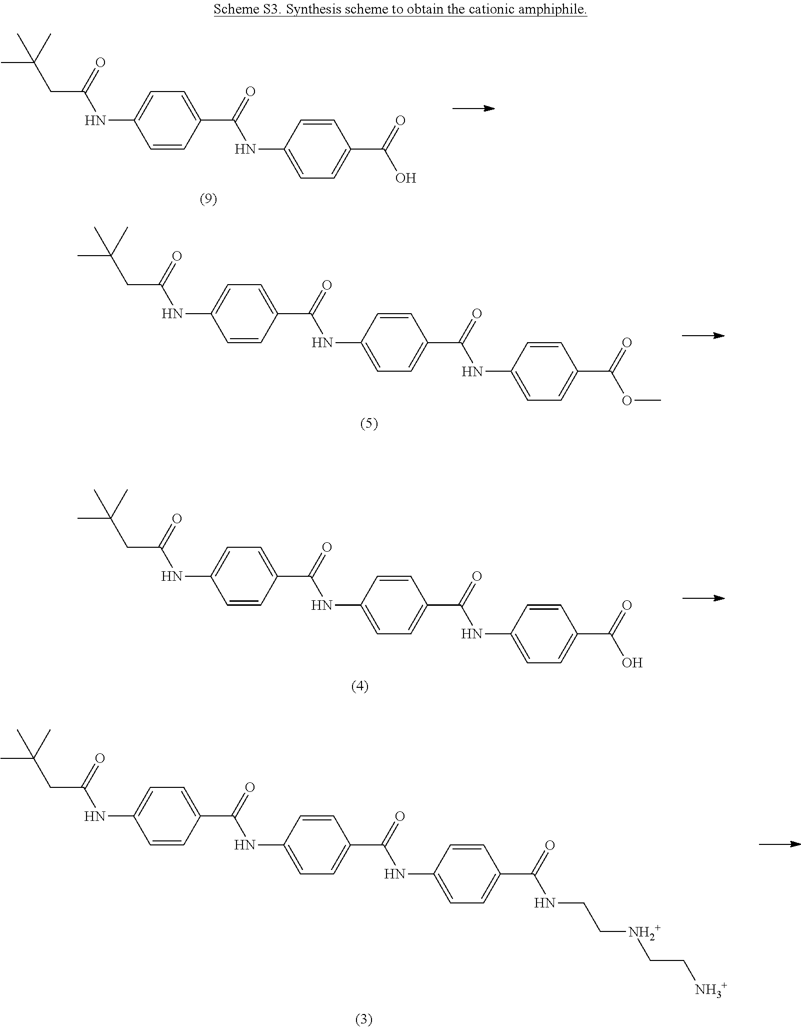

Methyl 4-(4-(4-(3,3-dimethylbutanamido)benzamido)benzamido)benzoate (5): EDC (4.23 mmol) and DMAP (4.23 mmol) were added to a solution of compound 9 (1.41 mmol) and methyl 4-aminobenzoate (4.23 mmol) in dimethylformamide (20 mL). The solution was stirred for 24 h at 50° C. After the reaction, the solvent was removed in vacuum, and the remaining residue was precipitated with water. The collected crude mixture was further washed with methanol and dried in vacuum (yield: 75%). ¹H NMR (400 MHz, DMSO-d): δ=7.97 (m, 8H), 7.78 (d, 2H), 3.85 (s, 3H), 2.25 (s, 2H), 1.05 (s, 9H) ppm.

4-(4-(4-(3,3-dimethylbutanamido)benzamido)benzamido)benzoic acid (4): 10M LiOH (10 mL) was added to a stirred solution of compound 5 (1.05 mmol) in tetrahydrofuran (20 mL), and ethanol (10 mL). The mixture was refluxed for 12 h and then neutralized with an aqueous HCl solution to obtain a precipitate. The crude product was purified by reprecipitation with chloroform and ethanol and dried under vacuum (yield: 93%). ¹H NMR (400 MHz, DMSO-d): δ=7.94 (m, 8H), 7.78 (d, 2H), 2.25 (s, 2H), 1.05 (s, 9H) ppm.

1-(2-(4-(4-(4-(3,3-dimethylbutanamido)benzamido)benzamido)benzamido)ethyl)ethane-1,2-diaminium (3): A solution of compound 4 (0.64 mmol), BBT (1.27 mmol), EDC (1.92 mmol), and DMAP (1.92 mmol) in dimethylformamide (20 mL) was stirred at room temperature for 48 h. After the reaction, the solvent was removed in vacuum, and the remaining residue was washed with ethyl acetate several times. The isolated compound was then reacted with TFA (500 μL) in methylene chloride (15 mL) for 24 h. The volatile fraction was evaporated under reduced pressure. Diethyl ether was added to collect the product by filtration (yield: 72%). ¹H NMR (400 MHz, DMSO-d): δ=7.98 (m, 10H), 7.76 (d, 2H), 3.58 (m, 2H), 3.39 (m, 2H), 3.13 (m, 4H), 2.25 (s, 2H), 1.04 (s, 9H) ppm. ¹³C NMR (400 MHz, DMSO-d): δ=171.1, 167.1, 165.7, 142.9, 129.1, 128.7, 128.4, 120.1, 119.4, 118.7, 50.1, 47.4, 44.6, 35.8, 31.4, 30.1 ppm. MS (MALDI-ToF) m/z calculated: 558.30; [M+H]⁺. Found: 559.29.

Synthesis of Materials for Förster Resonance Energy Transfer

Molecular exchange was measured by Förster resonance energy transfer (FRET) dark quenching when two nanofibers populations, one containing a donor fluorophore and the other containing a dark quencher, were introduced into the same suspension. EDANS ((5-((2-aminoethyl)amino)naphthalene-1-sulfonic acid)) was used as the donor fluorophore and DABCYL (4-(dimethylaminoazo)benzene-4-carboxylic acid) was used as the dark quencher.

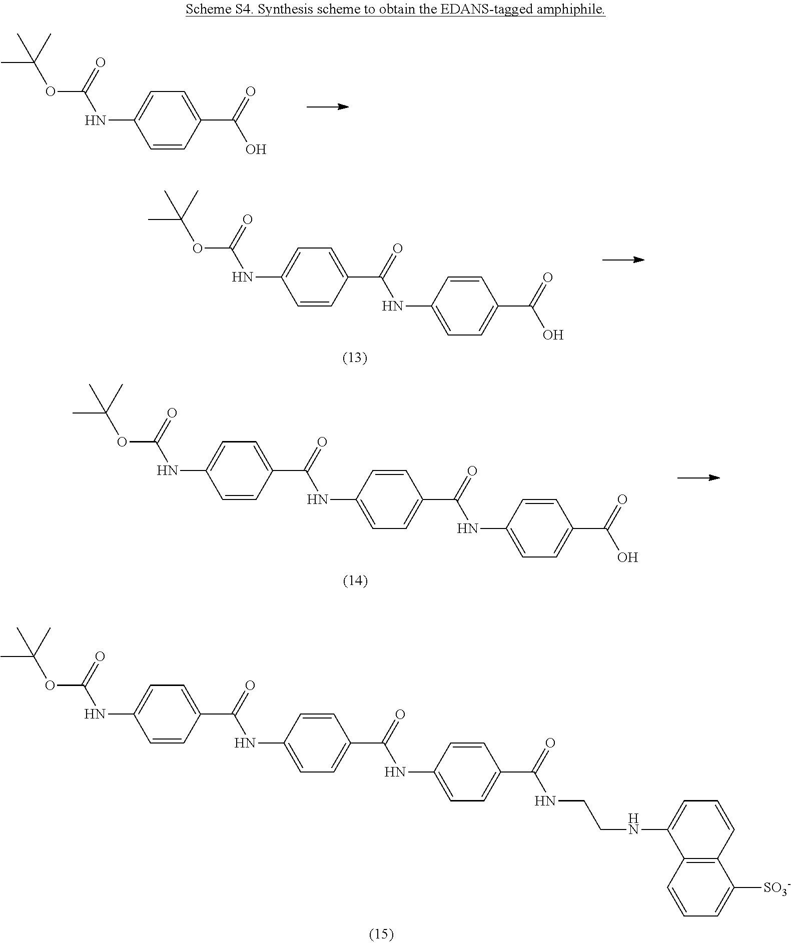

4-(4-(tert-Butoxycarbonyl)benzamido)benzoic acid (13): A solution of 4-(Boc-amino)benzoic acid (4.21 mmol), methyl 4-aminobenzoate (8.42 mmol), EDC (8.42 mmol), and DMAP (8.42 mmol) in chloroform (100 mL) were stirred at room temperature for 12 h. After the reaction, the solvent was evaporated under reduced pressure, and the remaining residue was precipitated with water. The mixture was filtered, and the precipitate was washed with methylene chloride several times. The solid material was dissolved in tetrahydrofuran (40 mL) and ethanol (20 mL). LiOH (21.1 mmol) in water (10 mL) was added to this solution, which was then refluxed at 70° C. for 3 h. The reaction mixture was shifted to room temperature and acidified to pH 2 with the addition of 5M HCl solution. The precipitate was filtered through a Buchner funnel and dried in vacuum (yield: 87%). ¹H NMR (400 MHz, DMSO-d): δ=7.93 (m, 6H), 7.66 (d, 2H), 1.49 (s, 9H) ppm.

4-(4-(4-(tert-Butoxycarbonyl)benzamido)benzamido)benzoic acid (14): A solution of compound 13 (2.11 mmol), methyl 4-aminobenzoate (4.22 mmol), EDC (4.22 mmol), and DMAP (4.22 mmol) were in dimethylformamide (30 mL) was stirred at room temperature for 24 h. After the reaction, the solvent was distilled off, and the remaining residue was washed with water and methanol. The solid material was dissolved in tetrahydrofuran (20 mL) and ethanol (10 mL). LiOH (10.5 mmol) in water (10 mL) was added to this solution, which was then refluxed at 70° C. for 6 h. The reaction mixture was shifted to room temperature and acidified to pH 2 with the addition of 5M HCl solution. The precipitate was collected on a Buchner funnel and dried in vacuum (yield: 82%). ¹H NMR (400 MHz, DMSO-d): δ=7.95 (m, 10H), 7.67 (d, 2H), 1.49 (s, 9H) ppm.

EDANS-tagged amphiphile, 5-((2-(4-(4-(4-(3,3-dimethylbutanamido)benzamido)benzamido) benzamido)ethyl)amino)naphthalene-1-sulfonic acid (15): A solution of compound 14 (0.21 mmol), EDC (0.25 mmol), and DMAP (0.25 mmol) in dimethylformamide (10 mL) was stirred for 30 min. EDANS (0.25 mmol) was then added into the solution and the solution was stirred for 24 h at room temperature. Water was poured into the solution to yield a precipitate, which was obtained by filtration on a Buchner funnel and washed with chloroform (yield: 64%). ¹H NMR (400 MHz, DMSO-d): δ=8.02 (m, 1H), 7.97 (m, 10H), 7.62 (d, 2H), 7.34 (m, 4H), 6.57 (d, 1H), 3.85 (m, 1H), 3.43 (m, 2H), 3.16 (m, 2H), 1.51 (s, 9H) ppm.

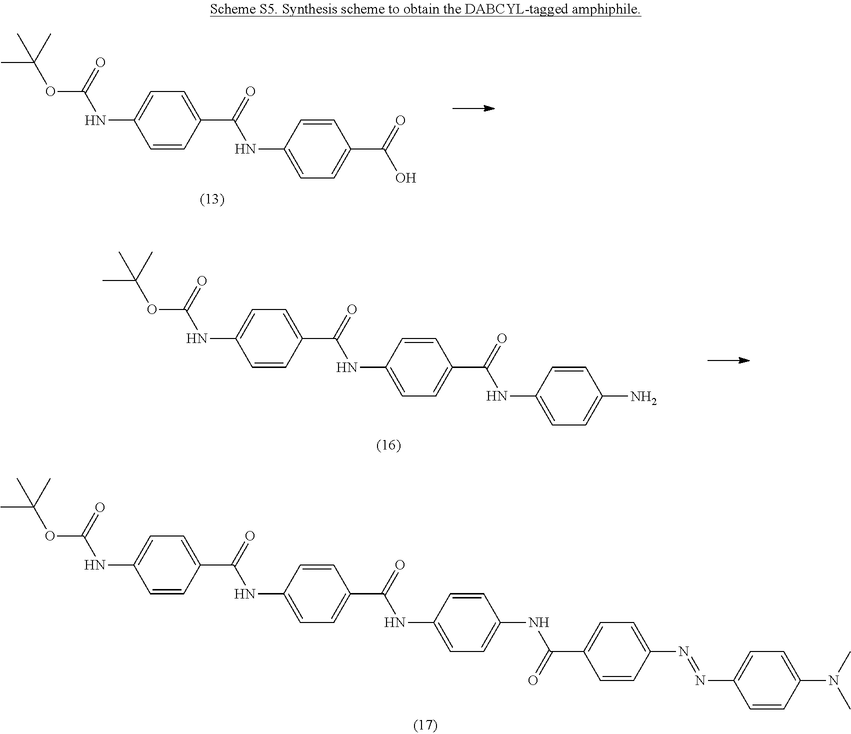 [SMILES: CC(C)(C)OC(=O)Nc1ccc(C(=O)Nc2ccc(C(=O)O)cc2)cc1 ; CC(C)(C)OC(=O)Nc1ccc(C(=O)Nc2ccc(C(=O)Nc3ccc(N)cc3)cc2)cc1 ; CN(C)c1ccc(/N=N/c2ccc(C(=O)Nc3ccc(NC(=O)c4ccc(NC(=O)c5ccc(NC(=O)OC(C)(C)C)cc5)cc4)cc3)cc2)cc1]

tert-Butyl 4-((4-((4-aminophenyl)carbamoyl)phenyl)carbamoyl)phenylcarbamate (16): A solution of compound 13 (0.56 mmol), 1,4-diaminobenzene (11.22 mmol), EDC (0.67 mmol), and DMAP (0.67 mmol) in dimethylformamide (80 mL) was stirred at 25° C. for 24 h.

The volatile fraction was removed under reduced pressure and the remaining residues were washed several times with methanol and filtered (yield: 59%). ¹H NMR (400 MHz, DMSO-d): δ=7.92 (m, 6H), 7.60 (d, 2H), 7.36 (d, 2H), 6.54 (d, 2H), 4.91 (s, 2H), 1.51 (s, 9H) ppm.

DABCYL-tagged amphiphile, (E)-4-((4-(dimethylamino)phenyl)diazenyl)-N-(4-(4-(4-(3,3-dimethylbutanamido)benzamido)benzamido)phenyl)benzamide (17): A solution of compound 16 (0.67 mmol), DABCYL (0.81 mmol), EDC (0.81 mmol), and DMAP (0.81 mmol) in dimethylformamide (10 mL) was stirred at 25° C. for 48 h. The solvent was then evaporated under reduced pressure. The crude mixtures were purified with water and chloroform (yield: 71%). ¹H NMR (400 MHz, DMSO-d): δ=7.94 (m, 12H), 7.62 (d, 4H), 7.45 (d, 2H), 6.66 (d, 2H), 3.10 (s, 6H), 1.51 (s, 9H) ppm.

Shear Alignment to Form Macroscopic Aramid Amphiphile Threads

A 2 wt % aqueous solution of 3 was bath sonicated for 24 h, rested for 12 h, annealed in a heating block at 80° C. for 10 h, and then slowly cooled to room temperature. This solution was then extruded into a bath of 40 mM sodium sulfate (Na₂SO₄) to produce macroscopic aramid amphiphile threads, as shown in FIGS. 89A-89B. The threads are then pulled out of the solution and dried before further analysis.

Chemical Characterization

Nuclear Magnetic Resonance

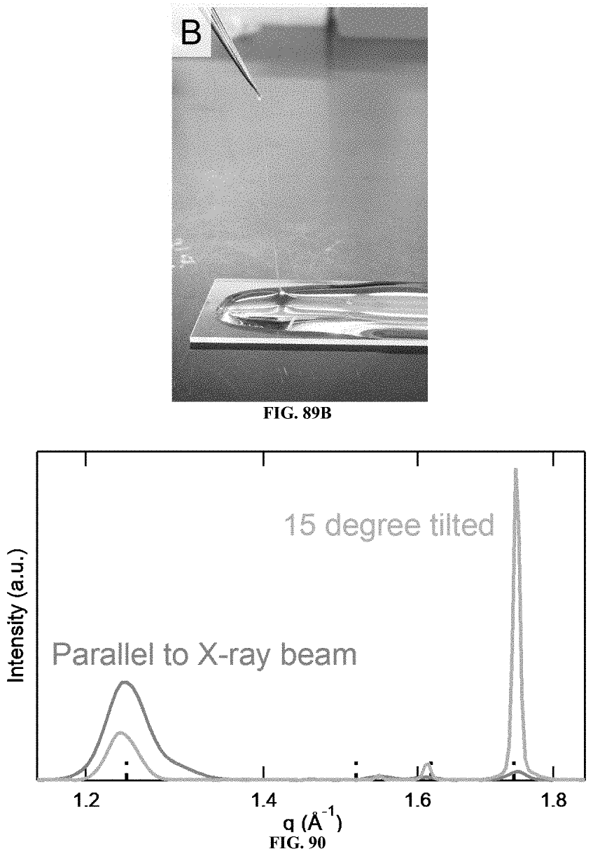

Proton (¹H) and carbon (¹³C) nuclear magnetic resonance (NMR) measurements were performed on a Bruker Avance III DPX 400. 20 mg of sample were dissolved in 500 μL deuterated dimethylsulfoxide (DMSO-d) for analysis. The chemical shifts were measured in parts per million (ppm) down-field from tetramethylsilane. Self-assembly behavior was also modulated and studied by addition of deuterated water to the DMSO-d solutions, as discussed with FIG. 90.

Solvent Effects:

The self-assembly behavior of aramid amphiphiles can be mediated by solvent variation as shown in FIG. 90. In good solvent, such as DMSO-d, the ¹H NMR spectrum of 1 indicates a monomeric state with well-resolved sharp peaks corresponding to aromatic H₁ and H₂ protons at δ=7.98 and 7.61 ppm. The aromatic proton peaks broaden as aggregation occurs when D₂O is titrated into the solution. The broadening of the aromatic H₁ and H₂ peaks is concomitant with the formation of strong intermolecular interactions upon self-assembly. At a D₂O content of 40%, the peaks of H₁ and H₂ shift to 7.82 and 7.47 ppm, respectively. The upfield shift of protons is observed when small intermolecular distances induce a magnetic shielding effect. The broadening of the aromatic proton peaks is consistent with slowing of conformational dynamics within the nanostructures as assembly occurs with increasing D₂O concentrations.

Mass Spectrometry

Molecular weights of amphiphiles were determined using a Bruker Omniflex matrix assisted laser desorption/ionization-time-of-flight (MALDI-ToF) instrument with a Reflectron accessory. A matrix solution was prepared by adding 15 mg of α-cyano-4-hydroxycinnamic acid to 1 mL of 1:1 water:acetonitrile by volume with 0.1% TFA, vortexing for one minute, centrifuging for 20 s, and retaining the supernatant. 10 μL of a 1 mg/mL amphiphile solution was then transferred into a centrifuge tube and diluted with the matrix solution to a 50 pmol/μL concentration. 1 μL of a 1 mg/mL calibrant solution (SpheriCal Peptide Low, Polymer Factory) in tetrahydrofuran was added to the solutions as an internal calibrant. 2 μL of each amphiphile solutions was pipetted and dried onto a sample plate for analysis.

Structural Characterization

Transmission X-Ray Scattering

Sample Preparation:

Small angle X-ray scattering (SAXS) samples were prepared by dissolving lyophilized powders of 1, 2, and 3 in DI water above the solubility limit. After centrifugation at 3,000 rpm, the supernatant was loaded into 2 mm diameter quartz capillary tubes (Hampton Research). Wide and medium angle X-ray scattering (WAXS, MAXS) was performed on macroscopic aramid amphiphile threads, prepared as described above.

Experimental Details:

SAXS measurements were performed at Beamline 12-ID-B of Advanced Photon Source at Argonne National Laboratory. The X-ray radiation energy was 13.3 keV. Two detectors, Pilatus 2M and Pilatus 300K (Dectris), were employed data collection. The detectors were set to cover q range of 0.005-2.7 Å⁻¹. The 2-D X-ray scattering patterns were background subtracted and processed using beamline software for reduction to 1-D data curves.

Fits were attempted using lamellar bilayer, cylinder, rectangular prism, and core-shell cylinder models. The lamellar bilayer model gave the best fit as shown in FIG. 85C. This model accommodates two scattering length densities (SLD), one corresponding to the hydrophobic domain including aliphatic tails and aramids, and the other corresponding to the hydrated hydrophilic head groups. The SLD of the tail and head group domains were calculated based on the molecular formulae and estimated densities and were input to the fitting parameters as 11.13×10⁻⁶ Å⁻² and 9.40×10⁻⁶ Å⁻², respectively. The solvent SLD (water) is 9.44×10⁻⁶ Å⁻². From this fitting, the hydrophobic core was found to be 2.8±0.1 nm and the hydrophilic head group thickness was found to be 1.1±0.4 nm, for a total bilayer thickness of 3.9±0.5 nm. The higher error in the hydrophilic region is likely due to its similar SLD as the solvent.

WAXS and MAXS measurements were performed on a SAXSLAB instrument using a Rigaku 002 microfocus X-ray source (CuK_{α} radiation, 1.5418 Å) and a DECTRIS PILATUS 300K detector. WAXS and MAXS profiles were measured at a sample-to-detector distance of 109 mm and 459 mm, respectively.

VESTA software was chosen for simulating the X-ray diffraction peaks shows in FIG. 88C. In this simulation, the space group 26: Pmc2₁ of poly(p-benzamide) is considered as the most relative packing structure in aramid nanofibers due to the constraint of the parallel amide bonding from the tri-aramid domain. Furthermore, reversing of the intensities for the two most significant peaks is observed when the thread is tilted on the X-ray beam axis. This observation indicates the two significant peaks correspond to orthogonal planes with a common intersection along the thread direction, and the assumption of the space group is confirmed. The black dotted lines of simulated peak position in FIG. 88C and FIG. 91 are generated from the unit cell with a=7.22 Å, b=5.05 Å and c=11.10 Å parameters.

Conventional Transmission Electron Microscopy

Conventional transmission electron microscopy (TEM) images were captured on a FEI Tecnai G2 Spirit TWIN microscope at an accelerating voltage of 120 kV. Grids were prepared by depositing 10 μL of a 1 mg/mL amphiphile solution onto a continuous carbon grid (Electron Microscopy Sciences, 200 mesh, copper) for 20 sec, blotting to remove the solution, depositing 10 μL of a 0.1% phosphotungstic acid solution onto the grid (Electron Microscopy Sciences), and blotting to remove the stain. See, FIGS. 92A-92C.

Cryogenic Transmission Electron Microscopy

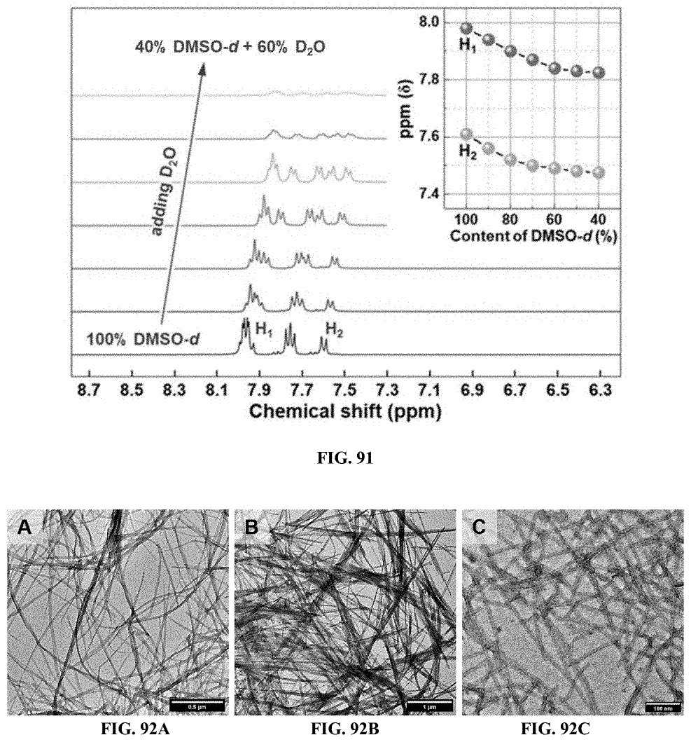

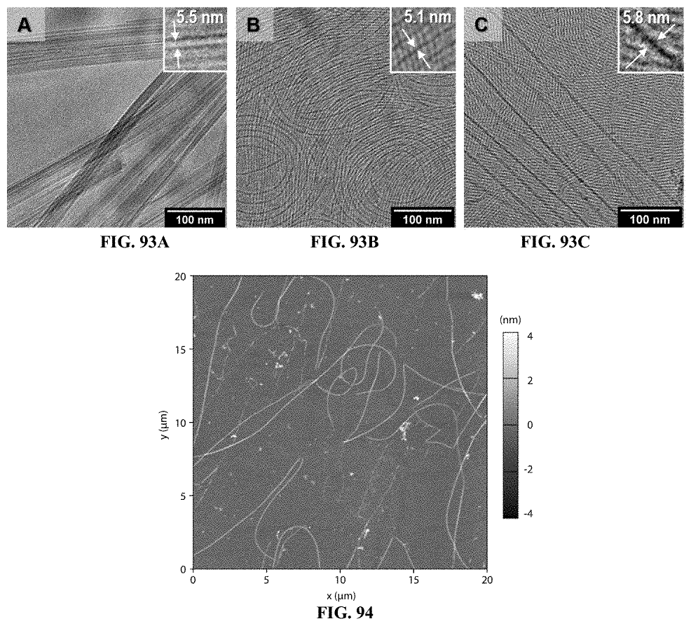

Cryogenic transmission electron microscopy (cryo-TEM) grids were prepared with an FEI Vitrobot Mark IV. Holey carbon grids (Ted Pella, 300 mesh, copper) were glow-discharged before a 3 μL drop of a 2 mg/mL amphiphile solution was pipetted onto the grids in a chamber with 100% humidity. The grids were blotted for 4 sec, and then plunged into C2H₆ (l) followed by N₂ (l). Images were captured in an FEI Tecnai Arctica microscope equipped with an autoloader at an accelerating voltage of 200 kV. The defocus in data collection ranged from −1.5 to −3.5 FIGS. 93A-93C show cryo-TEM micrographs of nanofibers of compounds 1, 2, and 3, respectively. FIG. 92A shows that bundling of 1 occurs, likely due to interfiber hydrogen bonding of the head groups. Interestingly, self-assembly of 2 leads to both high-aspect-ratio nanofibers and in some cases, complete nanofiber circles. Nanofiber widths in Cryo-TEM for 1, 2, and 3 nanofibers are measured as 5.5 nm, 5.1 nm, and 5.8 nm, respectively for n=25 measurements with approximately 5% error.

Atomic Force Microscopy

Compound 3 was chosen for analysis by atomic force microscopy (AFM) because of its favorable surface interaction with AFM substrates. A 2 wt % compound 3 solution was prepared for atomic force microscopy (AFM) following the sonication and heat treatment for making nanofiber thread solutions before alignment: bath sonication for 24 h, resting for 12 h, annealing in a heating block at 80° C. for 10 h, and then slow cooling to room temperature. The solution was then diluted to 0.01 wt % and a 100 μL droplet of this diluted solution was deposited onto a cleaned mica substrate and analyzed by AFM. The mica substrate was prepared through plane cleavage and cleaning with DI H₂O. After 3 h of incubating the amphiphile solution on the clean mica, the solution was removed and the used directly for AFM imaging (FIG. 94). Nanofibers were imaged in tapping mode in air using a Cypher (Asylum Research, Oxford Instruments) atomic force microscope. AC160TS-R3 cantilevers from Olympus were used (nominal spring constant 26 N/m and resonance frequency of 300 kHz in air). AFM images were recorded at 512 px×512 px at a scanning speed of 0.65 Hz.

Scanning Electron Microscopy

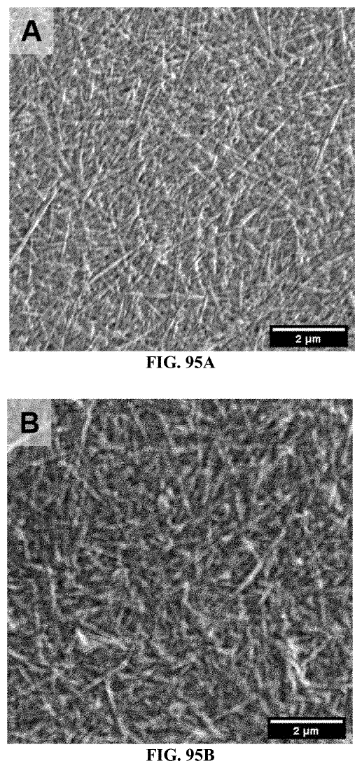

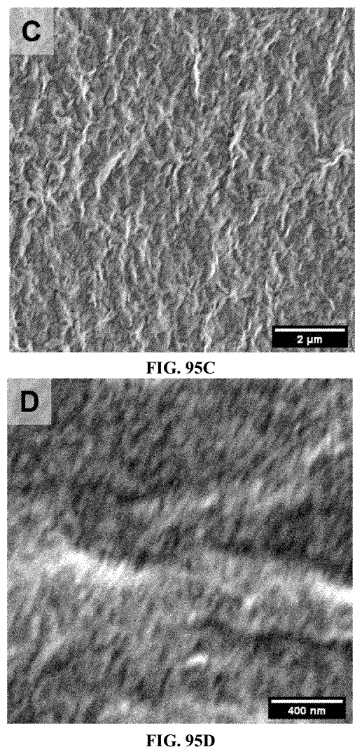

Scanning electron microscopy (SEM) images were recorded on a Zeiss MERLIN field emission microscope operating at a 1-3 kV accelerating voltage to resolve higher-order structure of the amphiphile assemblies in their post-assembled, dried state. A secondary electron detector set to 120-200 pA was used for imaging. 15 μL of 1 mg/mL amphiphile solutions were drop cast on copper tape affixed to SEM stubs and dried. In all cases, fiber morphologies are observed by SEM in the bulk after drying aramid amphiphile nanofibers from water. Compounds 1 and 2 show hierarchical bundling of fibers at the microscale. Compound 3 shows less fiber bundling at the microscale, but nanofibers can be discerned at higher magnifications. The SEM micrograph in FIG. 87C was coated with 10 nm Au by sputtering on a MS Q150T ES coater. The micrographs shown in FIGS. 95A-95D are uncoated.

Förster Resonance Energy Transfer

EDANS and DABCYL serve as a typical Förster resonance energy transfer (FRET) pair with a Förster radius of 3.3 nm. See below. When the donor and quencher approach the Förster radius, energy transfer from the donor to the quencher results in a reduction of fluorescence intensity through vibrational relaxation pathways. Therefore, decreases in fluorescence intensity correlate to molecular exchange between adjacent nanofibers (FIGS. 86A-86C).

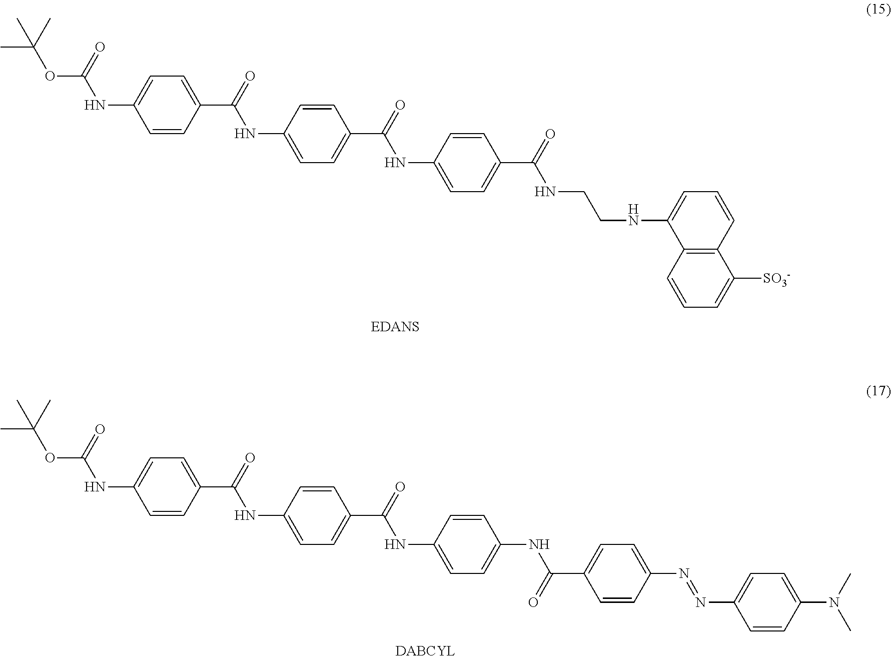

Sample preparation: EDANS and DABCYL were each covalently tethered to the head group region of an aramid amphiphile. Aramid amphiphiles were prepared at concentrations of 0.1 to 0.5 mM in water and co-assembled with 5 mol % EDANS-tagged or DABCYL-tagged analogues.

Experimental Details:

Fluorescence intensities were measured on a Varian Cary Eclipse spectrophotometer operating at an excitation wavelength of 334 nm with excitation and emission slits set at 5 nm. A fluorimeter scan rate of 600 nm/min was used, and the PMT detector voltage was 600 V.

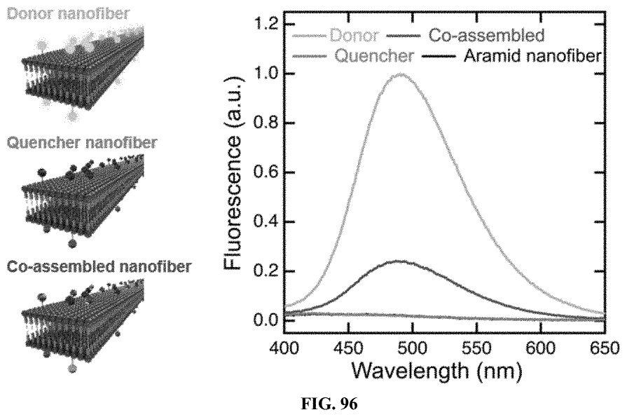

As a control, completely mixed co-assemblies of amphiphiles labeled with both a donor fluorophore and dark quencher show a 76% reduction in fluorescence intensity relative to assemblies labeled solely with the fluorophore (FIG. 96).

Stiffness Determination by Topographical Analysis of Nanofiber Contours

Sample Preparation:

Compound 3 was chosen for analysis by atomic force microscopy (AFM) because of its high solubility and favorable surface interaction with AFM substrates. DI water was added to a lyophilized sample of 3 to reach 30 mg/mL. A sonicator bath was used to accelerate solvation. After 24 h at room temperature, the solution was diluted to 0.03 mg/mL and deposited on a clean glass surface. The glass substrate was prepared through cleanings with DI H₂O and ethanol, drying with stream of N₂ (g), and activation by UV/ozone treatment. After 5 min of incubating the amphiphile solution on the clean glass, the surface was rinsed with DI water and used directly for AFM imaging.

Experimental Details:

Compound 3 nanofibers were imaged in tapping mode in water using a Bruker/JPK Nanowizard 4 atomic force microscope. BL-AC40-TS cantilevers from Olympus were used (nominal spring constant 0.1 N/m and resonance frequency of −25 kHz in water). AFM images were recorded at 512 px×512 px at a scanning speed of 10 Hz. AFM images were used to determine the persistence length and Young's modulus of the fibers. Fluctuations of fiber shape are statistically processed using the Easyworm software tool, which traces parametric splines to the contours of many fibers of the same sample (in this experiment, n=29 fibers). Parametric splines store the x-y coordinates of all the knots along the fiber. Each combination of two knots gives a secant length L, and the midpoint of this secant deviates from the fiber contour by a distance δ. The persistence length P is then obtained by least-square fitting the data to the worm-like chain model for semi-flexible polymers, <δ²>=L³/(48×P), for fibers equilibrating in 2-D. The persistence length reflects how much a fiber bends as a result of thermal fluctuations. A higher persistence length of a fiber corresponds to a lesser change in orientation over a given distance along its contour. The flexural rigidity F is the result of scaling the persistence length to the thermal energy according to F=P×k_{B}T. Finally, the Young's (elastic) modulus E is obtained using E=F/I, where 1 is the area moment of inertia, which reflects the resistance to bending of a cross-section. For the circular cross-section observed in AFM measurements, the moment of inertia, I=π·d⁴/64, where d is the fiber diameter. Heights of each nanofiber were estimated by analysis of nanofiber cross-sections observed in the AFM images. The AFM height measurements are consistent with cryo-TEM and SAXS measurements, and therefore d=3.7±0.5 nm was used to calculate 1.

Yield Strength Determination by Sonication-Induced Scission

Method:

A Qsonica Q500 sonicator with a 2-mm-diameter microtip was used to sonicate 10 mL of a 0.5 mg/mL aqueous solution of compound 3 nanofibers. A vibrational frequency of 20 kHz and amplitude of 25% were used during the experiment, which lasted for 2 h of “sonication on” time with a 5 sec on/3 sec off pulse. Sonicating power was held at 30 W·cm⁻² to ensure cavitation. The solution was held in an ice bath for the duration of the experiment to prevent solvent evaporation and tip breakage during sonication. Images of fragments after sonication were captured by conventional TEM (with stained grids prepared as described herein) and AFM.

Experimental Details:

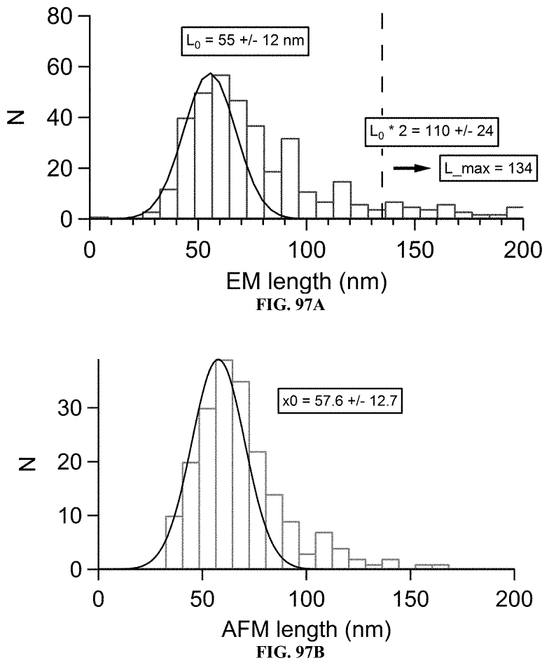

The yield (tensile) strength σ* by using a sonication-induced fibril scission technique. In short, sonication creates collapsing cavitation bubbles, causing fluid velocity fields to trap fibrils and exert shear forces on them. This leads to fibril extension in opposite directions and mechanically-induced rupture at the site of highest stress. The model developed by Huang et al. implies that the forces exerted on the fibril decrease dramatically with the fibril length. Hence there is a threshold length Lₗᵢₘ below which a fibril of a given cross-section will not break anymore. The length of hundreds of fibril fragments as a function of their cross-sectional size were plotted (see FIGS. 97A-97B), and σ was derived from the relationship:

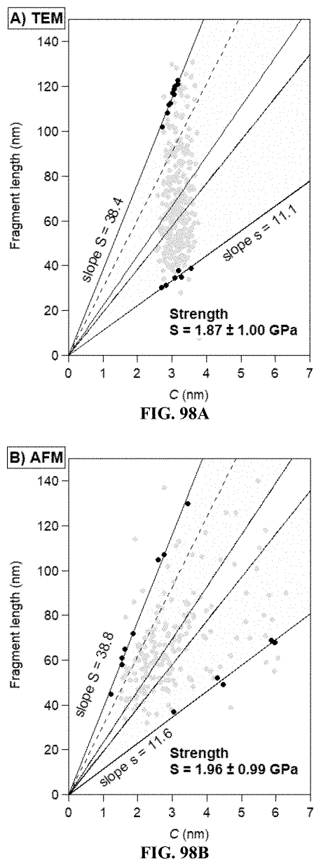

Lₗᵢₘ=αC\sqrt{σ} (1)

where α=7.10⁻⁴ is a prefactor that depends on the experimental conditions, and C reflects the cross-sectional size of the fibril fragments. For a rectangular cross-section fibril with long edge w (i.e. TEM width), and short edge h (i.e. AFM height), it is given by:

$$C = {[ \frac{γ}{2 {w}^{2}} [ ( ln ( γ + \sqrt{{γ}^{2} + 1} ) + ln ( {γ}^{- 1} + \sqrt{{γ}^{- 2} + 1} ) ] ]}^{- 1 / 2} \qquad ( 2 )$$

where γ=w/h is the aspect ratio. After prolonged sonication time, fibril length distribution reaches a plateau and the size of fragments that belong to a sample fall in a “terminal range” defined by [Lₗᵢₘ/2, Lₗᵢₘ]. However, an even broader distribution of fragment lengths L was expected because both the cross-sectional area and intrinsic strength can vary. This broadening of the terminal range is considered by determining the lines of best fit from the extremities of the distribution. The extremities were represented by the 5-10 data points corresponding to the smallest and longest aspect ratios L/C (see the black dots in FIGS. 98A-98B), discarding obvious outliers. The lowest slope s reflects the low boundary of the shortest terminal range (i.e. the smallest aspect ratio), and the highest slope S exposes the high boundary of the longest terminal range (i.e. the longest aspect ratio). The shortest and longest terminal ranges are thus defined by intervals [s, 2s] and [S/2, S], respectively. Lₗᵢₘ is the averaged top of any terminal range given by:

$$\frac{{L}_{lim}}{C} = \frac{2 s + S}{2} \pm \frac{2 s - S}{2} \qquad ( 3 )$$

Combining the results of Eq. 2 with Eq. 1 the tensile strength σ was obtained. This method is particularly solid to reveal at least the position of the lower edge of the terminal distribution, which corresponds to the lowest possible strength of the fibril sample. Using the absolute error±(2s−5)/2 provides a simple way to account both for any experimental source of error and for the strength variability within a given sample. In this study all fibril fragments have a similar cross-sectional area, with w=6.0±1.3 nm and h=3.1±0.5 nm. Consequently, most fragment lengths are distributed in one single terminal range

$$[ \frac{{L}_{lim}}{2} , {L}_{lim} ] ,$$

as displayed in the histogram of the fragment length distribution (see FIGS. 97A-97B). In order not to overestimate the strength, a cut-off value to discard all fibril fragments with L>134 nm was used. These fragments most likely escaped sonication-induced breakage because the initial fibril concentration was relatively high (˜0.5 mg/mL); as a result, their lengths did not end up into the terminal range. To calculate the cut-off, the most prominent peak of the distribution was assumed to correspond to Lₗᵢₘ/2 and fit a Gaussian to that peak, which gives Lₗᵢₘ/2=55±12 nm for TEM. Lₗᵢₘ=110±24 was then estimated for TEM and was used to derive the cut-off.

Here the fragment lengths were independently estimated in both TEM and AFM measurements. Both techniques give similar results, also translating in similar strength values. For the determination of the cross-sectional parameter C, the AFM-determined mean height of the fibril fragments h=1.96±0.55 nm was used in the analysis of the TEM data (FIG. 98A), and the TEM-determined mean width of the fibril fragments w=6.0±1.3 nm was used in the analysis of the AFM data (FIG. 98B). Note that the mean AFM height of the fibril fragments is lower than that of non-sonicated fibrils, possibly because non-sonicated fibrils are higher-order assemblies and sonication leads to partial disassembly of the several protofilaments that form a “mature” fibril. The highest and lowest lines in this plot identify Lₗᵢₘ and the smallest fragments produced by sonication, respectively, and the yellow region considers a broadening of these bounds due to variations in cross-sectional area and intrinsic strength.

References, each of which is incorporated by reference in its entirety.
1. Morton O. A new day dawning?: Silicon Valley sunrise. Nature 443, 19 (2006).
2. Hoffert M I, et al. Energy implications of future stabilization of atmospheric CO2 content. Nature 395, 881 (1998).
3. Lewis N S, Nocera D G. Powering the planet: Chemical challenges in solar energy utilization. Proc. Natl. Acad. Sci. U.S.A. 103, 15729-15735 (2006).
4. Gray H B. Powering the planet with solar fuel. Nat. Chem. 1, 7 (2009).
5. Cook T R, Dogutan D K, Reece S Y, Surendranath Y, Teets T S, Nocera D G. Solar Energy Supply and Storage for the Legacy and Nonlegacy Worlds. Chem. Rev. 110, 6474-6502 (2010).
6. Kudo A, Miseki Y. Heterogeneous photocatalyst materials for water splitting. Chem. Soc. Rev. 38, 253-278 (2009).
7. Walter M G, et al. Solar Water Splitting Cells. Chem. Rev. 110, 6446-6473 (2010).
8. Albero J, Garcia H. Photocatalytic CO2 Reduction. In: Heterogeneous Photocatalysis: From Fundamentals to Green Applications (ed\hat{ }(eds Colmenares J C, Xu Y-J). Springer Berlin Heidelberg (2016).
9. Grätzel M. Photoelectrochemical cells. Nature 414, 338 (2001).
10. Esswein A J, Nocera DG. Hydrogen Production by Molecular Photocatalysis. Chem. Rev. 107, 4022-4047 (2007).
11. Morris A J, Meyer G J, Fujita E. Molecular Approaches to the Photocatalytic Reduction of Carbon Dioxide for Solar Fuels. Acc. Chem. Res. 42, 1983-1994 (2009).
12. Cieśla P, Kocot P, Mytych P, Stasicka Z. Homogeneous photocatalysis by transition metal complexes in the environment. J. Mol. Catal. A: Chem. 224, 17-33 (2004).
13. Lazarides T, McCormick T, Du P, Luo G, Lindley B, Eisenberg R. Making Hydrogen from Water Using a Homogeneous System Without Noble Metals. J. Am. Chem. Soc. 131, 9192-9194 (2009).
14. Yang J, Yoon M-C, Yoo H, Kim P, Kim D. Excitation energy transfer in multiporphyrin arrays with cyclic architectures: towards artificial light-harvesting antenna complexes. Chem. Soc. Rev. 41, 4808-4826 (2012).
15. Hartgerink J D, Beniash E, Stupp S I. Self-Assembly and Mineralization of Peptide-Amphiphile Nanofibers. Science 294, 1684 (2001).
16. Whitesides G M, Grzybowski B. Self-Assembly at All Scales. Science 295, 2418 (2002).
17. Zhang S. Fabrication of novel biomaterials through molecular self-assembly. Nat. Biotechnol. 21, 1171 (2003).
18. Faul C F J, Antonietti M. Ionic Self-Assembly: Facile Synthesis of Supramolecular Materials. Adv. Mater. 15, 673-683 (2003).
19. Chakrabarty R, Mukherjee P S, Stang P J. Supramolecular Coordination: Self-Assembly of Finite Two- and Three-Dimensional Ensembles. Chem. Rev. 111, 6810-6918 (2011).
20. Zayed J M, Nouvel N, Rauwald U, Scherman O A. Chemical complexity-supramolecular self-assembly of synthetic and biological building blocks in water. Chem. Soc. Rev. 39, 2806-2816 (2010).
21. Wasielewski M R. Self-Assembly Strategies for Integrating Light Harvesting and Charge Separation in Artificial Photosynthetic Systems. Acc. Chem. Res. 42, 1910-1921 (2009).
22. Weingarten A S, et al. Self-assembling hydrogel scaffolds for photocatalytic hydrogen production. Nat. Chem. 6, 964 (2014).
23. Weingarten A S, Kazantsev R V, Palmer L C, Fairfield D J, Koltonow A R, Stupp S I. Supramolecular Packing Controls H₂ Photocatalysis in Chromophore Amphiphile Hydrogels. J. Am. Chem. Soc. 137, 15241-15246 (2015).
24. Tian J, et al. Supramolecular metal-organic frameworks that display high homogeneous and heterogeneous photocatalytic activity for H2 production. Nat. Commun. 7, 11580 (2016).
25. Wang H-Y, Wang W-G, Si G, Wang F, Tung C-H, Wu L-Z. Photocatalytic Hydrogen Evolution from Rhenium(I) Complexes to [FeFe] Hydrogenase Mimics in Aqueous SDS Micellar Systems: A Biomimetic Pathway. Langmuir 26, 9766-9771 (2010).
26. Hansen M, Li F, Sun L, Konig B. Photocatalytic water oxidation at soft interfaces. Chem. Sci. 5, 2683-2687 (2014).
27. Limburg B, et al. Kinetics of Photocatalytic Water Oxidation at Liposomes: Membrane Anchoring Stabilizes the Photosensitizer. ACS Catal. 6, 5968-5977 (2016).
28. Uskoković V. Dynamic Light Scattering Based Microelectrophoresis: Main Prospects and Limitations. J. Dispers. Sci. Technol. 33, 1762-1786 (2012).
29. Panagiotopoulos A, Ladomenou K, Sun D, Artero V, Coutsolelos AG. Photochemical hydrogen production and cobaloximes: the influence of the cobalt axial N-ligand on the system stability. Dalton Trans. 45, 6732-6738 (2016).
30. Mikkelsen M, Jorgensen M, Krebs F C. The teraton challenge. A review of fixation and transformation of carbon dioxide. Energy Environ. Sci. 3, 43-81 (2010).
31. Adaev I S, Korostoshevskaya T V, Novikov V T, Lysyak T V. Electrochemical Behavior of Cobalt-Dimethylglyoxime Complex Co(DMG)2(H2O)2 in the Carbon Dioxide Reduction Process. Russ. J. Electrochem. 41, 1125-1129 (2005).
32. Bonin J, Chaussemier M, Robert M, Routier M. Homogeneous Photocatalytic Reduction of CO2 to CO Using Iron(0) Porphyrin Catalysts: Mechanism and Intrinsic Limitations. ChemCatChem 6, 3200-3207 (2014).
33. Liu X, Inagaki S, Gong J. Heterogeneous Molecular Systems for Photocatalytic CO2 Reduction with Water Oxidation. Angew. Chem. Int. Ed. 55, 14924-14950 (2016).
34. Wang Y, et al. Facile One-Step Synthesis of Hybrid Graphitic Carbon Nitride and Carbon Composites as High-Performance Catalysts for CO₂ Photocatalytic Conversion. ACS Appl. Mater. Interfaces 8, 17212-17219 (2016).
35. Yu L, et al. Enhanced Activity and Stability of Carbon-Decorated Cuprous Oxide Mesoporous Nanorods for CO2 Reduction in Artificial Photosynthesis. ACS Catal. 6, 6444-6454 (2016).
36. Wang H, Chen Y, Hou X, Ma C, Tan T. Nitrogen-doped graphenes as efficient electrocatalysts for the selective reduction of carbon dioxide to formate in aqueous solution. Green Chem. 18, 3250-3256 (2016).
37. Rao H, Schmidt L C, Bonin J, Robert M. Visible-light-driven methane formation from CO2 with a molecular iron catalyst. Nature 548, 74 (2017).
38. Zhang X, et al. Highly selective and active CO2 reduction electrocatalysts based on cobalt phthalocyanine/carbon nanotube hybrid structures. Nat. Commun. 8, 14675 (2017).
39. Shen J, et al. Electrocatalytic reduction of carbon dioxide to carbon monoxide and methane at an immobilized cobalt protoporphyrin. Nat. Commun. 6, 8177 (2015).
40. Lu Q, et al. A selective and efficient electrocatalyst for carbon dioxide reduction. Nat. Commun. 5, 3242 (2014).
41. Blankenship R E, et al. Comparing Photosynthetic and Photovoltaic Efficiencies and Recognizing the Potential for Improvement. Science 332, 805 (2011).
42. Bryden, F.; Boyle, R. W., A Mild, Facile. Synlett. 2013, 24, 1978.
43. Panagiotopoulos, A.; Ladomenou, K.; Sun, D.; Artero, V.; Coutsolelos, A. G. Dalton Trans. 2016, 45, 6732.
44. Manton, J. C.; Long, C.; Vos, J. G.; Pryce, M. T. Dalton Trans. 2014, 43, 3576.
45. Montalti, M., Credi, A., Prodi, L & Gandolfi, M. T. Handbook of Photochemistry 3^{rd} edition (CRC Press, 2006).

Other embodiments are within the scope of the following claims.

What is claimed is:

1. A compound of formula I: 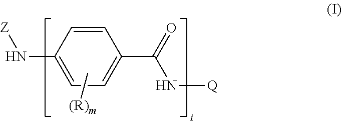
wherein
Z is substituted arylacyl including a first substituent;
Q is substituted aryl including a second substituent;
each R is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio;
m is 0, 1, 2, 3 or 4;
i is 1, 2, 3, 4, 5, 6, 7, 8, 9 or 10, wherein at least one of the first substituent and the second substituent is a hydrophobic group.

2. The compound of claim 1, wherein one of the first substituent and the second substituent is a hydrophobic group and the other of the first substituent and the second substituent is a hydrophilic group.

3. The compound of claim 1, wherein the substituted arylacyl is a substituted phenyl acyl.

4. The compound of claim 1, wherein the substituted aryl is a substituted phenyl.

5. The compound of claim 1, wherein the compound is anion, cationic or zwitterionic.

6. The compound of claim 1, wherein the compound includes a metal binding moiety.

7. The compound of claim 1, wherein the compound has the formula II: 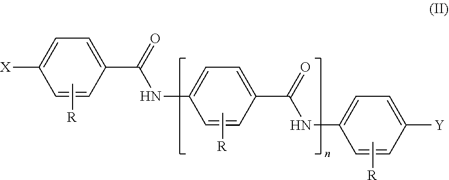
wherein
n is 0, 1, 2, 3, 4, 5, 6, 7 or 8;
X is a tail group; and
Y is a head group.

8. The compound of claim 7, wherein X is a substituted or unsubstituted, branched or linear alkyl group, alkenyl group, alkynyl group, fluorinated group, siloxane group, or aromatic group.

9. The compound of claim 7, wherein X is selected from the group consisting of:

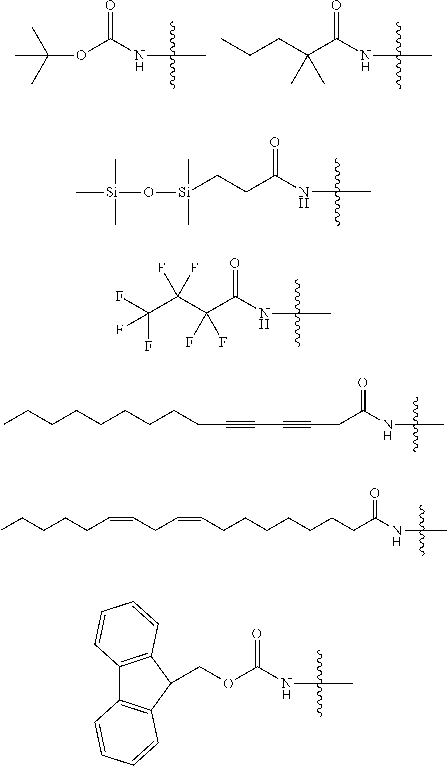

10. The compound of claim 7, wherein Y is an anionic group, a cationic group, a zwitterionic group, or an uncharged hydrophilic group.

11. The compound of claim 7, wherein Y is selected from the group consisting of oligo-ethylene glycol, a heavy metal chelator, an amino acid, a peptide,

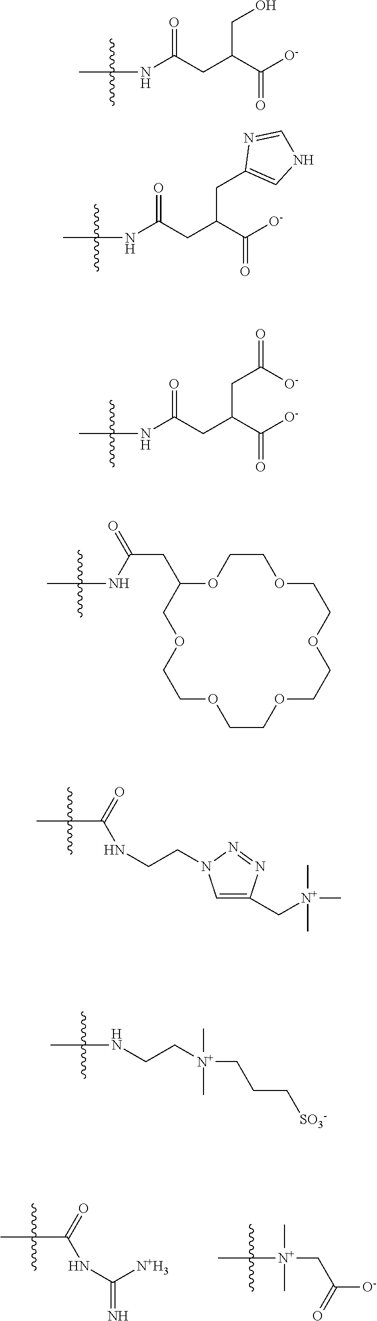

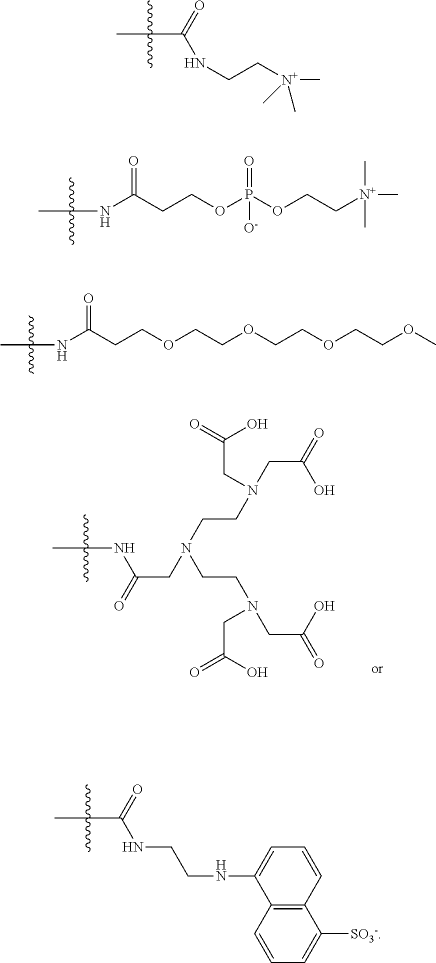

13. The compound of claim 1, wherein the compound is

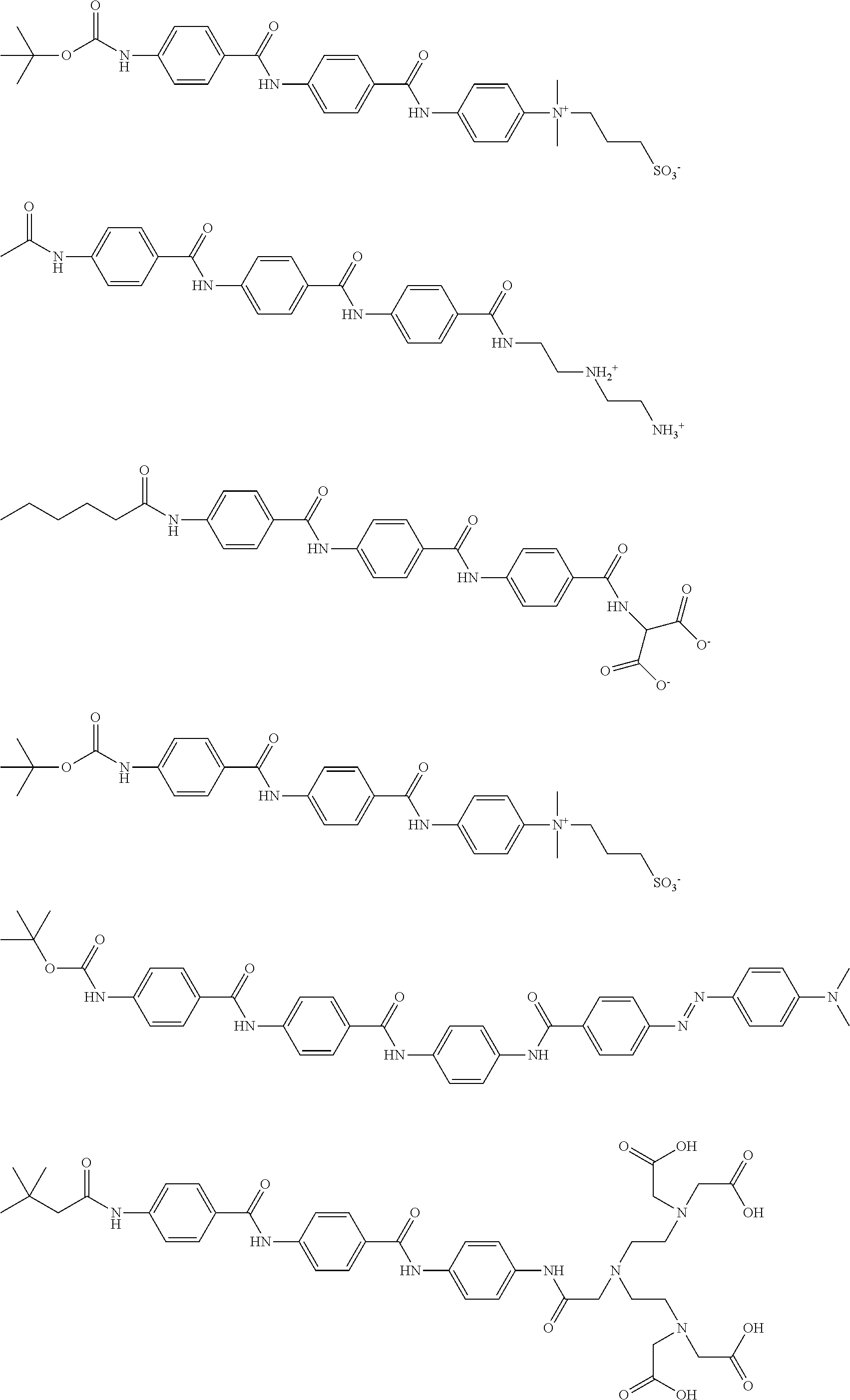

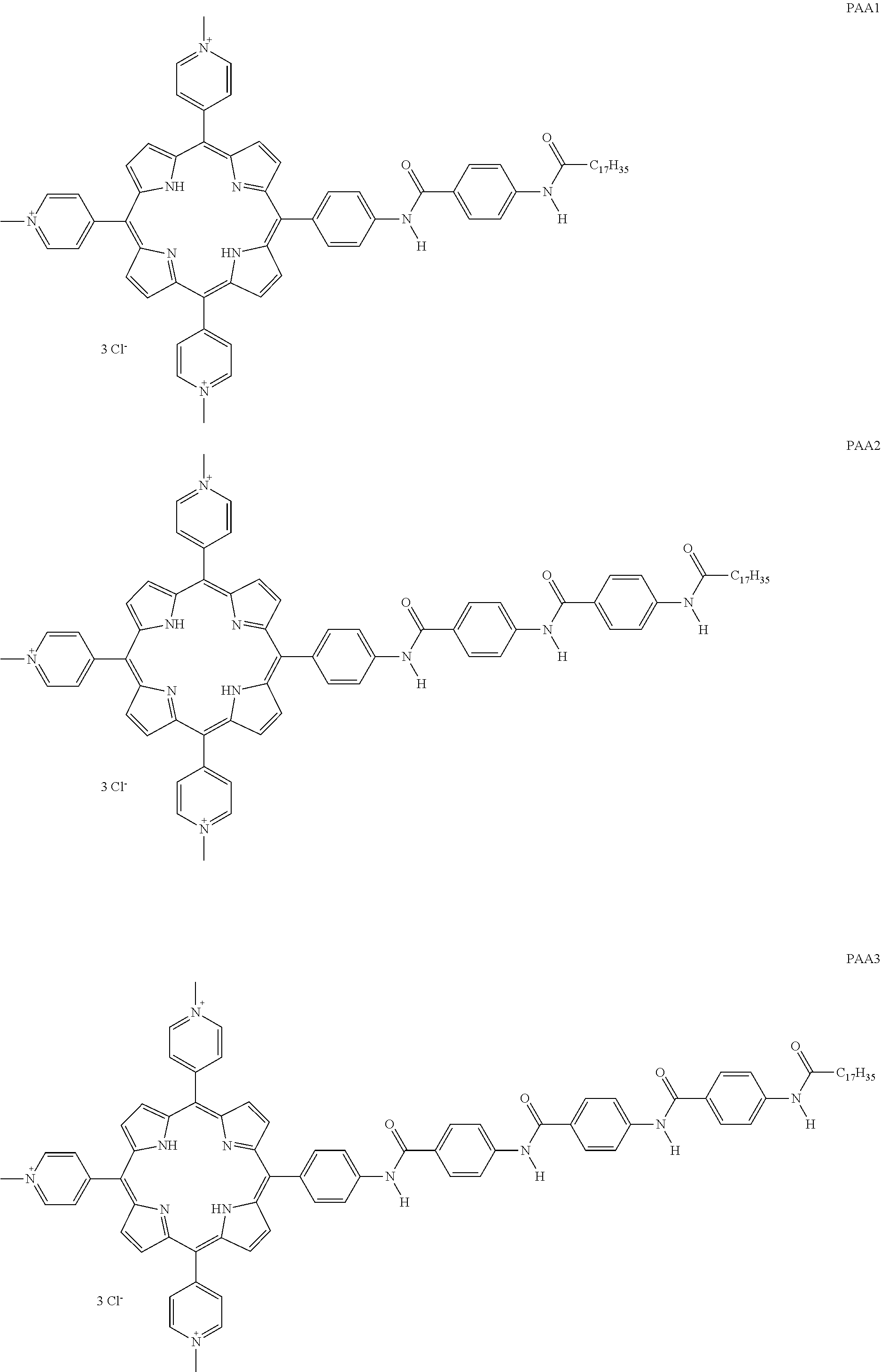

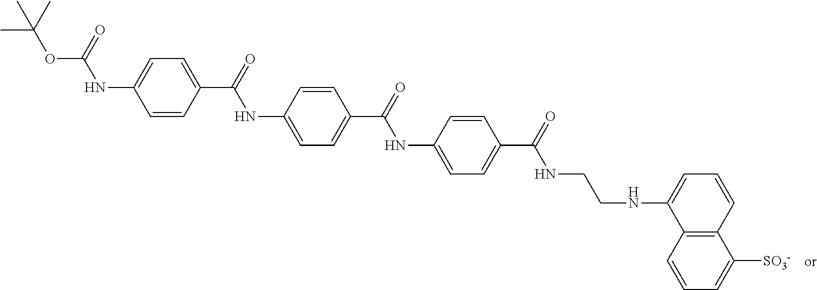

13. An assembly comprising a plurality of a compound of claim 1.

14. The assembly of claim 13, wherein the assembly is a vesicle, a ribbon, a nanofiber, or a micelle.

15. The assembly of claim 13, wherein the assembly is a fiber.

16. A metal complex comprising a metal ion and a compound of claim 1.

17. A method of forming an assembly comprising:
dispersing a plurality of a compound of claim 1; and
isolating an assembly of the plurality of the compound.

18. The method of claim 17, further comprising encapsulating a payload in the assembly.

19. The method of claim 17, further comprising shear aligning the assembly to form a fiber.
